(19) United States
(12) Patent Application Publication
(10) Pub. No.: US 2026/0158070 A1
(43) Pub. Date: Jun. 11, 2026

(54) COMPOSITIONS AND METHODS FOR THE TREATMENT AND DIAGNOSIS OF CANCERS ASSOCIATED WITH SURFACE K-RAS

(71) Applicants: UNIVERSITY OF MARYLAND, BALTIMORE, Baltimore, MD (US); UNIVERSITY OF MARYLAND, COLLEGE PARK, College Park, MD (US)

(72) Inventors: Alexander Sasha KRUPNICK, Owings Mills, MD (US); Anirban BANERJEE, Lutherville-Timonium, MD (US); Amy J. KARLSSON, Silver Spring, MD (US); Eric Reed LAZEAR, Carrboro, NC (US); Shaun Maxwell STEWART, Chadds Ford, PA (US)

(73) Assignees: UNIVERSITY OF MARYLAND, BALTIMORE, Baltimore, MD (US); UNIVERSITY OF MARYLAND, COLLEGE PARK, College Park, MD (US)

(21) Appl. No.: 19/535,255

(22) Filed: Feb. 10, 2026

Related U.S. Application Data
(63) Continuation of application No. PCT/US2024/042468, filed on Aug. 15, 2024.
(60) Provisional application No. 63/533,024, filed on Aug. 16, 2023.
(60) Provisional application No. 63/613,718, filed on Dec. 21, 2023.

(51) Int. Cl. A61K 35/17; A61K 39/00; A61K 40/11; A61K 40/31; A61K 40/42; A61K 47/68; A61P 35/00; C07K 16/28; C07K 16/40

(52) U.S. Cl. CPC A61K 35/17; A61K 40/11; A61K 40/31; A61K 40/4253; A61K 47/68037; A61K 47/6871; A61K 47/6889; A61P 35/00; C07K 16/2809; C07K 16/40; A61K 2039/505; A61K 2239/13; A61K 2239/21; A61K 2239/22; A61K 2239/29; C07K 2317/31; C07K 2317/565

(57) ABSTRACT

The present disclosure describes compositions and methods for treating and diagnosing cancers that express K-Ras on the external surface of cancer cells, and novel antibody-drug conjugates for use in such methods.

### CROSS REFERENCE TO RELATED APPLICATIONS

This application claims benefit of priority of U.S. Provisional Application No. 63/533,024, filed Aug. 16, 2023, and U.S. Provisional Application No. 63/613,718, filed Dec. 21, 2023.

### SEQUENCE LISTING

The instant application contains a Sequence Listing, which has been submitted electronically in XML file format and is hereby incorporated by reference in its entirety. The XML copy, created on Aug. 14, 2024, is named “93700-41 1218-SL.xml” and is 291,716 bytes in size.

### FIELD OF THE INVENTION

The present invention relates to compositions and compositions for use in methods for the treatment and diagnosis of cancers associated with expression of Surface K-Ras Antigen, including mutant Surface K-Ras Antigen. The present invention also relates to antibodies, bispecific antibodies, immune cells with chimeric antigen receptors, and antibody-drug conjugates used to treat and diagnose cancers associated with expression of Surface K-Ras Antigen, including mutant Surface K-Ras Antigen.

### BACKGROUND OF THE INVENTION

While a substantial experimental and clinical effort has been directed toward the targeting of Kirsten Rat Sarcoma Viral Oncogene Homolog (K-Ras or KRAS) using mutation-specific covalent small molecule inhibitors (Punekar et al., Nat. Rev. Clin. Oncol. 2022 October; 19(10):637-655), limited experimental data exists describing protein-based technology targeting this oncogenic pathway. This is largely due to the difficulties of delivering protein therapeutics across the cell membrane, where mutant K-Ras is believed to activate downstream signaling pathways in an unregulated manner (PMID: 21924373). Considering the accepted canonical belief that K-Ras is localized and functions exclusively in the intracellular domain, either on the inner leaflet of the cell membrane or within intracellular organelles (See, for example, Hancock, Nat Rev Mol Cell Biol. 2003 May; 4(5):373-84. doi: 10.1038/nrm1105. PMID: 12728271.), therapeutic and diagnostic approaches based on external cell surface presentation of mutant K-Ras protein (apart from those targeting small K-Ras peptides presented in HLA molecules), such as antibody-drug conjugates (ADCs) and chimeric antigen receptor T cells (CAR-T cells) have not been developed in cancers associated with K-Ras expression.

### SUMMARY

Disclosed herein are therapeutic and diagnostic approaches based on external cell surface presentation of Surface K-Ras Antigen, including mutant Surface K-Ras Antigen, and compositions for use of the same. For example, disclosed herein are compositions comprising a binding agent-therapeutic agent complex comprising a binding agent linked to a therapeutic agent, wherein the binding agent binds with specificity to a Surface K-Ras Antigen expressed on an external surface of a cancer cell, and wherein neither the composition nor the binding agent-therapeutic agent complex comprises an intracellular delivery compound. In one aspect, the binding agent is an antibody or antibody fragment. In one aspect, the binding agent is an antibody or antibody fragment that selectively binds the Surface K-Ras Antigen, wherein the Surface K-Ras Antigen comprises a mutation at residue 12, 13, or 61 based on the amino acid sequence set forth in SEQ ID NO: 1 or SEQ ID NO: 2. In one aspect, the binding agent is an antibody or antibody fragment that selectively binds the Surface K-Ras Antigen, wherein the Surface K-Ras Antigen comprises any one of the following mutations based on the amino acid sequence set forth in SEQ ID NO: 1 or SEQ ID NO: 2: G12A; G12D; G12C; G12V; G12R; G13D; Q61H; and Q61 L. In one aspect, the binding agent is an antibody or antibody fragment that selectively binds the Surface K-Ras Antigen, wherein the Surface K-Ras Antigen comprises the G12D mutation based on the amino acid sequence set forth in SEQ ID NO: 1 or SEQ ID NO: 2. In one aspect, the binding agent is an antibody or antibody fragment that selectively binds the Surface K-Ras Antigen, wherein the Surface K-Ras Antigen comprises the G12D mutation as set forth in SEQ ID NO: 295. In one aspect, the binding agent is an antibody or antibody fragment that selectively binds the Surface K-Ras Antigen, wherein the Surface K-Ras Antigen comprises the G12C mutation based on the amino acid sequence set forth in SEQ ID NO: 1 or SEQ ID NO: 2. In one aspect, the binding agent is an antibody or antibody fragment that selectively binds the Surface K-Ras Antigen, wherein the Surface K-Ras Antigen comprises the G12V mutation based on the amino acid sequence set forth in SEQ ID NO: 1 or SEQ ID NO: 2. In one aspect, the binding agent is an antibody fragment.

In one aspect, the binding agent is a peptide or protein. In one aspect, the binding agent is a peptide or protein that selectively binds the Surface K-Ras Antigen, wherein the Surface K-Ras Antigen comprises the G12D mutation based on the amino acid sequence set forth in SEQ ID NO: 1 or SEQ ID NO: 2. In one aspect, the binding agent is a peptide or protein that selectively binds the Surface K-Ras Antigen, wherein the Surface K-Ras Antigen comprises the G12D mutation as set forth in SEQ ID NO: 295. In one aspect, the binding agent is a peptide or protein that selectively binds the Surface K-Ras Antigen, wherein the Surface K-Ras Antigen comprises a mutation at residue 12, 13, or 61 based on the amino acid sequence set forth in SEQ ID NO: 1 or SEQ ID NO: 2. In one aspect, the binding agent is a peptide or protein that selectively binds the Surface K-Ras Antigen, wherein the Surface K-Ras Antigen comprises the G12C mutation based on the amino acid sequence set forth in SEQ ID NO: 1 or SEQ ID NO: 2. In one aspect, the binding agent is a peptide or protein that selectively binds the Surface K-Ras Antigen, wherein the Surface K-Ras Antigen comprises the G12V mutation based on the amino acid sequence set forth in SEQ ID NO: 1 or SEQ ID NO: 2. In one aspect, the binding agent is an antibody described in Table 1 or Table 2, or is an antibody selected by the methods set forth in Examples 4 or 5.

In one aspect, the therapeutic agent is selected from the group consisting of a cytotoxic agent, a cytostatic agent, a toxin, or a radionuclide. In one aspect, the therapeutic agent is selected from the group consisting of DNA-damaging agents (alkylating agents), antimetabolites, topoisomerase inhibitors, mitotic inhibitors, antitumor antibiotics, and microtubule-disrupting agents. In one aspect, the therapeutic agent is selected from the group consisting of calicheamicin, saporin, maytansinoid, auristatin, lidamycin, methotrexate, vinblastine, vincristine, pyrrolobenzodiazepines and other benzodiazepine derivatives, duocarmycins, tubulysins, α-amanitin or bouganin protein toxins, doxorubicin, etoposide, fluorouracil, Gemcitabine, paclitaxel, cisplatin, cyclophosphamide, amatoxin, carboplatin, spliceostatin C, docetaxel, thailanstatin A or any combination thereof.

In one aspect, the binding agent is linked to the therapeutic agent by a linker selected from the group consisting of a maleimidocaproyl linker, a peptide-based linker (including, but not limited to a valine-citrulline linker), a β-glucuronide linker, a succinimidyl 4-(N-maleimidomethyl)cyclohexane-1-carboxylate (SMCC) linker, a disulfide linker, or an acid sensitive linker. In one aspect, the therapeutic agent is a radionuclide. In one aspect, the radionuclide is a β-particle-emitting radionuclide or an α-particle emitting radionuclide. In one aspect, the radionuclide is an α-particle emitting radionuclide selected from the group consisting of astatine-211, bismuth-212, lead-212, bismuth-213, actinium-225, radium-223 and thorium-227. In one aspect, the radionuclide is a β-particle-emitting radionuclide. In one aspect, the β-particle-emitting radionuclide is selected from the group consisting of iodine-131, rhenium-186, yttrium-90, samarium-153, and lutetium-177.

Also disclosed herein are compositions for use in treating, inhibiting or reducing proliferation of or killing a cancer cell in a subject, wherein the subject has not been administered an intracellular delivery compound, or wherein the composition is not for administration or is not formulated for administration to the subject in conjunction with an intracellular delivery compound, and wherein the cancer cell expresses a Surface K-Ras Antigen on the external surface of the cancer cell. In one aspect, the cancer cells are selected from the group consisting of pancreatic cancer cells, lung cancer cells, cholangial cancer cells, ovarian cancer cells, endometrial cancer cells, or colorectal cancer cells. In one aspect, the composition is formulated for administration by intravenous injection or subcutaneous injection.

Also disclosed herein are methods of treating, inhibiting or reducing proliferation of or killing a cancer cell in a subject comprising administering a therapeutically-effective amount of any of the compositions described herein to the subject, wherein the subject is not administered an intracellular delivery compound in conjunction with administration of the composition, and wherein the cancer cell expresses a Surface K-Ras Antigen on the external surface of the cancer cell. In one aspect, the cancer cells are selected from the group consisting of pancreatic cancer cells, lung cancer cells, cholangial cancer cells, ovarian cancer cells, endometrial cancer cells, or colorectal cancer cells. In one aspect, the administering step is performed by intravenous injection or subcutaneous injection.

Also disclosed herein are methods of treating, inhibiting or reducing proliferation of or killing a cancer cell in a subject comprising administering a therapeutically-effective amount of a composition, wherein the composition comprises a means for selectively binding to a Surface K-Ras Antigen expressed on the external surface of the cancer cell. In one aspect, the means is an antibody. In one aspect, the means is an antibody or antibody fragment that selectively binds the Surface K-Ras Antigen, wherein the Surface K-Ras Antigen comprises a mutation at residue 12, 13, or 61 based on the amino acid sequence set forth in SEQ ID NO: 1 or SEQ ID NO: 2. In one aspect, the means is an antibody or antibody fragment that selectively binds the Surface K-Ras Antigen, wherein the Surface K-Ras Antigen comprises any one of the following mutations based on the amino acid sequence set forth in SEQ ID NO: 1 or SEQ ID NO: 2: G12A; G12D; G12C; G12V; G12R; G13D; Q61H; and Q61 L. In one aspect, the means is an antibody or antibody fragment that selectively binds the Surface K-Ras Antigen, wherein the Surface K-Ras Antigen comprises the G12D mutation based on the amino acid sequence set forth in SEQ ID NO: 1 or SEQ ID NO: 2. In one aspect, the means is an antibody or antibody fragment that selectively binds the Surface K-Ras Antigen, wherein the Surface K-Ras Antigen comprises the G12D mutation as set forth in SEQ ID NO: 295. In one aspect, the means is an antibody or antibody fragment that selectively binds the Surface K-Ras Antigen, wherein the Surface K-Ras Antigen comprises the G12C mutation based on the amino acid sequence set forth in SEQ ID NO: 1 or SEQ ID NO: 2. In one aspect, the means is an antibody or antibody fragment that selectively binds the Surface K-Ras Antigen, wherein the Surface K-Ras Antigen comprises the G12V mutation based on the amino acid sequence set forth in SEQ ID NO: 1 or SEQ ID NO: 2. In one aspect, the means is an antibody fragment.

In one aspect, the means is a peptide or protein. In one aspect, the means is a peptide or protein that selectively binds the Surface K-Ras Antigen, wherein the Surface K-Ras Antigen comprises the G12D mutation based on the amino acid sequence set forth in SEQ ID NO: 1 or SEQ ID NO: 2. In one aspect, the means is a peptide or protein that selectively binds the Surface K-Ras Antigen, wherein the Surface K-Ras Antigen comprises the G12D mutation as set forth in SEQ ID NO: 295. In one aspect, the means is a peptide or protein that selectively binds the Surface K-Ras Antigen, wherein the Surface K-Ras Antigen comprises a mutation at residue 12, 13, or 61 based on the amino acid sequence set forth in SEQ ID NO: 1 or SEQ ID NO: 2. In one aspect, the means is a peptide or protein that selectively binds the Surface K-Ras Antigen, wherein the Surface K-Ras Antigen comprises the G12C mutation based on the amino acid sequence set forth in SEQ ID NO: 1 or SEQ ID NO: 2. In one aspect, the means is a peptide or protein that selectively binds the Surface K-Ras Antigen, wherein the Surface K-Ras Antigen comprises the G12V mutation based on the amino acid sequence set forth in SEQ ID NO: 1 or SEQ ID NO: 2. In one aspect, the means is an antibody described in Table 1 or Table 2, or a fragment thereof or is an antibody selected by the methods set forth in Examples 4 or 5. In one aspect, the cancer cell is a pancreatic cancer cell, a lung cancer cell, or a colorectal cancer cell. In one aspect, the administering step is performed by intravenous injection or subcutaneous injection. In one aspect, the Surface K-Ras Antigen is a full length or truncated form of K-Ras with a mutation at any of the following amino acids/residues: 12 (including, but not limited to G12A, G12D, G12C, G12V, G12R); 13 (including, but not limited to G13D); and 61 (including, but not limited to Q61H, Q61L) based on the amino acid position in SEQ ID NO: 1 or SEQ ID NO: 2.

In one aspect, any of the methods of treating, inhibiting or reducing proliferation of or killing a cancer cell in a subject further comprises the step of administering to the subject an additional therapeutic agent prior to or concurrently with the administration of the composition, wherein the additional therapeutic agent is separate from the therapeutic agent present in the composition, and wherein the administration of the additional therapeutic agent leads to increased binding availability of the Surface K-Ras Antigen on the external surface of the cancer cell. In one aspect, the additional therapeutic agent is a K-Ras small molecule inhibitor. In one aspect, the K-Ras small molecule inhibitor specifically inhibits the activity of a form of K-Ras carrying a mutation that is present in the Surface K-Ras Antigen. In one aspect, the K-Ras small molecule inhibitor is MRTX1133 or RMC-6236. In one aspect, the Surface K-Ras Antigen comprises a G12D mutation. In one aspect the Surface K-Ras Antigen comprises the sequence set forth in SEQ ID NO: 295. In one aspect, the additional therapeutic agent is administered to the subject 1 day to 14 days prior to administration of the composition. In one aspect, the additional therapeutic agent is administered to the subject 3 days to 7 days prior to administration of the composition.

In one aspect, any of the methods of treating, inhibiting or reducing proliferation of or killing a cancer cell in a subject further comprises the step of administering to the subject a therapeutic agent prior to or concurrently with the administration of the composition, and wherein the administration of the therapeutic agent leads to increased expression of the Surface K-Ras Antigen on the external surface of the cancer cell. In one aspect, the therapeutic agent is a K-Ras small molecule inhibitor. In one aspect, the K-Ras small molecule inhibitor specifically inhibits the activity of a form of K-Ras carrying a mutation that is present in the Surface K-Ras Antigen. In one aspect, the K-Ras small molecule inhibitor is MRTX1133 or RMC-6236. In one aspect, the Surface K-Ras Antigen comprises a G12D mutation. In one aspect, the therapeutic agent is administered to the subject 1 day to 14 days prior to administration of the composition. In one aspect, the therapeutic agent is administered to the subject 3 days to 7 days prior to administration of the composition.

Also disclosed herein are chimeric antigen receptors comprising an extracellular domain, a transmembrane domain, and an endodomain, wherein the extracellular domain comprises a binding agent, wherein the binding agent binds with specificity to a Surface K-Ras Antigen expressed on an external surface of a cancer cell. In one aspect, the transmembrane domain is selected from the group consisting of CD3-zeta, CD28, CDE28a, CD4 or combinations thereof. In one aspect, the endodomain is selected from the group consisting of CD28, CD27, 4-1 BB, OX40, and/or ICOS. In one aspect, the binding agent is an antibody or antibody fragment. In one aspect, the binding agent is an antibody or antibody fragment that selectively binds the Surface K-Ras Antigen, wherein the Surface K-Ras Antigen comprises a mutation at residue 12, 13, or 61 based on the amino acid sequence set forth in SEQ ID NO: 1 or SEQ ID NO: 2. In one aspect, the binding agent is an antibody or antibody fragment that selectively binds the Surface K-Ras Antigen, wherein the Surface K-Ras Antigen comprises any one of the following mutations based on the amino acid sequence set forth in SEQ ID NO: 1 or SEQ ID NO: 2: G12A; G12D; G12C; G12V; G12R; G13D; Q61H; and Q61L. In one aspect, the binding agent is an antibody or antibody fragment that selectively binds the Surface K-Ras Antigen, wherein the Surface K-Ras Antigen comprises the G12D mutation based on the amino acid sequence set forth in SEQ ID NO: 1 or SEQ ID NO: 2. In one aspect, the binding agent is an antibody or antibody fragment that selectively binds the Surface K-Ras Antigen, wherein the Surface K-Ras Antigen comprises the G12D mutation as set forth in SEQ ID NO: 295. In one aspect, the binding agent is an antibody or antibody fragment that selectively binds the Surface K-Ras Antigen, wherein the Surface K-Ras Antigen comprises the G12C mutation based on the amino acid sequence set forth in SEQ ID NO: 1 or SEQ ID NO: 2. In one aspect, the binding agent is an antibody or antibody fragment that selectively binds the Surface K-Ras Antigen, wherein the Surface K-Ras Antigen comprises the G12V mutation based on the amino acid sequence set forth in SEQ ID NO: 1 or SEQ ID NO: 2. In one aspect, the binding agent is an antibody fragment. In one aspect, the Surface K-Ras Antigen possesses greater than 70% sequence identity to SEQ ID NO: 1 or SEQ ID NO: 2. In one aspect, the Surface K-Ras Antigen possesses greater than 80% sequence identity to SEQ ID NO: 1 or SEQ ID NO: 2. In one aspect, the Surface K-Ras Antigen possesses greater than 90% sequence identity to SEQ ID NO: 1 or SEQ ID NO: 2. In one aspect, the Surface K-Ras Antigen possesses greater than 70% sequence identity to SEQ ID NO: 1 or SEQ ID NO: 2. In one aspect, the Surface K-Ras Antigen possesses greater than 80% sequence identity to SEQ ID NO: 1 or SEQ ID NO: 2. In one aspect, the Surface K-Ras Antigen possesses greater than 90% sequence identity to SEQ ID NO: 1 or SEQ ID NO: 2 or greater than 95% sequence identity to SEQ ID NO: 1 or SEQ ID NO: 2. In one aspect, the Surface K-Ras Antigen possesses greater than 70% sequence identity to SEQ ID NO: 1 or SEQ ID NO: 2. In one aspect, the Surface K-Ras Antigen possesses greater than 80% sequence identity to SEQ ID NO: 1 or SEQ ID NO: 2. In one aspect, the Surface K-Ras Antigen possesses greater than 90% sequence identity to SEQ ID NO: 1 or SEQ ID NO: 2 or greater than 95% sequence identity to SEQ ID NO: 1 or SEQ ID NO: 2.

Also disclosed herein are vectors comprising a nucleotide sequence encoding any of the chimeric antigen receptors described herein.

Also disclosed herein are immune cells expressing any of the chimeric antigen receptors described herein. In one aspect, the immune cell is a cell from an individual. In one aspect, the immune cell is a T cell from an individual. In one aspect, the immune cell is selected from the group consisting of a T cell, a NK cell, a dendritic cell or a mixture thereof. In one aspect, the immune cell is a T cell. In one aspect, the immune cell is a CD4+ T cell or a CD8+ T cell.

Also disclosed herein are compositions comprising an immune cell that expresses a chimeric antigen receptor that targets a Surface K-Ras Antigens, including a mutant Surface K-Ras antigen, expressed on the extracellular surface of cancer cells. In one aspect, the immune cell is derived from a source that is autologous, syngeneic, allogenic or xenogeneic. In one aspect, the immune cell is a T cell, and wherein the T cell is derived from a source that is autologous, syngeneic, allogenic or xenogeneic. In one aspect, the immune cell is selected from the group consisting of a T cell, a NK cell, a dendritic cell or a mixture thereof. In one aspect, the immune cell is a T cell. In one aspect, the immune cell is a CD4+ T cell or a CD8+ T cell.

Also disclosed herein are immune cells and compositions comprising immune cells for use in treating a cancer in an individual, wherein the cancer comprises cancer cells that express a Surface K-Ras Antigen on an external surface of the cancer cells. In one aspect, the immune cells comprise a cell from the individual having the cancer. In one aspect, the immune cell is selected from the group consisting of a T cell, a NK cell, a dendritic cell or a mixture thereof. In one aspect, the immune cell is a CD4+ T cell or a CD8+ T cell. In one aspect, the cancer cells are selected from the group consisting of pancreatic cancer cells, lung cancer cells, cholangial cancer cells, ovarian cancer cells, endometrial cancer cells, or colorectal cancer cells. In one aspect, the subject has been administered an additional therapeutic agent, or wherein the immune cell is for concurrent administration with an additional therapeutic agent. In one aspect, the additional therapeutic agent is a K-Ras small molecule inhibitor. In one aspect, the K-Ras small molecule inhibitor specifically inhibits the activity of a form of K-Ras carrying a mutation that is present in the Surface K-Ras Antigen. In one aspect, the K-Ras small molecule inhibitor is MRTX1133 or RMC-6236. In one aspect, the Surface K-Ras Antigen comprises a G12D mutation. In one aspect, the therapeutic agent was administered to the subject 1 day to 14 days prior to administration of the immune cells. In one aspect, the therapeutic agent was administered to the subject 3 days to 7 days prior to administration of the immune cells.

Also disclosed herein are methods of treating an individual having a cancer comprising cancer cells that express a Surface K-Ras Antigen on an external surface of the cancer cells comprising administering to the individual a therapeutically effective amount of immune cells expressing any of the chimeric antigen receptors described herein. In one aspect, the immune cells comprise a cell from the individual having the cancer. In one aspect, the immune cells are T cells from the individual having the cancer. In one aspect, the immune cell is selected from the group consisting of a T cell, a NK cell, a dendritic cell or a mixture thereof. In one aspect, the immune cell is a T cell. In one aspect, the immune cell is a CD4+ T cell or a CD8+ T cell. In one aspect, the cancer cells are selected from the group consisting of pancreatic cancer cells, lung cancer cells, cholangial cancer cells, ovarian cancer cells, endometrial cancer cells, or colorectal cancer cells.

Also described herein are methods of treating an individual having a cancer comprising cancer cells that express a Surface K-Ras Antigen on an external surface of the cancer cells comprising administering to the individual a therapeutically effective amount of any of the compositions described herein. In one aspect, the cancer cells are selected from the group consisting of pancreatic cancer cells, lung cancer cells, cholangial cancer cells, ovarian cancer cells, endometrial cancer cells, or colorectal cancer cells.

In one aspect, any of the methods of treating an individual having a cancer comprising cancer cells that express a Surface K-Ras Antigen on an external surface of the cancer cells further comprises the step of administering to the individual a therapeutic agent prior to or concurrently with the administration of the immune cells expressing the chimeric antigen receptor, and wherein the administration of the therapeutic agent leads to increased binding availability of the Surface K-Ras Antigen on the external surface of the cancer cell. In one aspect, the therapeutic agent is a K-Ras small molecule inhibitor. In one aspect, the K-Ras small molecule inhibitor specifically inhibits the activity of a form of K-Ras carrying a mutation that is present in the Surface K-Ras Antigen. In one aspect, the K-Ras small molecule inhibitor is MRTX1133 or RMC-6236. In one aspect, the Surface K-Ras Antigen comprises a G12D mutation. In one aspect, the therapeutic agent is administered to the subject 1 day to 14 days prior to administration of the immune cells. In one aspect, the therapeutic agent is administered to the subject 3 days to 7 days prior to administration of the immune cells.

Also disclosed herein are methods of diagnosing a cancer associated with expression of a Surface K-Ras Antigen on an external surface of a cell, the method comprising the steps of: (a) obtaining a cell sample from a subject comprising a population of cells; (b) exposing the cell sample to an agent capable of binding the Surface K-Ras Antigen, wherein the population of cells are not lysed or otherwise permeabilized to avoid the agent reacting with intracellular K-Ras; and (c) measuring the presence of the agent on the external surface of the population of cells present in the cell sample. In one aspect, the Surface K-Ras Antigen is a mutant Surface K-Ras Antigen. In one aspect, the agent is an antibody or an antibody fragment that binds to the Surface K-Ras Antigen. In one aspect, the antibody or antibody fragment comprises a label. In one aspect, the label is fluorochrome. In one aspect, the method of steps (b) and (c) are performed on a sample previously obtained from the subject.

In one aspect, the methods of diagnosing a cancer associated with expression of a Surface K-Ras Antigen on an external surface of a cell further comprise a secondary antibody that binds to a portion of the antibody or antibody fragment, wherein the secondary antibody is labeled. In one aspect, step (c) is performed by applying the cell sample following step (b) to a flow cytometer. In one aspect, step (c) is performed by electron microscopy. In one aspect, prior to step (b), the cell sample is exposed to a cell-surface membrane dye. In one aspect, step (c) is performed by confocal microscopy. In one aspect, the cell sample is selected from the group consisting of pancreatic, colorectal, cholangial, ovarian, endometrial and lung cells.

Also disclosed herein are methods of diagnosing a cancer associated with expression of a Surface K-Ras Antigen, the method comprising the steps of: (a) obtaining a cell sample from a subject comprising a population of cells; (b) labeling proteins on the external surface of the cell with a first agent; (c) lysing the population of cells to yield a cell lysate sample; (d) capturing labeled proteins by applying the cell lysate sample to a surface coated with a second agent, wherein the second agent selectively binds the first agent; (e) removing the captured labeled proteins from the surface and removing the first agent form the captured labeled proteins to yield a cell-surface protein sample; (f) exposing the cell-surface protein sample to a third agent capable of selectively binding the Surface K-Ras Antigen; (g) exposing the cell surface protein sample to a fourth agent, wherein the fourth agent carries a detectable label and binds to a portion of the third agent; and (h) measuring the presence of the detectable label. In one aspect, steps (f)-(h) are performed by western blot. In one aspect, the method of steps (b) through (h) are performed on a sample previously obtained from the subject.

In one aspect, any of the methods of diagnosing a cancer associated with expression of a Surface K-Ras Antigen further comprise the step of subjecting the subject to the one or more of (e.g., one, two, three, four, five, six, seven, eight, nine, ten or more of the steps of): (i) administering a therapeutically-effective amount of any of the compositions described herein; (ii) administering using intravenous injection or subcutaneous injection a therapeutically-effective amount of any of the compositions described herein; (iii) administering a therapeutically-effective amount of a composition, wherein the composition comprises a means for selectively binding to a Surface K-Ras Antigen expressed on the external surface of the cancer cell; (iv) administering a therapeutically-effective amount of a composition, wherein the composition comprises a means for selectively binding to a Surface K-Ras Antigen expressed on the external surface of the cancer cell, wherein the means is an antibody; (v) administering a therapeutically-effective amount of a composition, wherein the composition comprises a means for selectively binding to a Surface K-Ras Antigen expressed on the external surface of the cancer cell, wherein the means is an antibody or antibody fragment that selectively binds the Surface K-Ras Antigen, wherein the Surface K-Ras Antigen comprises a mutation at residue 12, 13, or 61 based on the amino acid sequence set forth in SEQ ID NO: 1 or SEQ ID NO: 2; (vi) administering a therapeutically-effective amount of a composition, wherein the composition comprises a means for selectively binding to a Surface K-Ras Antigen expressed on the external surface of the cancer cell, wherein the means is an antibody or antibody fragment that selectively binds the Surface K-Ras Antigen, wherein the Surface K-Ras Antigen comprises any one of the following mutations based on the amino acid sequence set forth in SEQ ID NO: 1 or SEQ ID NO: 2: G12A; G12D; G12C; G12V; G12R; G13D; Q61H; and Q61L; (vii) administering a therapeutically-effective amount of a composition, wherein the composition comprises a means for selectively binding to a Surface K-Ras Antigen expressed on the external surface of the cancer cell, wherein the means is an antibody or antibody fragment that selectively binds the Surface K-Ras Antigen, wherein the Surface K-Ras Antigen comprises the G12D mutation based on the amino acid sequence set forth in SEQ ID NO: 1 or SEQ ID NO: 2; (viii) administering a therapeutically-effective amount of a composition, wherein the composition comprises a means for selectively binding to a Surface K-Ras Antigen expressed on the external surface of the cancer cell, wherein the means is an antibody or antibody fragment that selectively binds the Surface K-Ras Antigen, wherein the Surface K-Ras Antigen comprises the G12C mutation based on the amino acid sequence set forth in SEQ ID NO: 1 or SEQ ID NO: 2; (ix) administering a therapeutically-effective amount of a composition, wherein the composition comprises a means for selectively binding to a Surface K-Ras Antigen expressed on the external surface of the cancer cell, wherein the means is an antibody or antibody fragment that selectively binds the Surface K-Ras Antigen, wherein the Surface K-Ras Antigen comprises the G12V mutation based on the amino acid sequence set forth in SEQ ID NO: 1 or SEQ ID NO: 2; (x) administering a therapeutically-effective amount of a composition, wherein the composition comprises a means for selectively binding to a Surface K-Ras Antigen expressed on the external surface of the cancer cell, wherein the means is an antibody fragment; (xi) administering a therapeutically-effective amount of a composition, wherein the composition comprises a means for selectively binding to a Surface K-Ras Antigen expressed on the external surface of the cancer cell, wherein the means is a peptide or protein; (xii) administering a therapeutically-effective amount of a composition, wherein the composition comprises a means for selectively binding to a Surface K-Ras Antigen expressed on the external surface of the cancer cell, wherein the means is a peptide or protein that selectively binds the Surface K-Ras Antigen, wherein the Surface K-Ras Antigen comprises the G12D mutation based on the amino acid sequence set forth in SEQ ID NO: 1 or SEQ ID NO: 2; (xiii) administering a therapeutically-effective amount of a composition, wherein the composition comprises a means for selectively binding to a Surface K-Ras Antigen expressed on the external surface of the cancer cell, wherein the means is a peptide or protein that selectively binds the Surface K-Ras Antigen, wherein the Surface K-Ras Antigen comprises a mutation at residue 12, 13, or 61 based on the amino acid sequence set forth in SEQ ID NO: 1 or SEQ ID NO: 2; (xiv) administering a therapeutically-effective amount of a composition, wherein the composition comprises a means for selectively binding to a Surface K-Ras Antigen expressed on the external surface of the cancer cell, wherein the means is a peptide or protein that selectively binds the Surface K-Ras Antigen, wherein the Surface K-Ras Antigen comprises the G12C mutation based on the amino acid sequence set forth in SEQ ID NO: 1 or SEQ ID NO: 2; (xv) administering a therapeutically-effective amount of a composition, wherein the composition comprises a means for selectively binding to a Surface K-Ras Antigen expressed on the external surface of the cancer cell, wherein the means is a peptide or protein that selectively binds the Surface K-Ras Antigen, wherein the Surface K-Ras Antigen comprises the G12V mutation based on the amino acid sequence set forth in SEQ ID NO: 1 or SEQ ID NO: 2; (xvi) administering a therapeutically-effective amount of a composition, wherein the composition comprises a means for selectively binding to a Surface K-Ras Antigen expressed on the external surface of the cancer cell, wherein the means is an antibody described in Table 1 or Table 2, or a fragment thereof or is an antibody selected by the methods set forth in Examples 4 or 5; (xvii) administering a therapeutically-effective amount of a composition, wherein the composition comprises a means for selectively binding to a Surface K-Ras Antigen expressed on the external surface of the cancer cell, wherein the Surface K-Ras Antigen is a full length or truncated form of K-Ras with a mutation at any of the following amino acids/residues: 12 (including, but not limited to G12A, G12D, G12C, G12V, G12R); 13 (including, but not limited to G13D); and 61 (including, but not limited to Q61H, Q61 L) based on the amino acid position in SEQ ID NO: 1 or SEQ ID NO: 2; (xviii) administering a therapeutically-effective amount of a composition, wherein the composition comprises a means for selectively binding to a Surface K-Ras Antigen expressed on the external surface of the cancer cell, wherein the Surface K-Ras Antigen possesses greater than 70% sequence identity to SEQ ID NO: 1 or SEQ ID NO: 2; (xix) administering a therapeutically-effective amount of a composition, wherein the composition comprises a means for selectively binding to a Surface K-Ras Antigen expressed on the external surface of the cancer cell, wherein the Surface K-Ras Antigen possesses greater than 80% sequence identity to SEQ ID NO: 1 or SEQ ID NO: 2; (xx) administering a therapeutically-effective amount of a composition, wherein the composition comprises a means for selectively binding to a Surface K-Ras Antigen expressed on the external surface of the cancer cell, wherein the Surface K-Ras Antigen possesses greater than 90% sequence identity to SEQ ID NO: 1 or SEQ ID NO: 2 or greater than 95% sequence identity to SEQ ID NO: 1 or SEQ ID NO: 2; (xxi) administering to the individual a therapeutically effective amount of immune cells expressing any of the chimeric antigen receptors described herein; (xxii) administering to the individual a therapeutically effective amount of immune cells expressing the chimeric antigen receptor, wherein the immune cells comprise a cell from the individual having the cancer; (xxiii) administering to the individual a therapeutically effective amount of immune cells expressing any of the chimeric antigen receptors described herein; (xxii) administering to the individual a therapeutically effective amount of immune cells expressing the chimeric antigen receptor, wherein the immune cells are T cells from the individual having the cancer; (xxiv) administering to the individual a therapeutically effective amount of immune cells expressing any of the chimeric antigen receptors described herein; (xxii) administering to the individual a therapeutically effective amount of immune cells expressing the chimeric antigen receptor, wherein the immune cell is selected from the group consisting of a T cell, a NK cell, a dendritic cell or a mixture thereof; (xxv) administering to the individual a therapeutically effective amount of immune cells expressing any of the chimeric antigen receptors described herein; (xxii) administering to the individual a therapeutically effective amount of immune cells expressing the chimeric antigen receptor, wherein the immune cell is a T cell; (xxvi) administering to the individual a therapeutically effective amount of immune cells expressing any of the chimeric antigen receptors described herein; (xxvii) administering to the individual a therapeutically effective amount of immune cells expressing the chimeric antigen receptor, wherein the immune cell is a CD4+ T cell or a CD8+ T cell; (xxviii) administering a therapeutically-effective amount of a composition, wherein the composition comprises a means for selectively binding to a Surface K-Ras Antigen expressed on the external surface of the cancer cell, wherein the subject is not administered an intracellular delivery compound in conjunction with administration of the composition; or (xxix) combinations thereof, if the Surface K-Ras Antigen is expressed on an external surface of the cell. In one aspect, the therapeutic agent is therapeutically effective to inhibit growth or proliferation of the cancer cell or is otherwise cytotoxic to the cancer cell. In one aspect, the subject is not administered an intracellular delivery compound in conjunction with administration of the composition.

Also disclosed herein are therapeutically-effective amounts of any of the compositions disclosed herein for use in treating, inhibiting or reducing proliferation of or killing a cancer cell in a subject, wherein the subject has been administered an additional therapeutic agent, and wherein the administration of the additional therapeutic agent stimulates expression of Surface K-Ras Antigen on the external surface of the cancer cell. In one aspect, the subject has not been administered an intracellular delivery compound in conjunction with administration of the composition. In one aspect, the additional therapeutic agent is a K-Ras small molecule inhibitor. In one aspect, the additional therapeutic agent was administered to the subject 1 day to 14 days prior to administration of the composition. In one aspect, the additional therapeutic agent was administered to the subject 3 days to 7 days prior to administration of the composition. In one aspect, the cancer cells are selected from the group consisting of pancreatic cancer cells, lung cancer cells, cholangial cancer cells, ovarian cancer cells, endometrial cancer cells, or colorectal cancer cells. In one aspect, the additional therapeutic and the composition are administered to the subject at substantially the same time.

Also disclosed herein are bispecific antibodies comprising a first binding domain linked to a second binding domain, wherein the first binding domain selectively binds to a Surface K-Ras Antigen expressed on an external surface of a cancer cell, and wherein the second binding domain selectively binds to an antigen expressed on a surface of an immune effector cell. In one aspect, the first binding domain comprises a light chain variable region and a heavy chain (HC) variable region (VH). In one aspect, the first binding domain comprises a light chain (LC) variable region (VL) and constant region (FC) and a heavy chain variable region and constant region. In one aspect, the second binding domain comprises a light chain variable region and a heavy chain variable region. In one aspect, the second binding domain comprises a light chain variable and constant region and a heavy chain variable and constant region. In one aspect, the Surface K-Ras Antigen possess at least 60% homology to SEQ ID NO: 1 or SEQ ID NO: 2. In one aspect, the first binding domain selectively binds to a region on the Surface K-Ras Antigen that includes residues 12, 13, or 61 based on the amino acid sequence set forth in SEQ ID NO: 1 or SEQ ID NO: 2. In one aspect, the first binding domain selectively binds the Surface K-Ras Antigen with any one of the following mutations based on the amino acid sequence set forth in SEQ ID NO: 1 or SEQ ID NO: 2: G12A; G12D; G12C; G12V; G12R; G13D; Q61H; and Q61 L. In one aspect, the first binding domain selectively binds the Surface K-Ras Antigen with the G12D mutation based on the amino acid sequence set forth in SEQ ID NO: 1 or SEQ ID NO: 2. In one aspect, the first binding domain selectively binds the Surface K-Ras Antigen with the G12D mutation set forth in SEQ ID NO: 295. In one aspect, the first binding domain selectively binds the Surface K-Ras Antigen with the G12C mutation based on the amino acid sequence set forth in SEQ ID NO: 1 or SEQ ID NO: 2. In one aspect, the first binding domain selectively binds the Surface K-Ras Antigen with the G12V mutation based on the amino acid sequence set forth in SEQ ID NO: 1 or SEQ ID NO: 2. In one aspect, the antigen expressed on a surface of an immune effector cell is selected from the group consisting of TCRα, TCRβ, TCRδ, TCRγ, CD3β, CD3γ, CD3ε, CD3δ, CD3ζ, CD137, CD16, and CD64. In one aspect, the antigen expressed on a surface of an immune effector cell is CD3ε. In one aspect, the immune effector cell is selected from the group consisting of a T cell, a neutrophil, a macrophage, a monocyte and an NK cell.

Also disclosed herein are methods of treating, inhibiting or reducing proliferation of or killing a cancer cell in a subject comprising administering a therapeutically-effective amount of any of the bispecific antibodies disclosed herein, and wherein a Surface K-Ras Antigen is expressed on the external surface of the cancer cell. In one aspect, the cancer cells are selected from the group consisting of pancreatic cancer cells, lung cancer cells, cholangial cancer cells, ovarian cancer cells, endometrial cancer cells, or colorectal cancer cells. In one aspect, the administering step is performed by intravenous injection or subcutaneous injection. In one aspect, the Surface K-Ras Antigen is a full length or truncated form of K-Ras with a mutation at any of the following amino acids/residues: 12 (including, but not limited to G12A, G12D, G12C, G12V, G12R); 13 (including, but not limited to G13D); and 61 (including, but not limited to Q61H, Q61L) based on the amino acid position in SEQ ID NO: 1 or SEQ ID NO: 2. In a further aspect, the bispecific antibodies described herein are for use in treating, inhibiting or reducing proliferation of or killing a cancer cell in a subject.

Also disclosed herein are compositions comprising a means for selectively binding to a Surface K-Ras Antigen expressed on an external surface of a cancer cell, wherein the means is linked to a therapeutic agent, and wherein the composition does not include an intracellular delivery compound. In one aspect, the Surface K-Ras Antigen is a full length or truncated form of K-Ras with a mutation at any of the following amino acids/residues: 12 (including, but not limited to G12A, G12D, G12C, G12V, G12R); 13 (including, but not limited to G13D); and 61 (including, but not limited to Q61H, Q61L) based on the amino acid position in SEQ ID NO: 1 or SEQ ID NO: 2.

Also disclosed herein are bispecific antibodies comprising a first means for selectively binding to a Surface K-Ras Antigen expressed on an external surface of a cancer cell, and a second means for selectively binding to an antigen expressed on a surface of an immune effector cell, wherein the first means is linked to the second means. In one aspect, the Surface K-Ras Antigen is a full length or truncated form of K-Ras with a mutation at any of the following amino acids/residues: 12 (including, but not limited to G12A, G12D, G12C, G12V, G12R); 13 (including, but not limited to G13D); and 61 (including, but not limited to Q61H, Q61 L) based on the amino acid position in SEQ ID NO: 1 or SEQ ID NO: 2.

Also disclosed herein are chimeric antigen receptors comprising an extracellular domain, a transmembrane domain, and an endodomain, wherein the extracellular domain comprises a means for binding with specificity to a Surface K-Ras Antigen expressed on an external surface of a cancer cell. In one aspect, the Surface K-Ras Antigen is a full length or truncated form of K-Ras with a mutation at any of the following amino acids/residues: 12 (including, but not limited to G12A, G12D, G12C, G12V, G12R); 13 (including, but not limited to G13D); and 61 (including, but not limited to Q61H, Q61L) based on the amino acid position in SEQ ID NO: 1 or SEQ ID NO: 2.

### BRIEF DESCRIPTION OF THE DRAWINGS

FIG. 1a depicts the design of R11.1.6-based constructs used in Examples 1.

FIG. 1b is a graphical representation of the growth (left panel) and viability (right panel) of Panc-1 (K-Ras G12D mutant) and MiaPaca-2 (K-Ras G12C mutant) cells cultured in the presence of R11.1.6-based constructs described in FIG. 1a.

FIG. 1c is a graphical representation of the growth of lung cancer cell lines (left), colorectal cancer cell lines (middle), and RASless mouse embryonic fibroblast system (MEFs) (right) cultured in the presence of R11.1.6 vs. saline control. The figure demonstrates cell counts on left and center and absorbance of purple formazan indicated number of live cells in an MTT assay on right. ***p<0.001; **p<0.01; *p<0.05; ns p>0.05. All statistics performed by unpaired t-test in GraphPad Prism.

FIG. 1d is a graphical representation of the growth (left panel) and viability (right panel) of Panc-1 (K-Ras G12D mutant) and MiaPaca-2 (K-Ras G12C mutant) cultured in the presence of R11.1.6 compared to a small molecule inhibitor specific for K-Ras G12D mutation as well as the combination of the two. Data plotted relative to staining of tumor cells with anti-His-tag secondary only. ***p<0.001; **p<0.01; *p<0.05; ns p>0.05. All statistics performed by unpaired t-test in GraphPad Prism.

FIG. 1e is a graphical representation of the growth (left) and viability (right) of Panc-1 (K-Ras G12D mutant) and MiaPaca-2 (K-Ras G12C mutant) cultured in the presence of R11.1.6 compared to various mutated therapeutic proteins based on R11.1.6. Such proteins include a mutated form of R11.1.6 where the codons in the regions that contact the K-RAS mutant were scrambled (scrambled R11.1.6), M11.1.2 scaffolding protein that binds mouse serum albumin, and E11.4.1 that binds human EGFR. Data plotted relative to staining of tumor cells with anti-His-tag secondary only. ***p<0.001; **p<0.01; *p<0.05; ns p>0.05. All statistics performed by unpaired t-test in GraphPad Prism.

FIG. 1f is a graphical representation of surface binding of R11.1.6, scrambled R11.1.6, M11.1.2, and E11.4.1 to live Panc-1 and MiaPaca-2 cancer cells as defined by relative median fluorescence intensity by anti-His-tag secondary antibody staining. Data plotted relative to staining of tumor cells with anti-His-tag secondary only. ***p<0.001; **p<0.01; *p<0.05; ns p>0.05. All statistics performed by unpaired t-test in GraphPad Prism.

FIG. 1g is a graphical representation of flow cytometric staining for phosphorylation of AKT (left panel) and ERK (right panel) using phosphoflow specific antibodies at various time points after addition of R11.1.6 to cultures of Panc-1 (K-Ras G12D mutant) pancreatic cancer. ***p<0.001; **p<0.01; *p<0.05; ns p>0.05. All statistics performed by unpaired t-test in GraphPad Prism.

FIG. 1h is a graphical representation of localization of R11.1.6-based constructs via flow cytometric staining of anti-His antibodies. ***p<0.001; **p<0.01; *p<0.05; ns p>0.05. All statistics performed by unpaired t-test in GraphPad Prism.

FIG. 1i is a high-resolution confocal microscopy image describing localization of R11.1.6-based constructs on the outside of the cell by application of anti-His antibodies.

FIG. 2a Testing of anti-K-Ras antibodies for surface or total binding using RASless MEFS rescued with BRAFV^{600E}

FIG. 2b Testing of anti-K-Ras antibodies for surface or total binding using RASless MEFS rescued with BRAFV^{600E}

FIG. 2c Testing of Human Anti-K-Ras Antibody 1 for binding to RASless MEFS rescued with BRAF^{V600E}, G12D mutant version of K-Ras 4B, PANC-1, or MiaPaca-2 using flow cytometry.

FIG. 2d Testing of Human Anti-K-Ras Antibody 2 for binding to RASless MEFS rescued with BRAF^{V600E}, G12D mutant version of K-Ras 4B, PANC-1, or MiaPaca-2 using flow cytometry.

FIG. 2e Testing of Human Anti-K-Ras Antibody 1 for surface binding to RASless MEFs rescued with, wild-type K-Ras 4A or 4B, or mutant K-Ras 4B constructs using flow cytometry.

FIG. 2f shows representative histograms of surface K-Ras expression, as measured by Human Anti-K-Ras Antibody 1 staining, in multiple tumor cell lines. Top shows surface staining (black) and bottom shows total staining, as defined by both surface +intracellular expression (blue). Solid line is representative of Human Anti-K-Ras Antibody 1 staining and solid histogram representative of human IgG isotype control. Data representative of at least separate experiments.

FIG. 2g is a high-resolution confocal microscopy image describing localization of Human Anti-K-Ras Antibody 1 staining on the cell.

FIG. 2h shows representative histograms of surface K-Ras expression for the HL-10 and D2H12 antibodies on RASless MEFs, Panc-1, MiaPaca-2, and Capan-2 cell lines. Solid line is representative of anti-K-Ras antibody staining and solid histogram representative of human IgG isotype control.

FIG. 2i represents testing of antibodies for specific binding in western blotting to K-Ras (arrow at 21 KDa) using RASLess MEFs rescued with either BRAF^{V600E}, wild-type K-Ras 4A or 4B, or mutant K-Ras 4B constructs.

FIG. 2j represents three commercially available antibodies demonstrating both sensitive and specific binding for either K-Ras 4B G12D mutant or wild-type K-Ras 4A or 4B by western blot analysis. All blots loaded with equal amount of protein as determined by BSA protein assay and identified on each blot.

FIG. 2k shows western blot, RT-PCR, and surface flow cytometric analysis of Panc-1 tumor cells after treatment with K-Ras-specific or scrambled siRNA.

FIG. 2l shows surface expression of K-Ras, as measured by Human Anti-K-Ras Antibody 1 staining, in Panc-1 cells after surface MHC Class I expression was eliminated by CRISPR Cas-9 excision of P2 microglobulin as compared to the control, parental cells expressing MHC Class I. Top figure illustrates MHC Class I expression and bottom shows Human Anti-K-Ras Antibody 1 staining as a representative histogram. Bottom middle figure shows fold change of Human Anti-K-Ras Antibody 1 surface staining vs human IgG isotype control surface staining.

FIG. 2m shows western blot analysis for K-Ras and cyclin D in bulk cells as compared to proteins not pulled down (Non-Surface/Not Biotinylated) or pulled down (Surface Biotinylated) with streptavidin beads after live cell biotinylation of surface proteins. Equal amount of protein loaded into each lane as determined by BSA protein assay. Data representative of at least two experiments per cell line.

FIG. 3 is a graphical representation of tumor cell killing, defined as % of cells alive compared to untreated control cultures with various concentrations of saporin alone without targeting antibodies. Representative of 3 replicates per group. Arrow points to 4.5 nM saporin. This 4.5 nM concentration, conjugated to the secondary antibody, is utilized in the antibody-drug conjugate (ADC) assays described in Example 3.

FIG. 4 is a graphical representation of Panc-1 (G12D mutant pancreatic cell line) cell-specific killing, defined as % of cells alive compared to untreated control cultures with various concentrations of HL-10 vs. Rabbit IgG control antibody in a Fab-Zap assay. For some data the primary antibodies are bound to a secondary antibody containing saporin and in some data primary antibody alone is shown. Representative of 3 replicates per group.

FIG. 5 is a graphical representation of AsPC-1 (G12D mutant pancreatic cell line) cell-specific killing, defined as the percentage of cells alive compared to untreated control cultures with HL-10 vs Rabbit IgG control antibody in a Fab-Zap assay. For some data the primary antibodies are bound to a secondary antibody containing saporin and in some data primary antibody alone is shown. Representative of 3 replicates per group.

FIG. 6 is a graphical representation of LS180 (G12D mutant colorectal cell line) cell-specific killing, defined as the percentage of cells alive compared to untreated control cultures with various concentrations of HL-10 vs Rabbit IgG control antibody in a Fab-Zap assay. For some data the primary antibodies are bound to a secondary antibody containing saporin and in some data primary antibody alone is shown. Representative of 3 replicates per group.

FIG. 7 is a graphical representation of SK-LU-1 (G12D mutant pancreatic cell line) cell-specific killing, defined as the percentage of cells alive compared to untreated control cultures with various concentrations of HL-10 vs Rabbit IgG control antibody in a Fab-Zap assay. For some data the primary antibodies are bound to a secondary antibody containing saporin and in some data primary antibody alone is shown. Representative of 3 replicates per group.

FIG. 8 is a graphical representation of CAPAN-2 (G12V mutant pancreatic cell line) cell-specific killing, defined as the percentage of cells alive compared to untreated control cultures with various concentrations of D2H12 vs Rabbit IgG control antibody in a Fab-Zap assay. For some data the primary antibodies are bound to a secondary antibody containing saporin and in some data primary antibody alone is shown. Representative of 3 replicates per group.

FIG. 9 is a graphical representation of SW480 (G12V mutant colorectal cell line) cell-specific killing, defined as the percentage of cells alive compared to untreated control cultures with various concentrations of D2H12 vs Rabbit IgG control antibody in a Fab-Zap assay. For some data the primary antibodies are bound to a secondary antibody containing saporin and in some data primary antibody alone is shown. Representative of 3 replicates per group.

FIG. 10 is a graphical representation of NCI-H2444 (G12V mutant lung cancer cell line) cell-specific killing, defined as the percentage of cells alive compared to untreated control cultures with various concentrations of rabbit anti-human K-Ras G12V antibody (clone #D2H12) vs Rabbit IgG control antibody in a Fab-Zap assay. Representative of 3 replicates per group.

FIG. 11 is a graphical representation of MiaPaca (G12C mutant pancreatic cell line) cell-specific killing, defined as the percentage of cells alive compared to untreated control cultures with various concentrations of HL-10 vs Rabbit IgG control antibody in a Fab-Zap assay. For some data the primary antibodies are bound to a secondary antibody containing saporin and in some data primary antibody alone is shown. Representative of 3 replicates per group.

FIGS. 12a-12e are a graphical representations of Panc-1 (FIG. 12a), AsPC-1 (FIG. 12b), MiaPaca-2 (FIG. 12c), and BXPC3 (FIG. 12d) cell-specific killing, defined as the percentage of cells alive compared to untreated control cultures with various concentrations of Human Anti-K-Ras Antibody 1 vs human IgG control antibody in a Fab-Zap assay. For some data the primary antibodies are bound to a secondary antibody containing saporin and in some data primary antibody alone is shown. FIG. 12e is a control of saporin only. Representative of 3 replicates per group.

FIGS. 13a-13d are graphical representations of various RASless MEFs cell-specific killing, defined as the percentage of cells alive compared to untreated control cultures with various concentrations of Human Anti-K-Ras Antibody 1 vs human IgG control antibody in Fab-Zap assays, including MRF-BRAF (FIG. 13a), MEF(G12D) (FIG. 13b), MEF (wild-type KRAS-4A) (FIG. 13c), and MEF (KRAS-4B) (FIG. 13d). For some data the primary antibodies are bound to a secondary antibody containing saporin and in some data primary antibody alone is shown. Representative of 3 replicates per group.

FIG. 14 shows histograms of K-Ras expression, as measured by Human Anti-K-Ras Antibody 1 or Human Anti-K-Ras Antibody 2 staining, in primary human cells. Solid line is representative of anti-K-Ras antibody staining and solid histogram representative of human IgG isotype control. Cells from either normal or fibrotic lung tissue of a single donor were measured for surface (left) or total (right) K-Ras staining.

FIGS. 15a-15c are graphical representations of change in antibody binding availability of K-Ras on the surface of PANC-1 and MiaPaca cells with and without pretreatment with various therapeutic agents (5-FU, MRTX849, and MRTX1133).

FIG. 16a shows a graphical representation of percent survival of PANC-1 cells carrying the G12D K-Ras mutation exposed to surface K-Ras-targeted antibody-drug conjugates with and without pretreatment with MRTX1133.

FIG. 16b shows a graphical representation of the percent of viable cells as compared to untreated alive control treatment cells. Cells were treated with various combinations, as indicated, of IgG, an ADC of Human Anti-K-Ras Antibody 1, RMC-6236 and 5-FU. The dotted line indicates the maximum level of cell killing from treatment with 10⁷M saporin.

FIG. 17 shows a graphical representation of luminescence in Panc-1 cells expressing luciferase (or no Panc-1 cells for group “T cells only”) to demonstrate the effect of various bispecific antibody constructs on T cell-mediated killing.

FIG. 18 provides a schematic representation of an exemplary anti-K-Ras-anti-CD3 bispecific antibody construct.

FIGS. 19a-19d are a series of graphical representations showing results from an ELISA assessing binding of Human Anti-K-Ras Antibody 1 and Human Anti-K-Ras Antibody 2 to G12D mutant K-Ras loaded with GDP nucleotide (FIG. 19a) or GppNHp nucleotide (FIG. 19b), or wild type K-Ras loaded with GDP nucleotide (FIG. 19c) or GppNHp nucleotide (FIG. 19d).

FIGS. 20a-20h are a series of graphical representations showing cell-specific killing in various cell lines, defined as the percentage of cells alive compared to untreated control cultures with various concentrations of Human Anti-K-Ras Antibody 1, Human Anti-K-Ras Antibody 2, RSV, and saporin only control in a Fab-Zap assay.

FIGS. 21a-21b are a series of microscope images showing results from staining tumor tissue with Human Anti-K-Ras Antibody 1 in a PA1252 PDX mouse model (FIG. 21a) and in a PA0787 PDX mouse model (FIG. 21b) compared to a control antibody (isotype).

FIGS. 22a-22d are a series of graphical representations showing body weight (FIGS. 22a and 22c) and tumor volume in mm³ (FIGS. 22b and 22d) of Balb/c nude mice with implanted Panc-1 tumor cells and treated with various treatment regimens described in Table 5.

FIG. 23 is a heat map of the results of the HDX-MS experiment using K-Ras and human anti-K-Ras antibody 1.

FIG. 24 is a heat map of the results of the HDX-MS experiment using K-Ras and human anti-K-Ras antibody 2.

### DETAILED DESCRIPTION OF THE INVENTION

Unless otherwise specifically defined herein, all technical and scientific terms shall have the meaning attributed to it by one of ordinary skill in the relevant art.

As used herein, the term “Surface K-Ras Antigen” means a K-Ras peptide or protein that is expressed on the external surface of cells. Surface K-Ras Antigen includes full-length KRAS4B (SEQ ID NO: 1—MTEYKLVVVGAGGVGKSALTIQLIQNHFVDEYDPTIEDSYRKQVVIDGETCLLDILDTAGQ EEYSAMRDQYMRTGEGFLCVFAINNTKSFEDIHHYREQIKRVKDSEDVPMVLVGNKCDL PSRTVDTKQAQDLARSYGIPFIETSAKTRQGVDDAFYTLVREIRKHKEKMSKDGKKKKKK SKTKCVIM) or KRAS4A (SEQ ID NO: 2—MTEYKLVVVGAGGVGKSALTIQLIQNHFVDEYDPTIEDSYRKQVVIDGETCLLDILDTAGQ EEYSAMRDQYMRTGEGFLCVFAINNTKSFEDIHHYREQIKRVKDSEDVPMVLVGNKCDL PSRTVDTKQAQDLARSYGIPFIETSAKTRQRVEDAFYTLVREIRQYRLKKISKEEKTPGCV KIKKCIIM) and truncated forms thereof that are expressed on the external surface of cells. Surface K-Ras Antigen may further include full-length or truncated K-Ras peptide or protein (including forms KRAS4A and KRAS4B) with mutations at any of the following amino acids/residues or corresponding amino acids/residues thereof (based on SEQ ID NO: 1 or SEQ ID NO: 2): 12 (including, but not limited to G12A, G12D, G12C, G12V, G12R); 13 (including, but not limited to G13D); 61 (including, but not limited to Q61H, Q61 L) and any other mutation thereof associated with a cancer causing form of K-Ras peptide or protein. For example, K-Ras G12D has the amino acid sequence of MTEYKLVVVGADGVGKSALTIQLIQNHFVDEYDPTIEDSYRKQVVIDGETCLLDILDTAGQ EEYSAMRDQYMRTGEGFLCVFAINNTKSFEDIHHYREQIKRVKDSEDVPMVLVGNKCDL PSRTVDTKQAQDLARSYGIPFIETSAKTRQRVEDAFYTLVREIRQYRLKKISKEEKTPGCV KIKKCIIM (SEQ ID NO: 295). Surface K-Ras Antigen may further include other forms of K-Ras peptide or protein that are capable of being bound by various anti-K-Ras agents, including, for example, the antibodies, proteins and peptides listed in Table 1 and/or Table 2. Forms of K-Ras peptide or protein that are capable of being bound by various anti-K-Ras agents, include, for example, the antibodies, proteins and peptides listed in Table 1 and/or Table 2 include, but are not limited to, post-translationally modified K-Ras. For example, K-Ras post-translational modifications can include, but are not limited to, prenylation, post-prenylation, palmitoylation, ubiquitination, phosphorylation, SUMOylation, acetylation, nitrosylation, and combinations thereof. The antibodies, proteins and peptides of Table 1 and Table 2 are exemplary structures for selectively binding Surface K-Ras Antigen and may be used as the binding means or binding agent in any of the embodiments of the present disclosure directed to antibody-drug conjugates, chimeric antigen receptors, and bispecific antibodies. As used herein, the term “Surface K-Ras Antigen” does not include K-Ras-derived peptide-human leukocyte antigen (HLA) (major histocompatibility complex—MHC molecule) complexes or the individual K-Ras-derived peptides that are complexed with HLA and expressed on the surface.

TABLE 1
Exemplary Antibodies, Proteins and Peptides
that Bind Surface K-Ras Antigen.

| Antibody/ | | Recognition | | |
|---|---|---|---|---|
| binding | | as defined | | |
| Peptide | Source | by Source | tagged | Clone |
| Anti-Ras | Cell Signaling | Detects | None | Rabbit |
| G12D | Technology | G12D K-Ras | | monoclonal |
| antibody | (cat #14429) | mutation | | clone D8H7 |
| Anti-K-RAS | Iowa | K-Ras | None | Mouse |
| antibody | Hybridoma | | | monoclonal |
| | Center | | | clone 4E8 |
| Monoclonal | Sigma-Aldrich | K-Ras | None | Mouse |
| anti-K-Ras | (#cat | | | monoclonal |
| antibody | WH0003845M1) | | | clone 3B10- |
| | | | | 2F2 |
| Anti-K-RAS | GeneTex | Detects | None | rabbit |
| G12D | (cat # | G12D | | monoclonal- |
| antibody | GTX635362) | mutation | | Clone HL10 |
| Anti-K-RAS | Cell Signaling | Detects | None | rabbit |
| G12V | (cat # | G12V K-Ras | | monoclonal- |
| antibody | 14412S) | mutation | | Clone D2H12 |
| Engineered | Kauke et | Detects | His | R11.1.6 |
| anti-K-Ras | al 2017 | GTP-bound | tagged | |
| protein | | form of | | |
| | | G12D K-Ras | | |

TABLE 2
Additional Exemplary Antibodies, Proteins, and Peptides the Bind Surface
K-Ras Antigen.

| | VH | | VL | |
|---|---|---|---|---|
| anti-K-Ras | SEQ | | SEQ | |
| Antibody | ID NO: | VH Amino Acid Sequence | ID NO: | VL Amino Acid Sequence |
| Human | 9 | EVQLVQSGGGVVQPGRSLRLSCAASGFTSR | 13 | QSVVTQPPSVSAAPGQKVTISCSGSNS |
| Anti-K-Ras | | HPGMHWVRQAPGKGLEWVAVISHDGSKKYY | | NIGKNYVSWFQQVPGTAPKLLIFEDNQ |
| Antibody 1 | | ADSVKGRFTISRDNSKNTLFVQLSSLRPEDTA | | RPSGIPDRFSASKSGTSASLAISGLQSE |
| | | VYYCATSLYSSMDLWGQGTTVTVSSASTKG | | DEADYYCAAWDDKFGVHWVFGGGTK |
| | | PSVFPLAPSSKSTSGGTAALGCLVKDYFPEP | | LTVLGQPKAAPSVTLFPPSSEELQANK |
| | | VTVSWNSGALTSGVHTFPAVLQSSGLYSLSS | | ATLVCLISDFYPGAVTVAWKADSSPVK |
| | | VVTVPSSSLGTQTYICNVNHKPSNTKVDKKV | | AGVETTTPSKQSNNKYAASSYLSLTPE |
| | | EPKSCDKTHTCPPCPAPELLGGPSVFLFPPK | | QWKSHRSYSCQVTHEGSTVEKTVAPT |
| | | PKDTLMISRTPEVTCVVVDVSHEDPEVKFNW | | ECS |
| | | YVDGVEVHNAKTKPREEQYGSTYRVVSVLTV | | |
| | | LHQDWLNGKEYKCKVSNKALPAPIEKTISKAK | | |
| | | GQPREPQVYTLPPSREEMTKNQVSLTCLVKG | | |
| | | FYPSDIAVEWESNGQPENNYKTTPPVLDSDG | | |
| | | SFFLYSKLTVDKSRWQQGNVFSCSVMHEAL | | |
| | | HNHYTQKSLSLSPG | | |
| Human | 17 | EVQLLEPGGGVVQPGRSLRLSCTNSGFSFS | 21 | QSVLTQPASVSGSPGQSITISCTGTSN |
| Anti-K-Ras | | GYAMHWVRQAPGKGLEWVAVISFDGSHKYY | | DIGAYNYVSWYQQHPGKAPKLMIYDVN |
| Antibody 2 | | ADSVKGRFTISRDNSKNTLYLHMNSLRAEDT | | NRPSGVPDRFSGSKSGNMASLTISGLQ |
| | | AVYYCASGGNYYGSGTIVSHGMDVWGQGTT | | AEDDADYYCSSYTSSSTLVVFGGGTKL |
| | | VTVSSASTKGPSVFPLAPSSKSTSGGTAALG | | TVLGQPKAAPSVTLFPPSSEELQANKA |
| | | CLVKDYFPEPVTVSWNSGALTSGVHTFPAVL | | TLVCLISDFYPGAVTVAWKADSSPVKA |
| | | QSSGLYSLSSWVTVPSSSLGTQTYICNVNHKP | | GVETTTPSKQSNNKYAASSYLSLTPEQ |
| | | SNTKVDKKVEPKSCDKTHTCPPCPAPELLGG | | WKSHRSYSCQVTHEGSTVEKTVAPTE |
| | | PSVFLFPPKPKDTLMISRTPEVTCVVVDVSHE | | CS |
| | | DPEVKFNWYVDGVEVHNAKTKPREEQYGST | | |
| | | YRVVSVLTVLHQDWLNGKEYKCKVSNKALPA | | |
| | | PIEKTISKAKGQPREPQVYTLPPSREEMTKNQ | | |
| | | VSLTCLVKGFYPSDIAVEWESNGQPENNYKT | | |
| | | TPPVLDSDGSFFLYSKLTVDKSRWQQGNVFS | | |
| | | CSVMHEALHNHYTQKSLSLSPG | | |
| Human | 31 | EVQLVQSGGGVVQPGRSLRLSCAASGFTSS | 32 | QSVVTQPPSVSAAPGQKVTISCSGSNS |
| Anti-K-Ras | | NYGMHWVRQAPGKGLEWVAVISHDGSKKYY | | NIGKNYVSWFQQVPGTAPKLLIFEDNQ |
| Antibody 3 | | ADSVKGRFTISRDNSKNTLFVQLSSLRPEDTA | | RPSGIPDRFSASKSGTSASLAISGLQSE |
| | | VYYCATSLYSSMDLWGQGTTVTVSS | | DEADYYCAAWDDSLNAHWVFGGGTKL |
| | | | | TVL |
| Human | 33 | QVQLVESGGGVVQPGRSLRLSCTNSGFSFS | 34 | DIVMTQSPSTLSASVGDRVTITCRASQS |
| Anti-K-Ras | | GYAMHWVRQAPGKGLEWVAVISFDGSHKYY | | ISSWLAWYQQKPGKAPKVLIYKASSLE |
| Antibody 4 | | ADSVKGRFTISRDNSKNTLYLHMNSLRAEDT | | SGVPSRFSGSGSGTEFTLTISSLQPDD |
| | | AVYYCASGGNYYGSGTIVSHGLDFWGQGTT | | FAAYYCQHYNSYPYTFGQGTKLEIK |
| | | VTVSS | | |
| Human | 35 | QVQLVESGGGVVQPGRSLRLSCTNSGFSFS | 36 | SSELTQDPAVSVALGQTVRITCQGDSL |
| Anti-K-Ras | | GYAMHWVRQAPGKGLEWVAVISFDGSHKYY | | RSYYASWYQQKPGQAPVLVIYGKNNR |
| Antibody 5 | | ADSVKGRFTISRDNSKNTLYLHMNSLRAEDT | | PSGIPDRFSGSSSGNTASLTITGAQAED |
| | | AVYYCASGGNYYGSGTIVSHGMDWWGQGTT | | EADYYCNSRDSSGNHRGGGTKLTVL |
| | | VTVSS | | |
| Human | 37 | QVQLVESGGGVVQPGRSLRLSCSNSGFSFS | 38 | DIVMTQSPSSLSASVGDRVTITCRASQ |
| Anti-K-Ras | | GYTMHWVRQAPGKGLEWVAVISFDGSHKYY | | SISNYLNWYQQKPGKAPKLLIYAASSLQ |
| Antibody 6 | | ADSVKGRFTISRDNSKNTLYLHMNSLRAEDT | | SGVPSRFSGSGSGTDFTLTISSLQPED |
| | | AVYYCAKKMHYGSGAYHFDLWGQGTLVTVS | | FATYYCQQSYSSPFTFGPGTKVDIK |
| | | S | | |
| Human | 39 | EVQLLESGGGVVQPGRSLRLSCTNSGFSFS | 40 | QSVLTQPASVSGSPGQSITISCTGTSSD |
| Anti-K-Ras | | GYAMHWVRQAPGKGLEWVAVISFDGSHKYY | | VGGYNYVSWYQQHPGKAPKLMIYDVS |
| Antibody 7 | | ADSVKGRFTISRDNSKNTLYLHMNSLTAEDTA | | NRPSGVSNRFSGSKSGNTASLTISGLQ |
| | | VYYCASGGNYYGSGTIVSHGMDVWGQGTTV | | AEDEADYYCSSYTSSSTLVVFGGGTKL |
| | | TVSS | | TVL |
| Human | 41 | EVQLLEPGGGVVQPGRSLRLSCTNSGFSFS | 42 | QSALTQPRSVSGSPGQSVTISCTGTSS |
| Anti-K-Ras | | GYAMHWVRQAPGKGLEWVAVISFDGSHKYY | | NVGGYNHVSWYQQHPGKAPKVIIYDV |
| Antibody 8 | | ADSVKGRFTISRDNSKNTLYLHMNSLRAEDT | | NKRPSGVSHRFSGSKSANTASLTISGL |
| | | AVYYCASGGNYYGSGTIVSHGMDVWGQGTT | | QAEDEADYYCSSYTTSSTYVFGTGTKL |
| | | VTVSS | | TVL |
| Human | 43 | QVQLVESAGGVVQPGRSLRLSCTNSGFSFS | 44 | QSVLTQPASVSGSPGQSITISCTGTSSD |
| Anti-K-Ras | | GYAMHWVRQAPGKGLEWVAVISFDGSHKYY | | VGGYKYVSWYQQYPGKAPKLMIYDVS |
| Antibody 9 | | ADSVKGRFTISRDNSKNTLYLHMNSLTAEDTA | | KRPSGVSNRFSGSKSGNTASLTISGLQ |
| | | VYYCASGGNYYGSGTIVSHGMDVWGQGTTV | | AEDEADYYCNSYTSSRIYVFGTGTKLT |
| | | TVSS | | VL |
| Human | 45 | EVQLLEPGGGVVQPGRSLRLSCTNSGFSFS | 46 | QSVLTQPASVSGSPGQSITISCTGTSN |
| Anti-K-Ras | | GYAMHWVRQAPGKGLEWVAVISFDGSHKYY | | DIGAYNYVSWYQQHPGKAPKLMIYDVN |
| Antibody 10 | | ADSVKGRFTISRDNSKNTLYLHMNSLRAEDT | | NRPSGVPDRFSGSKSGNMASLTISGLQ |
| | | AVYYCASGGNYYGSGTIVSHGMDVWGQGTT | | AEDDADYYCSSYTSSSTLVVFGGGTKL |
| | | VTVSS | | TVL |
| Human | 47 | QVQLVQSGGGVVQPGRSLRLSCTNSGFSFS | 48 | QSVLTQPASVSGSPGQSITISCTGTSSD |
| Anti-K-Ras | | GYAMHWVRQAPGKGLEWVAVISFDGSHKYY | | IGGSNWVSWYQQHPGKAPKLMIYDVS |
| Antibody 11 | | ADSVKGRFTISRDNSKNTLYLHMNSVRAEDT | | KRPSGVSNRFSGSKSGNTASLTISGLQ |
| | | AVYYCASGGNHYGSGTIVSHGMDVRGQGTT | | AEDEADYYCSSYTSSSTYVFGTGTKLT |
| | | VTVSS | | VL |
| Human | 49 | QVQLVESGGGLVQPGGSLRLSCAASGFTFS | 50 | QAGLTQPASVSGSPGQSITISCTGTSS |
| Anti-K-Ras | | SYWMSWVRQAPGKGLEWVGFIRSKAYGGTT | | DVGGYNYVSWYQQHPGKAPKLMIYDV |
| Antibody 12 | | EYAASVKGRFTISRDDSKSIAYLQMNSLKTED | | SKRPSGVSNRFSGSKSGNTASLTISGL |
| | | TAVYYCTRDGGSYFDYWGQGTLVTVSS | | QAEDEADYYCSSYTSSSTYVFGTGTKV |
| | | | | TVL |
| Human | 51 | EVQLVQSGGGVVQPGRSLRLSCAASGFTSS | 52 | QSVVTQPPSVSAAPGQKVTISCSGSNS |
| Anti-K-Ras | | LNGMHWVRQAPGKGLEWVAVISHDGSKKYY | | NIGKNYVSWFQQVPGTAPKLLIFEDNQ |
| Antibody 13 | | ADSVKGRFTISRDNSKNTLFVQLSSLRPEDTA | | RPSGIPDRFSASKSGTSASLAISGLQSE |
| | | VYYCATSLYSSMDLWGQGTTVTVSS | | DEADYYCAAWDDYGSTHWVFGGGTK |
| | | | | LTVL |
| Human | 53 | EVQLVQSGGGVVQPGRSLRLSCAASGFTSR | 54 | QSVVTQPPSVSAAPGQKVTISCSGSNS |
| Anti-K-Ras | | QYGMHWVRQAPGKGLEWVAVISHDGSKKYY | | NIGKNYVSWFQQVPGTAPKLLIFEDNQ |
| Antibody 14 | | ADSVKGRFTISRDNSKNTLFVQLSSLRPEDTA | | RPSGIPDRFSASKSGTSASLAISGLQSE |
| | | VYYCATSLYSSMDLWGQGTTVTVSS | | DEADYYCAAWDDGRKVHWVFGGGTK |
| | | | | LTVL |
| Human | 55 | EVQLVQSGGGVVQPGRSLRLSCAASGFTSS | 56 | QSVVTQPPSVSAAPGQKVTISCSGSNS |
| Anti-K-Ras | | TYGMHWVRQAPGKGLEWVAVISHDGSKKYY | | NIGKNYVSWFQQVPGTAPKLLIFEDNQ |
| Antibody 15 | | ADSVKGRFTISRDNSKNTLFVQLSSLRPEDTA | | RPSGIPDRFSASKSGTSASLAISGLQSE |
| | | VYYCATSLYSSMDLWGQGTTVTVSS | | DEADYYCAAWDDSRTTHWVFGGGTKL |
| | | | | TVL |
| Human | 57 | EVQLVQSGGGVVQPGRSLRLSCAASGFTSR | 58 | QSVVTQPPSVSAAPGQKVTISCSGSNS |
| Anti-K-Ras | | PYGMHWVRQAPGKGLEWVAVISHDGSKKYY | | NIGKNYVSWFQQVPGTAPKLLIFEDNQ |
| Antibody 16 | | ADSVKGRFTISRDNSKNTLFVQLSSLRPEDTA | | RPSGIPDRFSASKSGTSASLAISGLQSE |
| | | VYYCATSLYSSMDLWGQGTTVTVSS | | DEADYYCAAWDDFHSEHWVFGGGTKL |
| | | | | TVL |
| Human | 59 | EVQLVQSGGGVVQPGRSLRLSCAASGFTSA | 60 | QSVVTQPPSVSAAPGQKVTISCSGSNS |
| Anti-K-Ras | | TYGMHWVRQAPGKGLEWVAVISHDGSKKYY | | NIGKNYVSWFQQVPGTAPKLLIFEDNQ |
| Antibody 17 | | ADSVKGRFTISRDNSKNTLFVQLSSLRPEDTA | | RPSGIPDRFSASKSGTSASLAISGLQSE |
| | | VYYCATSLYSSMDLWGQGTTVTVSS | | DEADYYCAAWDDSSDTHWVFGGGTKL |
| | | | | TVL |
| Human | 61 | EVQLVQSGGGVVQPGRSLRLSCAASGFTSS | 62 | QSVVTQPPSVSAAPGQKVTISCSGSNS |
| Anti-K-Ras | | FHGMHWVRQAPGKGLEWVAVISHDGSKKYY | | NIGKNYVSWFQQVPGTAPKLLIFEDNQ |
| Antibody 18 | | ADSVKGRFTISRDNSKNTLFVQLSSLRPEDTA | | RPSGIPDRFSASKSGTSASLAISGLQSE |
| | | VYYCATSLYSSMDLWGQGTTVTVSS | | DEADYYCAAWDDSGDVHWVFGGGTK |
| | | | | LTVL |
| Human | 63 | EVQLVQSGGGVVQPGRSLRLSCAASGFTSR | 64 | QSVVTQPPSVSAAPGQKVTISCSGSNS |
| Anti-K-Ras | | HPGMHWVRQAPGKGLEWVAVISHDGSKKYY | | NIGKNYVSWFQQVPGTAPKLLIFEDNQ |
| Antibody 19 | | ADSVKGRFTISRDNSKNTLFVQLSSLRPEDTA | | RPSGIPDRFSASKSGTSASLAISGLQSE |
| | | VYYCATSLYSSMDLWGQGTTVTVSS | | DEADYYCAAWDDYRGPHWVFGGGTK |
| | | | | LTVL |
| Human | 65 | EVQLVQSGGGVVQPGRSLRLSCAASGFTSR | 66 | QSVVTQPPSVSAAPGQKVTISCSGSNS |
| Anti-K-Ras | | HPGMHWVRQAPGKGLEWVAVISHDGSKKYY | | NIGKNYVSWFQQVPGTAPKLLIFEDNQ |
| Antibody 20 | | ADSVKGRFTISRDNSKNTLFVQLSSLRPEDTA | | RPSGIPDRFSASKSGTSASLAISGLQSE |
| | | VYYCATSLYSSMDLWGQGTTVTVSS | | DEADYYCAAWDDYRGPHWVFGGGTK |
| | | | | LTVL |
| Human | 67 | EVQLVQSGGGVVQPGRSLRLSCAASGFTSR | 68 | QSVVTQPPSVSAAPGQKVTISCSGSNS |
| Anti-K-Ras | | APGMHWVRQAPGKGLEWVAVISHDGSKKYY | | NIGKNYVSWFQQVPGTAPKLLIFEDNQ |
| Antibody 21 | | ADSVKGRFTISRDNSKNTLFVQLSSLRPEDTA | | RPSGIPDRFSASKSGTSASLAISGLQSE |
| | | VYYCATSLYSSMDLWGQGTTVTVSS | | DEADYYCAAWDDHNGEHWVFGGGTK |
| | | | | LTVL |
| Human | 69 | EVQLVQSGGGVVQPGRSLRLSCAASGFTSR | 70 | QSVVTQPPSVSAAPGQKVTISCSGSNS |
| Anti-K-Ras | | HPGMHWVRQAPGKGLEWVAVISHDGSKKYY | | NIGKNYVSWFQQVPGTAPKLLIFEDNQ |
| Antibody 22 | | ADSVKGRFTISRDNSKNTLFVQLSSLRPEDTA | | RPSGIPDRFSASKSGTSASLAISGLQSE |
| | | VYYCATSLYSSMDLWGQGTTVTVSS | | DEADYYCAAWDDKFGVHWVFGGGTK |
| | | | | LTVL |
| Human | 71 | EVQLVQSGGGVVQPGRSLRLSCAASGFTSR | 72 | QSVVTQPPSVSAAPGQKVTISCSGSNS |
| Anti-K-Ras | | KYGMHWVRQAPGKGLEWVAVISHDGSKKYY | | NIGKNYVSWFQQVPGTAPKLLIFEDNQ |
| Antibody 23 | | ADSVKGRFTISRDNSKNTLFVQLSSLRPEDTA | | RPSGIPDRFSASKSGTSASLAISGLQSE |
| | | VYYCATSLYSSMDLWGQGTTVTVSS | | DEADYYCAAWDDSPTIHWVFGGGTKL |
| | | | | TVL |
| Human | 73 | EVQLVQSGGGVVQPGRSLRLSCAASGFTSH | 74 | QSVVTQPPSVSAAPGQKVTISCSGSNF |
| Anti-K-Ras | | NKGMHWVRQAPGKGLEWVAVISHDGSKKYY | | NIGKNYVSWFQQVPGTAPKLLIFEDNQ |
| Antibody 24 | | ADSVKGRFTISRDNSKNTLFVQLSSLRPEDTA | | RPSGIPDRFSASKSGTSASLAISGLQSE |
| | | VYYCATSLYSSMDLWGQGTTVTVSS | | DEADYYCAAWDDFAGVHWVFGGGTK |
| | | | | LTVL |
| Human | 75 | EVQLVQSGGGVVQPGRSLRLSCAASGFTSY | 76 | QSVVTQPPSVSAAPGQKVTISCSGSNS |
| Anti-K-Ras | | KYGMHWVRQAPGKGLEWVAVISHDGSKKYY | | NIGKNYVSWFQQVPGTAPKLLIFEDNQ |
| Antibody 25 | | ADSVKGRFTISRDNSKNTLFVQLSSLRPEDTA | | RPSGIPDRFSASKSGTSASLAISGLQSE |
| | | VYYCASSLYSSMDLWGQGTTVTVSS | | DEADYYCAAWDDSFSEHWVFGGGTKL |
| | | | | TVL |
| Human | 77 | EVQLVQSGGGVVQPGRSLRLSCAASGFTSR | 78 | QSVVTQPPSVSAAPGQKVTISCSGSNS |
| Anti-K-Ras | | APGMHWVRQAPGKGLEWVAVISHDGSKKYY | | NIGKNYVSWFQQVPGTAPKLLIFEDNQ |
| Antibody 26 | | ADSVKGRFTISRDNSKNTLFVQLSSLRPEDTA | | RPSGIPDRFSASKSGTSASLAISGLQSE |
| | | VYYCATSLYSSMDLWGQGTTVTVSS | | DEADYYCAAWDDQNGVHWVFGGGTK |
| | | | | LTVL |
| Human | 79 | EVQLVQSGGGVVQPGRSLRLSCAASGFTSR | 80 | QSVVTQPPSVSAAPGQKVTISCSGSNS |
| Anti-K-Ras | | SPGMHWVRQAPGKGLEWVAVISHDGSKKYY | | NIGKNYVSWFQQVPGTAPKLLIFEDNQ |
| Antibody 27 | | ADSVKGRFTISRDNSKNTLFVQLSSLRPEDTA | | RPSGIPDRFSASKSGTSASLAISGLQSE |
| | | VYYCATSLYSSMDLWGQGTTVTVSS | | DEADYYCAAWDDLRGVHWVFGGGTK |
| | | | | LTVL |
| Human | 81 | EVQLVQSGGGVVQPGRSLRLSCAASGFTSR | 82 | QSVVTQPPSVSAAPGQKVTISCSGSNS |
| Anti-K-Ras | | HHGMHWVRQAPGKGLEWVAVISHDGSKKYY | | NIGKNYVSWFQQVPGTAPKLLIFEDNQ |
| Antibody 28 | | ADSVKGRFTISRDNSKNTLFVQLSSLRPEDTA | | RPSGIPDRFSASKSGTSASLAISGLQSE |
| | | VYYCATSLYSSMDLWGQGTTVTVSS | | DEADYYCAAWDDRHGTHWVFGGGTK |
| | | | | LTVL |
| Human | 83 | EVQLVQSGGGVVQPGRSLRLSCAASGFTSR | 84 | QSVVTQPPSVSAAPGQKVTISCSGSNS |
| Anti-K-Ras | | NPGMHWVRQAPGKGLEWVAVISHDGSKKYY | | NIGKNYVSWFQQVPGTAPKLLIFEDNQ |
| Antibody 29 | | ADSVKGRFTISRDNSKNTLFVQLSSLRPEDTA | | RPSGIPDRFSASKSGTSASLAISGLQSE |
| | | VYYCATSLYSSMDLWGQGTTVTVSS | | DEADYYCAAWDDSTGVHWVFGGGTK |
| | | | | LTVL |
| Human | 85 | EVQLVQSGGGVVQPGRSLRLSCAASGFTSR | 86 | QSVVTQPPSVSAAPGQKVTISCSGSNS |
| Anti-K-Ras | | TYGMHWVRQAPGKGLEWVAVISHDGSKKYY | | NIGKNYVSWFQQVPGTAPKLLIFEDNQ |
| Antibody 30 | | ADSVKGRFTISRDNSKNTLFVQLSSLRPEDTA | | RPSGIPDRFSASKSGTSASLAISGLQSE |
| | | VYYCATSLYSSMDLWGQGTTVTVSS | | DEADYYCAAWDDLPTDHWVFGGGTKL |
| | | | | TVL |
| Human | 87 | EVQLVQSGGGVVQPGRSLRLSCAASGFTSR | 88 | QSVVTQPPSVSAAPGQKVTISCSGSNS |
| Anti-K-Ras | | QYGMHWVRQAPGKGLEWVAVISHDGSKKYY | | NIGKNYVSWFQQVPGTAPKLLIFEDNQ |
| Antibody 31 | | ADSVKGRFTISRDNSKNTLFVQLSSLRPEDTA | | RPSGIPDRFSASKSGTSASLAISGLQSE |
| | | VYYCATSLYSSMDLWGQGTTVTVSS | | DEADYYCAAWDDYSGVHWVFGGGTK |
| | | | | LTVL |
| Human | 89 | EVQLVQSGGGVVQPGRSLRLSCAASGFTSS | 90 | QSVVTQPPSVSAAPGQKVTISCSGSNS |
| Anti-K-Ras | | PYGMHWVRQAPGKGLEWVAVISHDGSKKYY | | NIGKNYVSWFQQVPGTAPKLLIFEDNQ |
| Antibody 32 | | ADSVKGRFTISRDNSKNTLFVQLSSLRPEDTA | | RPSGIPDRFSASKSGTSASLAISGLQSE |
| | | VYYCATSLYSSMDLWGQGTTVTVSS | | DEADYYCAAWDDNPRDHWVFGGGTK |
| | | | | LTVL |
| Human | 91 | EVQLVQSGGGVVQPGRSLRLSCAASGFTSS | 92 | QSVVTQPPSVSAAPGQKVTISCSGSNS |
| Anti-K-Ras | | KPGMHWVRQAPGKGLEWVAVISHDGSKKYY | | NIGKNYVSWFQQVPGTAPKLLIFEDNQ |
| Antibody 33 | | ADSVKGRFTISRDNSKNTLFVQLSSLRPEDTA | | RPSGIPDRFSASKSGTSASLAISGLQSE |
| | | VYYCATSLYSSMDLWGQGTTVTVSS | | DEADYYCAAWDDLRGVHWVFGGGTK |
| | | | | LTVL |
| Human | 93 | EVQLVQSGGGVVQPGRSLRLSCAASGFTSK | 94 | QSVVTQPPSVSAAPGQKVTISCSGSNS |
| Anti-K-Ras | | KYGMHWVRQAPGKGLEWVAVISHDGSKKYY | | NIGKNYVSWFQQVPGTAPKLLIFEDNQ |
| Antibody 34 | | ADSVKGRFTISRDNSKNTLFVQLSSLRPEDTA | | RPSGIPDRFSASKSGTSASLAISGLQSE |
| | | VYYCATSLYSSMDLWGQGTTVTVSS | | DEADYYCAAWDDLGGDHWVFGGGTK |
| | | | | LTVL |
| Human | 95 | EVQLVQSGGGVVQPGRSLRLSCAASGFTSTL | 96 | QSVVTQPPSVSAAPGQKVTISCSGSNS |
| Anti-K-Ras | | PGMHWVRQAPGKGLEWVAVISHDGSKKYYA | | NIGKNYVSWFQQVPGTAPKLLIFEDNQ |
| Antibody 35 | | DSVKGRFTISRDNSKNTLFVQLSSLRPEDTAV | | RPSGIPDRFSASKSGTSASLAISGLQSE |
| | | YYCATSLYSSMDLWGQGTTVTVSS | | DEADYYCAAWDDSAGVHWVFGGGTK |
| | | | | LTVL |
| Human | 97 | EVQLVQSGGGVVQPGRSLRLSCAASGFTSK | 98 | QSVVTQPPSVSAAPGQKVTISCSGSNS |
| Anti-K-Ras | | MPGMHWVRQAPGKGLEWVAVISHDGSKKY | | NIGKNYVSWFQQVPGTAPKLLIFEDNQ |
| Antibody 36 | | YADSVKGRFTISRDNSKNTLFVQLSSLRPEDT | | RPSGIPDRFSASKSGTSASLAISGLQSE |
| | | AVYYCATSLYSSMDLWGQGTTVTVSS | | DEADYYCAAWDDEHGVHWVFGGGTK |
| | | | | LTVL |
| Human | 99 | EVQLVQSGGGVVQPGRSLRLSCAASGFTSS | 100 | QSVVTQPPSVSAAPGQKVTISCSGSNS |
| Anti-K-Ras | | NYGMHWVRQAPGKGLEWVAVISHDGSKKYY | | NIGKNYVSWFQQVPGTAPKLLIFEDNQ |
| Antibody 37 | | ADSVKGRFTISRDNSKNTLFVQLSSLRPEDTA | | RPSGIPDRFSASKSGTSASLAISGLQSE |
| | | VYYCAASLYSSMDLWGQGTTVTVSS | | DEADYYCAAWDDSLSAHWVFGGGTKL |
| | | | | TVL |
| Human | 101 | EVQLVQSGGGVVQPGRSLRLSCAASGFTSS | 102 | QSVVTQPPSVSAAPGQKVTISCSGSNS |
| Anti-K-Ras | | NYGMHWVRQAPGKGLEWVAVISHDGSKKYY | | NIGKNYVSWFQQVPGTAPKLLIFEDNQ |
| Antibody 38 | | ADSVKGRFTISRDNSKNTLFVQLSSLRPEDTA | | RPSGIPDRFSASKSGTSASLAISGLQSE |
| | | VYYCATSMVSSMDLWGQGTTVTVSS | | DEADYYCAAWDDSLSTHWVFGGGTKL |
| | | | | TVL |
| Human | 103 | EVQLVQSGGGVVQPGRSLRLSCAASGFTSS | 104 | QSVVTQPPSVSAAPGQKVTISCSGSNS |
| Anti-K-Ras | | NYGMHWVRQAPGKGLEWVAVISHDGSKKYY | | NIGKNYVSWFQQVPGTAPKLLIFEDNQ |
| Antibody 39 | | ADSVKGRFTISRDNSKNTLFVQLSSLRPEDTA | | RPSGIPDRFSASKSGTSASLAISGLQSE |
| | | VYYCATSMRSSMDLWGQGTTVTVSS | | DEADYYCAAWDDSLSAHWVFGGGTKL |
| | | | | TVL |
| Human | 105 | EVQLVQSGGGVVQPGRSLRLSCAASGFTSS | 106 | QSVVTQPPSVSAAPGQKVTISCSGSNS |
| Anti-K-Ras | | NYGMHWVRQAPGKGLEWVAVISHDGSKKYY | | NIGKNYVSWFQQVPGTAPKLLIFEDNQ |
| Antibody 40 | | ADSVKGRFTISRDNSKNTLFVQLSSLRPEDTA | | RPSGIPDRFSASKSGTSASLAISGLQSE |
| | | VYYCATSLYSSMDLWGQGTTVTVSS | | DEADYYCAAWDDSLSAHWVFGGGTKL |
| | | | | TVL |
| Human | 107 | EVQLVESGGGLVQPGGSLRLSCAASGFTFSS | 108 | AIQMTQSPSSLSASVGDRVTITCRASQ |
| Anti-K-Ras | | YSMNWVRQAPGKGLEWVSYISSSSSTIYYAD | | GIRNDLGWYQQKPGKAPKLLIYAASSL |
| Antibody 41 | | SVKGRFTISRDNAKNSLYLQMNSLRAEDTAV | | QSGVPSRFSGSGSGTDFTLTISSLQPE |
| | | YYCARGFYVRNWFDPWGQGTLVTVSS | | DFATYYCLQDHDYPLTFGQGTKVEIK |
| Human | 109 | EVQLVESGGGLVQPGGSLRLSCAASGFTFSS | 110 | DIQMTQSPSSLSASVGDRVTITCRASQ |
| Anti-K-Ras | | YAMSWVRQAPGKGLEWVSAISSSGSSTYYA | | GISSYLAWYQQKPGKAPKLLIYAASSLQ |
| Antibody 42 | | DSVKGRFTISRDNSKNTLYLQMNSLRAEDTA | | SGVPSRFSGSGSGTDFTLTISSLQPED |
| | | VYYCARDQGGYGYPGESWFDYWGQGTLVT | | FATYYCQQYYSYPFTFGQGTKVEIK |
| | | VSS | | |
| Human | 111 | EVQLVESGGGLVKPGGSLRLSCAASGFTFSS | 112 | DIQMTQSPSSLSASVGDRVTITCRASQ |
| Anti-K-Ras | | YSMNWVRQAPGKGLEWVSSISSSSSYIYYAD | | SISSYLNWYQQKPGKAPKLLIYAASSLQ |
| Antibody 43 | | SVKGRFTISRDNAKNSLYLQMNSLRAEDTAV | | SGVPSRFSGSGSGTDFTLTISSLQPED |
| | | YYCARAFYSYMDVWGQGTLVTVSS | | FATYYCQQSYSPPWTFGQGTKVEIK |
| Human | 113 | EVQLQESGPGLVKPPGTLSLTCAVSGGSISS | 114 | DIVMTQSPLSLPVTPGEPASISCRSSQS |
| Anti-K-Ras | | SNWWSWVRQPPGKGLEWIGEIYHSGSTNYN | | LLHSNGYNYLDWYLQKPGQSPQLLIYL |
| Antibody 44 | | PSLKSRVTISVDKSKNQFSLKLSSVTAADTAV | | GSNRASGVPDRFSGSGSGTDFTLKISR |
| | | YYCARERTILTGYYGFDYWGQGTLVTVSS | | VEAEDVGVYYCMQALQTPLTFGQGTK |
| | | | | VEIK |
| Human | 115 | EVQLVQSGAEVKKPGSSVKVSCKASGGTFS | 116 | SVLTQPPSVSAAPGQKVTISCSGSSSNI |
| Anti-K-Ras | | SYAISWVRQAPGQGLEWMGGIIPIFGTANYA | | GNNYVSWYQQLPGTAPKLLIYDNNKRP |
| Antibody 45 | | QKFQGRVTITADESTSTAYMELSSLRSEDTAV | | SGIPDRFSGSKSGTSATLGITGLQTGDE |
| | | YYCARYYDFWSGYPGGLFDWWGQGTLVTVS | | ADYYCGTWDSSLTGYVFGGGTKLTVL |
| | | S | | |
| Human | 117 | EVQLVQSGAEVKKPGSSVKVSCKASGGTFS | 118 | SVLTQPPSVSAAPGQKVTISCSGSSSNI |
| Anti-K-Ras | | SYAISWVRQAPGQGLEWMGGIIPIFGTANYA | | GNNYVSWYQQLPGTAPKLLIYDNNKRP |
| Antibody 46 | | QKFQGRVTITADESTSTAYMELSSLRSEDTAV | | SGIPDRFSGSKSGTSATLGITGLQTGDE |
| | | YYCARYYDFWSGYPGGLFDVWGQGTLVTVS | | ADYYCGTWDSSLTGWVFGGGTKLTVL |
| | | S | | |
| Human | 119 | EVQLVESGGGLVKPGGSLRLSCAASGFTFSS | 120 | LTQDPAVSVALGQTVRITCQGDSLRSY |
| Anti-K-Ras | | YSMNWVRQAPGKGLEWVSSISSSSSYIYYAD | | YASWYQQKPGQAPVLVIYGKNNRPSGI |
| Antibody 47 | | SVKGRFTISRDNAKNSLYLQMNSLRAEDTAV | | PDRFSGSSSGNTASLTITGAQAEDEAD |
| | | YYCARTNNYGYRYFDYWGQGTLVTVSS | | YYCNSRDSSGNHWVFGGGTKLTVL |
| Human | 121 | EVQLVESGGGLVKPGGSLRLSCAASGFTFSS | 122 | ELTQDPAVSVALGQTVRITCQGDSLRS |
| Anti-K-Ras | | YSMNWVRQAPGKGLEWVSSISSSSSYIYYAD | | YYASWYQQKPGQAPVLVIYGKNNRPS |
| Antibody 48 | | SVKGRFTISRDNAKNSLYLQMNSLRAEDTAV | | GIPDRFSGSSSGNTASLTITGAQAEDEA |
| | | YYCARATSSGYYYFDYWGQGTLVTVSS | | DYYCNSRDSTDNHLWVFGGGTKLTVL |
| Human | 123 | EVQLQESGPGLVKPPGTLSLTCAVSGGSISS | 124 | SVLTQPPSASGTPGQRVTISCSGSSSN |
| Anti-K-Ras | | SNWWSWVRQPPGKGLEWIGEIYHSGSTNYN | | IGSNYVYWYQQLPGTAPKLLIYRNNQR |
| Antibody 49 | | PSLKSRVTISVDKSKNQFSLKLSSVTAADTAV | | PSGVPDRFSGSKSGTSASLAISGLRSE |
| | | YYCARGSSSWYDLGPFDYWGQGTLVTVSS | | DEADYYCAAWDERLSGWVFGGGTKLT |
| | | | | VL |
| Human | 125 | EVQLVESGGGLVQPGGSLRLSCAASGFTFSS | 126 | SVLTQPPSVSAAPGQKVTISCSGSSSNI |
| Anti-K-Ras | | YSMNWVRQAPGKGLEWVSYISSSSSTIYYAD | | GNNYVSWYQQLPGTAPKLLIYDNNKRP |
| Antibody 50 | | SVKGRFTISRDNAKNSLYLQMNSLRAEDTAV | | SGIPDRFSGSKSGTSATLGITGLQTGDE |
| | | YYCARGKGIVGWGFFGMDVWGQGTLVTVSS | | ADYYCGTWDNSLSVWVVFGGGTKLTVL |
| Human | 127 | EVQLVESGGGLVQPGGSLRLSCAASGFTFSS | 128 | VLTQPPSASGTPGQRVTISCSGSSSNI |
| Anti-K-Ras | | YSMNWVRQAPGKGLEWVSYISSSSSTIYYAD | | GSNYVYWYQQLPGTAPKLLIYRNNQR |
| Antibody 51 | | SVKGRFTISRDNAKNSLYLQMNSLRAEDTAV | | PSGVPDRFSGSKSGTSASLAISGLRSE |
| | | YYCARSFGPYAFDVWGQGTLVTVSS | | DEADYYCAAWDDSLSGWVFGGGTKLT |
| | | | | VL |
| Human | 129 | EVQLQESGPGLVKPPGTLSLTCAVSGSSIWS | 130 | SVLTQPPSASGTPGQRVTISCSGSSSN |
| Anti-K-Ras | | SNWWSWVRQPPGKGLEWIGEIYHSGSTNYN | | IGSNYVYWYQQLPGTAPKLLIYRNNQR |
| Antibody 52 | | PSLKSRVTISVDKSKNQFSLKLSSVTAADTAV | | PSGVPDRFSGSKSGTSASLAISGLRSE |
| | | YYCARGSSSWYDLGPFDYWGQGTLVTVSS | | DEADYYCAAWDERLSGWVFGGGTKLT |
| | | | | VL |
| Human | 131 | EVQLQESGPGLVKPPGTLSLTCAVSGSNISS | 132 | SVLTQPPSASGTPGQRVTISCSGSSSN |
| Anti-K-Ras | | SNWWSWVRQPPGKGLEWIGEIYHSGSTNYN | | IGSNYVYWYQQLPGTAPKLLIYRNNQR |
| Antibody 53 | | PSLKSRVTISVDKSKNQFSLKLSSVTAADTAV | | PSGVPDRFSGSKSGTSASLAISGLRSE |
| | | YYCARGSSSWYDLGPFDYWGQGTLVTVSS | | DEADYYCAAWDERLSGWVFGGGTKLT |
| | | | | VL |
| Human | 133 | EVQLQESGPGLVKPPGTLSLTCAVSGSSIFSS | 134 | SVLTQPPSASGTPGQRVTISCSGSSSN |
| Anti-K-Ras | | NWWSWVRQPPGKGLEWIGEIYHSGSTNYNP | | IGSNYVYWYQQLPGTAPKLLIYRNNQR |
| Antibody 54 | | SLKSRVTISVDKSKNQFSLKLSSVTAADTAVY | | PSGVPDRFSGSKSGTSASLAISGLRSE |
| | | YCARGSSSWYDLGPFDYWGQGTLVTVSS | | DEADYYCAAWDERLSGWVFGGGTKLT |
| | | | | VL |
| Human | 135 | EVQLQESGPGLVKPPGTLSLTCAVSGSSIMS | 136 | SVLTQPPSASGTPGQRVTISCSGSSSN |
| Anti-K-Ras | | SNWWSWVRQPPGKGLEWIGEIYHSGSTNYN | | IGSNYVYWYQQLPGTAPKLLIYRNNQR |
| Antibody 55 | | PSLKSRVTISVDKSKNQFSLKLSSVTAADTAV | | PSGVPDRFSGSKSGTSASLAISGLRSE |
| | | YYCARGSSSWYDLGPFDYWGQGTLVTVSS | | DEADYYCAAWDERLSGWVFGGGTKLT |
| | | | | VL |
| Human | 137 | EVQLQESGPGLVKPPGTLSLTCAVSGSSIYSS | 138 | SVLTQPPSASGTPGQRVTISCSGSSSN |
| Anti-K-Ras | | NWWSWVRQPPGKGLEWIGEIYHSGSTNYNP | | IGSNYVYWYQQLPGTAPKLLIYRNNQR |
| Antibody 56 | | SLKSRVTISVDKSKNQFSLKLSSVTAADTAVY | | PSGVPDRFSGSKSGTSASLAISGLRSE |
| | | YCARGSSSWYDLGPFDYWGQGTLVTVSS | | DEADYYCAAWDERLSGWVFGGGTKLT |
| | | | | VL |
| Human | 139 | EVQLQESGPGLVKPPGTLSLTCAVSGGNIWS | 140 | SVLTQPPSASGTPGQRVTISCSGSSSN |
| Anti-K-Ras | | SNWWSWVRQPPGKGLEWIGEIYHSGSTNYN | | IGSNYVYWYQQLPGTAPKLLIYRNNQR |
| Antibody 57 | | PSLKSRVTISVDKSKNQFSLKLSSVTAADTAV | | PSGVPDRFSGSKSGTSASLAISGLRSE |
| | | YYCARGSSSWYDLGPFDYWGQGTLVTVSS | | DEADYYCAAWDERLSGWVFGGGTKLT |
| | | | | VL |
| Human | 141 | EVQLQESGPGLVKPPGTLSLTCAVSKGSIWA | 142 | SVLTQPPSASGTPGQRVTISCSGSSSN |
| Anti-K-Ras | | SHWWSWVRQPPGKGLEWIGEIYHSGSTNYN | | IGSNYVYWYQQLPGTAPKLLIYRNNQR |
| Antibody 58 | | PSLKSRVTISVDKSKNQFSLKLSSVTAADTAV | | PSGVPDRFSGSKSGTSASLAISGLRSE |
| | | YYCARGSSSWYDLGPFDYWGQGTLVTVSS | | DEADYYCAAWDERLSGWVFGGGTKLT |
| | | | | VL |
| Human | 143 | EVQLQESGPGLVKPPGTLSLTCAVSKGSIWS | 144 | SVLTQPPSASGTPGQRVTISCSGSSSN |
| Anti-K-Ras | | SNWWSWVRQPPGKGLEWIGEIYHSGSTNYN | | IGSNYVYWYQQLPGTAPKLLIYRNNQR |
| Antibody 59 | | PSLKSRVTISVDKSKNQFSLKLSSVTAADTAV | | PSGVPDRFSGSKSGTSASLAISGLRSE |
| | | YYCARGSSSWYDLGPFDYWGQGTLVTVSS | | DEADYYCAAWDERLSGWVFGGGTKLT |
| | | | | VL |
| Human | 145 | EVQLLESGGGLVQPGGSLRLSCAASGFTFST | 146 | IQMTQSPSSLSASVGDRVTITCRASQSI |
| Anti-K-Ras | | FSMNWVRQAPGKGLEWVSYISRTSKTIYYAD | | SSYLNWYQQKPGEAPKLLIYSASVLQS |
| Antibody 60 | | SVKGRFTISRDNSKNTLYLQMNSLRAEDTAV | | GVPSRFSGSGSGTDFTLTISSLQPEDF |
| | | YYCARGRFFDYWGQGTLVTVSS | | ATYYCQQSVMIPMTFGQGTKVE |
| Human | 147 | QIQLQESGPGLVKPSQSLSLTCTVTGYSITSD | 148 | DICKTQSHKFMSTSVGDRVSITCKASQ |
| Anti-K-Ras | | YAWNWIRQFPGNKLEWMGYINYSGSTSYNP | | DVGTAVAWYQQKPGQSPKLLIYWAST |
| Antibody 61 | | SLKSRISITRDTSKNQFFLQLISVTSEDTATYY | | RHTGVPDRFTGSESGTDFTLTISNVQS |
| | | CAGSGYVRGFAYWGQGTLVTVSA | | EDLADYFCQQYSSYPYTFGGGTKLEIK |
| Human | 149 | EVQLQQSGPELVKPGASVKMSCKTSGYTFT | 150 | ENVLTQSPAIMSASPGEKVTMTCSAGS |
| Anti-K-Ras | | DYTMHWVKQSHGKSLEWIGYINPNNGGTYY | | SVSYMHWYQQKSSTSPKLWIYDTSKLA |
| Antibody 62 | | NQRFKGKATLTVSRSSSTAYIELRSLTSDDSA | | SGVPGRFSGSGSGNSYSLTISSMEAED |
| | | VYYCARGDYYGGGYWGQGTTLRVSS | | VATYYCFQGSGYPLTFGDGTKLELK |
| Human | 151 | EVQLQQSGPERVKPGASVKMSCKASGYTFT | 152 | ENVLTQSPAIMSASPGEKVTMTCSASS |
| Anti-K-Ras | | DYSMHWVKQSHGKSLEWIGYINPNNGGASY | | SVSYMHWYQQKSSTSPKLWIYDTSKV |
| Antibody 63 | | NQKFKGKATLTVNKSSSTAYMELRSLTSEDS | | ASGVPGRFSGSGSGNSYSLTINSMEAE |
| | | AVYYCARGDYYGGGYWGQGTTLTVSS | | DVATYYCFQGSGYPLTFGAGTKLELK |
| Human | 153 | QVQLQQPGAEFVKPGASVKLSCKASGYTFTR | 154 | ENVLTQSPAIMSASPGEKVTMTCSAGS |
| Anti-K-Ras | | YWMHWVKQRPGRGLEWIGRIDPNSGGTTFN | | SVDYMHWYQQKSSTSPKLWIYDTSKL |
| Antibody 64 | | EKFKSKATLTVDKPSSTAYMQLSSLTSEDSAV | | ASGVPGRFSGSGSGNSYSLTISSMEAE |
| | | YYCAREVGGYWGQGTTLTVSS | | DVATYYCFQGSGYPLTFGAGTKLELR |
| Human | 155 | QVTLKESGPGILQPSQTLSLTCSFSGFSLSTF | 156 | NIVMTQSPKSMSMSVGERVTLSCKASE |
| Anti-K-Ras | | GMGVGWIRQPSGKGLEWLAHIWWDDDKNY | | NVGTYVSWYQQKPGQSPKLLIYGASN |
| Antibody 65 | | NPALKSRLTISKDTSKNQVFFKIANVDTADTAT | | RYTGVPDRFTGSGSGTDFILTISSVQAE |
| | | YYCVQLSTMGDGGYWGQGTLVTVSA | | DLADYHCGQSYNYPFTFGSGTKLEIK |
| Human | 157 | QVTLKESGPGILQPSQTLSLTCSFSGFSLSTF | 158 | NIVMTQSPKSMSMSVGERVTLSCKASE |
| Anti-K-Ras | | GVGVAWIRQPSGKGLEWLAHIWWDDDKNYD | | NVGTFVSWYQQKPDQSPKLLIYGASNR |
| Antibody 66 | | PALKSRLTISKDTSKNQVFLKIANVDTTDSATY | | YTGVPDRFTGSGSATDFTLTISSVQAE |
| | | YCARIGSTLITPGDASWGQGTLVTVSA | | DLADYHCGQSYSYPYTFGGGTKLEIK |
| Human | 159 | QVTLKESGPGILQPSQTLSLTCSFSGFSLSTF | 160 | DIVLTQSPATLSVTPGDRVSLSCRASQ |
| Anti-K-Ras | | GVGVAWIRQPSGKGLEWLAHIWWDDDKNYD | | SISDYLHWYQQKSHESPRLLIKFASQSI |
| Antibody 67 | | PALKSRLTISKDTSKNQVFLKIANVDTTDSATY | | SGIPSRFSGSGSGTDFTLSINSVETEDF |
| | | YCARIGSTLITPGDASWGQGTLVTVSA | | GMYFCQQSNNWPHMYTFGGGTKLEIK |
| Human | 161 | DIQLQESGPGLVKPSQSLSLTCSVTGYSITSD | 162 | DVLMTQTPLSLPVSLGDQASISCRSSQ |
| Anti-K-Ras | | YYWNWIRQFPGNKLEWMGYIGYDGTNNYNP | | TIVHGNGNTYLEWYLQKPGQSPKLLIYT |
| Antibody 68 | | SLKNRISITRDTSKNQFFLKLNSVTAEDTATYY | | VSNRFSGVPDRFSGSGSGTDFTLKISR |
| | | CARLWDYWGQGTTLTVSS | | VEAEDLGVYYCFQGSHAPYTFGGGTK |
| | | | | LEIK |
| Human | 163 | QVQLQQSGAELVKPGASVKLSCKASGYTFTS | 164 | DIVITQDELSNPVISGESVSISCRSSKSL |
| Anti-K-Ras | | YYMYWVKQRPGQGLEWIGEINPSNGGTNFN | | LYKDGKTYLNWFLQRPGQSPQLLIYLM |
| Antibody 69 | | EKFKSKATLTVDKSSGTAYMQLSSLTSEDSAI | | STRASGVSDRFSGSGSGTDFTLEISRV |
| | | YYCTRGGYGYWGQGTTLTVSS | | KAEDVGVYYCQQVVEYPRTFGSGTKL |
| | | | | EIK |
| Human | 165 | QVQLQESGPGLVKPSETLSLTCAVSGYSISS | 166 | DIQMTQSPSSLSASVGDRVTITCRASQ |
| Anti-K-Ras | | GYYWGWIRQPPGKGLEWIGSIYHSGSTYYNP | | SISSYLNWYQQKPGKAPKLLIYAASSLQ |
| Antibody 70 | | SLKSRVTISVDTSKNQFSLKLSSVTAADTAVY | | SGVPSRFSGSGSGTDFTLTISSLQPED |
| | | YCARQRYWSKSYFRPWGQGTLVTVSS | | FATYYCQQSDSYPLTFGGGTKVEIK |

As used herein, the term “binding agent” or “binding domain” refers to any molecule (peptide, protein, small molecular weight chemical, and nucleic acid) or portion of such molecule, respectively, that, by its structure, is able to selectively bind a target antigen. As used herein, the term “selectively bind” or “selective binding” means that the binding agent is able to bind to its target antigen amongst a population of non-target antigens in excess of the target antigen with affinity to the target antigen that is greater than affinity that would result from non-specific binding to the non-target antigens (to the extent any such non-specific binding occurs). Non-limiting examples of binding agents include antibodies, nanobodies, antibody fragments (e.g., scFvs), peptides, proteins, and aptamers.

As used herein, the term “binding availability” as it pertains to cell surface antigens should be understood to account for surface expression, binding partner affinity, or a combination thereof.

As used herein, an “immune effector cell” is a cell that is involved in the cytolytic immune response, including, for example, T cells, NK cells, monocytes, macrophages, or neutrophils.

As used herein, the term “cell penetrating peptides” is defined as sequence of amino acids that can lead to the internalization of the peptides, and molecules attached to the peptides, into the cytoplasm of a cell. Examples of cell penetrating peptides are R9 (SEQ ID NO: 3—RRRRRRRRR-NH2) and penetratin (SEQ ID NO: 4—RQIKIWFQNRRMKWKK-NH2).

As used herein, the term “intracellular delivery compound” refers to a compound which promotes or initiates crossing the cellular membrane of a material or composition where such material or composition would not otherwise cross the cellular membrane and be internalized in the absence of the intracellular delivery compound. Examples of intracellular delivery compounds are cell penetrating peptides, liposomes, nanoparticles, and dendrimers. In some embodiments, the intracellular delivery compound is a cell penetrating peptide. In some embodiments, the intracellular delivery compound is not a cell penetrating peptide.

As used herein, the terms “switch 1” and “switch 2” refer to regions that undergo conformational changes in the K-Ras protein and are known to be important in K-Ras function.

As used herein, the term “antibody” is defined as a protein having a set of immunoglobulin protein domains, commonly referred to as “heavy” and “light” chains, with defined paratopes that functionally recognize an epitope on a target antigen. The term antibody should be understood to encompass antibody fragments, mutant, variation, derivation, or engineered versions of a molecule that contains the functional paratope that recognizes the target epitope. Antibody fragments also include isolated fragments consisting of the variable regions of the heavy and light chains and recombinant single chain polypeptide molecules in which light and heavy variable regions are connected by a peptide linker (“scFv”). Antibody fragments also include F(ab′)2, Fab′, Fab, Fv, sFv, heavy-chain variable antibody (VHH, also referred to as a nanobody), single-domain antibody (e.g., containing a heavy chain variable domain of a heavy-chain antibody), and the like.

A “chimeric antibody” is a recombinant protein that contains the variable domains including the complementarity-determining regions (CDRs) of an antibody derived from one non-human species, while the constant domains of the antibody molecule are derived from those of a human antibody. A humanized antibody is a recombinant protein in which the CDRs from an antibody from one species are transferred from the heavy and light variable chains of that species antibody into human heavy and light variable domains. A human antibody generally refers to an antibody obtained from a transgenic animal that have been engineered to produce specific human antibodies in response to antigenic challenge.

As used herein, the terms “antibody drug conjugate” and “ADC” refer to an antibody that is linked to a functional molecule for the purpose of delivering the molecule to a structure (i.e. cell) containing the targeted antigen. The functional molecule is often referred to as a “payload” or “warhead”.

As used herein, the term “ADC linker” or “linker” refers to the chemical linkage between an antibody and a payload/warhead. These linkers may include the ability to be cleaved, either by proteases, pH conditions, reduction of disulfide bonds, and those that are cleavable either extra- or intracellularly. Other linkers are noncleavable and are broken down by the cell into their active form. A “linker” also refers to a peptide that links to polypeptides, such as that used in a bispecific antibody. Peptide linkers can be from about 2 amino acids to about 30 amino acids and all lengths and intermediate ranges therebetween.

As used herein, the terms “payload” or “warhead” refer to a molecule that is conjugated to an antibody in an ADC via a linker. In some embodiments, the ADC payload or warhead are therapeutic agents.

As used herein, the term “therapeutic agent” refers to any molecule that causes a therapeutic effect (as the term is defined herein). Particularly suitable therapeutic agents for use in an ADC include cytotoxic agents, cytostatic agents, radionuclides, or toxins. Cytotoxic agents are agents than can induce depletion, elimination, and/or killing of the target cell, such as microtubule-disrupting agents (e.g. maytansanoids, mertansine, emtansine, soravtansine, ravtansine, auristatins, MMAE (monomethyl auristatin E), MMAF (monomethyl auristatin F), tubulysins, and taxols), topoisomerase inhibitors (e.g. topotecan, govitecan, deruxtecan, rezetecan), and DNA damaging agents (e.g. exatecan, topoisomerase 1 inhibitors (SN-38), calicheamicin, anthramycin, tesirine, duocarmycin, doxorubicin, and pyrrolobenzodiazepine (PBD)-dimers). Cytostatic agents are agents that can inhibit cell growth and/or proliferation of the target cells. Radionuclides are nuclides that have excess nuclear energy, making them unstable, including a β-particle-emitting radionuclide or an α-particle emitting radionuclide. Examples of α-particle emitting radionuclide include astatine-211, bismuth-212, lead-212, bismuth-213, actinium-225, radium-223 and thorium-227. Examples of β-particle-emitting radionuclides include iodine-131, rhenium-186, yttrium-90, samarium-153, strontium-89, and lutetium-177. When the radionuclide degrades, it can release alpha, beta, or gamma radiation that damages cells in the immediate vicinity. It should be understood that there are many therapeutic agents that suitable for delivery via ADCs and the list here should be considered exemplary and not limiting as to the potential agents.

The term “therapeutic effect” refers to an improvement in clinical features. Non-limiting examples of clinical features are reduction in tumor size, inhibiting or limiting growth of tumor or proliferation of cancer cells, reducing or preventing metastases, preventing or reducing recurrence of cancer after remission, palliating symptoms associated with cancer, improvement of patient longevity, and delay in progression of clinical disease. Thus, a “therapeutically effective amount” is the amount of compositions of the present disclosure that case a therapeutic effect.

As used herein, the term “Chimeric Antigen Receptors” or “CARs” are artificial T cell receptors/immunoreceptors that are engineered to be reactive with antigen specificity. Traditionally, this involves engineering an antibody, or an antibody fragment or derivative (i.e. scFv), onto a transmembrane and intracellular effector domain through a “spacer” sequence. This allows signaling to be induced when the CAR engages with the cognate epitope that the antibody specifically recognizes. The transmembrane domain serves to anchor the CAR into the cellular membrane of the transduced cell. The intracellular effector domain (i.e., endodomain) engages the normal signaling machinery of the transduced cell. T cells are transduced to express the CAR by introduction by a vector (see below).

As used herein, the term “vector” refers to a vehicle used to deliver DNA or protein to a cell. Vectors typically have DNA encoding the desired gene incorporated into the vector DNA. Vectors can include, but are not limited to, plasmids, viruses, and bacteria. In the case of CARs, vectors typically comprise viruses engineered to introduce the CAR genetic components into the desired cells.

As used herein, a “[percent](%) sequence identity” with respect to a reference polypeptide sequence means the percentage of amino acid residues in a candidate sequence that are identical with the amino acid residues in the reference polypeptide sequence after aligning the sequences and introducing gaps, if necessary, to achieve the maximum percent sequence identity, and not considering any conservative substitutions as part of the sequence identity. A “conservative substitution” means replacement of an amino acid residue for another amino acid residue with similar properties such as size, charge and hydrophobicity.

### Antibody-Drug Conjugates

In one aspect of the present disclosure, the use of antibody-drug conjugates (ADCs) to treat cancers associated with expression of Surface K-Ras Antigens is provided. ADC's generally comprise three parts: (1) a binding means for selectively binding one or more Surface K-Ras Antigens (e.g., antibody); (2) a payload or warhead (generally a therapeutic agent); and (3) a linker means for conjugating the binding means to the payload.

The binding means used in the ADC of the present invention should possess target specificity for Surface K-Ras Antigens that are expressed on the target cancer cells, but not on healthy cells. This function can be performed with antibodies, antibody fragments or peptides such as those set forth in Table 1 and Table 2. The antibodies should possess high binding affinity and low immunogenicity and cross-reactivity while still retaining the properties to allow linkage to the warhead. The antibodies may be chimeric, humanized, or fully human and may also be monospecific, bispecific, trispecific, or multi specific. In one embodiment, the binding agent is an antibody or antibody fragment. In one embodiment, the binding agent is an antibody or antibody fragment that selectively binds the Surface K-Ras Antigen, wherein the Surface K-Ras Antigen comprises a mutation at residue 12, 13, or 61 based on the amino acid sequence set forth in SEQ ID NO: 1 or SEQ ID NO: 2. In another embodiment, the binding agent is an antibody or antibody fragment that selectively binds the Surface K-Ras Antigen, wherein the Surface K-Ras Antigen comprises any one of the following mutations based on the amino acid sequence set forth in SEQ ID NO: 1 or SEQ ID NO: 2: G12A; G12D; G12C; G12V; G12R; G13D; Q61H; and Q61L. In another embodiment, the binding agent is an antibody or antibody fragment that selectively binds the Surface K-Ras Antigen, wherein the Surface K-Ras Antigen comprises any one of the following mutations based on the amino acid sequence set forth in SEQ ID NO: 1 or SEQ ID NO: 2, but either does not bind an antigen of such sequence without one of these mutations or if able to bind such sequence without such mutation, does so with an affinity that would not be considered clinically relevant or therapeutically effective: G12A; G12D; G12C; G12V; G12R; G13D; Q61H; and Q61L In any of the above embodiments, the binding agent is an antibody or antibody fragment that binds a Surface K-Ras Antigen that possesses greater than 70% sequence identity to SEQ ID NO: 1 or SEQ ID NO: 2, greater than 80% sequence identity to SEQ ID NO: 1 or SEQ ID NO: 2, greater than 90% sequence identity to SEQ ID NO: 1 or SEQ ID NO: 2, or any percentage of sequence identity to SEQ ID NO: 1 or SEQ ID NO: 2 between 70% and 99% (e.g., 75%, 80%, 85%, 90%, 95%, 96%, 97%, 98%, or 99% sequence identity to SEQ ID NO: 1 or SEQ ID NO: 2). In some embodiments, the binding agent is an antibody or antibody fragment that binds a Surface K-Ras Antigen that possesses greater than 75% sequence identity to SEQ ID NO: 1 or SEQ ID NO: 2. In some embodiments, the binding agent is an antibody or antibody fragment that binds a Surface K-Ras Antigen that possesses greater than 80% sequence identity to SEQ ID NO: 1 or SEQ ID NO: 2. In some embodiments, the binding agent is an antibody or antibody fragment that binds a Surface K-Ras Antigen that possesses greater than 85% sequence identity to SEQ ID NO: 1 or SEQ ID NO: 2. In some embodiments, the binding agent is an antibody or antibody fragment that binds a Surface K-Ras Antigen that possesses greater than 90% sequence identity to SEQ ID NO: 1 or SEQ ID NO: 2. In some embodiments, the binding agent is an antibody or antibody fragment that binds a Surface K-Ras Antigen that possesses greater than 95% sequence identity to SEQ ID NO: 1 or SEQ ID NO: 2. In some embodiments, the binding agent is an antibody or antibody fragment that binds a Surface K-Ras Antigen that possesses greater than 96% sequence identity to SEQ ID NO: 1 or SEQ ID NO: 2. In some embodiments, the binding agent is an antibody or antibody fragment that binds a Surface K-Ras Antigen that possesses greater than 97% sequence identity to SEQ ID NO: 1 or SEQ ID NO: 2. In some embodiments, the binding agent is an antibody or antibody fragment that binds a Surface K-Ras Antigen that possesses greater than 98% sequence identity to SEQ ID NO: 1 or SEQ ID NO: 2. In some embodiments, the binding agent is an antibody or antibody fragment that binds a Surface K-Ras Antigen that possesses greater than 99% sequence identity to SEQ ID NO: 1 or SEQ ID NO: 2. Binding agents suitable for use in the ADC embodiments disclosed herein include those listed in Table 1 and/or Table 2 as applicable for a given cancer-expressing mutation.

The specific antigens used for developing antibodies of the present invention may encompass the regions of K-Ras that are known to include mutations found in cancers, such as those found at residues 12, 13 and 61. Examples 4 and 5 herein describe specific methods for developing antibodies of the present disclosure for use in ADCs.

Linkers used in the present ADCs must provide a stable interaction between the antibody and the payload to avoid premature release of the payload and unintended off-target effects. The linker also should maintain the payload in an inactive, non-toxic state when bound to the antibody, but should be able to release the payload upon cellular internalization. Thus, particularly suitable linkers for conjugating the binding agent to the payload include linkers that are cleavable in intracellular conditions, such that the cleavage of the linker releases the cytotoxic or cytostatic moiety from the antibody intracellularly (e.g. within an endosomal or lysosmal compartment), or non-cleavable linkers, such that the cytostatic or cytotoxic moiety, or a derivative thereof, is released from the antibody after antibody degradation (e.g. within the lysosome or via the proteasome). Cleavable linkers can include those cleavable at low pH (i.e. sensitive to hydrolysis when exposed to certain pH environments), such as acid-labile linkers (e.g. a hydrazone, semicarbazone, thiosemicarbazone, cis-aconitic amide, orthoester, acetal, ketal, or the like). Such acid-labile linkers are relatively stable at neutral pH, such as in blood and the extracellular environment, and are relatively unstable at pH 5.5 or below, the approximate pH of the lysosome. Cleavable linkers can also include those cleaved by lysosomal or endosomal proteases (e.g. cathepsin B) or enzymes (β-glucuronidase), such as peptidyl linkers (e.g. Val-Ala, Val-Citrulline, Gly-Gly-Phe-Gly, Gly-Phe-Leu-Gly, and Phe-Leu) or β-glucuronide linkers. Cleavable linkers can also include those that are cleavable under reducing conditions (e.g. a disulfide bond), such as disulfide linkers (e.g. SATA (N-succinimidyl-S-acetylthioacetate), SMCC (Succinimidyl-4-(N-maleimidomethyl)cyclohexane-1-carboxylate), SMPT (N-succinimidyl-oxycarbonyl-alpha-methyl-alpha-(2-pyridyl-dithio)toluene), SPDB (N-succinimidyl-3-(2-pyridyldithio)butyrate), and SPDP (N-succinimidyl-3-(2-pyridyldithio)propionate). Non-cleavable linkers can include those that are not readily cleaved by proteases, cleaved at low pH, or released under reducing conditions, such as maleimido-alklene- or maleimide-aryl linker. In one embodiment, the linker is any one or more of a maleimidocaproyl linker, a peptide-based linker (including, but not limited to a valine-citrulline linker), a β-glucuronide linker, a SMCC linker, a disulfide linker, or an acid sensitive linker.

A wide variety of therapeutic agents can be administered as the payload in the subject ADCs. Alternatively, such agents may be separately administered concurrently with, prior to or after administration of the ADC. Therapeutic agents of the present ADCs include, for example, drugs, toxins, oligonucleotides, immunomodulators, hormones, hormone antagonists, enzymes, enzyme inhibitors, radionuclides, and angiogenesis inhibitors (in addition to those listed above). Therapeutic agents further include, for example, cytotoxic drugs such as vinca alkaloids, anthracyclines such as doxorubicin, 2-PDox or pro-2-PDox, gemcitabine, epipodophyllotoxins, taxanes, antimetabolites, alkylating agents, antibiotics, SN-38, COX-2 inhibitors, antimitotics, anti-angiogenic and pro-apoptotic agents, particularly doxorubicin, methotrexate, taxol, CPT-11, camptothecans, proteosome inhibitors, mTOR inhibitors, HDAC inhibitors, and tyrosine kinase inhibitors. Other useful anti-cancer cytotoxic drugs for administering concurrently or sequentially, or for the preparation of ADCs include nitrogen mustards, alkyl sulfonates, nitrosoureas, triazenes, folic acid analogs, COX-2 inhibitors, antimetabolites, pyrimidine analogs, purine analogs, platinum coordination complexes, mTOR inhibitors, tyrosine kinase inhibitors, proteosome inhibitors, HDAC inhibitors, camptothecins, hormones, and the like.

The method of conjugating the linker to the antibody may include random stochastic conjugation (e.g. conjugation to surface exposed lysines) or site-specific conjugation (e.g. conjugation to cysteines of reduced interchain disulfide bonds, conjugation performed during glycan-remodeling, or conjugation to included unnatural amino acids or specific amino acid sequences). As will be appreciated by skilled artisans, the number of therapeutic agents linked to an antibody molecule may vary, such that a collection of ADCs may be heterogeneous in nature, where some antibodies contain one linked agent, some two, and some three and so on (and some none). The degree of heterogeneity will depend upon, among other things, the chemistries used for linking the therapeutic agents. In one embodiment, the number of therapeutic agents conjugated to an individual anti-K-Ras antibody, or antigen-binding fragment thereof, can range from one to eight or more moieties.

ADCs are usually administered to patients via intravenous injection but could also be administered via subcutaneous injection. The starting dose for a first-in-human clinical trial is usually determined by the preclinical pharmacology and non-clinical pharmacokinetics and toxicology data. Typically, starting doses for ADCs are at or near 1 mg/kg and may be tested up to 20 mg/kg. Typical dosing intervals for ADCs are once every one, two, or three weeks. The optimal dose and dosing interval is affected by the antibody, the linker, the cytotoxic or cytostatic agent, and the conjugation chemistry used to generate and individual ADC. Typically, the ADC is provided as a lyophilized powder that is reconstituted into solutions (e.g., normal saline, dextrose or similar solutions) before injection.

In certain embodiments the ADC and one or more other antibodies may be administered as separate antibodies, either sequentially or concurrently. In alternative embodiments, antibodies or antibody fragments may be administered as a single bispecific or multispecific antibody.

### Bispecific Antibodies

In another aspect of the present disclosure, a composition for treating cancers expressing a Surface K-Ras antigen comprises a bispecific antibody. Immune cell redirecting bispecific antibodies are a therapeutic modality where a designed antibody is used to bring an immune cell into close proximity to a target-expressing cell to stimulate immune cell killing of the target cell. A bispecific antibody is engineered to have specificity to the target cell via an antibody, Fab or scFv (or other antibody fragment) specific to the target antigen, and specificity to the immune cell via an antibody, Fab or scFv (or other antibody fragment) specific to the immune cell. Immune cell redirecting bispecific antibodies can include T cell redirecting bispecific antibodies, natural killer (NK) cell redirecting bispecific antibodies, and macrophage redirecting bispecific antibodies. Typically, but not exclusively, T cell redirecting bispecific antibodies bring T cells into close proximity to a target-expressing cell to stimulate T cell killing of the target cell via the T cell surface protein CD3.

The most common bispecific antibody formats are the single-chain variable fragment (scFv) with no Fc fragment or the full-length IgG-like asymmetric antibody. However, many other formats are currently being used and developed. See Table 3 for bispecific antibody formats that are contemplated herein.

TABLE 3
Exemplary Antibody Structures and configurations for
use in bispecific and trispecific embodiments.

| Bispecific | |
|---|---|
| Antibody | |
| Type | Description |
| Bispecific | Bispecific or biparatopic molecule with a classical |
| IgG (type 1) | IgG structure and mutations in the CH3 domain of |
| | the heavy chain to favor heterodimerization of the |
| | heavy chains with different VH domains. This |
| | molecule contains no light chain modifications that |
| | encourage correct light chain pairing. |
| Bispecific | Bispecific or biparatopic molecule with a classical |
| IgG (type 2) | IgG structure and mutations in the CH3 domain of |
| | the heavy chain to favor heterodimerization of the |
| | heavy chains with different VH domains. This |
| | molecule contains a common light chain that pairs |
| | with both heavy chains. |
| Bispecific | Bispecific or biparatopic molecule with a classical |
| IgG (type 3) | IgG structure, a common light chain and two heavy |
| | chains with different VH domains but no mutations |
| | to favor correct heavy chain pairing. |
| Bispecific | Bispecific or biparatopic molecule with a classical |
| IgG (type 4) | IgG structure consisting of mutations in the CH3 |
| | domain of the heavy chain to favor heterodimerization |
| | of the heavy chains with different VH domains and |
| | with mutations in one of the Fab arms to favor |
| | correct light chain pairing. |
| Bispecific | Bispecific or biparatopic molecule with a classical |
| IgG (type 5) | IgG structure consisting of mutations in the CH3 |
| | domain of the heavy chain to favor heterodimerization |
| | of the heavy chains with different VH domains and |
| | with the CH1 and CL domains in one Fab arm |
| | crossed-over to favor light chain pairing. |
| Bispecific | The final molecule is a bispecific or biparatopic |
| IgG (type 6) | molecule with a classical IgG structure and |
| | mutations in the CH3 domain of the heavy chain to |
| | favor heterodimerization of the heavy chains with |
| | different VH domains. This is obtained by producing |
| | the two monoclonal antibodies independently and |
| | mixing together under specific conditions that favor |
| | the recombination of the different heavy chains to |
| | form a heterodimeric species. |
| IgG-scFv | Fusion of an IgG to a scFv of a second specificity |
| (type 1) | at the C-terminus of the IgG heavy chain. This |
| | can be a direct fusion or with a linker of any |
| | length or composition. |
| IgG-scFv | An IgG fused to a scFv of a second specificity at |
| (type 2) | the N-terminus of the heavy chain and two further |
| | scFvs at the C-terminus of the heavy chain of a |
| | third and fourth specificity respectively. Linkers |
| | between IgG and scFvs can be of any length or |
| | composition. |
| IgG-scFv | Fusion of an IgG to a scFv of a second specificity |
| (type 3) | at the C-terminus of the IgG light chain. This |
| | can be a direct fusion or with a linker of any |
| | length or composition. |
| IgG-scFv | A fusion of an IgG with a scFv where the scFv is |
| (type 4) | inserted in between the Fab and Fc domains with a |
| | linker of any length or composition. The scFv is |
| | of a different specificity to the Fab. |
| Fv-IgG | An IgG with additional VH and VL domains of a |
| | second specificity fused to the N-terminus of the |
| | heavy and light chains respectively via linkers |
| | of any length of composition. |
| Fab-IgG | An IgG with an additional Fab fused to the |
| (type 1) | N-terminus of one heavy chain. Mutations are |
| | present in the CH3 domain to favor correct heavy |
| | chain pairing and the CH1 and CL domains of the |
| | second Fab on the longer heavy chain are |
| | crossed-over to favor correct light chain pairing. |
| | The second Fab on the longer heavy chain is also |
| | of a different specificity to the other Fab arms. |
| Fab-IgG | An IgG with a Fab of a second specificity fused |
| (type 2) | to the N-terminus of the heavy chain wherein |
| | the additional Fab contains mutations in the CH1 |
| | and CL domains to favor correct pairing of the |
| | light chain. |
| Fab-IgG | An IgG with a Fab of a second specificity fused |
| (type 3) | to the C-terminus of the heavy chain. The IgG |
| | contains mutations in the CH3 domain to favor |
| | heavy chain heterodimerization this creating an |
| | asymmetric molecule with only one copy of the |
| | additional Fab domain. The additional Fab |
| | domain also contains a cross-over of CH1 and CL |
| | domains to favor correct light chain pairing. |
| Diabody | A dimer of scFvs with different specificities |
| | where the VH of specificity to target A is on |
| | the same chain as the VL to target B and vice |
| | versa. The chains can be linked covalently or |
| | non-covalently. Domains or peptides to encourage |
| | correct formation of the molecule may or may not |
| | be included. |
| Diabody-Fc | Fusion of a diabody to an Fc domain of any |
| (type 1) | species, subtype or allotype via a linker of |
| | any length of composition. |
| Diabody-Fc | Fusion of a diabody to an Fc domain of any |
| (type 2) | species, subtype or allotype via a linker of |
| | any length of composition whereby the Fc domain |
| | contains CH3 domain mutations to favor formation |
| | of a heterodimer. |
| Tandem dAb | Two domain antibodies (dAbs) fused together |
| (type 2) | directly or with a linker of any length and |
| | composition where both dAbs are different, i.e. |
| | creating a monovalent bispecific. |
| Tandem | Fusion of a tandem dAb (type 2) to an Fc domain |
| dAb-Fc | of any species, subtype or allotype via a |
| | linker of any length of composition. |
| Triple dAb | Three domain antibodies (dAbs) fused together |
| (type 2) | directly or with a linker of any length and |
| | composition where the first and third dAbs |
| | are the same but the second is of a different |
| | specificity. |
| Triple dAb | Three domain antibodies (dAbs) fused together |
| (type 3) | directly or with a linker of any length and |
| | composition where all dAbs are different, i.e. |
| | creating a monovalent trispecific. |
| Tandem | Two scFvs linked together in tandem with a |
| scFv | linker between them of any length or composition. |
| Tandem | A tandem scFv fused to an Fc domain either |
| scFv-scFc | directly or via a linker of any length and |
| | composition, wherein the Fc domain is a single |
| | chain Fc (scFc) consisting of hinge, CH2 and |
| | CH3 domains followed by a sufficiently long |
| | linker and then an additional hinge, CH2 |
| | and CH3. |
| Heterodimeric | A heterodimeric molecule with mutations in |
| Fab/scFv-Fc | the Fc domain to favor formation of the |
| | heterodimeric species. One heavy chain |
| | contains a typical heavy chain with separate |
| | light chain (i.e. a Fab) while the second heavy |
| | chain contains a scFv fused to the N-terminus |
| | of the Fc domain either directly or via a |
| | linker of any length and composition. |
| scFv-TCR | A scFv fused at its C-terminus to a T-cell |
| fusion | receptor beta chain which heterodimerizes |
| | with a separate alpha chain. |
| VHH | A VHH antibody (or nanobody) is the antigen |
| | binding fragment of heavy chain only |
| | antibodies. |

In one embodiment, a bispecific antibody of the present disclosure comprises a first binding domain linked to a second binding domain. In one embodiment, a bispecific antibody of the present disclosure comprises bispecific antibody having a format selected from the group of a Bispecific IgG (type 1), Bispecific IgG (type 2), Bispecific IgG (type 3), Bispecific IgG (type 4), Bispecific IgG (type 5), Bispecific IgG (type 6), IgG-scFv (type 1), IgG-scFv (type 2), IgG-scFv (type 3), IgG-scFv (type 4), Fv-IgG, Fab-IgG (type 1), Fab-IgG (type 2), Fab-IgG (type 3), Diabody, Diabody-Fc (type 1), Diabody-Fc (type 2), Tandem dAb (type 2), Tandem dAb-Fc, Triple dAb (type 2), Triple dAb (type 3), Tandem scFv, Tandem scFv-scFc, Heterodimeric Fab/scFv-Fc, and scFv-TCR fusion.

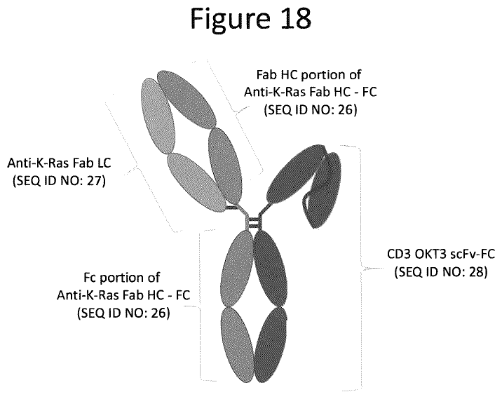

The first binding domain comprises a first means for selectively binding to a Surface K-Ras Antigen. Structures suitable for selectively binding to a surface K-Ras Antigen include the following: any of the Fab regions of the antibodies listed in Table 1 and/or Table 2; any scFv constructs comprising the light chain variable region and heavy chain variable region of any of the antibodies listed in Table 1 and/or Table 2; any of the Fab-Fc regions of the antibodies listed in Table 1 and/or Table 2; any scFv constructs comprising the light chain variable region and heavy chain variable region linked to the Fc region of any of the antibodies listed in Table 1 and/or Table 2 (in each case with or without “hole” or “knob” mutations in the Fc region including any of the Fc mutations similar to those disclosed for the bispecific construct in Example 13); the Anti-K-Ras Fab light chain (SEQ ID NO: 27) and the Fab heavy chain portion and Fc portion (SEQ ID NO: 26) shown in FIG. 18; an scFv comprising the light chain variable region of SEQ ID NO: 13 linked to the heavy chain variable region of SEQ ID NO: 9 of Human Anti-K-Ras Antibody 1 (comprising the heavy chain CDRs 1, 2, and 3 corresponding to SEQ ID NOS: 10, 11, and 12, respectively, and the light chain CDRs 1, 2 and 3 corresponding to SEQ ID NOS: 14, 15, and 16); an scFv comprising the light chain variable region of SEQ ID NO: 21 linked to the heavy chain variable region of SEQ ID NO: 17 of Human Anti-K-Ras Antibody 2 (comprising the heavy chain CDRs 1, 2, and 3 corresponding to SEQ ID NOS: 18, 19, and 20, respectively, and the light chain CDRs 1, 2 and 3 corresponding to SEQ ID NOS: 22, 23, and 24); the Fab region of SEQ ID NOS: 9 and 13 with or without the Fc portions; the Fab region of SEQ ID NOS: 17 and 21 with or without the Fc portions. The first binding domain may also include a second means for binding to a different region or epitope of the Surface K-Ras Antigen. Structures suitable for binding to a Surface K-Ras Antigen are set forth above, except that the second means must bind to a different region of the Surface K-Ras Antigen than the first means. Thus, any structure corresponding to the first means cannot be the same structure corresponding to the second means. It should be understood that the structures identified for binding a Surface K-Ras Antigen here as applied to bispecific antibodies could also be used in compositions of the present disclosure involving antibody drug conjugates

The second binding domain comprises a means for selectively binding to an antigen on the surface of an immune effector cell. Structures suitable for binding to an antigen of an immune effector cell includes the CD3 scFv-Fc polypeptide shown in FIG. 18 (SEQ ID NO: 28), any Fab construct, scFv construct or other functional antibody fragment that selective binds to TCRα, TCRβ, TCRδ, TCRγ, CD3β, CD3γ, CD3δ, CD3ζ, CD137, CD16, and CD64, including functional CD3 antibodies. Exemplary CD3 binding domains (e.g., VH and VL domains) are set forth in Table 4. Similar to the first binding domain, the second binding domain may also include a second means for binding to another antigen on the surface of an immune effector cell. Structures suitable for binding to an antigen on the surface of an immune effector cell are set forth above, except that the second means must bind to a different region of the antigen than the first means. Thus, any structure corresponding to the first means cannot be the same structure corresponding to the second means.

TABLE 4
Exemplary Antibodies that Bind CD3 Antigen.

| | VH | | VL | |
|---|---|---|---|---|
| | SEQ | | SEQ | |
| CD3 | ID | | ID | |
| Antibody | NO: | VH Amino Acid Sequence | NO: | VL Amino Acid Sequence |
| acapatamab | 167 | EVQLVESGGGLVQPGGSLKLSCAASG | 168 | QTVVTQEPSLTVSPGGTVTLTCGSSTGAVTSGNY |
| | | FTFNKYAMNWVRQAPGKGLEWVARIR | | PNWVQQKPGQAPRGLIGGTKFLAPGTPARFSGS |
| | | SKYNNYATYYADSVKDRFTISRDDSKN | | LLGGKAALTLSGVQPEDEAEYYCVLWYSNRWVF |
| | | TAYLQMNNLKTEDTAVYYCVRHGNFG | | GGGTKLTVL |
| | | NSYISYWAYWGQGTLVTVSS | | |
| alnuctamab | 169 | EVQLLESGGGLVQPGGSLRLSCAASG | 170 | QAVVTQEPSLTVSPGGTVTLTCGSSTGAVTTSNY |
| | | FTFSTYAMNWVRQAPGKGLEWVSRIR | | ANWVQEKPGQAFRGLIGGTNKRAPGTPARFSGS |
| | | SKYNNYATYYADSVKGRFTISRDDSKN | | LLGGKAALTLSGAQPEDEAEYYCALWYSNLWVF |
| | | TLYLQMNSLRAEDTAVYYCVRHGNFG | | GGGTKLTVL |
| | | NSYVSWFAYWGQGTLVTVSS | | |
| blinatumomab | 171 | DIKLQSGAELARPGASVKMSCKTSGYT | 172 | DIQLTQSPAIMSASPGEKVTMTCRASSSVSYMNW |
| | | FTRYTMHWVKQRPGQGLEWIGYINPS | | YQQKSGTSPKRWIYDTSKVASGVPYRFSGSGSG |
| | | RGYTNYNQKFKDKATLTTDKSSSTAYM | | TSYSLTISSMEAEDAATYYCQQWSSNPLTFGAGT |
| | | QLSSLTSEDSAVYYCARYYDDHYCLDY | | KLELK |
| | | WGQGTTLTVSS | | |
| cevostamab | 173 | EVQLVQSGAEVKKPGASVKVSCKASG | 174 | DIVMTQSPDSLAVSLGERATINCKSSQSLLNSRTR |
| | | FTFTSYYIHWVRQAPGQGLEWIGWIYP | | KNYLAWYQQKPGQSPKLLIYWTSTRKSGVPDRF |
| | | ENDNTKYNEKFKDRVTITADTSTSTAYL | | SGSGSGTDFTLTISSLQAEDVAVYYCKQSFILRTF |
| | | ELSSLRSEDTAVYYCARDGYSRYYFDY | | GQGTKVEIK |
| | | WGQGTLVTVSS | | |
| cibisatamab | 175 | QVQLVQSGAEVKKPGASVKVSCKASG | 176 | QAVVTQEPSLTVSPGGTVTLTCGSSTGAVTTSNY |
| | | YTFTEFGMNWVRQAPGQGLEWMGWI | | ANWVQEKPGQAFRGLIGGTNKRAPGTPARFSGS |
| | | NTKTGEATYVEEFKGRVTFTTDTSTST | | LLGGKAALTLSGAQPEDEAEYYCALWYSNLWVF |
| | | AYMELRSLRSDDTAVYYCARWDFAYY | | GGGTKLTVL |
| | | VEAMDYWGQGTTVTVSS | | |
| dafsolimab | 177 | QVQLQQSGAELARPGASVKMSCKASG | 178 | QIVLTQSPAIMSASPGEKVTMTCSASSSVSYMHW |
| | | YTFTSYTMHWVKQRPGQGLEWIGYIN | | YQQKSGTSPKRWIYDTSKLASGVPARFSGSGSG |
| | | PSSGYTNYIQRFKDKATLTADKSSSTA | | TSYSLTISSMEAEDAATYYCQQWSSNPLTFGAGT |
| | | YMQVSSLTSEDSAVYYCARGSRYDYY | | KLELK |
| | | GMDYWGQGTSVTVSS | | |
| duvortuxizumab | 179 | EVQLVESGGGLVQPGGSLRLSCAASG | 180 | QAVVTQEPSLTVSPGGTVTLTCRSSTGAVTTSNY |
| | | FTFSTYAMNWVRQAPGKGLEWVGRIR | | ANWVQQKPGQAPRGLIGGTNKRAPWTPARFSGS |
| | | SKYNNYATYYADSVKGRFTISRDDSKN | | LLGGKAALTITGAQAEDEADYYCALWYSNLWVFG |
| | | SLYLQMNSLKTEDTAVYYCVRHGNFG | | GGTKLTVL |
| | | NSYVSWFAYWGQGTLVTVSS | | |
| elranatamab | 181 | EVQLVESGGGLVQPGGSLRLSCAASG | 182 | DIVMTQSPDSLAVSLGERATINCKSSQSLFNVRSR |
| | | FTFSDYYMTWVRQAPGKGLEWVAFIR | | KNYLAWYQQKPGQPPKLLISWASTRESGVPDRF |
| | | NRARGYTSDHNPSVKGRFTISRDNAKN | | SGSGSGTDFTLTISSLQAEDVAVYYCKQSYDLFTF |
| | | SLYLQMNSLRAEDTAVYYCARDRPSYY | | GSGTKLEIK |
| | | VLDYWGQGTTVTVSS | | |
| eluvixtamab | 183 | EVQLVESGGGLVQPGGSLKLSCAASG | 184 | QTVVTQEPSLTVSPGGTVTLTCGSSTGAVTSGNY |
| | | FTFNKYAMNWVRQAPGKGLEWVARIR | | PNWVQQKPGQAPRGLIGGTKFLAPGTPARFSGS |
| | | SKYNNYATYYADSVKDRFTISRDDSKN | | LLGGKAALTLSGVQPEDEAEYYCVLWYSNRWVF |
| | | TAYLQMNNLKTEDTAVYYCVRHGNFG | | GGGTKLTVL |
| | | NSYISYWAYWGQGTLVTVSS | | |
| emerfetamab | 185 | EVQLVESGGGLVQPGGSLKLSCAASG | 186 | QTVVTQEPSLTVSPGGTVTLTCGSSTGAVTSGNY |
| | | FTFNKYAMNWVRQAPGKGLEWVARIR | | PNWVQQKPGQAPRGLIGGTKFLAPGTPARFSGS |
| | | SKYNNYATYYADSVKDRFTISRDDSKN | | LLGGKAALTLSGVQPEDEAEYYCVLWYSNRWVF |
| | | TAYLQMNNLKTEDTAVYYCVRHGNFG | | GGGTKLTVL |
| | | NSYISYWAYWGQGTLVTVSS | | |
| emfizatamab | 187 | EVQLVESGGGLVQPGGSLRLSCAASG | 188 | DVVMTQSPSTLSASVGDRVTINCQASESISSWLA |
| | | FTISTNAMSWVRQAPGKGLEWIGVITG | | WYQQKPGKAPKLLIYEASKLASGVPSRFSGSGSG |
| | | RDITYYASWAKGRFTISRDNSKNTLYL | | TEFTLTISSLQPDDFATYYCQGYFYFISRTYVNSF |
| | | QMNSLRAEDTAVYYCARDGGSSAITSN | | GGGTKVEIK |
| | | NIWGQGTLVTVSS | | |
| emirodatamab | 189 | EVQLVESGGGLVQPGGSLKLSCAASG | 190 | QTVVTQEPSLTVSPGGTVTLTCGSSTGAVTSGNY |
| | | FTFNKYAMNWVRQAPGKGLEWVARIR | | PNWVQQKPGQAPRGLIGGTKFLAPGTPARFSGS |
| | | SKYNNYATYYADSVKDRFTISRDDSKN | | LLGGKAALTLSGVQPEDEAEYYCVLWYSNRWVF |
| | | TAYLQMNNLKTEDTAVYYCVRHGNFG | | GGGTKLTVL |
| | | NSYISYWAYWGQGTLVTVSS | | |
| epcoritamab | 191 | EVKLVESGGGLVQPGGSLRLSCAASG | 192 | QAVVTQEPSFSVSPGGTVTLTCRSSTGAVTTSNY |
| | | FTFNTYAMNWVRQAPGKGLEWVARIR | | ANWVQQTPGQAFRGLIGGTNKRAPGVPARFSGS |
| | | SKYNNYATYYADSVKDRFTISRDDSKS | | LIGDKAALTITGAGQADDESIYFCALWYSNLWVFG |
| | | SLYLQMNNLKTEDTAMYYCVRHGNFG | | GGTKLTVL |
| | | NSYVSWFAYWGQGTLVTVSS | | |
| etevritamab | 193 | EVQLVESGGGLVQPGGSLKLSCAASG | 194 | QTVVTQEPSLTVSPGGTVTLTCGSSTGAVTSGNY |
| | | FTFNKYAMNWVRQAPGKGLEWVARIR | | PNWVQQKPGQAPRGLIGGTKFLAPGTPARFSGS |
| | | SKYNNYATYYADSVKDRFTISRDDSKN | | LLGGKAALTLSGVQPEDEAEYYCVLWYSNRWVF |
| | | TAYLQMNNLKTEDTAVYYCVRHGNFG | | GGGTKLTVL |
| | | NSYISYWAYWGQGTLVTVSS | | |
| flotetuzumab | 195 | EVQLVESGGGLVQPGGSLRLSCAASG | 196 | QAVVTQEPSLTVSPGGTVTLTCRSSTGAVTTSNY |
| | | FTFSTYAMNWVRQAPGKGLEWVGRIR | | ANWVQQKPGQAPRGLIGGTNKRAPWTPARFSGS |
| | | SKYNNYATYYADSVKDRFTISRDDSKN | | LLGGKAALTITGAQAEDEADYYCALWYSNLWVFG |
| | | SLYLQMNSLKTEDTAVYYCVRHGNFG | | GGTKLTVL |
| | | NSYVSWFAYWGQGTLVTVSS | | |
| foralumab | 197 | QVQLVESGGGVVQPGRSLRLSCAASG | 198 | EIVLTQSPATLSLSPGERATLSCRASQSVSSYLAW |
| | | FKFSGYGMHWVRQAPGKGLEWVAVI | | YQQKPGQAPRLLIYDASNRATGIPARFSGSGSGT |
| | | WYDGSKKYYVDSVKGRFTISRDNSKN | | DFTLTISSLEPEDFAVYYCQQRSNWPPLTFGGGT |
| | | TLYLQMNSLRAEDTAVYYCARQMGYW | | KVEIK |
| | | HFDLWGRGTLVTVSS | | |
| glofitamab | 199 | EVQLLESGGGLVQPGGSLRLSCAASG | 200 | QAVVTQEPSLTVSPGGTVTLTCGSSTGAVTTSNY |
| | | FTFSTYAMNWVRQAPGKGLEWVSRIR | | ANWVQEKPGQAFRGLIGGTNKRAPGTPARFSGS |
| | | SKYNNYATYYADSVKGRFTISRDDSKN | | LLGGKAALTLSGAQPEDEAEYYCALWYSNLWVF |
| | | TLYLQMNSLRAEDTAVYYCVRHGNFG | | GGGTKLTVLSS |
| | | NSYVSWFAYWGQGTLVTVSS | | |
| gresonitamab | 201 | EVQLVESGGGLVQPGGSLKLSCAASG | 202 | QTVVTQEPSLTVSPGGTVTLTCGSSTGAVTSGNY |
| | | FTFNKYAMNWVRQAPGKGLEWVARIR | | PNWVQQKPGQAPRGLIGGTKFLAPGTPARFSGS |
| | | SKYNNYATYYADSVKDRFTISRDDSKN | | LLGGKAALTLSGVQPEDEAEYYCVLWYSNRWVF |
| | | TAYLQMNNLKTEDTAVYYCVRHGNFG | | GGGTKLTVL |
| | | NSYISYWAYWGQGTLVTVSS | | |
| imvotamab | 203 | QVQLVQSGAEVKKPGASVKVSCKASG | 204 | DIQMTQSPSSLSASVGDRVTITCSASSSVSYMNW |
| | | YTFISYTMHWVRQAPGQGLEWMGYIN | | YQQKPGKAPKRLIYDTSKLASGVPSRFSGSGSGT |
| | | PRSGYTHYNQKLKDKATLTADKSASTA | | DFTLTISSLQPEDFATYYCQQWSSNPPTFGGGTK |
| | | YMELSSLRSEDTAVYYCARSAYYDYD | | VEIK |
| | | GFAYWGQGTLVTVSS | | |
| linvoseltamab | 205 | EVQLVESGGGLVQPGRSLRLSCAASG | 206 | DIQMTQSPSSLSASVGDRVTITCRASQSISSYLNW |
| | | FTFDDYSMHWVRQAPGKGLEWVSGIS | | YQQKPGKAPKLLIYAASSLQSGVPSRFSGSGSGT |
| | | WNSGSKGYADSVKGRFTISRDNAKNS | | DFTLTISSLQPEDFATYYCQQSYSTPPITFGQGTR |
| | | LYLQMNSLRAEDTALYYCAKYGSGYG | | LEIK |
| | | KFYHYGLDVWGQGTTVTVSS | | |
| mosunetuzumab | 207 | EVQLVQSGAEVKKPGASVKVSCKASG | 208 | DIVMTQSPDSLAVSLGERATINCKSSQSLLNSRTR |
| | | YTFTNYYIHWVRQAPGQGLEWIGWIYP | | KNYLAWYQQKPGQPPKLLIYWASTRESGVPDRF |
| | | GDGNTKYNEKFKGRATLTADTSTSTAY | | SGSGSGTDFTLTISSLQAEDVAVYYCTQSFILRTF |
| | | LELSSLRSEDTAVYYCARDSYSNYYFD | | GQGTKVEIK |
| | | YWGQGTLVTVSS | | |
| muromonab | 209 | QVQLQQSGAELARPGASVKMSCKASG | 210 | QIVLTQSPAIMSASPGEKVTMTCSASSSVSYMNW |
| | | YTFTRYTMHWVKQRPGQGLEWIGYIN | | YQQKSGTSPKRWIYDTSKLASGVPAHFRGSGSG |
| | | PSRGYTNYNQKFKDKATLTTDKSSSTA | | TSYSLTISGMEAEDAATYYCQQWSSNPFTFGSGT |
| | | YMQLSSLTSEDSAVYYCARYYDDHYCL | | KLEIN |
| | | DYWGQGTTLTVSS | | |
| nivatrotamab | 211 | QVQLVQSGGGVVQPGRSLRLSCKASG | 212 | DIQMTQSPSSLSASVGDRVTITCSASSSVSYMNW |
| | | YTFTRYTMHWVRQAPGKCLEWIGYINP | | YQQTPGKAPKRWIYDTSKLASGVPSRFSGSGSG |
| | | SRGYTNYNQKFKDRFTISRDNSKNTAF | | TDYTFTISSLQPEDIATYYCQQWSSNPFTFGCGTK |
| | | LQMDSLRPEDTGVYFCARYYDDHYSL | | LQIT |
| | | DYWGQGTPVTVSS | | |
| obrindatamab | 213 | EVQLVESGGGLVQPGGSLRLSCAASG | 214 | QAVVTQEPSLTVSPGGTVTLTCRSSTGAVTTSNY |
| | | FTFSTYAMNWVRQAPGKGLEWVGRIR | | ANWVQQKPGQAPRGLIGGTNKRAPWTPARFSGS |
| | | SKYNNYATYYADSVKDRFTISRDDSKN | | LLGGKAALTITGAQAEDEADYYCALWYSNLWVFG |
| | | SLYLQMNSLKTEDTAVYYCVRHGNFG | | GGTKLTVL |
| | | NSYVSWFAYWGQGTLVTVSS | | |
| odronextamab | 215 | EVQLVESGGGLVQPGRSLRLSCAASG | 216 | EIVMTQSPATLSVSPGERATLSCRASQSVSSNLA |
| | | FTFDDYTMHWVRQAPGKGLEWVSGIS | | WYQQKPGQAPRLLIYGASTRATGIPARFSGSGSG |
| | | WNSGSIGYADSVKGRFTISRDNAKKSL | | TEFTLTISSLQSEDFAVYYCQHYINWPLTFGGGTK |
| | | YLQMNSLRAEDTALYYCAKDNSGYGH | | VEIK |
| | | YYYGMDWWGQGTTVTVAS | | |
| otelixizumab | 217 | EVQLLESGGGLVQPGGSLRLSCAASG | 218 | DIQLTQPNSVSTSLGSTVKLSCTLSSGNIENNYVH |
| | | FTFSSFPMAWVRQAPGKGLEWVSTIST | | WYQLYEGRSPTTMIYDDDKRPDGVPDRFSGSIDR |
| | | SGGRTYYRDSVKGRFTISRDNSKNTLY | | SSNSAFLTIHNVAIEDEAIYFCHSYVSSFNVFGGG |
| | | LQMNSLRAEDTAVYYCAKFRQYSGGF | | TKLTVL |
| | | DYWGQGTLVTVSS | | |
| pacanalotamab | 219 | EVQLVESGGGLVQPGGSLKLSCAASG | 220 | QTVVTQEPSLTVSPGGTVTLTCGSSTGAVTSGNY |
| | | FTFNKYAMNWVRQAPGKGLEWVARIR | | PNWVQQKPGQAPRGLIGGTKFLAPGTPARFSGS |
| | | SKYNNYATYYADSVKDRFTISRDDSKN | | LLGGKAALTLSGVQPEDEAEYYCVLWYSNRWVF |
| | | TAYLQMNNLKTEDTAVYYCVRHGNFG | | GGGTKLTVL |
| | | NSYISYWAYWGQGTLVTVSS | | |
| pasotuxizumab | 221 | EVQLVESGGGLVQPGGSLKLSCAASG | 222 | QTVVTQEPSLTVSPGGTVTLTCGSSTGAVTSGNY |
| | | FTFNKYAMNWVRQAPGKGLEWVARIR | | PNWVQQKPGQAPRGLIGGTKFLAPGTPARFSGS |
| | | SKYNNYATYYADSVKDRFTISRDDSKN | | LLGGKAALTLSGVQPEDEAEYYCVLWYSNRWVF |
| | | TAYLQMNNLKTEDTAVYYCVRHGNFG | | GGGTKLTVL |
| | | NSYISYWAYWGQGTLVTVSS | | |
| pavurutamab | 223 | EVQLVESGGGLVQPGGSLKLSCAASG | 224 | QTVVTQEPSLTVSPGGTVTLTCGSSTGAVTSGNY |
| | | FTFNKYAMNWVRQAPGKGLEWVARIR | | PNWVQQKPGQAPRGLIGGTKFLAPGTPARFSGS |
| | | SKYNNYATYYADSVKDRFTISRDDSKN | | LLGGKAALTLSGVQPEDEAEYYCVLWYSNRWVF |
| | | TAYLQMNNLKTEDTAVYYCVRHGNFG | | GGGTKLTVL |
| | | NSYISYWAYWGQGTLVTVSS | | |
| plamotamab | 225 | EVQLVESGGGLVQPGGSLRLSCAASG | 226 | QAVVTQEPSLTVSPGGTVTLTCGSSTGAVTTSNY |
| | | FTFSTYAMNWVRQAPGKGLEWVGRIR | | ANWVQQKPGKSPRGLIGGTNKRAPGVPARFSGS |
| | | SKYNNYATYYADSVKGRFTISRDDSKN | | LLGGKAALTISGAQPEDEADYYCALWYSNHWVF |
| | | TLYLQMNSLRAEDTAVYYCVRHGNFG | | GGGTKLTVLEPK |
| | | DSYVSWFAYWGQGTLVTVSS | | |
| runimotamab | 227 | EVQLVQSGAEVKKPGASVKVSCKASG | 228 | DIQMTQSPSSLSASVGDRVTITCRASQDVNTAVA |
| | | YTFTNYYIHWVRQAPGQGLEWIGWIYP | | WYQQKPGKAPKLLIYSASFLYSGVPSRFSGSRSG |
| | | GDGNTKYNEKFKGRATLTADTSTSTAY | | TDFTLTISSLQPEDFATYYCQQHYTTPPTFGQGTK |
| | | LELSSLRSEDTAVYYCARDSYSNYYFD | | VEIK |
| | | YWGQGTLVTVSS | | |
| solitomab | 229 | DVQLVQSGAEVKKPGASVKVSCKASG | 230 | DIVLTQSPATLSLSPGERATLSCRASQSVSYMNW |
| | | YTFTRYTMHWVRQAPGQGLEWIGYIN | | YQQKPGKAPKRWIYDTSKVASGVPARFSGSGSG |
| | | PSRGYTNYADSVKGRFTITTDKSTSTA | | TDYSLTINSLEAEDAATYYCQQWSSNPLTFGGGT |
| | | YMELSSLRSEDTATYYCARYYDDHYCL | | KVEIK |
| | | DYWGQGTTVTVSS | | |
| talquetamab | 231 | EVQLVESGGGLVQPGGSLRLSCAASG | 232 | QTVVTQEPSLTVSPGGTVTLTCRSSTGAVTTSNY |
| | | FTFNTYAMNWVRQAPGKGLEWVARIR | | ANWVQQKPGQAPRGLIGGTNKRAPGTPARFSGS |
| | | SKYNNYATYYAASVKGRFTISRDDSKN | | LLGGKAALTLSGVQPEDEAEYYCALWYSNLWVF |
| | | SLYLQMNSLKTEDTAVYYCARHGNFG | | GGGTKLTVL |
| | | NSYVSWFAYWGQGTLVTVSS | | |
| tarlatamab | 233 | EVQLVESGGGLVQPGGSLKLSCAASG | 234 | QTVVTQEPSLTVSPGGTVTLTCGSSTGAVTSGNY |
| | | FTFNKYAMNWVRQAPGKGLEWVARIR | | PNWVQQKPGQAPRGLIGGTKFLAPGTPARFSGS |
| | | SKYNNYATYYADSVKDRFTISRDDSKN | | LLGGKAALTLSGVQPEDEAEYYCVLWYSNRWVF |
| | | TAYLQMNNLKTEDTAVYYCVRHGNFG | | GGGTKLTVL |
| | | NSYISYWAYWGQGTLVTVSS | | |
| tebentafusp | 235 | EVQLVESGGGLVQPGGSLRLSCAASG | 236 | AIQMTQSPSSLSASVGDRVTITCRASQDIRNYLN |
| | | YSFTGYTMNWVRQAPGKGLEWVALIN | | WYQQKPGKAPKLLIYYTSRLESGVPSRFSGSGSG |
| | | PYKGVSTYNQKFKDRFTISVDKSKNTA | | TDYTLTISSLQPEDFATYYCQQGNTLPWTFGQGT |
| | | YLQMNSLRAEDTAVYYCARSGYYGDS | | KVEIK |
| | | DWYFDVWGQGTLVTVSS | | |
| teclistamab | 237 | EVQLVESGGGLVQPGGSLRLSCAASG | 238 | QTVVTQEPSLTVSPGGTVTLTCRSSTGAVTTSNY |
| | | FTFNTYAMNWVRQAPGKGLEWVARIR | | ANWVQQKPGQAPRGLIGGTNKRAPGTPARFSGS |
| | | SKYNNYATYYAASVKGRFTISRDDSKN | | LLGGKAALTLSGVQPEDEAEYYCALWYSNLWVF |
| | | SLYLQMNSLKTEDTAVYYCARHGNFG | | GGGTKLTVL |
| | | NSYVSWFAYWGQGTLVTVSS | | |
| teplizumab | 239 | QVQLVQSGGGVVQPGRSLRLSCKASG | 240 | DIQMTQSPSSLSASVGDRVTITCSASSSVSYMNW |
| | | YTFTRYTMHWVRQAPGKGLEWIGYINP | | YQQTPGKAPKRWIYDTSKLASGVPSRFSGSGSG |
| | | SRGYTNYNQKVKDRFTISRDNSKNTAF | | TDYTFTISSLQPEDIATYYCQQWSSNPFTFGQGT |
| | | LQMDSLRPEDTGVYFCARYYDDHYCL | | KLQIT |
| | | DYWGQGTPVTVSS | | |
| tepoditamab | 241 | QVQLVQSGGGVVQPGRSLRLSCVASG | 242 | DIQMTQSPSSLSASVGDRVTITCRASQSISSYLNW |
| | | FTFSSYGMHWVRQAPGKGLEWVAAIW | | YQQKPGKAPKLLIYAASSLQSGVPSRFSGSGSGT |
| | | YNARKQDYADSVKGRFTISRDNSKNTL | | DFTLTISSLQPEDFATYYCQQSYSTPPTFGQGTKV |
| | | YLQMNSLRAEDTAVYYCTRGTGYNWF | | EIK |
| | | DPWGQGTLVTVSS | | |
| tidutamab | 243 | EVQLVESGGGLVQPGGSLRLSCAASG | 244 | QAVVTQEPSLTVSPGGTVTLTCGSSTGAVTTSNY |
| | | FTFSTYAMNWVRQAPGKGLEWVGRIR | | ANWVQQKPGKSPRGLIGGTNKRAPGVPARFSGS |
| | | SKYNNYATYYADSVKGRFTISRDDSKN | | LLGGKAALTISGAQPEDEADYYCALWYSNHWVF |
| | | TLYLQMNSLRAEDTAVYYCVRHGNFG | | GGGTKLTVL |
| | | DSYVSWFAYWGQGTLVTVSS | | |
| ubamatamab | 245 | EVQLVESGGGLVQPGRSLRLSCAASG | 246 | DIQMTQSPSSLSASVGDRVTITCRASQSISTYLNW |
| | | FTFDDYSMHWVRQAPGKGLEWVSGIS | | YQQKPGKAPKLLIYTASSLQSGVPSRFSGSGSGT |
| | | WNSGSKGYADSVKGRFTISRDNAKNS | | DFTLTISSLQPEDFATYYCQQSYSTPPITFGQGTR |
| | | LYLQMNSLRAEDTALYYCAKYGSGYG | | LEIK |
| | | KFYHYGLDVWGQGTTVTVSS | | |
| vepsitamab | 247 | EVQLVESGGGLVQPGGSLKLSCAASG | 248 | QTVVTQEPSLTVSPGGTVTLTCGSSTGAVTSGNY |
| | | FTFNKYAMNWVRQAPGKGLEWVARIR | | PNWVQQKPGQAPRGLIGGTKFLAPGTPARFSGS |
| | | SKYNNYATYYADSVKDRFTISRDDSKN | | LLGGKAALTLSGVQPEDEAEYYCVLWYSNRWVF |
| | | TAYLQMNNLKTEDTAVYYCVRHGNFG | | GGGTKLTVL |
| | | NSYISYWAYWGQGTLVTVSS | | |
| vibecotamab | 249 | EVQLVESGGGLVQPGGSLRLSCAASG | 250 | QAVVTQEPSLTVSPGGTVTLTCGSSTGAVTTSNY |
| | | FTFSTYAMNWVRQAPGKGLEWVGRIR | | ANWVQQKPGKSPRGLIGGTNKRAPGVPARFSGS |
| | | SKYNNYATYYADSVKGRFTISRDDSKN | | LLGGKAALTISGAQPEDEADYYCALWYSNHWVF |
| | | TLYLQMNSLRAEDTAVYYCVRHGNFG | | GGGTKLTVLEPK |
| | | DSYVSWFAYWGQGTLVTVSS | | |
| visilizumab | 251 | QVQLVQSGAEVKKPGASVKVSCKASG | 252 | DIQMTQSPSSLSASVGDRVTITCSASSSVSYMNW |
| | | YTFISYTMHWVRQAPGQGLEWMGYIN | | YQQKPGKAPKRLIYDTSKLASGVPSRFSGSGSGT |
| | | PRSGYTHYNQKLKDKATLTADKSASTA | | DFTLTISSLQPEDFATYYCQQWSSNPPTFGGGTK |
| | | YMELSSLRSEDTAVYYCARSAYYDYD | | VEIK |
| | | GFAYWGQGTLVTVSS | | |
| vixtimotamab | 253 | QVQLVQSGAEVKKPGASVKVSCKASG | 254 | DIQMTQSPSSLSASVGDRVTITCRSSTGAVTTSNY |
| | | YTFTSYDINWVRQAPGQGLEWMGWM | | ANWVQQKPGKAPKALIGGTNKRAPGVPSRFSGS |
| | | NPNSGNTGFAQKFQGRVTMTRDTSTS | | LIGDKATLTISSLQPEDFATYYCALWYSNLWVFGQ |
| | | TVYMELSSLRSEDTAVYYCARDRANTD | | GTKVEIK |
| | | YSLGMDVWGQGTLVTVSS | | |
| voxalatamab | 255 | EVQLVESGGGLVQPGGSLRLSCAASG | 256 | QTVVTQEPSLTVSPGGTVTLTCRSSTGAVTTSNY |
| | | FTFNTYAMNWVRQAPGKGLEWVARIR | | ANWVQQKPGQAPRGLIGGTNKRAPGTPARFSGS |
| | | SKYNNYATYYAASVKGRFTISRDDSKN | | LLGGKAALTLSGVQPEDEAEYYCALWYSNLWVF |
| | | SLYLQMNSLKTEDTAVYYCARHGNFG | | GGGTKLTVL |
| | | NSYVSWFAYWGQGTLVTVSS | | |
| brenetafusp | 257 | EVQLVESGGGLVQPGGSLRLSCAASG | 258 | AIQMTQSPSSLSASVGDRVTITCRASQDIRNYLN |
| | | YSFTGYTMNWVRQAPGKGLEWVALIN | | WYQQKPGKAPKLLIYYTSRLESGVPSRFSGSGSG |
| | | PYKGVSTYNQKFKDRFTISVDKSKNTA | | TDYTLTISSLQPEDFATYYCQQGNTLPWTFGQGT |
| | | YLQMNSLRAEDTAVYYCARSGYYGDS | | KVEIK |
| | | DWYFDVWGQGTLVTVSS | | |
| cabotamig | 259 | EVQLVESGGGLVQPGGSLRLSCAASG | 260 | DIQMTQSPSSLSASVGDRVTITCRASQDIRNYLN |
| | | YSFTGYTMNWVRQAPGKGLEWVALIN | | WYQQKPGKAPKLLIYYTSRLESGVPSRFSGSGSG |
| | | PYKGVSTYNQKFKDRFTISVDKSKNTA | | TDYTLTISSLQPEDFATYYCQQGNTLPWTFGQGT |
| | | YLQMNSLRAEDTAVYYCARSGYYGDS | | KVEIK |
| | | DWYFDVWGQGTLVTVSS | | |
| ciduvectamig | 261 | QVQLQQSGPRLVRPSQTLSLTCAISGD | 262 | QSALTQPASVSGSPGQSITISCTGTSSNIGTYKFV |
| | | SVFNNNAAWSWIRQSPSRGLEWLGRT | | SWYQQHPDKAPKVLLYEVSKRPSGVSSRFSGSK |
| | | YYRSKWLYDYAVSVKSRITVNPDTSRN | | SGNTASLTISGLQAEDQADYHCVSYAGSGTLLFG |
| | | QFTLQLNSVTPEDTALYYCARGYSSSF | | GGTKLTVL |
| | | DYWGQGTLVTVSS | | |
| emfizatamab | 263 | EVQLVESGGGLVQPGGSLRLSCAASG | 264 | DVVMTQSPSTLSASVGDRVTINCQASESISSWLA |
| | | FTISTNAMSWVRQAPGKGLEWIGVITG | | WYQQKPGKAPKLLIYEASKLASGVPSRFSGSGSG |
| | | RDITYYASWAKGRFTISRDNSKNTLYL | | TEFTLTISSLQPDDFATYYCQGYFYFISRTYVNSF |
| | | QMNSLRAEDTAVYYCARDGGSSAITSN | | GGGTKVEIK |
| | | NIWGQGTLVTVSS | | |
| etentamig | 265 | EVQLVESGGGLVQPGRSLRLSCAASG | 266 | EIVMTQSPATLSVSPGERATLSCRASQSVSSNLA |
| | | FTFDDYAMHWVRQAPGKGLEWVSGIS | | WYQQKPGQAPRLLIYGASTRATGIPARFSGSGSG |
| | | WNSGSIGYADSVKGRFTISRDNAKNSL | | TEFTLTISSLQSEDFAVYYCQQYNNWPWTFGQGT |
| | | YLQMNSLRAEDTALYYCAKDSRGYGD | | KVEIK |
| | | YRLGGAYWGQGTLVTVSS | | |
| forimtamig | 267 | EVQLLESGGGLVQPGGSLRLSCAASG | 268 | QAVVTQEPSLTVSPGGTVTLTCGSSTGAVTTSNY |
| | | FQFSSYAMNWVRQAPGKGLEWVSRIR | | ANWVQEKPGQAFRGLIGGTNKRAPGTPARFSGS |
| | | SKYNNYATYYADSVKGRFTISRDDSKN | | LLGGKAALTLSGAQPEDEAEYYCALWYSNLWVF |
| | | TLYLQMNSLRAEDTAVYYCVRHTTFPS | | GGGTKLTVL |
| | | SYVSYYGYWGQGTLVTVSS | | |
| gocatamig | 269 | EVQLVESGGGLVQPGGSLKLSCAASG | 270 | QTVVTQEPSLTVSPGGTVTLTCASSTGAVTSGNY |
| | | FTFNKYAINWVRQAPGKGLEWVARIRS | | PNWVQQKPGQAPRGLIGGTKFLVPGTPARFSGS |
| | | KYNNYATYYADQVKDRFTISRDDSKNT | | LLGGKAALTLSGVQPEDEAEYYCTLWYSNRWVF |
| | | AYLQMNNLKTEDTAVYYCVRHANFGN | | GGGTKLTVL |
| | | SYISYWAYWGQGTLVTVSS | | |
| linclatamig | 271 | EVQLVQSGAEVKKPGASVKVSCKASG | 272 | DIVMTQSPDSLAVSLGERATINCKSSQSLLNSRTR |
| | | FTFTSYYIHWVRKAPGQGLEWIGWIYP | | KNYLAWYQEKPGQPPKLLIYWASTRESGVPDRFS |
| | | ENDNTKYNEKFKDRVTITADTSTSTAYL | | GSGSGTDFTLTISSLQAEDVAVYYCTQSFILRTFG |
| | | ELSSLRSEDTAVYYCARDGYSRYYFDY | | QGTKVEIK |
| | | WGQGTLVTVSS | | |
| mipletamig | 273 | QVQLVQSGPEVKKPGSSVKVSCKASG | 274 | DIQMTQSPSTLSASVGDRVTMTCSASSSVSYMN |
| | | YTFSRSTMHWVRQAPGQGLEWIGYIN | | WYQQKPGKAPKRWIYDSSKLASGVPSRFSGSGS |
| | | PSSAYTNYNQKFKDRVTITADKSTSTA | | GTDYTLTISSLQPDDFATYYCQQWSRNPPTFGGG |
| | | YMELSSLRSEDTAVYYCARPQVHYDY | | TKVEIK |
| | | NGFPYWGQGTLVTVSS | | |
| nebratamig | 275 | EVQLVESGGGLVQPGGSLRLSCAASG | 276 | DVVMTQSPSTLSASVGDRVTINCQASESISSWLA |
| | | FTISTNAMSWVRQAPGKGLEWIGVITG | | WYQQKPGKAPKLLIYEASKLASGVPSRFSGSGSG |
| | | RDITYYASWAKGRFTISRDNSKNTLYL | | TEFTLTISSLQPDDFATYYCQGYFYFISRTYVNSF |
| | | QMNSLRAEDTAVYYCARDGGSSAITSN | | GGGTKVEIK |
| | | NIWGQGTLVTVSS | | |
| obertamig | 277 | QVQLQQSGPRLVRPSQTLSLTCAISGD | 278 | QSALTQPASVSGSPGQSITISCTGTSSNIGTYKFV |
| | | SVFNNNAAWSWIRQSPSRGLEWLGRT | | SWYQQHPDKAPKVLLYEVSKRPSGVSSRFSGSK |
| | | YYRSKWLYDYAVSVKSRITVNPDTSRN | | SGNTASLTISGLQAEDQADYHCVSYAGSGTLLFG |
| | | QFTLQLNSVTPEDTALYYCARGYSSSF | | GGTKLTVL |
| | | DYWGQGTLVTVSS | | |
| obrixtamig | 279 | EVQLVESGGGLVQPGGSLKLSCAASG | 280 | EAVVTQEPSLTVSPGGTVTLTCRSSTGAVTTSNY |
| | | FTFNTYAMNWVRQAPGKGLEWVARIR | | ANWVQEKPGQLPRGLIGGTNKRAPWVPARFSGS |
| | | SKYNNYATYYADSVKDRFTISRDDSKN | | LLGGKAALTLSGAQPEDEAEYFCALWYSNLWVF |
| | | TAYLQMNNLKTEDTAVYYCVRHGNFG | | GGGTKLTVL |
| | | NSYVSWFAYWGQGTLVTVSA | | |
| podentamig | 281 | EVQLVESGGGLVQPGGSLKLSCAASG | 282 | QTVVTQEPSLTVSPGGTVTLTCASSTGAVTSGNY |
| | | FTFNKYAINWVRQAPGKGLEWVARIRS | | PNWVQQKPGQAPRGLIGGTKFLVPGTPARFSGS |
| | | KYNNYATYYADQVKDRFTISRDDSKNT | | LLGGKAALTLSGVQPEDEAEYYCTLWYSNRWVF |
| | | AYLQMNNLKTEDTAVYYCVRHANFGN | | GGGTKLTVL |
| | | SYISYWAYWGQGTLVTVSS | | |
| rezetamig | 283 | EVQLVESGGGLVQPGRSLRLSCAASG | 284 | EIVMTQSPATLSVSPGERATLSCRASQSVSSNLA |
| | | FTFHNYAMHWVRQAPGKGLEWVSGIS | | WYQQKPGQAPRLLIYGASTRATGIPARFSGSGSG |
| | | WNSGSIGYADSVKGRFTISRDNAKNSL | | TEFTLTISSLQSEDFAVYYCQQYNNWPWTFGQGT |
| | | YLQMNSLRAEDTALYYCAKDSRGYGD | | KVEIK |
| | | YSLGGAYWGQGTLVTVSS | | |
| umizortamig | 285 | DVQLQESGPSLVKPSQSLSLTCTVTGY | 286 | DILMTQSPSSMSVSLGDTVSITCHSSQDINSNIGW |
| | | SITSDFAWNWIRQFPGNKLEWMGYISY | | LQQRPGKSFKGLIYHGTNLDDEVPSRFSGSGSGA |
| | | SGNTRYNPSLKSRISITRDTSKNQFFLQ | | DYSLTISSLESEDFADYYCVQYAQFPWTFGGGTK |
| | | LNSVTIEDTATYYCVTAGRGFPYWGQG | | LEIK |
| | | TLVTVSA | | |
| vonsetamig | 287 | EVQLVESGGGLVQPGRSLRLSCAASG | 288 | DIQMTQSPSSLSASVGDRVTITCRASQSISSYLNW |
| | | FTFDDYSMHWVRQAPGKGLEWVSGIS | | YQQKPGKAPKLLIYAASSLQSGVPSRFSGSGSGT |
| | | WNSGSIGYADSVKGRFTISRDNAKNSL | | DFTLTISSLQPEDFATYYCQQSYSTPPITFGQGTR |
| | | YLQMNSLRAEDTALYYCAKYGSGYGK | | LEIK |
| | | FYYYGMDVWGQGTTVTVSS | | |
| xaluritamig | 289 | EVQLVESGGGLVQPGGSLRLSCAASG | 290 | QAVVTQEPSLTVSPGGTVTLTCGSSTGAVTTSNY |
| | | FTFSTYAMNWVRQAPGKGLEWVGRIR | | ANWVQQKPGKSPRGLIGGTNKRAPGVPARFSGS |
| | | SKYNNYATYYADSVKGRFTISRDDSKN | | LLGGKAALTISGAQPEDEADYYCALWYSNHWVF |
| | | TLYLQMNSLRAEDTAVYYCVRHGNFG | | GGGTKLTVL |
| | | DSYVSWFAYWGQGTLVTVSS | | |
| zubotamig | 291 | EVKLVESGGGLVQPGGSLRLSCAASG | 292 | QAVVTQEPSFSVSPGGTVTLTCRSSTGAVTTSNY |
| | | FTFNTYAMNWVRQAPGKGLEWVARIR | | ANWVQQTPGQAFRGLIGGTNKRAPGVPARFSGS |
| | | SKYNNYATYYADSVKDRFTISRDDSKS | | LIGDKAALTITGAQADDESIYFCALWYSNLWVFGG |
| | | SLYLQMNNLKTEDTAMYYCVRGGNFG | | GTKLTVL |
| | | NSYVSWFAYWGQGTLVTVSS | | |

### Chimeric Antigen Receptor-T Cells

In another aspect of the present disclosure, a composition for treating cancers expressing a Surface K-Ras antigen includes an engineered immune cell expressing a chimeric antigen receptor. In one embodiment, a chimeric antigen receptor comprises an extracellular domain, a transmembrane domain, and an endodomain.

The extracellular domain is a binding agent, wherein the binding agent that binds with specificity to a Surface K-Ras Antigen expressed on an external surface of a cancer cell. In one embodiment, the binding agent is an antibody or antibody fragment. In another embodiment, the binding agent is an antibody or antibody fragment that selectively binds the Surface K-Ras Antigen, wherein the Surface K-Ras Antigen comprises a mutation at residue 12, 13, or 61 based on the amino acid sequence set forth in SEQ ID NO: 1 or SEQ ID NO: 2. In another embodiment, the binding agent is an antibody or antibody fragment that selectively binds the Surface K-Ras Antigen, wherein the Surface K-Ras Antigen comprises any one of the following mutations based on the amino acid sequence set forth in SEQ ID NO: 1 or SEQ ID NO: 2: G12A; G12D (SEQ ID NO: 295); G12C; G12V; G12R; G13D; Q61H; and Q61 L. In another embodiment, the binding agent is an antibody or antibody fragment that selectively binds the Surface K-Ras Antigen, wherein the Surface K-Ras Antigen comprises any one of the following mutations based on the amino acid sequence set forth in SEQ ID NO: 1 or SEQ ID NO: 2, but either does not bind an antigen of such sequence without one of these mutations or if able to bind such sequence without such mutation, does so with an affinity that would not be considered clinically relevant or therapeutically effective: G12A; G12D (SEQ ID NO: 295); G12C; G12V; G12R; G13D; Q61H; and Q61 L. In any of the above embodiments, the binding agent is an antibody or antibody fragment that binds a Surface K-Ras Antigen that possesses greater than 70% sequence identity to SEQ ID NO: 1 or SEQ ID NO: 2, greater than 80% sequence identity to SEQ ID NO: 1 or SEQ ID NO: 2, greater than 90% sequence identity to SEQ ID NO: 1 or SEQ ID NO: 2, or any percentage of sequence identity to SEQ ID NO: 1 or SEQ ID NO: 2 between 70% and 99%. In any of the above embodiments, the binding agent is an antibody or antibody fragment that binds a Surface K-Ras Antigen having the sequence of K-Ras G12D (SEQ ID NO: 295). Binding agents suitable for use in the CAR-T cell embodiments disclosed herein include those listed in Table 1 and/or Table 2 as applicable for a given cancer-expressing mutation.

In any of the foregoing embodiments, the chimeric antigen receptor may have a transmembrane domain selected from the group consisting of CD3-zeta, CD28, CDE28a, CD4 or combinations thereof.

In any of the foregoing embodiments, the chimeric antigen receptor may have an endodomain that is selected from the group consisting of CD28, CD27, 4-1 BB, OX40, and/or ICOS.

In certain embodiments, immune cells are transformed with a vector that expresses a nucleotide sequence encoding the CARs described herein. The vector may be any vector that can express the CAR proteins within an immune cell. The vector may further comprise control sequences that allow it to be replicated and/or expressed in both prokaryotic and eukaryotic cells. One of skill in the art would further understand the conditions under which to incubate all of the above-described host cells to maintain them and to permit replication of a vector. Also understood and known are techniques and conditions that would allow large-scale production of vectors, as well as production of the nucleic acids encoded by vectors and their cognate polypeptides, proteins, or peptides.

The immune cells can be autologous, syngeneic, allogenic or xenogeneic cells. A vector encoding any of the foregoing chimeric antigen receptors is transfected into an immune cell to yield a CAR-T cell or CAR-Immune cell, which will be administered to a subject having a cancer expressing a Surface K-Ras Antigen. In one embodiment, the immune cell is derived from the individual to which the vector will be transfected. In another embodiment, the immune cell is derived from a different individual. In any of the foregoing embodiments, the immune cells is selected from a T cell, a natural killer (NK) cell, a dendritic cell or a mixture thereof. In another embodiment, the immune cell is a CD4+ T cell or a CD8+ T cell.

In some embodiments, administration of CAR-T cells will require leukapheresis. Patient blood cells will be collected, and the collected cells may be enriched, or depleted to achieve populations of desired cells. In a preferred embodiment, patient T cells will be isolated. The isolated patient T cells will be expanded, modified with the engineered CAR receptor, and infused into patient by methods known to those skilled in the arts.

### Methods of Treatment, Administration, Formulation and Dosing

In one aspect, a method of treating a subject with a cancer associated with expression of Surface K-Ras Antigen are provided. These methods comprise administering a therapeutically-effective amount of a composition comprising a binding agent that binds with specificity to a Surface K-Ras Antigen expressed on the external surface of a cancer cell. In one embodiment, the binding agent is an antibody or antibody fragment. In another embodiment, the binding agent is an antibody or antibody fragment that selectively binds the Surface K-Ras Antigen, wherein the Surface K-Ras Antigen comprises a mutation at residue 12, 13, or 61 based on the amino acid sequence set forth in SEQ ID NO: 1 or SEQ ID NO: 2. In another embodiment, the binding agent is an antibody or antibody fragment that selectively binds the Surface K-Ras Antigen, wherein the Surface K-Ras Antigen comprises any one of the following mutations based on the amino acid sequence set forth in SEQ ID NO: 1 or SEQ ID NO: 2: G12A; G12D; G12C; G12V; G12R; G13D; Q61H; and Q61L. In another embodiment, the binding agent is an antibody or antibody fragment that selectively binds the Surface K-Ras Antigen, wherein the Surface K-Ras Antigen comprises any one of the following mutations based on the amino acid sequence set forth in SEQ ID NO: 1 or SEQ ID NO: 2, but either does not bind an antigen of such sequence without one of these mutations or if able to bind such sequence without such mutation, does so with an affinity that would not be considered clinically relevant or therapeutically effective: G12A; G12D; G12C; G12V; G12R; G13D; Q61H; and Q61 L In any of the above embodiments, the binding agent is an antibody or antibody fragment that binds a Surface K-Ras Antigen that possesses greater than 70% sequence identity to SEQ ID NO: 1 or SEQ ID NO: 2, greater than 80% sequence identity to SEQ ID NO: 1 or SEQ ID NO: 2, greater than 90% sequence identity to SEQ ID NO: 1 or SEQ ID NO: 2, or any percentage of sequence identity to SEQ ID NO: 1 or SEQ ID NO: 2 between 70% and 99%. Binding agents suitable for use in the methods of treatment embodiments disclosed herein include those listed in Table 1 and/or Table 2 as applicable for a given cancer-expressing mutation, Human Anti-K-Ras Antibody 1 and Human Anti-K-Ras Antibody 2. In any of the above method embodiments, the binding agent may be linked to another binding agent that binds to an antigen on the surface of an immune effector cell as discussed herein with respect to embodiments for compositions comprising bispecific antibodies and herein incorporating all features and embodiments disclosed therewith. In any of the above method embodiments, the binding agent may be linked to therapeutic agent that is cytotoxic to the subject's cancer cells as discussed herein with respect to embodiments for compositions comprising antibody-drug conjugates and herein incorporating all features and embodiments disclosed therewith. In any of the above method embodiments, the binding agent may be a portion of a chimeric antigen receptor expressed in an immune effector cell as discussed herein with respect to embodiments for compositions comprising CAR-T cells and herein incorporating all features and embodiments disclosed therewith. In any of the above embodiments, the method does not include administration of an intracellular delivery compound.

The amount of compositions embodied in this invention, may vary according to the needs of the patient, but should be provided at a therapeutically-effective amount.

Certain embodiments of this invention include the co-administration of the compositions embodied in this invention with other treatments such as chemotherapy, surgery, radiotherapy, immunomodulators, and other therapeutic agents. Similarly, we have discovered that treating K-Ras expressing cells with chemotherapy (such as 5-FU) and small molecule inhibitors of K-Ras activity lead to increased binding availability of Surface K-Ras Antigen or otherwise increased expression thereof (See Examples 11, 12a, and 12b).

Thus, in one embodiment, a method of treating, inhibiting or reducing proliferation of or killing a cancer cell in a subject comprising administering a therapeutic agent to the subject prior to or concurrently with the administration of any of the foregoing compositions embodied in this invention. The therapeutic agent should be understood in this instance to be separate and apart of any therapeutic agent that may be associated with a composition embodied in this invention. In one specific embodiment, the therapeutic agent is a chemotherapy such as 5-FU. In another specific embodiment, the therapeutic agent is a small molecule inhibitor of K-Ras, including, but not limited to MRTX1133 (activity against the G12D mutant K-Ras), MRTX849 (activity against the G12C mutant K-Ras), or RMC-6236 (activity against multiple GTP-bound RAS proteins, including K-RAS WT and K-RAS G12 mutants). In another specific embodiment, multiple therapeutic agents are used. For example, in one specific embodiment, the one or more therapeutic agents are chemotherapy and a small molecule inhibitor of K-Ras. In another specific embodiment, the one or more therapeutic agents are 5-FU chemotherapy agent and a small molecule inhibitor of K-Ras, including, but not limited to MRTX1133 (activity against the G12D mutant K-Ras), MRTX849 (activity against the G12C mutant K-Ras), or RMC-6236 (activity against multiple GTP-bound RAS proteins, including K-RAS WT and K-RAS G12 mutants). More generally, the K-Ras small molecule inhibitor is one that specifically inhibits the activity of a form of K-Ras carrying a mutation that is present in the Surface K-Ras Antigen targeted by the compositions embodied by this invention, including any one of the known K-Ras mutations described herein.

The therapeutic agent is administered to the subject 1 day to 21 days prior to administration of the composition and any specific day between 1 day and 21 days prior to administration and any intermediate range there between (e.g., 3 days to 7 days, 7 days to 14 days, 10 days to 21 days, etc.).

The method or route of administration and dose for embodiments involving pretreatment or concurrent treatment with additional therapeutic agents will be that manner in which such therapeutic agent is normally administered and dosed under standard treatment conditions for indications for which the therapeutic agent is routinely used. In some embodiments, the dose may be reduced from what is considered to be the indicated or approved dose.

The method or route of administration for the compositions embodied in this invention is any that are suitable as determined by a skilled physician/clinician. Non-limiting examples of method of administration include injection, transfusion, or implantation. Non-limiting examples of the route of administration include parenteral, intravenous, tumoral, arterial, muscular, peritoneal, and/or subcutaneous.

The dosage and frequency of dosing may vary according to clinical conditions. The dose and dose frequency can be determined by any physician or clinician skilled in the art. In some cases, higher or lower doses may be appropriate, if an effective dose is administered. The frequency of dosing may only require a single dose or multiple doses. For CAR-T administration, the number of cells administered may vary but preferred embodiments would be 10⁴-10⁹ cells/kg.

The compositions of the present disclosure can be used for treatment of various cancers associated with expression of mutations in K-Ras or K-Ras overexpression, such as pancreatic cancers (including pancreatic ductal adenocarcinoma (PDAC), lung cancers (including non-small cell lung cancer (NSCLC), cholangial cancers, ovarian cancers, endometrial cancers, or colorectal cancers.

It should be understood that any disclosure or embodiment directed to a method of treatment, inhibition or reduction of a cancer is equally applicable to medical uses for the same.

### Diagnostics

Because the therapeutic compositions and methods of the present disclosure are dependent on the surface expression of K-Ras antigen (Surface K-Ras Antigen) in cancer cells as opposed to intracellular expression of K-Ras, it is important to first determine whether the subject's cancer expresses this particular antigen on the surface. Thus, the present disclosure further provides methods and compositions for diagnosing cancers expressing Surface K-Ras Antigen. In general, the compositions for diagnosing may comprise any binding agent or other compound that can specifically detect Surface K-Ras Antigen on a cell and the method employed must be able to distinguish or separate Surface K-Ras Antigen from K-Ras antigen present inside the cell. With these considerations in mind, one of ordinary skill in the art should be able to identify many different methods of diagnosis and the specific embodiments of the present disclosure should therefore only be understood as exemplary and not limiting.

The binding agents described herein for use with ADCs and CAR-T cells may also present useful agents in detecting Surface K-Ras Antigens in cancer cells. However, any agents that cannot be transported across the cell membrane (or can be modified to not cross the cell membrane or otherwise enter the cell) and that have specificity to Surface K-Ras Antigen could be useful as a diagnostic tool. In one embodiment, the binding agent is an antibody or antibody fragment. In another embodiment, the binding agent is an antibody or antibody fragment that selectively binds the Surface K-Ras Antigen, wherein the Surface K-Ras Antigen comprises a mutation at residue 12, 13, or 61 based on the amino acid sequence set forth in SEQ ID NO: 1 or SEQ ID NO: 2. In another embodiment, the binding agent is an antibody or antibody fragment that selectively binds the Surface K-Ras Antigen, wherein the Surface K-Ras Antigen comprises any one of the following mutations based on the amino acid sequence set forth in SEQ ID NO: 1 or SEQ ID NO: 2: G12A; G12D; G12C; G12V; G12R; G13D; Q61H; and Q61L. In another embodiment, the binding agent is an antibody or antibody fragment that selectively binds the Surface K-Ras Antigen, wherein the Surface K-Ras Antigen comprises any one of the following mutations based on the amino acid sequence set forth in SEQ ID NO: 1 or SEQ ID NO: 2, but either does not bind an antigen of such sequence without one of these mutations or if able to bind such sequence without such mutation, does so with an affinity that would not be considered clinically relevant or therapeutically effective: G12A; G12D; G12C; G12V; G12R; G13D; Q61H; and Q61L In any of the above embodiments, the binding agent is an antibody or antibody fragment that binds a Surface K-Ras Antigen that possesses greater than 70% sequence identity to SEQ ID NO: 1 or SEQ ID NO: 2, greater than 80% sequence identity to SEQ ID NO: 1 or SEQ ID NO: 2, greater than 90% sequence identity to SEQ ID NO: 1 or SEQ ID NO: 2, or any percentage of sequence identity to SEQ ID NO: 1 or SEQ ID NO: 2 between 70% and 99%. Binding agents suitable for use in the diagnostic embodiments disclosed herein include those listed in Table 1 and/or Table 2 as applicable for a given cancer-expressing mutation, including Human Anti-K-Ras Antibody 1 and Human Anti-K-Ras Antibody 2. In any of the above embodiments, the method does not include administration of an intracellular delivery compound.

In one embodiment, a diagnostic method of the present disclosure involves (a) obtaining a cell sample from a subject comprising a population of cells; (b) exposing the cell sample to an agent capable of binding the Surface K-Ras Antigen, wherein the population of cells are not lysed or otherwise permeabilized to avoid the agent reacting with intracellular forms of K-Ras; and (c) measuring the presence of the agent on the external surface of the population of cells present in the cell sample. In one embodiment, the Surface K-Ras Antigen is a mutant Surface K-Ras Antigen. The diagnostic method of the preceding embodiment may be performed on a sample already obtained from the subject.

In one embodiment, the step of obtaining a cell sample from a subject comprising a population of cells comprises guided biopsy, such as a fine needle aspiration biopsy. In this method, a thin, hollow needle is inserted through the skin and into the target tissue sample. This may be done by CT guidance, ultrasound guidance, or by tactile sensation. In addition, a lesion may be accessed within the gastrointestinal track using endoscopy such as colonoscopy, upper endoscopy, or endoscopic retrograde cholangiopancreatography. This biopsy needle is used to aspirate out a small sample of cells (and fluid) to be used for analysis. In addition, a larger needle may be utilized to obtain a large core needle biopsy that may be used to obtain a larger mass of cells. However, the desired result of this fine needle biopsy or FNA is to obtain a single cell suspension where the cells remain intact in order to assess cell-surface antigen expression.

In another embodiment, the step of obtaining a cell sample from a solid tumor can be performed with the following steps: (1) cutting tissue into small segments (e.g., approximately 1 mg); (2) suspending the segments in media followed by dissociation (e.g., using a MACS dissociator or another device capable to creating a single cell suspension); (3) digesting the dissociated samples in media supplemented with in collagenase and DNase; (4) repeat dissociation of step 2 to retrieve single cells; (5) wash the cells to eliminate digestion enzyme and DNase; (6) subject cells to strainer and resuspend in the appropriate media. It should be understood that there are many alternative methods by which to achieve a cell suspension from a solid tumor or sample and the embodiments described herein should only be considered as exemplary and not limiting.

The cell sample may comprise pancreatic cells, lung cells, cholangial cells, ovarian cells, endometrial cells, or colorectal cells.

The cell sample can then be exposed to an agent(s) capable of binding the Surface K-Ras Antigen. In this step, care should be taken to avoid lysis or otherwise subjecting the cells to conditions that would allow the agent reacting with intracellular forms of K-Ras. Preferably, the agent (such as a binding agent of the present disclosure) should be an agent that is not capable of crossing the cell membrane without the use intracellular delivery compounds. In one embodiment, the agent is an antibody or antibody fragment or other protein or peptide that binds to Surface K-Ras Antigen. In a further embodiment, the antibody or antibody fragment comprises a label, such a fluorochrome. In other embodiments, the method comprises applying a first agent and exposing the sample to a secondary binding agent that binds to a portion of the first agent, wherein the secondary binding agent is labeled.

The step of measuring can then be performed by methods commonly used in the art to determine the interaction between the agent and Surface K-Ras Antigen, such as applying the cell sample to a flow cytometer or electron microscopy.

Confocal microscopy can also be used and would further comprise an additional step wherein the cell sample is exposed to a cell-surface membrane dye prior to exposure with the agent reacting with Surface K-Ras Antigen.

In another embodiment, the diagnostic method comprises the steps of: (a) obtaining a cell sample from a subject comprising a population of cells; (b) labeling proteins on the external surface of the cell with a first agent; (c) lysing the population of cells to yield a cell lysate sample; (d) capturing labeled proteins by applying the cell lysate sample to a surface coated with a second agent, wherein the second agent selectively binds the first agent; (e) removing the captured labeled proteins from the surface and removing the first agent form the captured labeled proteins to yield a cell-surface protein sample; (f) exposing the cell-surface protein sample to a third agent capable of selectively binding the Surface K-Ras Antigen; (g) exposing the cell surface protein sample to a fourth agent, wherein the fourth agent carries a detectable label and binds to a portion of the third agent; and (h) measuring the presence of the detectable label. One example of this embodiment includes a Western blot. The diagnostic method of the preceding embodiment may be performed on a sample already obtained from the subject.

Upon diagnosis of a cancer expressing a Surface K-Ras Antigen, any of the foregoing therapeutic compositions and methods may be used with the patient.

### EXEMPLARY EMBODIMENTS OF THE PRESENT DISCLOSURE

Embodiment 1: A composition comprising: a binding agent-therapeutic agent complex comprising a binding agent linked to a therapeutic agent, wherein the binding agent binds with specificity to a Surface K-Ras Antigen expressed on an external surface of a cancer cell, and wherein neither the composition nor the binding agent-therapeutic agent complex comprises an intracellular delivery compound.

The composition of Embodiment 1, wherein the binding agent is an antibody or antibody fragment.

The composition of Embodiment 1, wherein the binding agent is an antibody or antibody fragment that selectively binds Surface K-Ras Antigen with a mutation at residue 12, 13, or 61 based on the amino acid sequence set forth in SEQ ID NO: 1 or SEQ ID NO: 2.

The composition of Embodiment 1, wherein the binding agent is an antibody or antibody fragment that selectively binds Surface K-Ras Antigen with any one of the following mutations based on the amino acid sequence set forth in SEQ ID NO: 1 or SEQ ID NO: 2: G12A; G12D; G12C; G12V; G12R; G13D; Q61H; and Q61L.

The composition of Embodiment 1, wherein the binding agent is an antibody or antibody fragment that selectively binds Surface K-Ras Antigen with the G12D mutation based on the amino acid sequence set forth in SEQ ID NO: 1 or SEQ ID NO: 2.

The composition of Embodiment 1, wherein the binding agent is an antibody or antibody fragment that selectively binds the Surface K-Ras Antigen, wherein the Surface K-Ras Antigen comprises the G12C mutation based on the amino acid sequence set forth in SEQ ID NO: 1 or SEQ ID NO: 2.

The composition of Embodiment 1, wherein the binding agent is an antibody or antibody fragment that selectively binds the Surface K-Ras Antigen, wherein the Surface K-Ras Antigen comprises the G12V mutation based on the amino acid sequence set forth in SEQ ID NO: 1 or SEQ ID NO: 2.

The composition of Embodiment 1, wherein the binding agent is an antibody fragment.

The composition of Embodiment 1, wherein the binding agent is a peptide or protein.

The composition of Embodiment 1, wherein the binding agent is a peptide or protein that selectively binds the Surface K-Ras Antigen, wherein the Surface K-Ras Antigen comprises the G12D mutation based on the amino acid sequence set forth in SEQ ID NO: 1 or SEQ ID NO: 2.

The composition of Embodiment 1, wherein the binding agent is a peptide or protein that selectively binds the Surface K-Ras Antigen, wherein the Surface K-Ras Antigen comprises a mutation at residue 12, 13, or 61 based on the amino acid sequence set forth in SEQ ID NO: 1 or SEQ ID NO: 2.

The composition of Embodiment 1, wherein the binding agent is a peptide or protein that selectively binds the Surface K-Ras Antigen, wherein the Surface K-Ras Antigen comprises the G12C mutation based on the amino acid sequence set forth in SEQ ID NO: 1 or SEQ ID NO: 2.

The composition of Embodiment 1, wherein the binding agent is a peptide or protein that selectively binds the Surface K-Ras Antigen, wherein the Surface K-Ras Antigen comprises the G12V mutation based on the amino acid sequence set forth in SEQ ID NO: 1 or SEQ ID NO: 2.

The composition of Embodiment 1, wherein the binding agent is an antibody described in Table 1 or Table 2, or is an antibody selected by the methods set forth in Examples 4 or 5.

The composition of any of the applicable foregoing embodiments of Embodiment 1, wherein the therapeutic agent is selected from the group consisting of a cytotoxic agent, a cytostatic agent, a toxin, or a radionuclide.

The composition of any of the applicable foregoing embodiments of Embodiment 1, wherein the therapeutic agent is selected from the group consisting of DNA-damaging agents (alkylating agents), antimetabolites, topoisomerase inhibitors, mitotic inhibitors, antitumor antibiotics, and microtubule-disrupting agents.

The composition of any of the applicable foregoing embodiments of Embodiment 1, wherein the therapeutic agent is selected from the group consisting of calicheamicin, saporin, maytansinoid, auristatin, lidamycin, methotrexate, vinblastine, vincristine, pyrrolobenzodiazepines and other benzodiazepine derivatives, duocarmycins, tubulysins, α-amanitin or bouganin protein toxins, doxorubicin, etoposide, fluorouracil, Gemcitabine, paclitaxel, cisplatin, cyclophosphamide, amatoxin, carboplatin, spliceostatin C, docetaxel, thailanstatin A or any combination thereof.

The composition of any of the applicable foregoing embodiments of Embodiment 1, wherein the binding agent is linked to the therapeutic agent by a linker selected from the group consisting of a maleimidocaproyl linker, a peptide-based linker (including, but not limited to a valine-citrulline linker), a β-glucuronide linker, a SMCC linker, a disulfide linker, or an acid sensitive linker.

The composition of any of the applicable foregoing embodiments of Embodiment 1, wherein the therapeutic agent is a radionuclide.

The composition of the applicable foregoing embodiments of Embodiment 1, wherein the radionuclide is a β-particle-emitting radionuclide or an α-particle emitting radionuclide.

The composition of the applicable foregoing embodiments of Embodiment 1, wherein the radionuclide is an α-particle emitting radionuclide selected from the group consisting of astatine-211, bismuth-212, lead-212, bismuth-213, actinium-225, radium-223 and thorium-227.

The composition of the applicable foregoing embodiments of Embodiment 1, wherein the radionuclide is a β-particle-emitting radionuclide.

The composition of the applicable foregoing embodiments of Embodiment 1, wherein the β-particle-emitting radionuclide is selected from the group consisting of iodine-131, rhenium-186, yttrium-90, samarium-153, and lutetium-177.

Embodiment 2: A method of treating, inhibiting or reducing proliferation of or killing a cancer cell in a subject comprising administering a therapeutically-effective amount of a composition of any of the embodiments of Embodiment 1, 7, and a composition comprising the bispecific antibodies of any of the embodiments of Embodiment 16 to the subject, wherein the subject is not administered an intracellular delivery compound in conjunction with administration of the composition, and wherein a Surface K-Ras Antigen is expressed on the external surface of the cancer cell.

The method of Embodiment 2, wherein the cancer cells are selected from the group consisting of pancreatic cancer cells, lung cancer cells, cholangial cancer cells, ovarian cancer cells, endometrial cancer cells, or colorectal cancer cells.

The method of any of the applicable foregoing embodiments of Embodiment 2, wherein the administering step is performed by intravenous injection or subcutaneous injection.

Embodiment 3: A method of treating, inhibiting or reducing proliferation of or killing a cancer cell in a subject comprising administering a therapeutically-effective amount of a composition, wherein the composition comprises a means for selectively binding to a Surface K-Ras Antigen expressed on the external surface of the cancer cell.

The method of Embodiment 3, wherein the means is an antibody.

The method of Embodiment 3, wherein the means is an antibody or antibody fragment that selectively binds the Surface K-Ras Antigen, wherein the Surface K-Ras Antigen comprises a mutation at residue 12, 13, or 61 based on the amino acid sequence set forth in SEQ ID NO: 1 or SEQ ID NO: 2.

The method of Embodiment 3, wherein the means is an antibody or antibody fragment that selectively binds the Surface K-Ras Antigen, wherein the Surface K-Ras Antigen comprises any one of the following mutations based on the amino acid sequence set forth in SEQ ID NO: 1 or SEQ ID NO: 2: G12A; G12D; G12C; G12V; G12R; G13D; Q61H; and Q61 L.

The method of Embodiment 3, wherein the means is an antibody or antibody fragment that selectively binds the Surface K-Ras Antigen, wherein the Surface K-Ras Antigen comprises the G12D mutation based on the amino acid sequence set forth in SEQ ID NO: 1 or SEQ ID NO: 2.

The method of Embodiment 3, wherein the means is an antibody or antibody fragment that selectively binds the Surface K-Ras Antigen, wherein the Surface K-Ras Antigen comprises the G12C mutation based on the amino acid sequence set forth in SEQ ID NO: 1 or SEQ ID NO: 2.

The method of Embodiment 3, wherein the means is an antibody or antibody fragment that selectively binds the Surface K-Ras Antigen, wherein the Surface K-Ras Antigen comprises the G12V mutation based on the amino acid sequence set forth in SEQ ID NO: 1 or SEQ ID NO: 2.

The method of Embodiment 3, wherein the means is an antibody fragment.

The method of Embodiment 3, wherein the means is a peptide or protein.

The method of Embodiment 3, wherein the means is a peptide or protein that selectively binds the Surface K-Ras Antigen, wherein the Surface K-Ras Antigen comprises the G12D mutation based on the amino acid sequence set forth in SEQ ID NO: 1 or SEQ ID NO: 2.

The method of Embodiment 3, wherein the means is a peptide or protein that selectively binds the Surface K-Ras Antigen, wherein the Surface K-Ras Antigen comprises a mutation at residue 12, 13, or 61 based on the amino acid sequence set forth in SEQ ID NO: 1 or SEQ ID NO: 2.

The method of Embodiment 3, wherein the means is a peptide or protein that selectively binds the Surface K-Ras Antigen, wherein the Surface K-Ras Antigen comprises the G12C mutation based on the amino acid sequence set forth in SEQ ID NO: 1 or SEQ ID NO: 2.

The method of Embodiment 3, wherein the means is a peptide or protein that selectively binds the Surface K-Ras Antigen, wherein the Surface K-Ras Antigen comprises the G12V mutation based on the amino acid sequence set forth in SEQ ID NO: 1 or SEQ ID NO: 2.

The method of Embodiment 3, wherein the means is an antibody described in Table 1 or Table 2, or a fragment thereof or is an antibody selected by the methods set forth in Examples 4 or 5.

The method of Embodiment 3, wherein the cancer cell is a pancreatic cancer cell, a lung cancer cell, or a colorectal cancer cell.

The method of any of the applicable foregoing embodiments of Embodiment 3, wherein the administering step is performed by intravenous injection or subcutaneous injection.

The method of any of the applicable foregoing embodiments of Embodiment 3, wherein the Surface K-Ras Antigen is a full length or truncated form of K-Ras with a mutation at any of the following amino acids/residues: 12 (including, but not limited to G12A, G12D, G12C, G12V, G12R); 13 (including, but not limited to G13D); and 61 (including, but not limited to Q61H, Q61 L) based on the amino acid position in SEQ ID NO: 1 or SEQ ID NO: 2.

Embodiment 4: A chimeric antigen receptor comprising an extracellular domain, a transmembrane domain, and an endodomain, wherein the extracellular domain comprises a binding agent, wherein the binding agent binds with specificity to a Surface K-Ras Antigen expressed on an external surface of a cancer cell.

The chimeric antigen receptor of Embodiment 4, wherein the transmembrane domain is selected from the group consisting of CD3-zeta, CD28, CDE28a, CD4 or combinations thereof.

The chimeric antigen receptor of any of the applicable foregoing embodiments of Embodiment 4, wherein the endodomain is selected from the group consisting of CD28, CD27, 4-1 BB, OX40, and/or ICOS.

The chimeric antigen receptor of any of the applicable foregoing embodiments of Embodiment 4, wherein the binding agent is an antibody or antibody fragment.

The chimeric antigen receptor of any of the applicable foregoing embodiments of Embodiment 4, wherein the binding agent is an antibody or antibody fragment that selectively binds the Surface K-Ras Antigen, wherein the Surface K-Ras Antigen comprises a mutation at residue 12, 13, or 61 based on the amino acid sequence set forth in SEQ ID NO: 1 or SEQ ID NO: 2.

The chimeric antigen receptor of any of the applicable foregoing embodiments of Embodiment 4, wherein the binding agent is an antibody or antibody fragment that selectively binds the Surface K-Ras Antigen, wherein the Surface K-Ras Antigen comprises any one of the following mutations based on the amino acid sequence set forth in SEQ ID NO: 1 or SEQ ID NO: 2: G12A; G12D; G12C; G12V; G12R; G13D; Q61H; and Q61L.

The chimeric antigen receptor of any of the applicable foregoing embodiments of Embodiment 4, wherein the binding agent is an antibody or antibody fragment that selectively binds the Surface K-Ras Antigen, wherein the Surface K-Ras Antigen comprises the G12D mutation based on the amino acid sequence set forth in SEQ ID NO: 1 or SEQ ID NO: 2.

The chimeric antigen receptor of any of the applicable foregoing embodiments of Embodiment 4, wherein the binding agent is an antibody or antibody fragment that selectively binds the Surface K-Ras Antigen, wherein the Surface K-Ras Antigen comprises the G12C mutation based on the amino acid sequence set forth in SEQ ID NO: 1 or SEQ ID NO: 2.

The chimeric antigen receptor of any of the applicable foregoing embodiments of Embodiment 4, wherein the binding agent is an antibody or antibody fragment that selectively binds the Surface K-Ras Antigen, wherein the Surface K-Ras Antigen comprises the G12V mutation based on the amino acid sequence set forth in SEQ ID NO: 1 or SEQ ID NO: 2.

The chimeric antigen receptor of any of the applicable foregoing embodiments of Embodiment 4, wherein the binding agent is an antibody fragment.

The chimeric antigen receptor of any of the applicable foregoing embodiments of Embodiment 4, wherein the Surface K-Ras Antigen possesses greater than 70% sequence identity to SEQ ID NO: 1 or SEQ ID NO: 2.

The chimeric antigen receptor of any of the applicable foregoing embodiments of Embodiment 4, wherein the Surface K-Ras Antigen possesses greater than 80% sequence identity to SEQ ID NO: 1 or SEQ ID NO: 2.

The chimeric antigen receptor of any of the applicable foregoing embodiments of Embodiment 4, wherein the Surface K-Ras Antigen possesses greater than 90% sequence identity to SEQ ID NO: 1 or SEQ ID NO: 2.

The composition of any of the applicable foregoing embodiments of Embodiment 1, wherein the Surface K-Ras Antigen possesses greater than 70% sequence identity to SEQ ID NO: 1 or SEQ ID NO: 2.

The composition of any of the applicable foregoing embodiments of Embodiment 1, wherein the Surface K-Ras Antigen possesses greater than 80% sequence identity to SEQ ID NO: 1 or SEQ ID NO: 2.

The composition of any of the applicable foregoing embodiments of Embodiment 1, wherein the Surface K-Ras Antigen possesses greater than 90% sequence identity to SEQ ID NO: 1 or SEQ ID NO: 2 or greater than 95% sequence identity to SEQ ID NO: 1 or SEQ ID NO: 2.

The method of any of the applicable foregoing embodiments of Embodiment 3, wherein the Surface K-Ras Antigen possesses greater than 70% sequence identity to SEQ ID NO: 1 or SEQ ID NO: 2.

The method of any of the applicable foregoing embodiments of Embodiment 3, wherein the Surface K-Ras Antigen possesses greater than 80% sequence identity to SEQ ID NO: 1 or SEQ ID NO: 2.

The method of any of any of the applicable foregoing embodiments of Embodiment 3, wherein the Surface K-Ras Antigen possesses greater than 90% sequence identity to SEQ ID NO: 1 or SEQ ID NO: 2 or greater than 95% sequence identity to SEQ ID NO: 1 or SEQ ID NO: 2.

Embodiment 5: A vector comprising a nucleotide sequence encoding the chimeric antigen receptor of any of the applicable foregoing embodiments of Embodiment 4.

Embodiment 6: An immune cell expressing the chimeric antigen receptor of any of the applicable embodiments of Embodiment 4.

The immune cell of Embodiment 6, wherein the immune cell is a cell from an individual.

The immune cell of Embodiment 6, wherein the immune cell is a T cell from an individual.

The immune cell of Embodiment 6, wherein the immune cell is selected from the group consisting of a T cell, a NK cell, a dendritic cell or a mixture thereof.

The immune cell of Embodiment 6, wherein the immune cell is a T cell.

The immune cell of Embodiment 6, wherein the immune cell is a CD4+ T cell or a CD8+ T cell.

Embodiment 7: A composition comprising an immune cell that expresses a chimeric antigen receptor that targets Surface K-Ras Antigen expressed on the extracellular surface of cancer cells.

The composition of Embodiment 7, wherein the Surface K-Ras Antigen is a mutant Surface K-Ras Antigen.

The composition of Embodiment 7, wherein the immune cell is derived from a source that is autologous, syngeneic, allogenic or xenogeneic.

The composition of Embodiment 7, wherein the immune cell is a T cell, and wherein the T cell is derived from a source that is autologous, syngeneic, allogenic or xenogeneic.

The composition of Embodiment 7, wherein the immune cell is selected from the group consisting of a T cell, a NK cell, a dendritic cell or a mixture thereof.

The composition of Embodiment 7, wherein the immune cell is a T cell.

The composition of Embodiment 7, wherein the immune cell is a CD4+ T cell or a CD8+ T cell.

Embodiment 8: A method of treating an individual having a cancer comprising cancer cells that express a Surface K-Ras Antigen on an external surface of the cancer cells comprising administering to the individual a therapeutically effective amount of immune cells expressing the chimeric antigen receptor of any of the embodiments of Embodiment 4.

The method of Embodiment 8, wherein the Surface K-Ras Antigen is a mutant Surface K-Ras Antigen.

The method of Embodiment 8, wherein the immune cells comprise a cell from the individual having the cancer.

The method of Embodiment 8, wherein the immune cells are T cells from the individual having the cancer.

The method of Embodiment 8, wherein the immune cell is selected from the group consisting of a T cell, a NK cell, a dendritic cell or a mixture thereof.

The method of Embodiment 8, wherein the immune cell is a T cell.

The method of Embodiment 8, wherein the immune cell is a CD4+ T cell or a CD8+ T cell.

The method of Embodiment 8, wherein the cancer cells are selected from the group consisting of pancreatic cancer cells, lung cancer cells, cholangial cancer cells, ovarian cancer cells, endometrial cancer cells, or colorectal cancer cells.

Embodiment 9: A method of treating an individual having a cancer comprising cancer cells that express a Surface K-Ras Antigen on an external surface of the cancer cells comprising administering to the individual a therapeutically effective amount of the composition of any of the embodiments of Embodiment 7.

The method of Embodiment 9, wherein the cancer cells are selected from the group consisting of pancreatic cancer cells, lung cancer cells, cholangial cancer cells, ovarian cancer cells, endometrial cancer cells, or colorectal cancer cells.

Embodiment 10: A method of diagnosing a cancer associated with an expression of Surface K-Ras Antigen on an external surface of a cell, the method comprising the steps of: (a) obtaining a cell sample from a subject comprising a population of cells; (b) exposing the cell sample to an agent capable of binding the Surface K-Ras Antigen, wherein the population of cells are not lysed or otherwise permeabilized to avoid the agent reacting with intracellular forms of K-Ras; and (c) measuring the presence of the agent on the external surface of the population of cells present in the cell sample.

The method of Embodiment 10, wherein the Surface K-Ras Antigen is a mutant Surface K-Ras Antigen.

The method of Embodiment 10, wherein the agent is an antibody or an antibody fragment that binds to the Surface K-Ras Antigen.

The method of Embodiment 10, wherein the antibody or antibody fragment comprises a label and in some embodiments, the label is fluorochrome.

The method of Embodiment 10, further comprising a secondary antibody that binds to a portion of the antibody or antibody fragment, wherein the secondary antibody is labeled.

The method of any of the applicable embodiments of Embodiment 10, wherein step (c) is performed by applying the cell sample following step (b) to a flow cytometer.

The method of any of the applicable embodiments of Embodiment 10, wherein step (c) is performed by electron microscopy.

The method of any of the applicable embodiments of Embodiment 10, wherein prior to step (b), the cell sample is exposed to a cell-surface membrane dye, and wherein step (c) is performed by confocal microscopy.

The method of any of the applicable embodiments of Embodiment 10, wherein the cell sample is selected from the group consisting of pancreatic, colorectal, cholangial, ovarian, endometrial and lung cells.

Embodiment 11: A method of diagnosing a cancer associated with expression of a Surface K-Ras Antigen, the method comprising the steps of: (a) obtaining a cell sample from a subject comprising a population of cells; (b) labeling proteins on the external surface of the cell with a first agent; (c) lysing the population of cells to yield a cell lysate sample; (d) capturing labeled proteins by applying the cell lysate sample to a surface coated with a second agent, wherein the second agent selectively binds the first agent; (e) removing the captured labeled proteins from the surface and removing the first agent form the captured labeled proteins to yield a cell-surface protein sample; (f) exposing the cell-surface protein sample to a third agent capable of selectively binding the Surface K-Ras Antigen; (g) exposing the cell surface protein sample to a fourth agent, wherein the fourth agent carries a detectable label and binds to a portion of the third agent; and (h) measuring the presence of the detectable label.

The method of Embodiment 11, wherein steps (f)-(h) are performed by western blot.

The method of any of the applicable embodiments of Embodiments 10 or 11 further comprising the step of subjecting the subject to the method of any of the embodiments of Embodiments 2, 3, 8, or 9 if a mutant form of K-Ras is expressed on an external surface of the cell as a mutant Surface K-Ras Antigen.

The composition of any of the applicable embodiments of Embodiment 1, wherein the therapeutic agent is therapeutically effective to inhibit growth or proliferation of the cancer cell or is otherwise cytotoxic to the cancer cell.

The method of any of the embodiments of Embodiment 3, wherein the subject is not administered an intracellular delivery compound in conjunction with administration of the composition.

Embodiment 12: The method of any of the embodiments of Embodiment 2 further comprising the step of administering to the subject an additional therapeutic agent prior to or concurrently with the administration of the composition, wherein the additional therapeutic agent is separate from the therapeutic agent present in the composition.

The method of Embodiment 12, wherein the administration of the additional therapeutic agent leads to increased binding availability of the Surface K-Ras Antigen on the external surface of the cancer cell

The method of Embodiment 12, wherein the additional therapeutic agent is a K-Ras small molecule inhibitor.

The method of Embodiment 12, the additional therapeutic agent is a K-Ras small molecule inhibitor and wherein the K-Ras small molecule inhibitor specifically inhibits the activity of a form of K-Ras carrying a mutation that is present in the Surface K-Ras Antigen.

The method of Embodiment 12, wherein the additional therapeutic agent is a K-Ras small molecule inhibitor, wherein the K-Ras small molecule inhibitor is MRTX1133 or RMC-6236.

The method of any of the applicable embodiments of Embodiment 12, wherein the Surface K-Ras Antigen comprises a G12D mutation.

The method of any of the applicable embodiments of Embodiment 12, wherein the additional therapeutic agent is administered to the subject 1 day to 14 days prior to administration of the composition.

The method of any of the applicable embodiments of Embodiment 12, wherein the additional therapeutic agent is administered to the subject 3 days to 7 days prior to administration of the composition.

Embodiment 13: The method of any of the embodiment of Embodiment 3, 9 or 17, further comprising the step of administering to the subject a therapeutic agent prior to or concurrently with the administration of the composition, and wherein the administration of the therapeutic agent leads to increased expression of the Surface K-Ras Antigen on the external surface of the cancer cell.

The method of Embodiment 13, wherein the therapeutic agent is a K-Ras small molecule inhibitor.

The method of Embodiment 13, wherein the therapeutic agent is a K-Ras small molecule inhibitor, wherein the K-Ras small molecule inhibitor specifically inhibits the activity of a form of K-Ras carrying a mutation that is present in the Surface K-Ras Antigen.

The method of Embodiment 13, wherein the therapeutic agent is a K-Ras small molecule inhibitor, wherein the K-Ras small molecule inhibitor is MRTX1133 or RMC-6236.

The method of any of the applicable embodiments of Embodiment 13, wherein the Surface K-Ras Antigen comprises a G12D mutation.

The method of any of the applicable embodiments of Embodiment 13, wherein the therapeutic agent is administered to the subject 1 day to 14 days prior to administration of the composition.

The method of any of the applicable embodiments of Embodiment 13, wherein the therapeutic agent is administered to the subject 3 days to 7 days prior to administration of the composition.

Embodiment 14: The method of any of the embodiments of Embodiment 8, further comprising the step of administering to the individual a therapeutic agent prior to or concurrently with the administration of the immune cells expressing the chimeric antigen receptor.

The method of Embodiment 14, wherein the administration of the therapeutic agent leads to increased binding availability of the Surface K-Ras Antigen on the external surface of the cancer cell.

The method of Embodiment 14, wherein the therapeutic agent is a K-Ras small molecule inhibitor.

The method of Embodiment 14, wherein the therapeutic agent is a K-Ras small molecule inhibitor, wherein the K-Ras small molecule inhibitor specifically inhibits the activity of a form of K-Ras carrying a mutation that is present in the Surface K-Ras Antigen.

The method of Embodiment 14, wherein the therapeutic agent is a K-Ras small molecule inhibitor wherein the K-Ras small molecule inhibitor is MRTX1133 or RMC-6236.

The method of any of the applicable embodiments of Embodiment 14, wherein the Surface K-Ras Antigen comprises a G12D mutation.

The method of any of the applicable embodiments of Embodiment 14, wherein the therapeutic agent is administered to the subject 1 day to 14 days prior to administration of the immune cells.

The method of any of the applicable embodiments of Embodiment 14, wherein the therapeutic agent is administered to the subject 3 days to 7 days prior to administration of the immune cells.

Embodiment 15: A therapeutically-effective amount of a composition according to any of the embodiments of Embodiment 1 or 7 or a composition comprising the bispecific antibody of Embodiment 16 for use in treating, inhibiting or reducing proliferation of or killing a cancer cell in a subject, wherein the subject has been administered an additional therapeutic agent in addition to the composition either prior to or concurrently with the composition.

The composition for use according to Embodiment 15, wherein the administration of the additional therapeutic agent stimulates expression of Surface K-Ras Antigen on the external surface of the cancer cell.

The composition for use according to Embodiment 15, wherein the subject has not been administered an intracellular delivery compound in conjunction with administration of the composition.

The composition for use according to any of the applicable embodiments of Embodiment 15, wherein the additional therapeutic agent is a K-Ras small molecule inhibitor.

The composition for use according to any of the applicable embodiments of Embodiment 15, wherein the additional therapeutic agent was administered to the subject 1 day to 14 days prior to administration of the composition.

The composition for use according to any of the applicable embodiments of Embodiment 15, wherein the additional therapeutic agent was administered to the subject 3 days to 7 days prior to administration of the composition.

The composition for use according to any of the applicable embodiments of Embodiment 15, wherein the cancer cells are selected from the group consisting of pancreatic cancer cells, lung cancer cells, cholangial cancer cells, ovarian cancer cells, endometrial cancer cells, or colorectal cancer cells.

The composition for use according to any of the applicable embodiments of Embodiment 15, wherein the additional therapeutic and the composition are administered to the subject at substantially the same time.

Embodiment 16: A bispecific antibody comprising: a first binding domain linked to a second binding domain, wherein the first binding domain selectively binds to a Surface K-Ras Antigen expressed on an external surface of a cancer cell, and wherein the second binding domain selectively binds to an antigen expressed on a surface of an immune effector cell.

The bispecific antibody of Embodiment 16, wherein the first binding domain comprises a light chain variable region and a heavy chain variable region.

The bispecific antibody of Embodiment 16, wherein the first binding domain comprises a light chain variable and constant region and a heavy chain variable and constant region.

The bispecific antibody of any of the embodiments of Embodiment 16, wherein the second binding domain comprises a light chain variable region and a heavy chain variable region.

The bispecific antibody of any of the embodiments of Embodiment 16, wherein the second binding domain comprises a light chain variable and constant region and a heavy chain variable and constant region.

The bispecific antibody of any of the embodiments of Embodiment 16, wherein the Surface K-Ras Antigen possess at least 60% homology to SEQ ID NO: 1 or SEQ ID NO: 2.

The bispecific antibody of any of the embodiments of Embodiment 16, wherein the first binding domain selectively binds to a region on the Surface K-Ras Antigen that includes residues 12, 13, or 61 based on the amino acid sequence set forth in SEQ ID NO: 1 or SEQ ID NO: 2.

The bispecific antibody of any of the embodiments of Embodiment 16, wherein the first binding domain selectively binds the Surface K-Ras Antigen with any one of the following mutations based on the amino acid sequence set forth in SEQ ID NO: 1 or SEQ ID NO: 2: G12A; G12D; G12C; G12V; G12R; G13D; Q61H; and Q61L.

The bispecific antibody of any of the embodiments of Embodiment 16, wherein the first binding domain selectively binds the Surface K-Ras Antigen with the G12D mutation based on the amino acid sequence set forth in SEQ ID NO: 1 or SEQ ID NO: 2.

The bispecific antibody of any of the embodiments of Embodiment 16, wherein the first binding domain selectively binds the Surface K-Ras Antigen with the G12C mutation based on the amino acid sequence set forth in SEQ ID NO: 1 or SEQ ID NO: 2.

The bispecific antibody of any of the embodiments of Embodiment 16, wherein the first binding domain selectively binds the Surface K-Ras Antigen with the G12V mutation based on the amino acid sequence set forth in SEQ ID NO: 1 or SEQ ID NO: 2.

The bispecific antibody of any of the embodiments of Embodiment 16, wherein the first binding domain selectively binds the Surface K-Ras Antigen with the G12D mutation based on the amino acid sequence set forth in SEQ ID NO: 1 or SEQ ID NO: 2.

The bispecific antibody of any of the embodiments of Embodiment 16, wherein the antigen expressed on a surface of an immune effector cell is selected from the group consisting of TCRα, TCRβ, TCRδ, TCRγ, CD3β, CD3γ, CD3ε, CD3δ, CD3β, CD137, CD16, and CD64.

The bispecific antibody of any of the embodiments of Embodiment 16, wherein the antigen expressed on a surface of an immune effector cell is CD3ε.

The bispecific antibody of any of the embodiments of Embodiment 16, wherein the immune effector cell is selected from the group consisting of a T cell, a neutrophil, a macrophage, a monocyte and an NK cell.

Embodiment 17: A method of treating, inhibiting or reducing proliferation of or killing a cancer cell in a subject comprising administering a therapeutically-effective amount of a bispecific antibody of any of the embodiments of Embodiment 16, and wherein a Surface K-Ras Antigen is expressed on the external surface of the cancer cell.

The method of Embodiment 17, wherein the subject is not administered an intracellular delivery compound in conjunction with administration of the bispecific antibody.

The method of Embodiment 17, wherein the cancer cells are selected from the group consisting of pancreatic cancer cells, lung cancer cells, cholangial cancer cells, ovarian cancer cells, endometrial cancer cells, or colorectal cancer cells.

The method of any of the embodiments of Embodiment 17, wherein the administering step is performed by intravenous injection or subcutaneous injection.

The method of any of embodiments of Embodiment 17, wherein the Surface K-Ras Antigen is a full length or truncated form of K-Ras with a mutation at any of the following amino acids/residues: 12 (including, but not limited to G12A, G12D, G12C, G12V, G12R); 13 (including, but not limited to G13D); and 61 (including, but not limited to Q61H, Q61L) based on the amino acid position in SEQ ID NO: 1 or SEQ ID NO: 2.

Embodiment 18: A composition comprising: a means for selectively binding to a Surface K-Ras Antigen expressed on an external surface of a cancer cell, wherein the means is linked to a therapeutic agent, and wherein the composition does not include an intracellular delivery compound.

The composition of Embodiment 18, wherein the Surface K-Ras Antigen is a full length or truncated form of K-Ras with a mutation at any of the following amino acids/residues: 12 (including, but not limited to G12A, G12D, G12C, G12V, G12R); 13 (including, but not limited to G13D); and 61 (including, but not limited to Q61H, Q61L) based on the amino acid position in SEQ ID NO: 1 or SEQ ID NO: 2.

Embodiment 19: A bispecific antibody comprising: a first means for selectively binding to a Surface K-Ras Antigen expressed on an external surface of a cancer cell, and a second means for selectively binding to an antigen expressed on a surface of an immune effector cell, wherein the first means is linked to the second means.

The bispecific antibody of Embodiment 19, wherein the Surface K-Ras Antigen is a full length or truncated form of K-Ras with a mutation at any of the following amino acids/residues: 12 (including, but not limited to G12A, G12D, G12C, G12V, G12R); 13 (including, but not limited to G13D); and 61 (including, but not limited to Q61H, Q61L) based on the amino acid position in SEQ ID NO: 1 or SEQ ID NO: 2.

Embodiment 20: A chimeric antigen receptor comprising an extracellular domain, a transmembrane domain, and an endodomain, wherein the extracellular domain comprises a means for binding with specificity to a Surface K-Ras Antigen expressed on an external surface of a cancer cell.

The chimeric antigen receptor of Embodiment 20, wherein the Surface K-Ras Antigen is a full length or truncated form of K-Ras with a mutation at any of the following amino acids/residues: 12 (including, but not limited to G12A, G12D, G12C, G12V, G12R); 13 (including, but not limited to G13D); and 61 (including, but not limited to Q61H, Q61L) based on the amino acid position in SEQ ID NO: 1 or SEQ ID NO: 2.

### EXAMPLES

### Example 1: Inhibition of K-Ras Signaling from the Cell Surface Provides Control of Tumor Growth

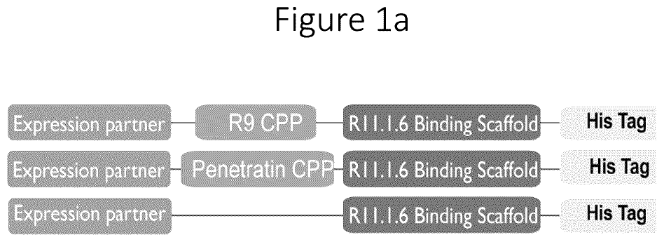

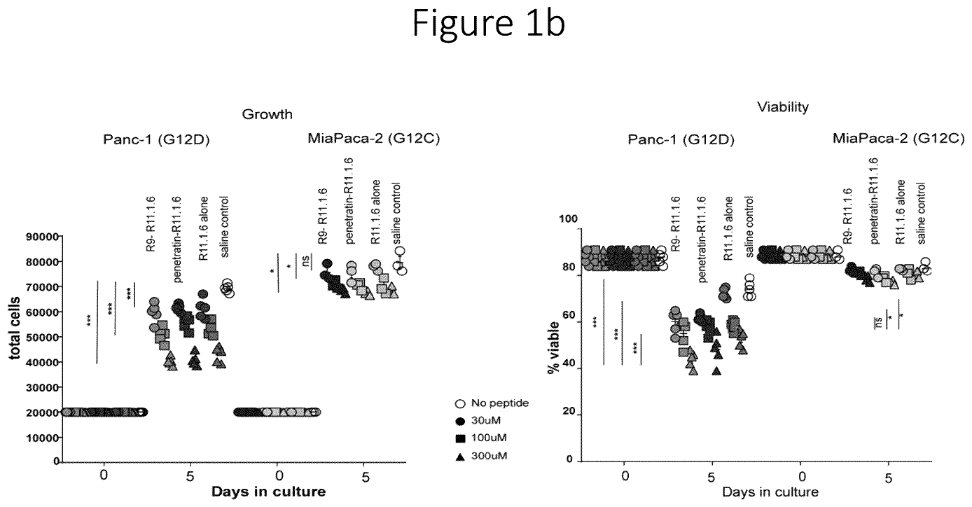

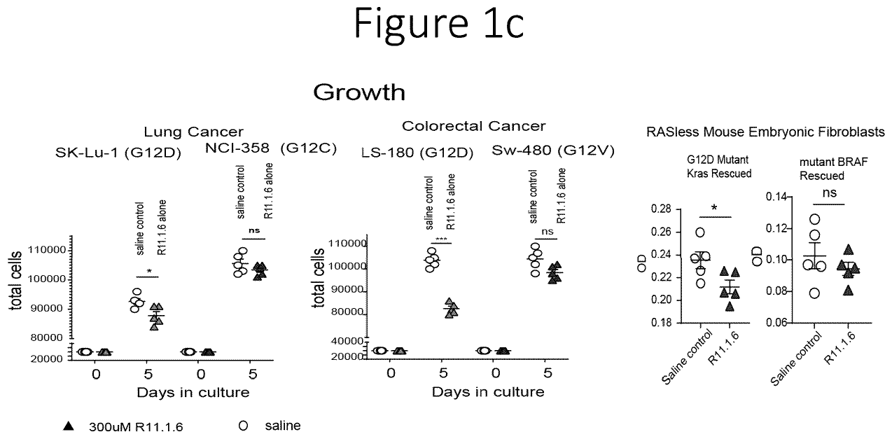

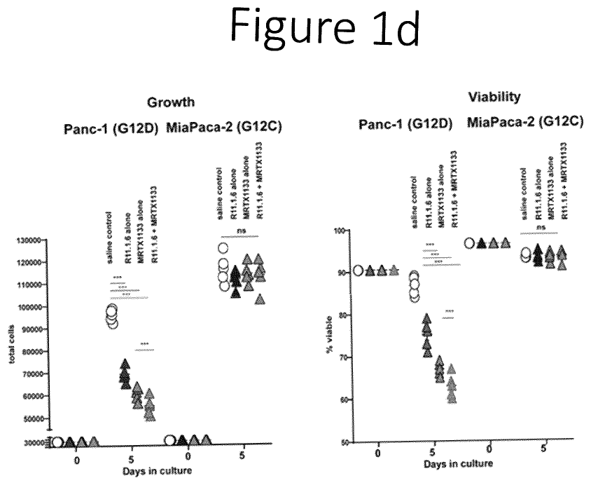

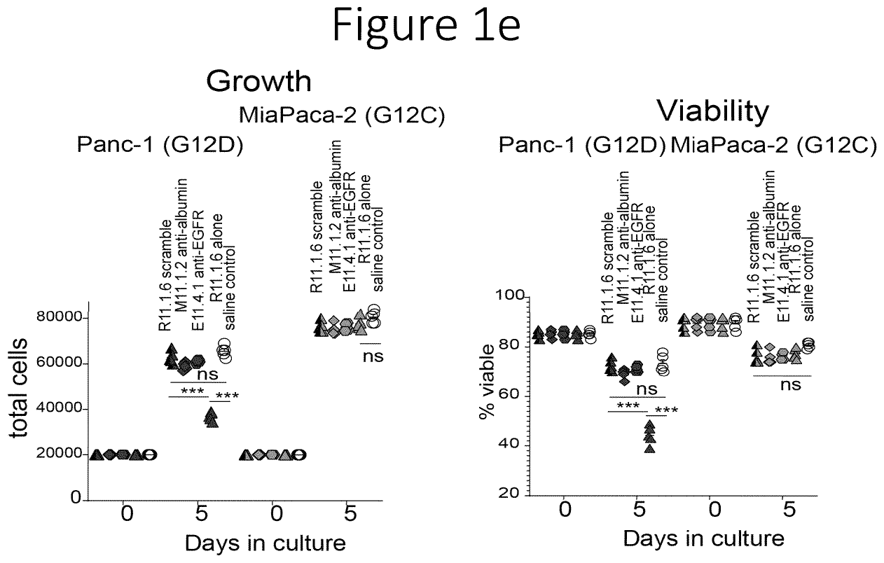

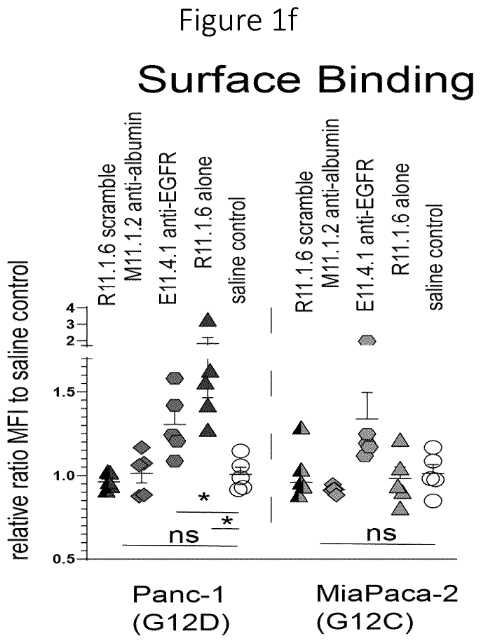

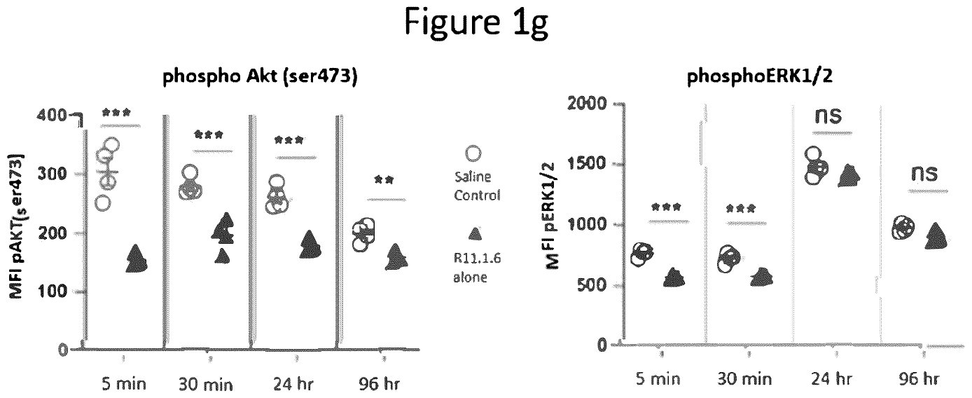

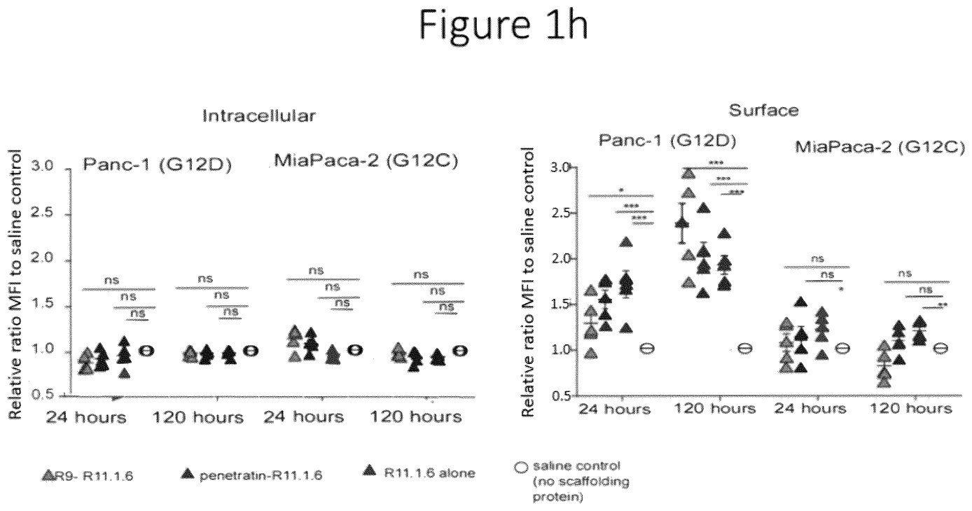

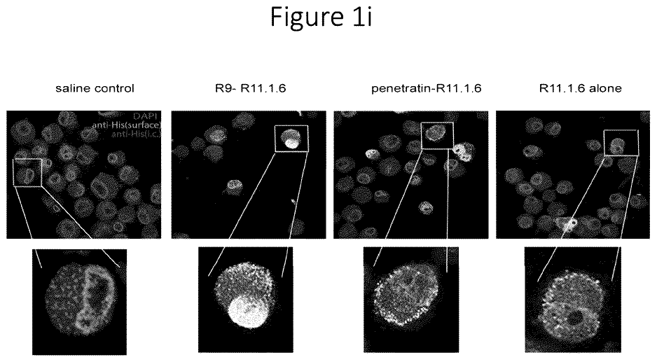

We took advantage of a charge-neutralized variant of the Sso7d protein from the hyperthermophilic archaeon Sulfolobus solfataricus, known as R11.1.6 (SEQ ID NO: 5—ATVKFTHQGEEKQVDISKIKWVIRWGQYIWFKYDEDGGAKGWGYVSEKDAPKELLQMLK KR) that binds switch I of G12D mutant K-Ras with high affinity and specificity. (See, for example, Traxlmayr et al., J Biol Chem. 2016 Oct. 21; 291(43):22496-22508. doi: 10.1074/jbc.M116.741314.) To deliver this antagonistic scaffold intracellularly we coupled it to cell penetrating peptides (CPP): either R9 (R9-R11.1.6), or penetratin (penetratin-R11.1.6). Additionally, we used R11.1.6 in the absence of any cell penetrating peptide as an internal control (FIG. 1a). We next evaluated growth as well as viability of two pancreatic cancer cell lines driven by either G12D (Panc-1) or G12C (MiaPaca-2) mutant K-Ras in vitro. We plated 20,000 cells/well in Dulbecco's modified Eagles media (DMEM) with 10% fetal bovine serum (FBS) in a flat bottom 96 well plate and counted cells after 5 days of culture. Viability of cells were calculated based on Trypan blue dye exclusion method, where dead cells were blue due to uptake of the viability dye and live cells were not stained. Viability was measured by using Countess II FL cell viability and cell counter instrument by Invitrogen (ThermoFisher USA). We noted inhibition of tumor cell expansion in a G12D mutation-specific fashion (FIG. 1b—growth left panel and viability right panel). Surprisingly, R11.1.6 altered Panc-1 growth and viability to a similar degree with and without being coupled to a cell penetrating peptide. Similar data was replicated in K-Ras-driven lung and colorectal cancer cell lines (FIG. 1c). R11.1.6-mediated inhibition of Panc-1 growth and viability was like the G12D inhibitor MRTX1133 (PMID: 36216931) and using the two agents in combination augmented the therapeutic effect (FIG. 1d).

Further evaluation of growth inhibition was performed with control Sulfolobus solfataricus-derived proteins of similar molecular weight. These included a mutated form of R11.1.6 (where the codons in the regions that contact the K-RAS mutant are scrambled to prevent binding) (SEQ ID NO: 6—ATVKFTHQGEEKQVDISKIKFVWRKGYVRIWGYDEDGGWGAGKYVSEKDAPKELLQMLK KR), M11.1.2 (a scaffolding protein that binds mouse serum albumin) (SEQ ID NO: 7—ATVKYTYRGEEKRVDISKIKWVNRWGQHLAFKYDKGGGAAGYGWVSEKDAPKELLQML EKR), and E11.4.1 (which binds human EGFR) (SEQ ID NO: 8—ATVKFTYQGEEKQVDISKIMYVIRGGQRIAFGYDEGDGAWGDGIVSEKDAPKELLQMLEK Q). Compared to R11.1.6, none of these control constructs could inhibit tumor growth (FIG. 1e) despite the ability of some constructs, such as E11.4.1, to bind to the surface of tumor cells (FIG. 1f). In addition, inhibition of tumor growth by wild-type R11.1.6 correlated with a canonical decrease in signaling through the AKT and ERK pathways of signal transduction. We detected lower phosphorylation of Akt (at Serine 473 reside) and ERK1/2 (threonine 202, tyrosine 204) by phosphor flow cytometry method. We started with 200,000 cells in a 96 well V-bottom plate and used BD phosflow buffer set III (BD biosciences, catalog number 558050) to stabilize and optimize the phosphor moiety. Next, we stained the cells with anti-phospho-Akt (ser473, Clone D9E) PE conjugated (Cell Signaling Technologies, Catalog number 5315S) at a concentration of 0.125 ug/100 ul or anti-phosho-ERK1/2, PE conjugated (ThermoFisher USA catalog number 12-9109-42) at a concentration of 0.125 ug/100 ul. Cells were washed in FACS staining buffer and run in a LSR Fortessa II flow cytometer (BD Biosciences, USA). Acquisition was done was using FACS DIVA software (BD Biosciences) and analysis was performed in FlowJo software (Tree Star, OR, USA). This was a significant finding as both Akt and ERK1/2 are important downstream molecules of K-Ras signaling, like that described for small molecule inhibitors of K-Ras (PMID: 12509763) (FIG. 1g).

Surprisingly, inhibition was evident even in the absence of cell penetrating peptides even though it is assumed that both mutant and wild-type K-Ras signal from various intracellular locations only. However, we considered that the small 7 kDa size of this scaffolding protein and disorganized cell membrane of malignantly transformed cells may allow for cytoplasmic penetration even in the absence of a cell penetrating peptide. To evaluate this possibility, we explored the cellular location of the R11.1.6 constructs by flow cytometry and confocal microscopy. For flow cytometry experiments, cells were stained first with Live/Dead far red fixable staining kit (ThermoFisher USA, catalog number L10120). Specifically, 0.2-0.4×10⁶ cells were plated in a V bottom plate and 100 ul of the dye (1:1000 dilution of stock concentration following manufacturer's protocol) was added to each well and cells incubated in room temperature for 15 mins. Next cells were washed with PBS containing 2.5% FBS and 0.1% sodium azide (FACS buffer). Cells were then surface stained by adding 100 ul/well of anti-His PE labelled antibody (mouse anti-human antibody, BioLegend, CA, USA Catalog #362603) at a concentration of 10 μg/ml. Cells were washed twice with FACS buffer and fixed by cytofix buffer (BD biosciences catalog number 554655). The same antibody and reagents were used for intracellular staining except for that we permeabilized the cells prior to adding the PE anti-His antibody with BD perm/wash reagent (BD Biosciences, Catalog number 554723). Cells were acquired using LSR Fortessa II flow cytometer using FACS DIVA software (BD Biosciences USA) and analyzed using FlowJo (Tree Star USA). For confocal microscopy experiments, we surface stained cells in a similar way as we did for flow cytometry experiments. After completion of staining cells were carefully spread over a 25 mm×75 mm slide (1 mm thickness) and mounted with Prolong Gold antifade reagent with DAPI (Cell signaling Technologies, USA, catalog number 8961). Cells were imaged using a Nikon A1 spinning disk microscope. Analysis was done using ImageJ software (NIH developed and licensed). Since all constructs contain a polyhistidine-tag (His-tag) (FIG. 1a), we tested this using fluorescently labeled anti-His-tag antibodies. To this end Panc-1 (G12D mutant K-Ras) or MiaPaca-2 (G12C mutant K-Ras) cell lines were cultured with either R9-R11.1.6, penetratin-R11.1.6, or R11.1.6 (without cell penetrating peptide) for either 24 or 96 hours and stained by fluorescently labeled secondary antibody recognizing the His-tag to determine its subcellular location. As shown in FIG. 1h, compared to saline control, which was defined as 1, no R11.1.6 was evident inside the tumor cells. However increased binding to the tumor cell surface was evident for R11.1.6 in Panc-1, but not MiaPaca-2 cancer cells. Given that R11.1.6 without cell penetrating peptide was able to inhibit tumor cell growth, we were surprised that none of the constructs crossed the cell membrane but rather bound to the cell surface by both flow cytometry (FIG. 1h) or high-resolution confocal microscopy (Figure ii). Such binding seemed to be mutation specific as little construct was observed on the surface of MiaPaca-2 which carries the G12C mutation that does not bind R11.1.6 (FIGS. 1f, 1h, 1i). Taken together our data suggest that inhibition of tumor cell growth and viability seems to be mediated by mutation-specific binding of a K-Ras antagonist to the cell surface. This was surprising because K-Ras has been considered to locate exclusively to the intracellular cell membrane and subcellular organelles (PMID: 21924373) and has never been described on the cell surface.

### Example 2: Functional K-Ras is Expressed on the Extracellular Surface of Both Malignant and Non-Transformed Cells

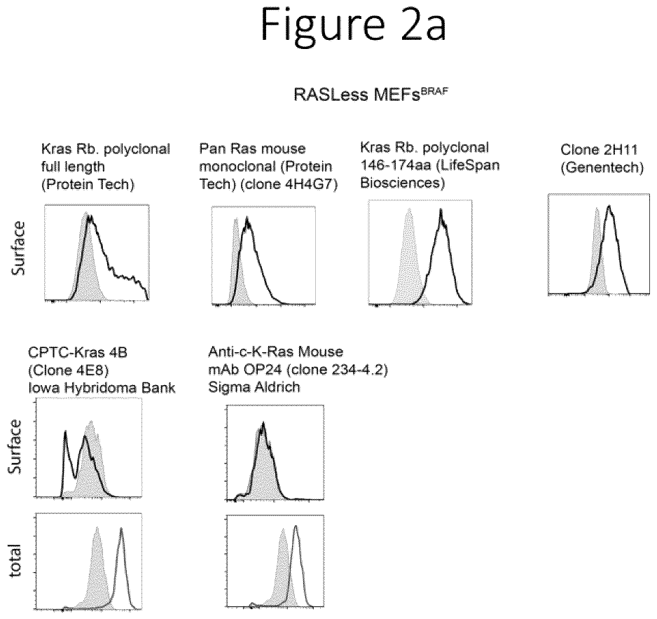

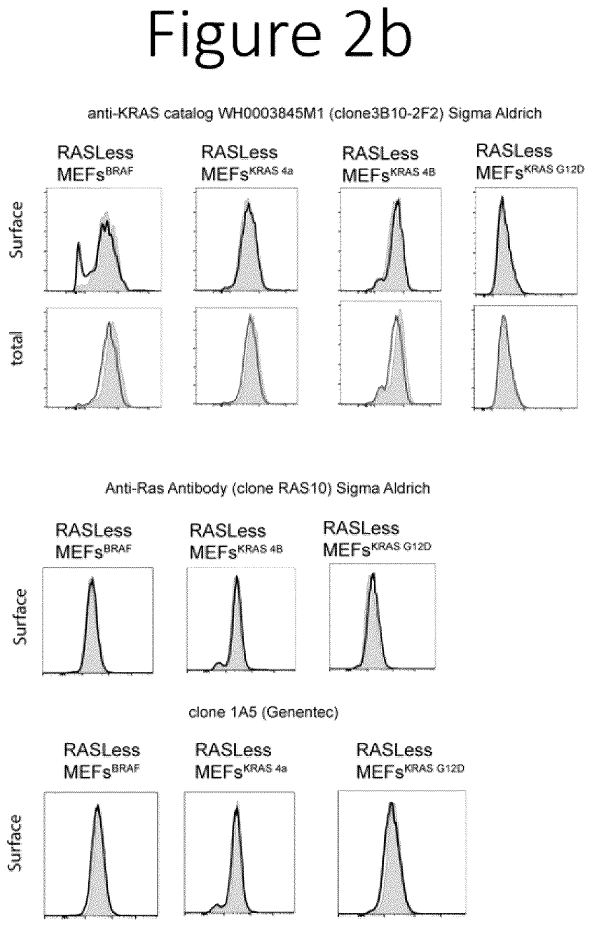

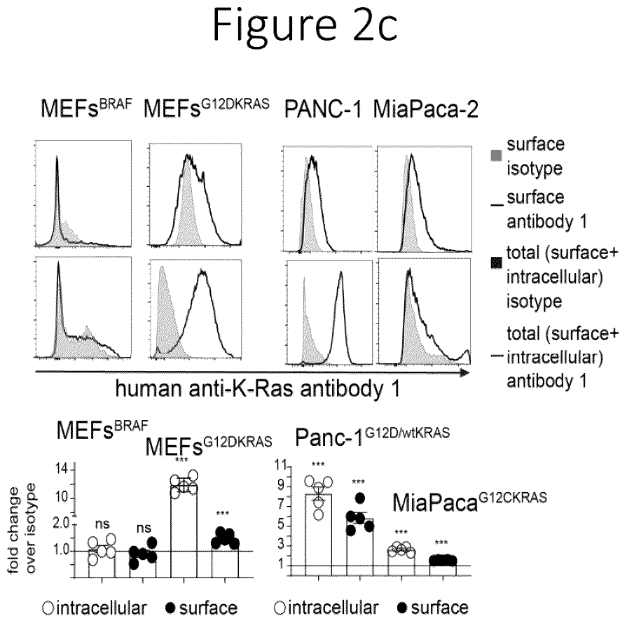

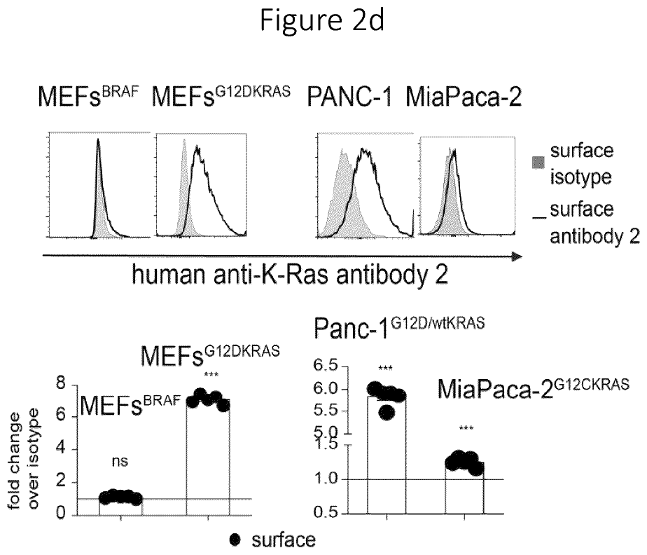

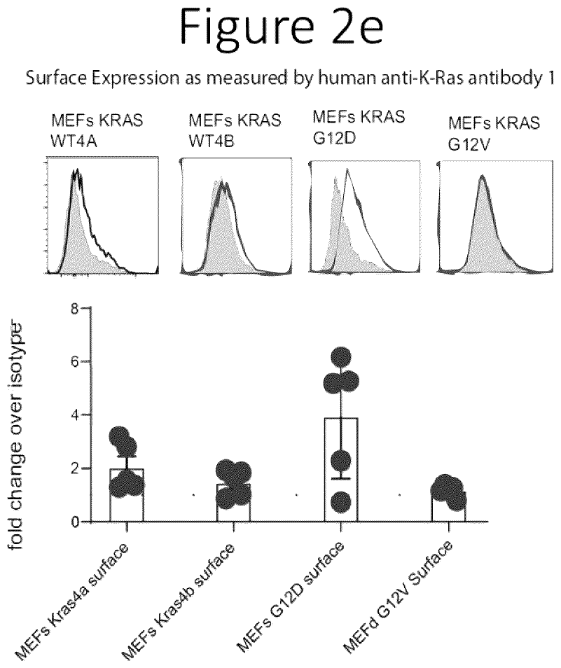

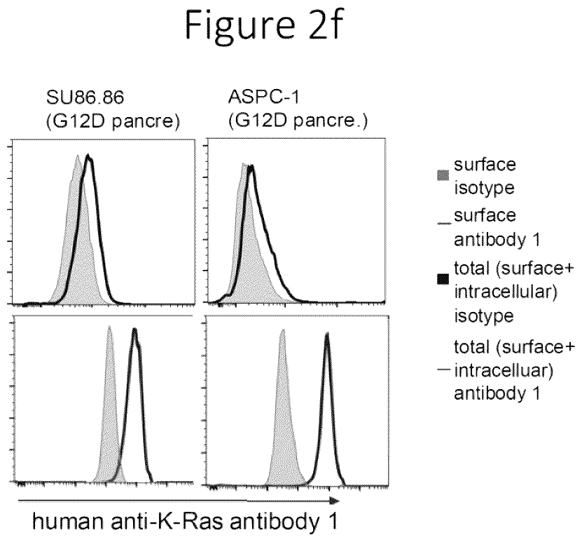

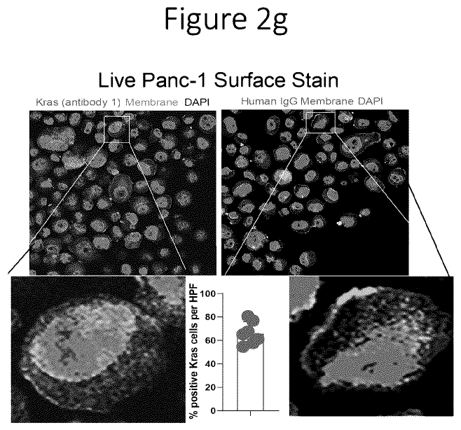

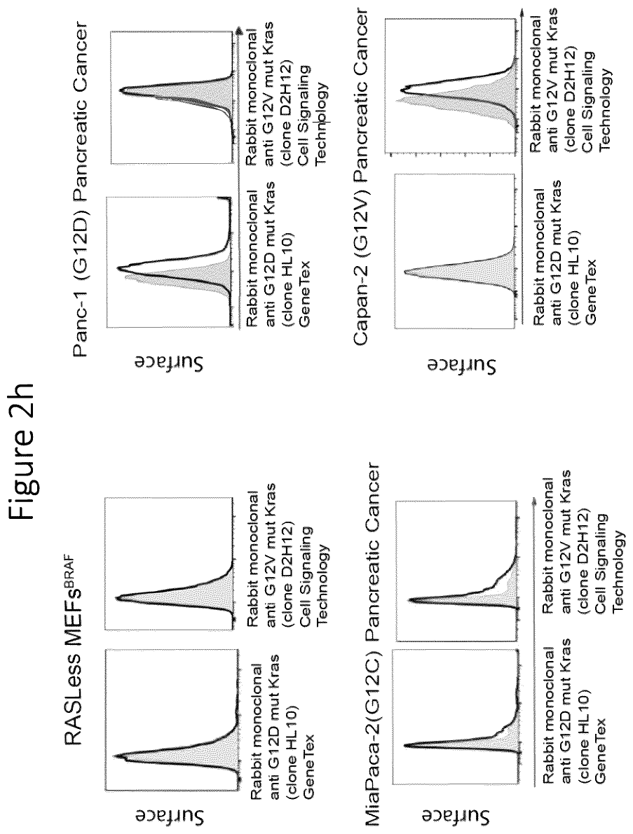

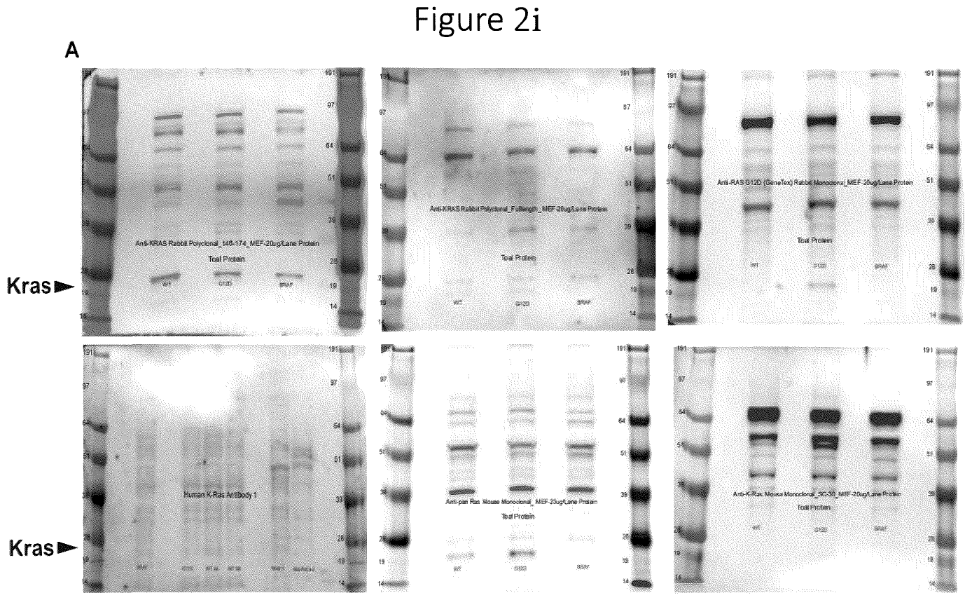

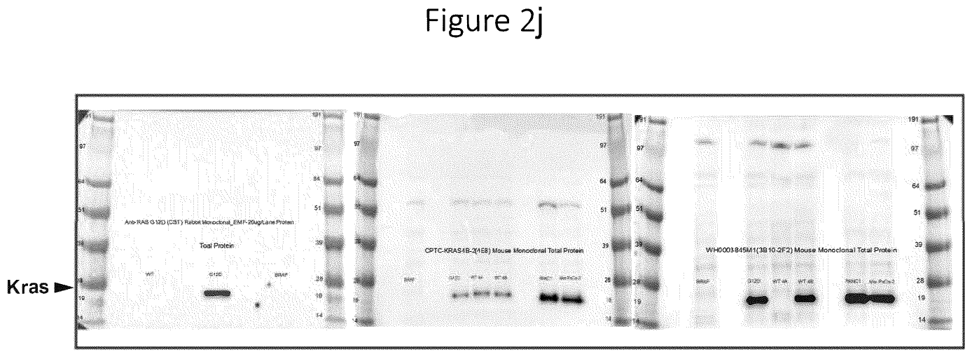

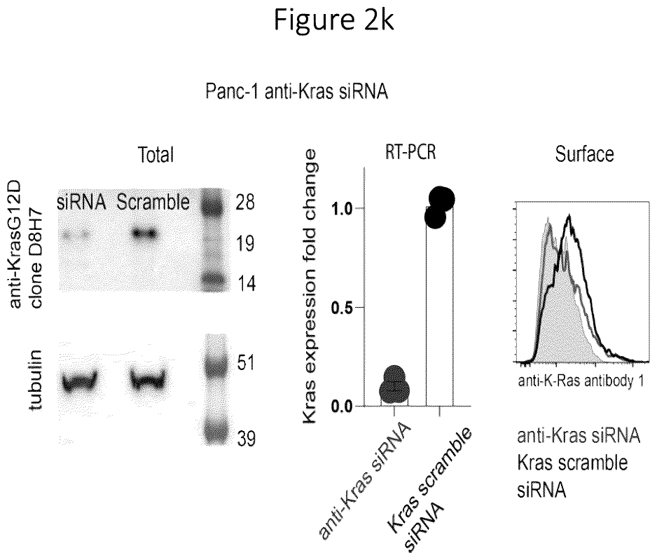

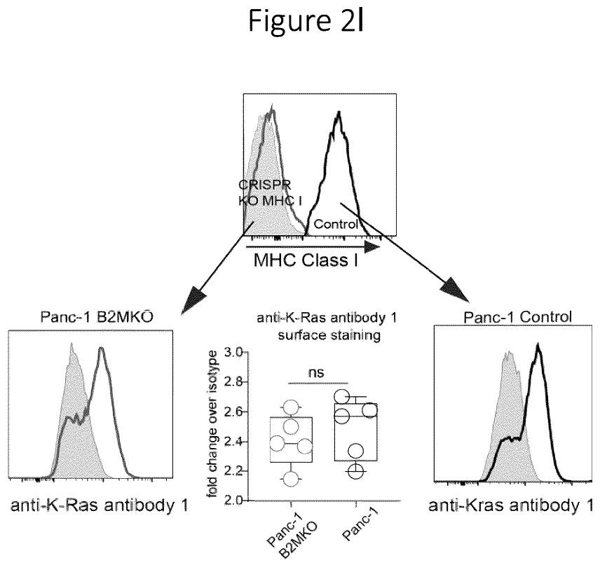

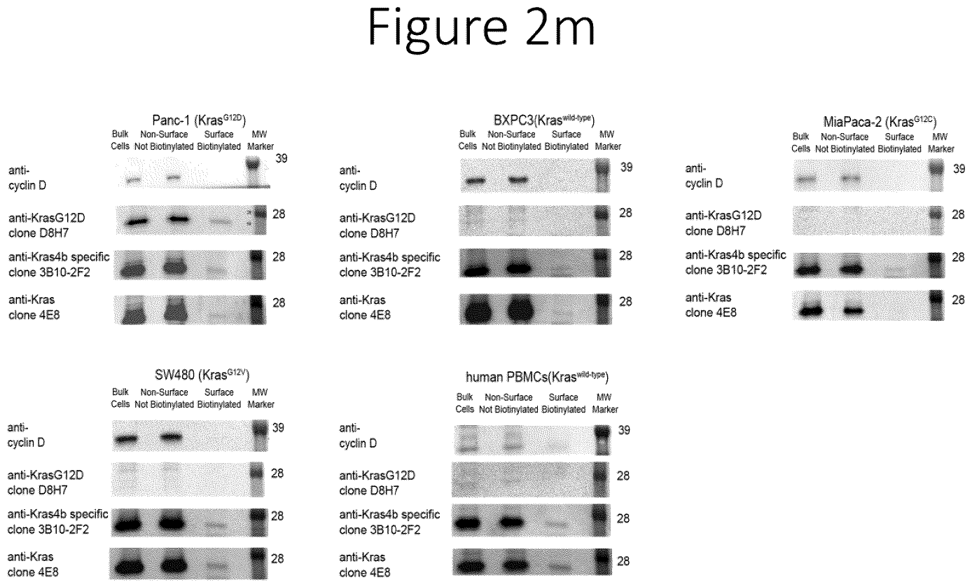

K-Ras has been defined as an intracellular protein without known surface expression. No previous report of K-Ras on the surface of cells has been reported in the scientific literature. Since both the specificity and sensitivity of available anti-K-Ras antibodies has previously been brought into question (PMID: 28951536), we performed an extensive validation effort of multiple commercially available and public domain-described antibodies for use in our studies. For this we, took advantage of the RASless mouse embryonic fibroblast system (MEFs) where MEFs lacking all RAS proteins are rescued by lentiviral transduction of either wild type RAS, mutant RAS, or the V600E mutant variant of BRAF which can allow for survival and proliferation in the absence of all endogenous Ras proteins (PMID:33977488 and cancer.gov/research/key-initiatives/ras/ras-central/blog/2017/rasless-mefs-drug-screens). We tested antibodies by flow cytometry according to the protocol described in Example 1. Most antibodies tested displayed some non-specific binding and reacted with BRAF^{V600E} rescued RASless MEFs (FIG. 2a-b). Some antibodies were able to detect Surface K-Ras Antigen on the cell surface in a sensitive and specific manner with a preferential binding to the G12D mutant version. For example, Human Anti-K-Ras Antibody 1 (Full Heavy Chain (HC)—SEQ ID NO: 9—EVQLVQSGGGVVQPGRSLRLSCAASGFTSRHPGMHWVRQAPGKGLEWVAVISHDGSK KYYADSVKGRFTISRDNSKNTLFVQLSSLRPEDTAVYYCATSLYSSMDLWGQGTTVTVSS ASTKGPSVFPLAPSSKSTSGGTAALGCLVKDYFPEPVTVSWNSGALTSGVHTFPAVLQS SGLYSLSSVVTVPSSSLGTQTYICNVNHKPSNTKVDKKVEPKSCDKTHTCPPCPAPELLG GPSVFLFPPKPKDTLMISRTPEVTCVVVDVSHEDPEVKFNWYVDGVEVHNAKTKPREEQ YGSTYRVVSVLTVLHQDWLNGKEYKCKVSNKALPAPIEKTISKAKGQPREPQVYTLPPSR EEMTKNQVSLTCLVKGFYPSDIAVEWESNGQPENNYKTTPPVLDSDGSFFLYSKLTVDKS RWQQGNVFSCSVMHEALHNHYTQKSLSLSPG; HC CDR1—SEQ ID NO: 10—GFTSRHPG; HC CDR2—SEQ ID NO: 11—ISHDGSKK; HC CDR3—SEQ ID NO: 12—ATSLYSSMDL; Full Light Chain (LC)—SEQ ID NO: 13—QSVVTQPPSVSAAPGQKVTISCSGSNSNIGKNYVSWFQQVPGTAPKLLIFEDNQRPSGIP DRFSASKSGTSASLAISGLQSEDEADYYCAAWDDKFGVHWVFGGGTKLTVLGQPKAAP SVTLFPPSSEELQANKATLVCLISDFYPGAVTVAWKADSSPVKAGVETTTPSKQSNNKYA ASSYLSLTPEQWKSHRSYSCQVTHEGSTVEKTVAPTECS; LC CDR1—SEQ ID NO: 14—NSNIGKNY; LC CDR2—SEQ ID NO: 15—EDN; LC CDR3—SEQ ID NO 16—AAWDDKFGVHWV) and Human Anti-K-Ras Antibody 2 (Full HC—SEQ ID NO: 17—EVQLLEPGGGVVQPGRSLRLSCTNSGFSFSGYAMHWVRQAPGKGLEWVAVISFDGSHK YYADSVKGRFTISRDNSKNTLYLHMNSLRAEDTAVYYCASGGNYYGSGTIVSHGMDVWG QGTTVTVSSASTKGPSVFPLAPSSKSTSGGTAALGCLVKDYFPEPVTVSWNSGALTSGV HTFPAVLQSSGLYSLSSVVTVPSSSLGTQTYICNVNHKPSNTKVDKKVEPKSCDKTHTCP PCPAPELLGGPSVFLFPPKPKDTLMISRTPEVTCVVVDVSHEDPEVKFNWYVDGVEVHN AKTKPREEQYGSTYRVVSVLTVLHQDWLNGKEYKCKVSNKALPAPIEKTISKAKGQPREP QVYTLPPSREEMTKNQVSLTCLVKGFYPSDIAVEWESNGQPENNYKTTPPVLDSDGSFF LYSKLTVDKSRWQQGNVFSCSVMHEALHNHYTQKSLSLSPG; HC CDR1—SEQ ID NO: 18—GFSFSGYA; HC CDR2—SEQ ID NO: 19—ISFDGSHK; HC CDR3—SEQ ID NO: 20—ASGGNYYGSGTIVSHGMDV; Full LC—SEQ ID NO: 21—QSVLTQPASVSGSPGQSITISCTGTSNDIGAYNYVSWYQQHPGKAPKLMIYDVNNRPSG VPDRFSGSKSGNMASLTISGLQAEDDADYYCSSYTSSSTLVVFGGGTKLTVLGQPKAAP SVTLFPPSSEELQANKATLVCLISDFYPGAVTVAWKADSSPVKAGVETTTPSKQSNNKYA ASSYLSLTPEQWKSHRSYSCQVTHEGSTVEKTVAPTECS; LC CDR1—SEQ ID NO: 22—SNDIGAYNY; LC CDR2—SEQ ID NO: 23—DVN; LC CDR3—SEQ ID NO: 24—SSYTSSSTLVV) staining was evident on the surface of K-Ras rescued MEFs as well as Panc-1 and MiaPaca-2 (FIG. 2c-d). Further characterization of Human Anti-K-Ras Antibody 1 binding using the RASless MEFs demonstrated binding to K-Ras4A, K-Ras4B, and G12D K-Ras 4B, but not G12V K-Ras 4B (FIG. 2e). Additional G12D K-Ras tumor cell lines also showed surface staining with Human Anti-K-Ras Antibody 1 (FIG. 2f). All staining was performed at 10 μg/ml of antibody concentration and a volume of 100ul for 30 minutes on ice in V-bottom 96 well plates. Surface staining of live Panc-1 cells with Human Anti-K-Ras Antibody 1 was also observed by high resolution confocal microscopy (FIG. 2g). Confocal microscopy was done using identical conditions to FIG. 1i in Example 1.

Surface staining was also observed with antibodies anti-G12V mutant K-Ras (clone D2H12) and anti-G12D mutant K-Ras (clone HL10) (FIG. 2h). No staining was evident on RASless MEFS rescued with either BRAF^{V600E} or MiaPaca-2, a K-Ras G12C mutant cancer cell line.

A panel of antibodies demonstrated reactivity to MEFs rescued with BRAF^{V600E} or demonstrated multiple non-specific bands in addition to the 22 kDa K-Ras band (FIG. 2i). Only rabbit anti-G12D mutant K-Ras (clone D8H7, Cell Signaling Technology), mouse anti-K-Ras (clone 4E8, Iowa Hybridoma Bank) and mouse anti-K-Ras (clone 3B10-2F2 Sigma Aldridge) demonstrated both sensitivity and specificity to the G12D mutant K-Ras 4B, Pan K-Ras, and the 4B splice variant of K-Ras by western blot, respectively (FIG. 2j). For western blot experiments, cells were lysed in situ for 30 minutes at 4° C. by the addition of 10× cell lysis buffer containing 20 mM Tris-HCl (pH 7.5), 150 mM NaCl 1 mM Na2EDTA, 1 mM EGTA, 1% Triton X-100, and protease inhibitor cocktail. The resulting cell lysates were cleared by centrifugation at 18,800 g. After 3 washes with cell lysis buffer, the proteins were resolved on a 10% Tris-Glycine SDS-PAGE gel (Bio-Rad) and transferred to an Immobilon-P PVDF membrane. Next, the membranes were blocked with 3% BSA in PBS with 0.1% Tween 20 for 1 hour at 20° C. Proteins were then detected with respective primary antibodies followed by the appropriate secondary antibodies conjugated to horseradish peroxidase. The horseradish peroxidase activity was detected with Pierce enhanced chemiluminescence substrate (Thermo Fisher Scientific) according to the manufacturer's instructions. The chemiluminescence signal was acquired with the ChemiDoc MP Imaging System and analyzed with the Image Lab 5.1 software application (both from Bio-Rad).

For K-Ras knockdown experiments, we took advantage of the in vitro SiRNA technique to silence K-RAS gene expression. Cells were trypsinized and plated in 500 ul of a 24 well plate with a final SiRNA concentration of 25 nM in DharmaFECT™ transfection reagent according to manufacturer's protocol. K-Ras protein was knocked down by utilizing siRNA targeting multiple splice variants of K-Ras mRNA (Dharmacon™ Reagents, ON-TARGETplus Human SMARTpool K-RAS siRNA) in the Panc-1 cell line and demonstrated downregulation by both RT-PCR as well as western blotting of intracellular K-Ras. Surface staining of K-Ras by Human Anti-K-Ras Antibody 1 in siRNA-treated cell lines was also decreased (FIG. 2k).

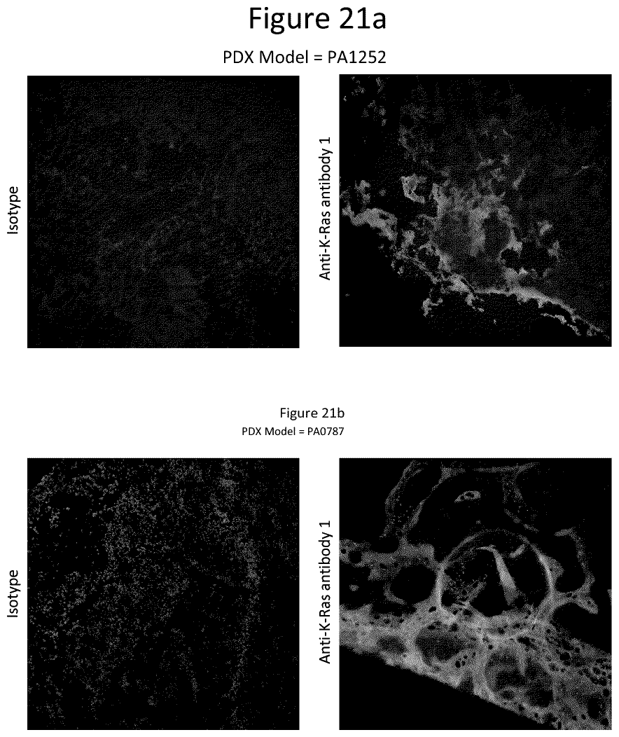

It has been previously described that peptide fragments of both mutant and wild-type K-Ras can be presented at the cell surface in the context of self MHC Class I (PMID: 36099883, PMID: 27959684, PMID: 34272369). We created an MHC Class I-deficient Panc-1 cell line through CRISPR-Cas-9 excision of beta-2 microglobulin and noted identical staining of such mutants with Human Anti-K-Ras Antibody 1 compared to the MHC Class I-sufficient parental cell line (FIG. 21).

Proteins on the cell that were exposed to the extracellular environment were treated with reactive biotin esters (Pierce Cell Surface Protein Biotinylation and Isolation kit, #A44390) in multiple cell lines and separated from cellular extracts with streptavidin beads based on established methodology (PMID: 19341246). Like flow cytometric data described above, K-Ras was detected in the surface fraction by western blot analysis using K-Ras-specific antibodies, albeit in much smaller amounts than inside the cell membrane (FIG. 2m).

Taken together our data shows that both tumors as well as normal tissue can express K-Ras on their surface and binding such surface K-Ras with a protein therapeutic antagonist can ameliorate the growth of cancer in a mutation-specific manner.

### Example 3: Validation of Antibody Drug Conjugates (ADC) for Selective Killing of Cancer Cell Lines Through the Use of Mutation-Specific Antibodies

The use of Antibody Drug Conjugates (ADC) for selective tumor killing is an established method for delivering a cytotoxic payload to cells expressing a defined surface target. This approach comprises an antibody conjugated to the cytotoxic payload via a linker that is directed toward a target antigen expressed on the cancer cell surface. Such methodology reduces systemic exposure, and therefore toxicity, and offers great opportunity for targeted killing of cancer cells (reviewed in, for example, Drago et al. Nat Rev Clin Oncol. 2021 June; 18(6):327-344 doi.org/10.1038/s41571-021-00470-8). To work as a selective ADC agent on target cells, the ADC (which comprises the targeting antibody and toxin) needs to: a) recognize the tumor antigen selectively and, b) be able to kill the target cell at a concentration that is not toxic to cells not recognized by the antibody. The use of mutant Surface K-Ras Antigen as a target for ADC has never been explored.

Saporin is a 30 kDa ribosome-inhibitor which is unable to cross a cell membrane unaided (except at extremely high concentrations). However, when it is conjugated to an internalizing antibody, as previously described (DOI: 10.3390/toxins14030184), it can be used to kill target cells precisely and specifically with little to no off-target toxicity (See, for example, Lund et. al., mAbs, 6(4), 1038-1050, doi.org/10.4161/mabs.28207). Saporin has been used as an ADC reagent for targeting tumor-expressed surface molecules such as breast cancer-associated Her-2, malignant melanoma associated CSPG-4, breast cancer associated EGFR, and FRα (See, for example, Hoffmann et. al. Sci Rep 10, 8869 (2020), doi.org/10.1038/s41598-020-65860-x) and is thus a validated warhead for ADCs. The commercially available Fab-ZAP assay (Advanced Targeting Systems Inc.) allows one to test the utility of antibodies to surface antigens for ADC by precisely delivering saporin based on a saporin-linked secondary antibody reactive to primary antibody specific to a tumor surface antigen. For the experiments described below, we utilized the Fab-ZAP rabbit kit [IT-57, KIT-57](atsbio.com/products/it57/), consisting of a chemical conjugate of goat anti-rabbit monovalent antibody and the ribosome-inactivating protein, saporin. The antibodies used in this kit are affinity-purified polyclonal antibodies against both the heavy and light chain of rabbit IgG.

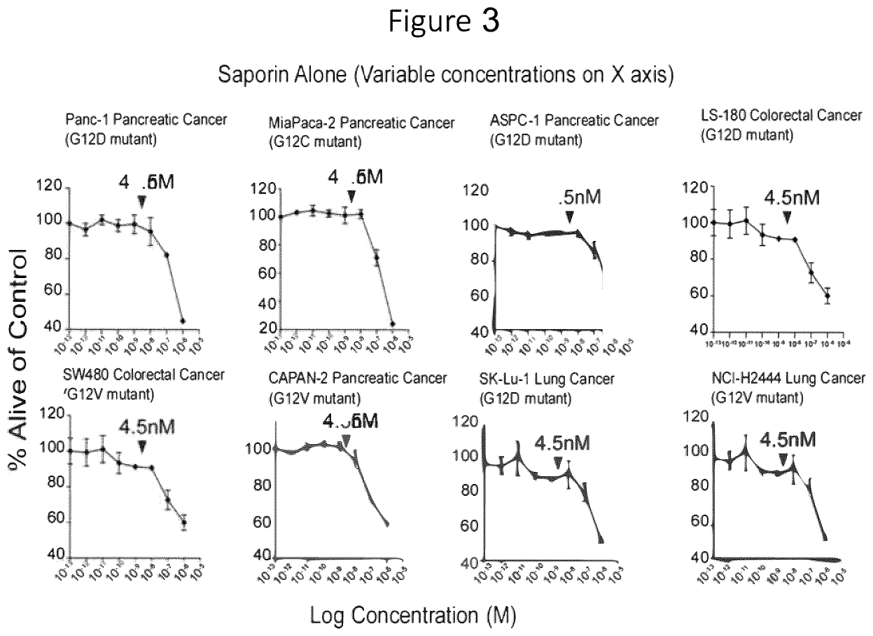

To study the potential of utilizing mutant K-Ras expressed at the cell surface, called mutant Surface K-Ras Antigen, as a target for ADCs, we used a rabbit anti-human K-Ras G12D antibody (GenTex, clone HL-10) and a rabbit anti-human K-Ras G12V antibody (Cell Signaling Technology, clone D2H12), as well as tumor cell lines expressing surface mutant K-Ras as described in Example 2. We specifically utilized Panc-1 (pancreatic carcinoma carrying a K-RAS G12D mutation), Capan-2 (pancreatic carcinoma carrying a K-RAS G12V mutation), AsPC-1 (pancreatic cell line carrying a K-RAS G12D mutation), LS180 (colon cancer cell line carrying a K-RAS G12D mutation), SW480 (colon cancer cell lines carrying a K-RAS G12V mutation), Capan-2 (pancreatic cancer cell line carrying a K-RAS G12V mutation), and two lung cancer cell lines (SK-LU-1 carrying a K-RAS G12D mutation and NCI-H2444 carrying a K-RAS G12V mutation). Serial dilutions of the antibodies or irrelevant rabbit IgG isotype control antibody were pre-incubated with 4.5 nM FabZAP reagent and then added to the indicated cell lines per manufacturer instructions in the Fab-ZAP kit. Cells were plated in a round bottom 96 well plate according to manufacturer's protocol with a total volume of 100 ul in each well (including Fab-Zap and different concentration of antibodies or respective controls). After 5 days incubation at 37 C in a 5% CO2 incubator, the percentage of viable cells compared to control wells with 4.5 nM FabZAP alone was determined using XTT assay according to the described protocol (Advanced Targeting Systems Inc cytotoxic assay protocol). Briefly, upon completion of the incubation, each well was supplemented with 50 ul of the XTT reagent to make the total volume 150 ul. Plates were then incubated at 37° C. for a minimum of 30 mins and absorbance was read at 450 nm in a plate reader. This assay relies on detecting the number of viable cells remaining on the day of development as measured via cell metabolism of a colorimetric molecule within the developing reagents. The mean absorbance in the untreated wells is depicted as 100% with 3 replicates in each treated group presenting a percentage in respect to the percent control. As depicted below in FIGS. 3-11 the cytotoxicity data is analyzed by comparing well readings of the treated wells (target antibody and Fab-ZAP) to those of the controls (Fab-ZAP only and saporin only) expressed as a percentage. GraphPad Prism software was used to analyze data and graph % alive as a function of primary antibody concentration.

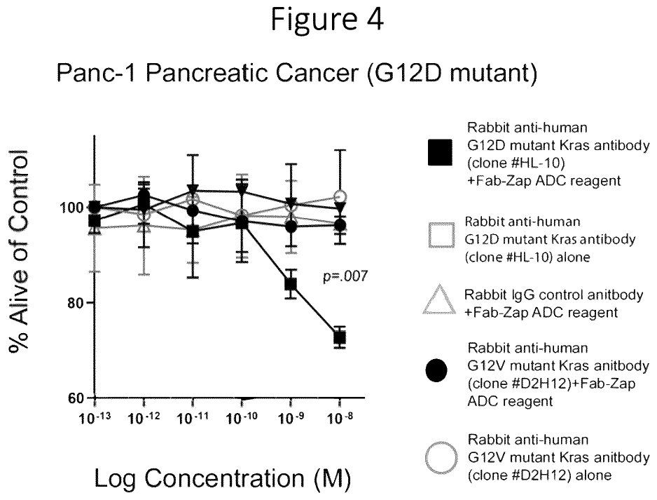

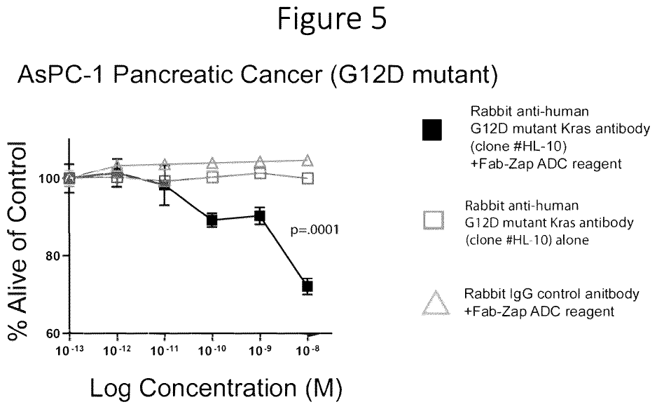

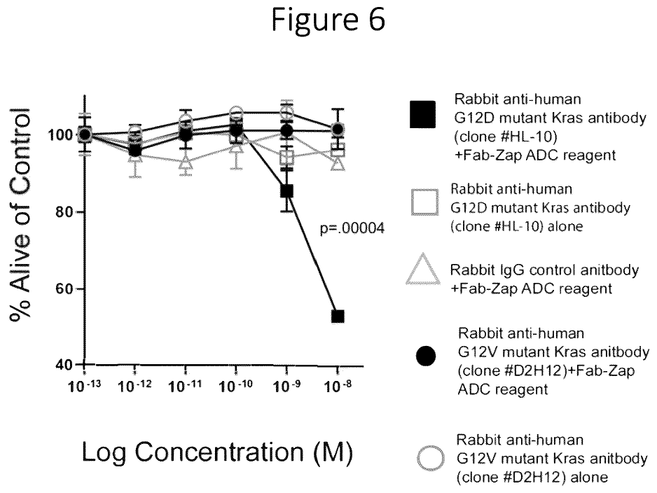

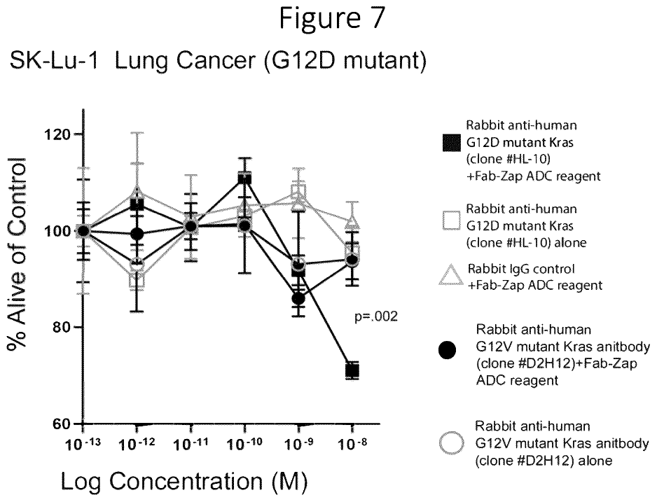

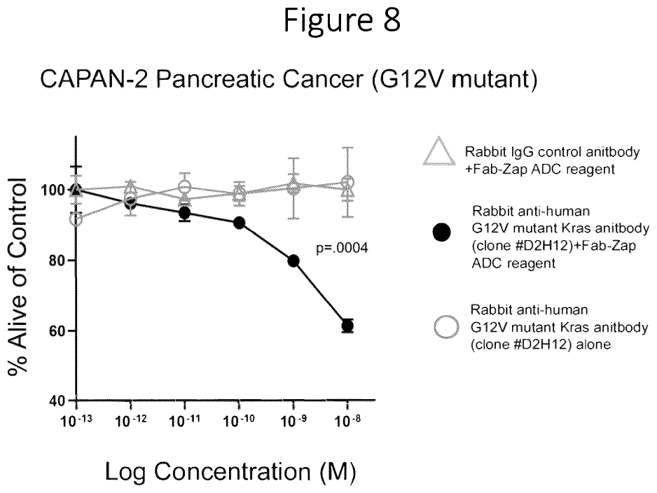

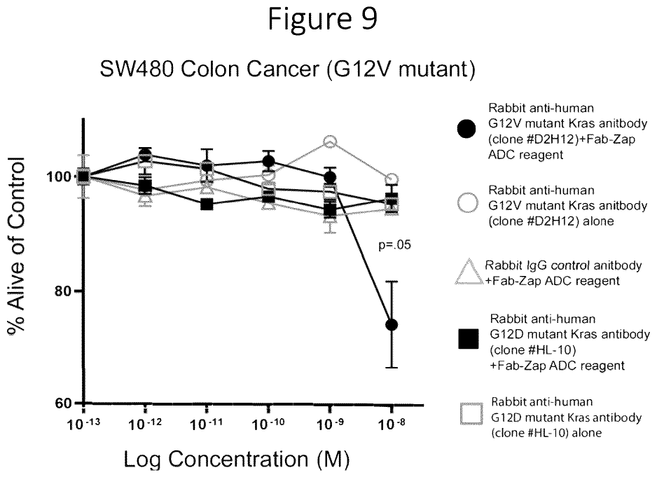

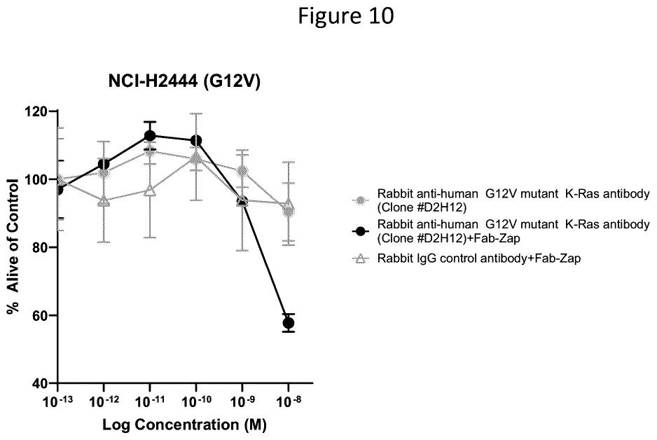

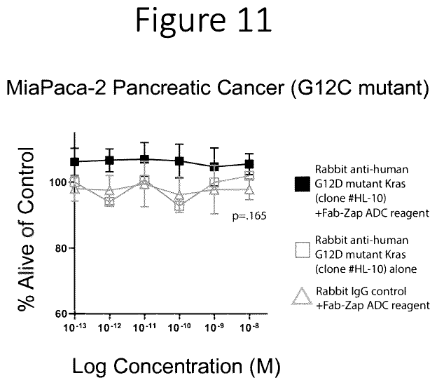

Saporin alone did not cause cell death at concentrations below 100 nM (FIG. 3) and thus no toxicity is evident at the 4.5 nM concentration used in the ADC assay without a targeting agent (arrow in FIG. 3). To determine if mutant K-Ras-specific targeting antibodies could be used for ADC in the presence of otherwise non-toxic 4.5 nM saporin targeting reagent, we next incubated Panc-1 (K-Ras G12D mutant) with variable concentrations of rabbit-anti human G12D mutant K-Ras antibody conjugated to the Fab-Zap reagent and noted concentration-specific death (FIG. 4). At identical conditions rabbit IgG conjugated to Fab-Zap control did not result in killing (FIG. 4). No cytotoxicity was evident when anti-G12V mutant K-Ras specific antibody conjugated to Fab-Zap was used in this K-Ras G12D mutant tumor or when the anti-G12D mutant K-Ras antibody was used alone (FIG. 4). As described above, saporin alone did not show any cytotoxicity until reaching concentrations of 100 nM (FIG. 3). Similar results were evident for other K-Ras G12D mutant cancers, such as AsPC-1 pancreatic cancer cell line (FIG. 5), LS180 colorectal cancer cell lines (FIG. 6), as well as SK-LU-1 lung cancer cell line (FIG. 7). For G12V mutant cancers, such as Capan-2 pancreatic cancer (FIG. 8), SW480 colorectal cancer (FIG. 9), and NCI-H2444 lung cancer cell line (FIG. 10) similar results were evident with the use of a K-Ras G12V-specific antibody conjugated to Fab-Zap, but anti-K-Ras G12D-mutation specific antibody did not result in ADC induced cell death in these K-Ras G12V-expressing tumors. In addition, the use of MiaPaCa-2, a K-Ras G12C expressing pancreatic cancer cell line, did not lead to ADC induced death with the K-Ras G12D-specific antibody conjugated to Fab-Zap (FIG. 11).

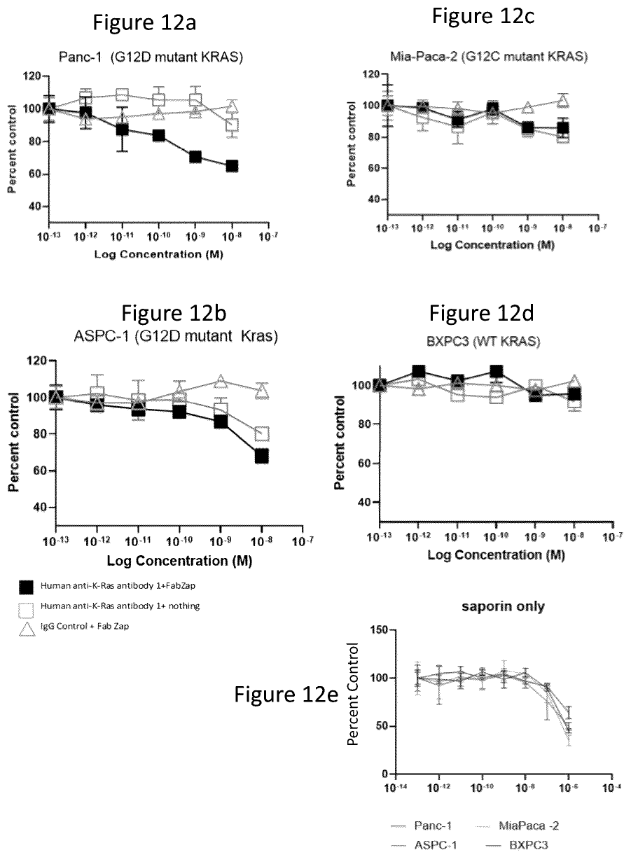

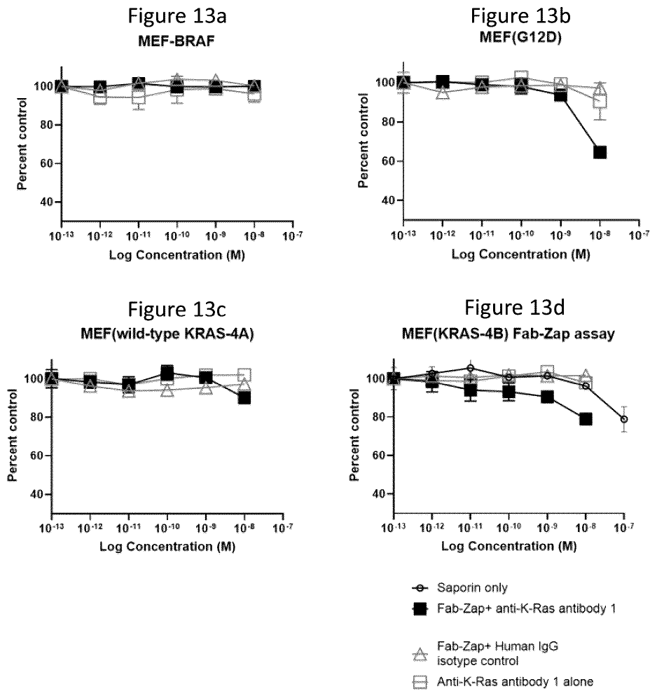

In another experiment using Human Anti-K-Ras Antibody 1 showed similar results in the K-Ras G12D mutant cancers of Panc-1 (FIG. 12a) and ASPC-1 (FIG. 12b). For G12C mutant cancers, such as Mia-Paca-2 (FIG. 12c), and for wildtype cancers, such as MXPC3 (FIG. 12d) similar results were observed, but anti-K-Ras G12D-mutation specific antibody did not result in ADC induced cell death in these K-Ras G12V-expressing tumors. A control of saporin only showed killing in Panc-1, ASPC-1, Mia-Paca-2, and BXPC3 cell lines (FIG. 12e). Finally, to demonstrate specificity, RASless MEFs cells rescued with BRAF, (G12D) K-Ras 4B, K-Ras-4A, or K-Ras 4B were tested in the surrogate Fab-Zap assay as above with Human Anti-K-Ras Antibody 1, except using a human secondary Fab-Zap reagent (atsbio.com/products/it5l/). Isotype IgG+FabZap or Human Anti-K-Ras Antibody 1 alone showed no increase in cytotoxicity. Human Anti-K-Ras Antibody 1+FabZap did not affect survival of MEF-BRAF or MEF-K-Ras-4A but did lead to cytotoxicity in MEF-(G12D) K-Ras 4B and MEF-K-Ras-4B (FIG. 13).

Taken together, our data suggest that the use of surface binding antibodies specific for mutant forms of Surface K-Ras Antigen can offer a viable method for generation of ADCs selective for tumor killing without off-target killing of cells not expressing the same mutant forms of Surface K-Ras Antigen on their cell surface.

### Example 4: Development of Novel Antibodies to Surface-Expressed K-Ras

Antibodies specific for K-Ras bound to either GDP or GTP will be developed by immunizing mice with GDP-bound K-Ras or GppNHp-bound K-Ras (GppNHp is a non-hydrolyzable analog of GTP). K-Ras protein will be loaded with either nucleotide prior to immunization of mice. As an additional strategy to develop mutant specific antibodies, a peptide containing the G12D mutation and spanning 5-17 of K-Ras will be used to immunize additional mice (Ac-KLVVVGADGVGKSC-amide) (SEQ ID NO 13). To minimize responses that bind wild-type K-Ras, some mice will first undergo subtractive immunization with wild-type K-Ras and treated with cyclophosphamide to kill B cells responding to wild-type K-Ras. All mice will then be immunized interperitoneally with protein or peptides mixed with complete or incomplete Freund's adjuvant. The resulting spleen cells and/or lymph nodes from immunized mice will be fused with NS1 myeloma cells to form hybridomas. Subsequent antibody clones will be screened by ELISA for binding to wild type K-Ras bound to GDP, wild type K-RAS bound to GppNHp, (G12D) K-Ras bound to GDP, and (G12D) K-Ras bound to GppNHp and by flow cytometry for binding to surface-expressed K-Ras on AsPC1 (G12D homozygous), SU.86.86 (G12D homozygous), and/or BxPC3 (K-Ras wild type) cells. Preferred antibody clones will recognize only the K-Ras antigen used to immunize mice. Antibody clones will be screened prior to fusion, after fusion, and again after subsequent subcloning.

### Example 5: Panning for Cell Surface-Expressed K-Ras Using Human Antibody Libraries

Another method for developing antibodies specific for mutant K-Ras, including K-Ras with mutations such as G12D, G12V, G12C, G13D, and G12R, is to pan libraries of pre-existing human polyclonal antibodies. Two existing libraries of human polyclonal antibodies will be used, one containing antibody sequences from naïve human donors and the other containing antibody sequences from autoimmune patient human donors. These antibodies were cloned and engineered into single chain variable fragments and introduced into phage display libraries that can be panned and screened for mutant K-Ras-specific antibodies that bind Surface K-Ras Antigen expressed on the cell surface.

Several different strategies will be utilized to select for antibodies that recognize either GDP or GTP bound mutant K-Ras. In all strategies, the libraries will be initially depleted of antibodies that bind wild type K-Ras protein when bound to whichever nucleotide is being screened for. The antibody library will then be panned for antibodies that bind to mutant K-Ras bound to either GDP or GppNHp. After the initial round of depletion/selection, up to three additional rounds of screening may be used. Each successive round of screening will employ depletion of antibodies that bind wild type K-Ras and selection of mutant K-Ras antibodies, either as protein or as expressed on the surface of cells as a Surface K-Ras Antigen. After the first round of selection, panning will be performed in the presence of excess competing wild type K-Ras protein or cells expressing wild type K-Ras to further minimize selection of phage binding to wild type K-Ras. To enrich for antibodies binding specific K-Ras epitopes, some rounds of panning will be eluted with specific proteins containing the desired epitopes. These specific proteins will include R11.1.6 and antibodies specific for mutant K-Ras, such as K-Ras G12D.

### Example 6: Proof of Concept for Development of a CAR-T Cell Targeting K-Ras

The use of Chimeric Antigen Receptor (CAR) T cells for selective tumor killing is an established method for targeting T cells to tumor cells expressing a defined surface target. To investigate the potential of surface K-Ras as a target for CAR T cells, the Human Anti-K-Ras Antibody 1 and rabbit anti-human K-Ras G12D antibody (HL-10 antibody) will be engineered into single chain antibody fragments. The single chain antibody fragments will contain an 18 amino acid “linker” of GSTSGSGKPGSGEGSTKG (SEQ ID NO: 25) which will be used to link the light and heavy chains. The scFv will be inserted into a second-generation CAR cassette containing a GM-CSF signaling peptide, a “hinge” region, the CD28 transmembrane and costimulatory domain, and the CD3(activation domain. The resulting scFv-CAR gene will be introduced into a lentiviral vector. Target T cells will then be activated, transduced with the scFv-CAR gene, and expanded.

Two measures of CAR-T cells activity will be assessed: cytokine secretion and cell killing. To measure cytokine secretion, expanded anti-K-Ras CAR-T cells will be incubated at 1:1 ratio with cells expressing K-Ras on the surface. After 16 hours of incubation the supernatant of the cells will be removed. Quantities of secreted IL-2 and IFN-γ in the supernatant will be measured by ELISA. To measure cell killing, adherent target cells will be grown to confluence before addition of 105 CAR-T cells. The cells will be co-cultured for 2-3 days with the impedance of the cell layer measured over time. Total cytolysis is calculated by the change in impedance across the time of the experiment.

### Example 7: Isolation of Cell Samples for Diagnostic Testing of Surface K-Ras Expression in Patient Samples

Human tissue from cancerous sites, as well as adjacent normal tissue, were banked right after surgical procedure. Tissues were immediately processed according to the following protocol to isolate single cells. Briefly, each tissue was cut into very small segments using a sterile blade. Then tissues were resuspended in RPM11640 complete media (with 10% FBS, 1× penicillin-streptomycin) and run in MACS dissociator (Milteny Biotech) for 2 cycles of 1 min each to initiate dissociation. Next the tissues were digested in digestion media consisting of IMDM media, supplemented with collagenase type 2 (0.5 mg/ml) and DNase (0.5ul/sample). The total digestion media volume used was 7 ml for each tissue size of 1 mg. Next the tissues, immersed in digestion media was shaken at 37° C. for 35 min at 850 rpm.

Following digestion, the tissues were run again in gentle MACS dissociator to break up the digested tissue and retrieve single cells. Cells were next washed twice in 1×PBS to eliminate any digesting enzyme and DNase. Cells were passed through a 70 uM strainer and resuspended in RPMI complete media.

### Example 8: Diagnostic Testing for Surface K-Ras Expression by Flow Cytometry

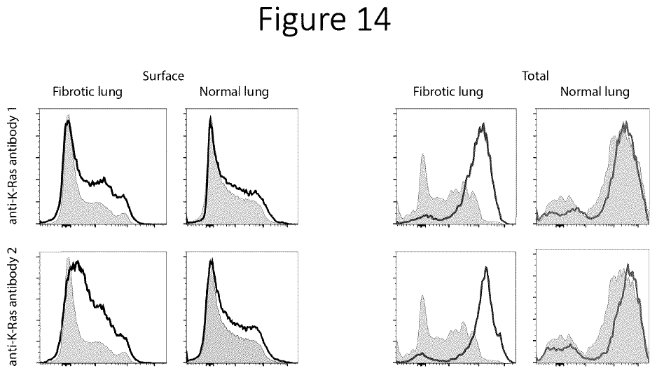

Single cell suspensions obtained from patients as set forth in Example 1 can be used for flow cytometric analysis. Flow cytometric analysis of surface K-Ras from primary human tissue was performed using Human Anti-K-Ras Antibody 1 and human K-Ras antibody 2 (FIG. 14). In this example, cells obtained from fibrotic lung tissue show an increase in K-Ras surface expression compared to cells from normal lung tissue.

### Example 9: Diagnostic Testing for Surface K-Ras Expression by Western Blot

Once patient samples have been obtained using the methods of Example 1, western blot analysis for surface K-Ras expression may be analyzed. Cell surface proteins can be labelled and subsequently isolated using Pierce™ Cell Surface Biotinylation and Isolation Kit (ThermoFisher Scientific, Catalog number: A44390). Cells are first labeled with EZ-Link Sulfo-NHS-SS-Biotin, a thiol-cleavable amine-reactive biotinylation reagent. Cells are then lysed, and the labeled proteins can be captured with NeutrAvidin Agarose. The flowthrough can then be collected from agarose column representing the non-biotinylated fraction which was the intracellular protein after the cells were lysed. NeutrAvidin Agarose-bound biotinylated protein fractions can be eluted using a reducing buffer with protease inhibitors cocktail. Dithiothreitol (DTT) will be used in the elution to reduce the disulfide bonds in the biotin label, resulting in the release of the bound proteins without the biotin label.

Eluted surface biotinylated and the intracellular flow through protein fractions can then utilized to run western blots with different K-Ras antibodies (see Example 2, FIG. 2m). Proteins can be eluted in buffer containing 20 mM Tris-HCl (pH 7.5), 150 mM NaCl 1 mM Na2EDTA, 1 mM EGTA, 1% Triton X-100, and protease inhibitors cocktail. Performance of gel electrophoresis and western blotting will be known to any of ordinary skill in the art.

### Example 9: Diagnostic Testing for Surface K-Ras Expression by Electron Microscopy

Live Panc-1 cells can be pre-labelled with either anti-KRAS antibody or IgG control followed by staining with gold labelled secondary. Cells can then be post-fixed in 2% osmium tetroxide in Sorensen's buffer for 1 h, dehydrated in an ascending ethanol series (30%,50%,70%,90%,100%), and embedded in Epon/Araldite resin. Samples will then be stained in a premixed solutions of uranyl acetate and lead citrate and examined at 200V using a F20 electron microscope having field emission gun (FEG) source of electrons. Negative-stain data collection is then implied with a 4k×4k Ultra Scan CCD camera running Gatan GIF Quantum with DualEELS and High-Speed Spectrum Imaging.

### Example 10: Diagnostic Testing for Surface K-Ras Expression by Confocal Microscopy

Surface KRAS identification by confocal microscopy was performed using a combination of anti-KRAS antibody and a cell-surface membrane dye using CellBrite® Steady Membrane Staining Kit (Biotium Inc. Fremont, CA). Briefly, live cells were labelled using the CellBrite® Steady 488 dye (green fluorescence). Cells were labelled using 1× of dye in cell culture media followed by incubation in 37 degree c. incubator for 30 mins. Cells were then washed immediately with culture media twice which had 10% FBS supplemented to quench any unbound dye. Next cells were subjected to surface K-Ras staining using Human Anti-K-Ras Antibody 1 on ice for 30 minutes followed by a secondary anti-human PE (red fluorescence) antibody staining for 30 mins on ice as well. It was ensured that cells were kept healthy and live till this step of staining. Finally, cells were washed and overlaid on slides and mounted with ProLong™ Gold Antifade Mounting solution with DAPI (ThermoFisher Scientific, Catalog number: P36931).

The images were acquired using a spinning disk confocal (Zeiss LSM 880) microscope equipped with Airyscan and Imaged with ZEISS Airyscan 2 in Multiplex mode for efficient super-resolution imaging of a large field of view. Z-slices were set to 0.2 micron unless otherwise noted. For high-resolution imaging, we either used Plan-Apochromat 40×/1.3 or for confocal modality performance, we used the 63×1.4NA Plan-Apochromat objective and the 568 nm excitation and 570-615 nm emission filters were used. The confocal images were deconvolved utilizing the constrained iterative deconvolution routines n (Zen software version 2.3, Carl Zeiss, Obekochen, Germany).

Confocal microscopy images revealed distinct KRAS staining (FIG. 2g) when stained with anti-KRAS antibody (left panel) whereas there was no red staining visible with the control antibody (right panel). Membrane dye and DAPI served as cellular markers to differentiate surface and intracellular region.

### Example 11: Increasing Surface K-Ras Availability Through Treatment Combinations

We examined the effect on K-Ras surface expression after treating tumor cell lines with chemotherapy agents (fluorouracil (5-FU)), or mutation-specific K-Ras small molecule inhibitors (G12D-specific—MRTX1133, G12C-specific—MRTX849).

For 5-FU treatment, 100,000 PANC-1 cells were seeded into 24 well replicate plates. Cells were allowed to grow for 24 hours before 10 uM of 5-FU was added to the culture media. At 0-, 72-, and 120-hours cells were dislodged by using TryPLE express and resuspended in buffer containing PBS pH 7.2, 5% FCS. Cells were stained with human K-Ras antibody 1. All staining was performed at 10 μg/ml of antibody concentration and a volume of 100ul for 30 minutes on ice in V-bottom 96 well plates. Surface K-Ras staining was quantitated by measuring the MFI of cells mock treated or treated with 5-FU. The fold change was calculated by normalizing to the cells stained with isotype control. As shown in FIG. 15a, treatment with 5-FU increased the availability of surface K-Ras in Panc-1 cells, with increasing amounts of K-Ras available the longer the treatment was allowed to continue. Treatment with 5-FU chemotherapy, and potentially other chemotherapies, increased the availability of surface K-Ras.

For treatment with mutation-specific K-Ras small molecule inhibitors, 100,000 PANC-1 cells were seeded into one set of 24 well replicate plates and 100,000 MiaPaca-2 cells were seeded into a second set of plates. Cells were allowed to grow for 24 hours before 10 nM of MRTX1133 or 10 nM of MRTX849 were added to the culture media. At 0-, 72-, and 120-hours cells were dislodged by using TryPLE express and resuspended in buffer containing PBS pH 7.2, 5% FCS. Cells were stained with human K-Ras antibody 1. All staining was performed at 10 ug/mI of antibody concentration and a volume of 100ul for 30 minutes on ice in V-bottom 96 well plates. Surface K-Ras staining was quantitated by measuring the MFI of cells mock treated or treated with either MRTX1133 or MRTX849. The fold change was calculated by normalizing to the cells stained with isotype control. As shown in FIGS. 15b-15c, treatment with MRTX1133 increased the availability of surface K-Ras on PANC-1 cells but not MiaPaca-2 cells. As shown in FIGS. 15a-15c, treatment with MRTX849 increased the availability of surface K-Ras on MiaPaca-2 cells, but not PANC-1. The results demonstrated that treatment with mutation-specific small molecule inhibitor effects the availability of K-Ras on the cell surface only when the appropriate K-Ras mutation is present in the cell. Similarly to treatment with 5-FU, increasing amounts of K-Ras were available the longer the treatment with an appropriate mutation-specific K-Ras small molecule inhibitor was allowed to continue. Taken together, several different methods increase the availability of surface K-Ras.

### Example 12a: Increased K-Ras Surface Availability Due to K-Ras Small Molecule Treatment Enhances Killing with Surface K-Ras Targeted Antibody Drug Conjugates (ADC)

In this example, we used the commercially available human Fab-ZAP assay (Advanced Targeting Systems Inc.), similar to Example 3, to test whether increasing surface K-Ras availability with MRTX1133 would potentiate killing with surface K-Ras targeted ADCs. For the experiments described below, we utilized the Fab-ZAP human kit [IT-51, KIT-51](atsbio.com/products/it51/), consisting of a chemical conjugate of goat anti-rabbit monovalent antibody and the ribosome-inactivating protein, saporin. The antibodies used in this kit are affinity-purified polyclonal antibodies against both the heavy and light chain of rabbit IgG.

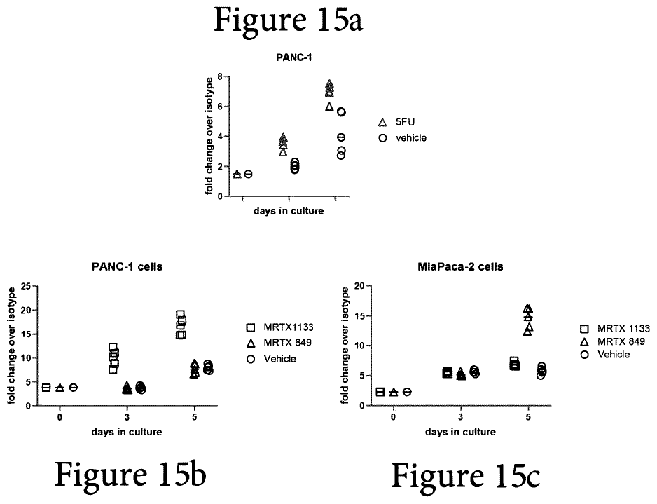

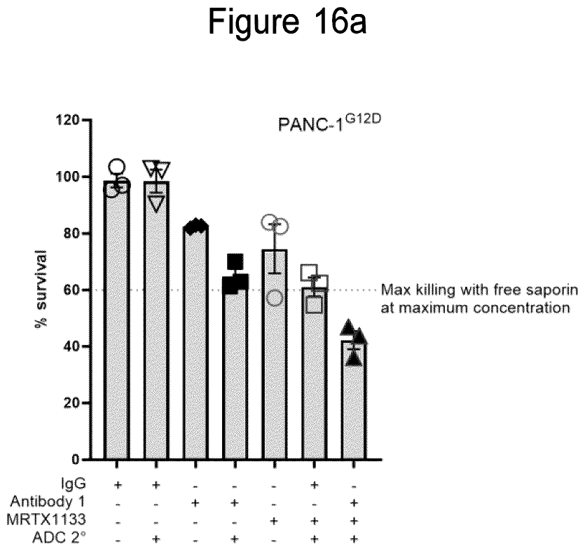

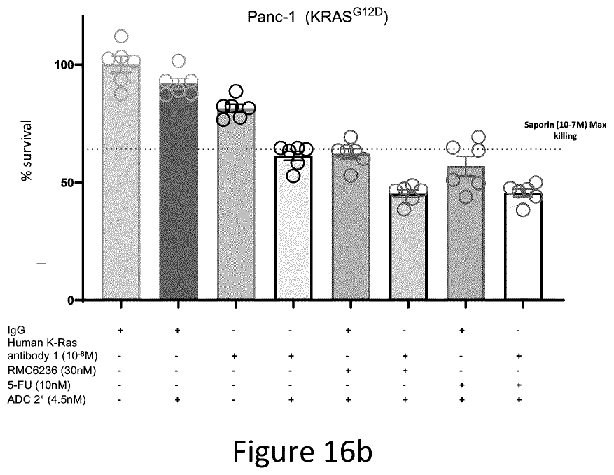

In one experiment, we tested whether increasing surface K-Ras availability with the small molecule inhibitor MRTX1133 would potentiate killing with surface K-Ras targeted ADCs. For a description of MRTX1133, see, for example, Wang et al., Journal of Medicinal Chemistry 2022 65 (4), 3123-3133. DOI: 10.1021/acs.jmedchem.1c01688. PANC-1 cells were seeded at 100,000 cells/well in 24 well replicate plates. Cells were allowed to grow for 24 hours before 10 nM of MRTX1133 was added to the culture media. At 0-, 72-, and 120-hours serial dilutions of the antibodies, that were pre-incubated with 4.5 nM FabZAP reagent, were added to the PANC-1 cell line per manufacturer instructions in the Fab-Zap kit. After 5 days incubation at 37 C in a 5% CO2 incubator, the percentage of viable cells compared to control wells with 4.5 nM FabZAP alone was determined using XTT assay according to the described protocol (Advanced Targeting Systems Inc cytotoxic assay protocol). Briefly, upon completion of the incubation, each well was supplemented with 50 ul of the XTT reagent to make the total volume 150 ul. Plates were then incubated at 37 C for at least 30 mins, and absorbance was read at 450 nm in a plate reader. This assay relies on detecting the number of viable cells remaining on the day of development as measured via cell metabolism of a colorimetric molecule within the developing reagents. The mean absorbance in the untreated wells is depicted as 100% with 3 replicates in each treated group presenting a percentage in respect to the percent control. As depicted in FIG. 16a, the cytotoxicity data is analyzed by comparing well readings of the treated wells to those of the controls (IgG only) expressed as a percentage. GraphPad Prism software was used to analyze data and graph % alive.

Saporin alone did not cause cell death at concentrations below 100 nM (FIG. 3) and thus no toxicity is evident at the 4.5 nM concentration used in the ADC assay without a targeting agent. For comparison that maximum killing of the cells with 100 uM of free saporin is indicated by the dotted line. MRTX1133 alone, or anti-K-Ras antibody with Fab-ZAP alone both were able to kill cells (FIG. 16a). When MRTX1133 and anti-Kras antibody with Fab-Zap were combined, the effect was greater than either agent alone (FIG. 16a). This demonstrates that the combination of small molecule inhibitors with surface K-Ras-targeted ADCs increase tumor cell killing.

### Example 12b: Chemotherapy Agent Enhances Killing with Surface K-Ras Targeted Antibody Drug Conjugates (ADC)

In this example, we used the commercially available Fab-ZAP assay (Advanced Targeting Systems Inc.), described in Example 12a, to test whether increasing surface K-Ras availability with RMC-6236 would potentiate killing with surface K-Ras targeted ADCs. One combination was an ADC of human anti-K-Ras antibody 1 with the chemotherapeutic agent fluorouracil (5-FU), and a second combination was an ADC of human anti-K-Ras antibody 1 with the small molecule inhibitor named RMC-6236. For a description of RMC-6236, see, for example, Arbour K et al., Ann Oncol 2023; 34:S458.

PANC-1 cells were seeded at 10,000 cells/well in 96 well replicate plates. Cells were allowed to grow for 24 hours before treatment with 30 nM of RMC-6236, 10 uM of 5-FU, or a combination of RMC-6236 and 5-FU. At 24 hours after the PANC-1 cells were seeded, treatment groups received either human K-Ras antibody 1 at a fixed concentration of 10⁻⁸M, or IgG at a fixed concentration of 10⁻⁸M, that were pre-incubated with fixed concentration of 4.5 nM FabZAP reagent, per manufacturer instructions in the Fab-Zap kit. After 5 days incubation at 37° C. in a 5% CO2 incubator, the percentage of viable cells that survived treatment compared to control wells with Fab-ZAP alone was determined using XTT assay according to the described protocol (Advanced Targeting Systems Inc cytotoxic assay protocol). Briefly, upon completion of the incubation, each well was supplemented with 50 ul of the XTT reagent to make the total volume 150 ul. Plates were then incubated at 37° C. for at least 30 mins, and absorbance was read at 450 nm in a plate reader. This assay relies on detecting the number of viable cells remaining on the day of development as measured via cell metabolism of a colorimetric molecule within the developing reagents. The mean absorbance in the control (PANC-1 cells treated with IgG) is depicted as 100% with 3 replicates in each treated group presenting a percentage in respect to the percent control. The cytotoxicity data is analyzed by comparing well readings of the treated wells to those of the controls (IgG only treatments) expressed as a percentage. GraphPad Prism software was used to analyze data and graph % alive.

For comparison, the maximum cell killing with 10⁻⁷M of free saporin is indicated by the dotted line. Treatment with Human K-RAS Antibody 1+FabZap, RMC-6236, and the combination of Human K-Ras Antibody 1+FabZap and RMC-6236 killed PANC-1 cells equivalently to the max killing achieved with high concentrations of free saponin (FIG. 16B). However, when PANC-1 cells were treated with a combination of RMC-6236 and Human K-Ras Antibody 1+FabZap or 5-FU and Human K-Ras Antibody 1+FabZap, the effect was greater than either agent alone (FIG. 16B).

Taken together, these results show that the combination of small molecule inhibitors and chemotherapy agents with surface K-Ras-targeted ADCs increased tumor cell killing.

### Example 13: Surface K-Ras-Targeting Bispecific Antibodies Increase T Cell Killing of Surface K-Ras-Expressing Cells

To demonstrate whether bispecific antibodies would be sufficient to induce T cell-mediated killing of surface K-Ras expressing cells, we designed an asymmetric bispecific antibody (Anti-K-Ras antibody 1×CD3). As shown in FIG. 18, the bispecific antibody was designed with the Fab-Fc region of Human Anti-K-Ras Antibody 1 (Anti-K-Ras Fab HC-Fc/Anti-K-Ras Fab LC) (Fc disabled with N297G and T366S/L368A/Y407V) and an scFv of a CD3-specific antibody linked to a disabled Fc (N297G and T366W) (Anti-K-Ras Fab HC—FC—EVQLVQSGGGVVQPGRSLRLSCAASGFTSRHPGMHWVRQAPGKGLEWVAVISHDGSK KYYADSVKGRFTISRDNSKNTLFVQLSSLRPEDTAVYYCATSLYSSMDLWGQGTTVTVSS ASTKGPSVFPLAPSSKSTSGGTAALGCLVKDYFPEPVTVSWNSGALTSGVHTFPAVLQS SGLYSLSSVVTVPSSSLGTQTYICNVNHKPSNTKVDKKVEPKSCDKTHTCPPCPAPELLG GPSVFLFPPKPKDTLMISRTPEVTCVVVDVSHEDPEVKFNWYVDGVEVHNAKTKPREEQ YGSTYRVVSVLTVLHQDWLNGKEYKCKVSNKALPAPIEKTISKAKGQPREPQVYTLPPSR EEMTKNQVSLSCAVKGFYPSDIAVEWESNGQPENNYKTTPPVLDSDGSFFLVSKLTVDK SRWQQGNVFSCSVMHEALHNHYTQKSLSLSPG (SEQ ID NO: 26) and Anti-K-Ras Fab LC-QSVVTQPPSVSAAPGQKVTISCSGSNSNIGKNYVSWFQQVPGTAPKLLIFEDNQRPSGIP DRFSASKSGTSASLAISGLQSEDEADYYCAAWDDKFGVHWVFGGGTKLTVLGQPKAAP SVTLFPPSSEELQANKATLVCLISDFYPGAVTVAWKADSSPVKAGVETTTPSKQSNNKYA ASSYLSLTPEQWKSHRSYSCQVTHEGSTVEKTVAPTECS (SEQ ID NO: 27) and CD3 OKT3 scFv-FC—DIKLQQSGAELARPGASVKMSCKTSGYTFTRYTMIWVKQRPGQGLEWIGYINPSRGYTN YNQKFKDKATLTTDKSSSTAYMQLSSLTSEDSAVYYCARYYDDHYCLDYWGQGTTLTVS SVEGGSGGSGGSGGSGGVDDIQLTQSPAIMSASPGEKVTMTCRASSSVSYMNWYQQK SGTSPKRWIYDTSKVASGVPYRFSGSGSGTSYSLTISSMEAEDAATYYCQQWSSNPLTF GAGTKLELKGGGGSEPKSSDKTHTCPPCPAPELLGGPSVFLFPPKPKDTLMISRTPEVTC VVVDVSHEDPEVKFNWYVDGVEVHNAKTKPREEQYGSTYRVVSVLTVLHQDWLNGKEY KCKVSNKALPAPIEKTISKAKGQPREPQVYTLPPSREEMTKNQVSLWCLVKGFYPSDIAV EWESNGQPENNYKTTPPVLDSDGSFFLYSKLTVDKSRWQQGNVFSCSVMHEALHNHYT QKSLSLSPG (SEQ ID NO: 28)). A control bispecific (control IgG×CD3) was also made with the Fab region of a human anti-RSV antibody and the same CD3 OKT3 scFv—Fc domain disclosed above (RSV Fab HC-Fc—QVTLRESGPALVKPTQTLTLTCTFSGFSLSTAGMSVGWIRQPPGKALEWLADIWWDDKK HYNPSLKDRLTISKDTSKNQVVLKVTNMDPADTATYYCARDMIFNFYFDVWGQGTTVTVS SASTKGPSVFPLAPSSKSTSGGTAALGCLVKDYFPEPVTVSWNSGALTSGVHTFPAVLQ SSGLYSLSSVVTVPSSSLGTQTYICNVNHKPSNTKVDKKVEPKSCDKTHTCPPCPAPELL GGPSVFLFPPKPKDTLMISRTPEVTCVVVDVSHEDPEVKFNWYVDGVEVHNAKTKPREE QYGSTYRVVSVLTVLHQDWLNGKEYKCKVSNKALPAPIEKTISKAKGQPREPQVYTLPPS REEMTKNQVSLSCAVKGFYPSDIAVEWESNGQPENNYKTTPPVLDSDGSFFLVSKLTVD KSRWQQGNVFSCSVMHEALHNHYTQKSLSLSPG (SEQ ID NO: 29) and RSV Fab LC—DIQMTQSPSTLSASVGDRVTITCSASSRVGYMHWYQQKPGKAPKLLIYDTSKLASGVPSR FSGSGSGTEFTLTISSLQPDDFATYYCFQGSGYPFTFGGGTKVEIKRTVAAPSVFIFPPSD EQLKSGTASVVCLLNNFYPREAKVQWKVDNALQSGNSQESVTEQDSKDSTYSLSSTLTL SKADYEKHKVYACEVTHQGLSSPVTKSFNRGEC (SEQ ID NO: 30)).

To test target cell killing, Panc-1 luciferase-expressing cell lines (Panc-1 L) were used. These cells constitutively express luciferase, and luciferase activity can be used as a proxy for the number of living target cells. Importantly, the T cells that are co-cultured with the target cells do not express luciferase and therefore do not contribute to the luciferase signal. This allows luciferase expression to be a measure of only target cell viability. Briefly, 50,000 Panc-1 L cells were plated in each well of a 96 well plate optical polymerase black plate (ThermoFisher #165305) in a volume of 100ul. T cells were isolated from human PBMC and added in the ratio of 1:1 (50,000 cells) or 2:1 (100,000 cells) in a volume of 100ul for a total well volume of 200ul. Respective bispecific (Human Anti-K-Ras Antibody 1×CD3 bispecific and control IgG (RSV) X CD3 bispecific) were added to the wells ranging from a concentration of 10⁷ to 10⁻¹⁰ M. Each group was plated as 5 replicate wells. One group had no T cells added (Panc-1 L only) and one group had T cells alone without Panc-1 L as controls. Cells were cultured in plates for 72 hours at 37° C. in a 5% CO₂ incubator followed by analysis. On the day of analysis, 100ul of media was removed from all wells and 100 ul of reagent D-luciferin (1× concentration) was added. Wells were mixed thoroughly and after 5 minutes, luminescence was measured by reading the plate in Tecan Spark Multimode Microplate reader. Luminescence of each well was recorded, and the intensity was proportional to the number of living cells in each well. We used GraphPad prism for statistical analysis and representation of data.

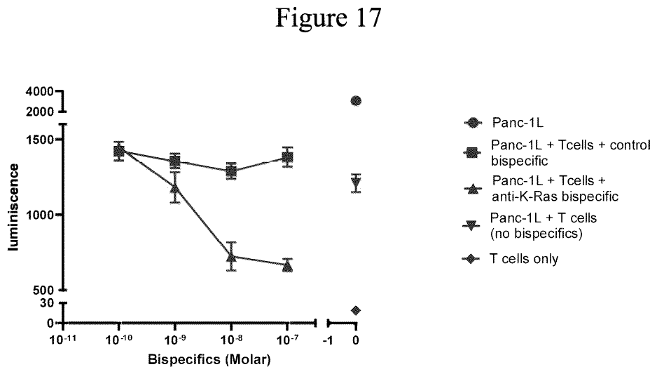

As depicted in FIG. 17, co-culture of target Panc-1 L and human T cells with the Human Anti-K-Ras Antibody 1×CD3 bispecific lead to concentration dependent decreases in viable cells, as measured by luciferase expression. In contrast, co-culture of target Panc-1 L and human T cells with the control IgG×CD3 bispecific did not lead to changes in cell viability compared to co-culture of target Panc-1 L and human T cells without a bispecific antibody. Panc-1 L cells alone had the highest viable cells, indicating that human T cells kill target cells at a low level when co-cultured, as would be expected. Together this demonstrates that incubation of target cells and T cells with a surface K-Ras targeted bispecific leads to increased killing of target cells in a dose-dependent manner.

### Example 13: Assessing Antibody Binding to K-Ras Antigen

To demonstrate binding of antibodies to K-Ras, Enzyme Linked Immunosorbant Assays (ELISAs) were used. Plates were coated with 1 μg/ml Neutravidin (neutralite avidin or deglycosylated chicken avidin) overnight at 4° C. to capture biotinylated proteins. Plates were blocked with 1% BSA/PBS for 60 min. Plates where loaded with biotinylated (G12D)K-Ras-GDP, (G12D)K-Ras-GppNHp, K-Ras-GDP, K-Ras-GppNHp, or BSA at 1 μg/ml for 60 min. Purified antibodies were tested for K-Ras binding from concentrations between 1-0.001 ug/ml for 60 min. Antibody detection was carried out using anti-human IgG-HRP and TMB staining. Between incubations plates were wash three times with PBST. Average values of duplicate measurements with their standard deviations are shown.

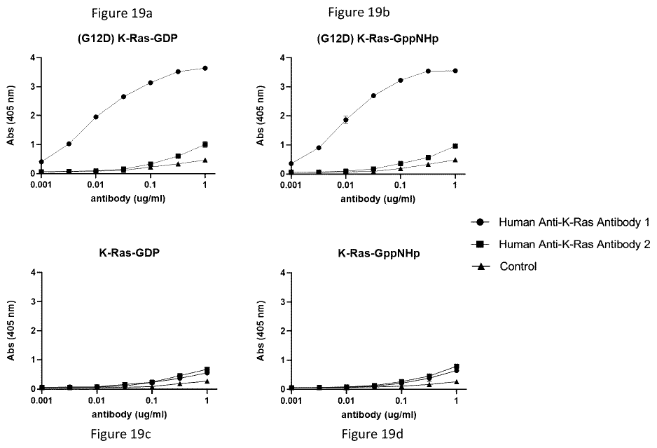

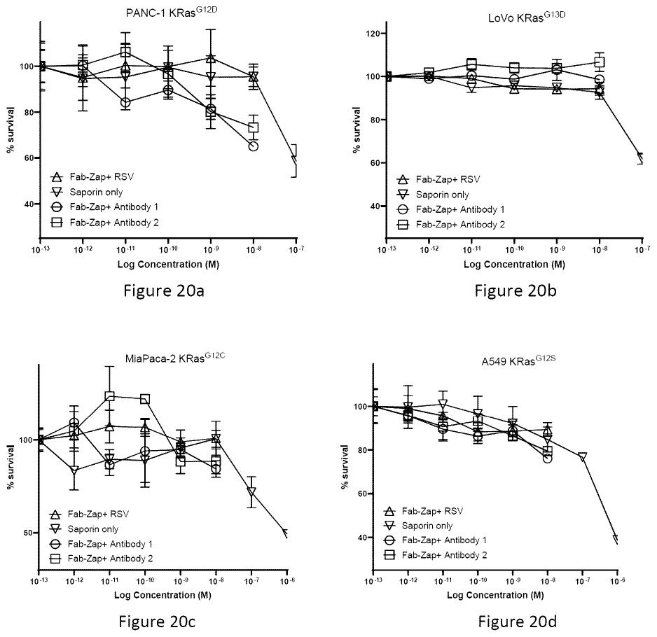

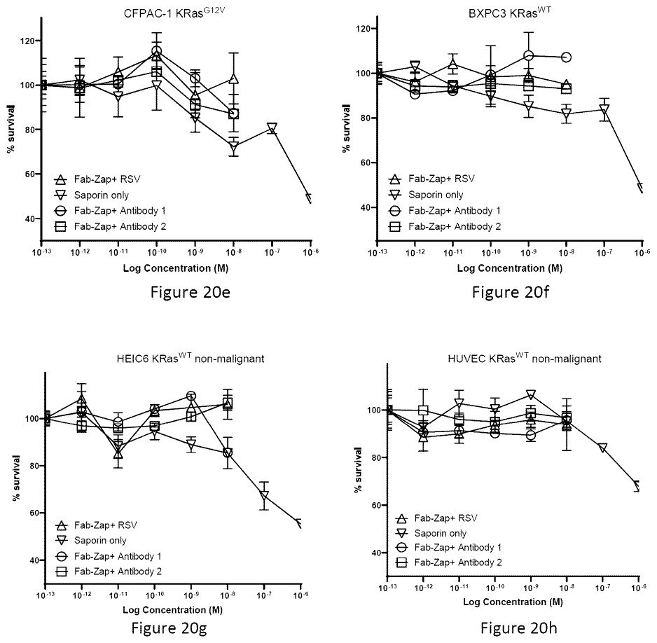

Both Human Anti-K-Ras Antibody 1 and 2 bound at a low level to wild type K-Ras, either loaded with GDP or GppNHp loaded nucleotides (FIGS. 19c-19d). Similarly, Human Anti-K-Ras Antibody 2 bound (G12D) K-Ras at a low level, regardless of the loaded nucleotide. In contrast, Human Anti-K-Ras Antibody 1 bound at a high level to (G12D) K-Ras (with either nucleotide loaded), demonstrating preferential binding of Human Anti-K-Ras Antibody 1 for G12D mutant K-Ras (FIGS. 19a-19d).

### Example 14: Validation of Antibody Drug Conjugates (ADC) for Selective Killing of Cancer Cell Lines Through the Use of Mutation-Specific Antibodies

To study the potential of utilizing mutant K-Ras expressed at the cell surface as a target for ADCs, we used a human anti-K-Ras antibody 1 (antibody 1) and a human anti-K-Ras antibody 2 (antibody 2), as well as tumor cell lines expressing surface mutant K-Ras. Specifically, Panc-1 K-Ras^{G12D} (pancreatic carcinoma carrying a K-RAS G12D mutation), LoVo K-Ras^{G13D}, MiaPaca-2 K-Ras^{G12C} (same as Mia-Paca-2), A549 K-Ras^{G12S} CFPAC-1 K-Ras^{G12V}, BXPC3 K-Ras^{WT}, HEIC6 K-Ras^{WT} (non-malignant cell line), and HUVEC K-Ras^{WT} (non-malignant cell line) in Fab-ZAP assays. An antibody specific for RSV was used as a negative control for K-Ras binding.

Serial dilutions of the antibodies were pre-incubated with 4.5 nM FabZAP reagent and then added to the indicated cell lines per manufacturer instructions in the Fab-ZAP kit. Cells were plated in a round bottom 96 well plate according to manufacturer's protocol with a total volume of 100 ul in each well (including FabZap and different concentration of antibodies or respective controls). After 5 days incubation at 37 C in a 5% CO2 incubator, the percentage of viable cells compared to control wells with 4.5 nM FabZAP alone was determined using XTT assay according to the described protocol (Advanced Targeting Systems Inc cytotoxic assay protocol). Briefly, upon completion of the incubation, each well was supplemented with 50 ul of the XTT reagent to make the total volume 150 ul. Plates were then incubated at 37° C. for a minimum of 30 mins and absorbance was read at 450 nm in a plate reader. This assay relies on detecting the number of viable cells remaining on the day of development as measured via cell metabolism of a colorimetric molecule within the developing reagents. The mean absorbance in the untreated wells is depicted as 100% with 3 replicates in each treated group presenting a percentage in respect to the percent control.

### Example 15: Assessing In Vivo Staining of Tumor Tissue by Anti-K-Ras Antibodies

To demonstrate specific recognition of surface K-Ras in in vivo tumors, tumor fragments from patient derived xenograft (PDX) model PA0787 and PA1252 (both derived from resected human pancreatic cancer expressing the G12D Kras mutation) (Crown Bio) were inoculated into nude Balb/c mice, which lack T and B cells, and allow for the growth and engraftment of human tumors. Each mouse was inoculated subcutaneously in the right upper/lower flank with primary human tumor xenograft model tumor fragment (2-3 mm in diameter) for tumor development. Once the tumors reached approximately 500 mm³ the mice were injected with antibodies as described below.

Once tumors reached 500 mm³ the mice were randomized and injected either with Human Anti-K-Ras Antibody 1 or an isotype control antibody at a dose of 10 mg/kg in a volume of 10 ul/g via tail vein injection. 30 minutes later the mice were euthanized, and the tumors were harvested and frozen in optimal cutting temperature (OCT) freezing media for immunohistochemical evaluation.

The tumors were sectioned into 5 um sections on a standard dermatome and processed for immunohistochemistry. Briefly, cryosections were fixed with cold acetone/methanol (at a 1:1 dilution) solution for 5 minutes. After washing sections in PBS for 15 minutes, sections were blocked with 10% normal goat serum at room temperature for 45 minutes and incubated with secondary goat anti-human IgG (Alexa fluro 488 Invitrogen A-11013) at room temperature for 45 minutes. ProLong™ Gold Antifade Reagent with DAPI (Catalog P36931 ThermoFisher Scientific) was added before putting the cover slides on. Images were acquired with the Leica Microsystems DM6B-Z.

As can be seen in FIGS. 21a-21b, both PDX model PA0787 and PA1252 demonstrate better tumor staining with Human Anti-Kras Antibody 1 compared to staining with the isotype control antibody. Thus, in vivo treatment with anti-K-Ras antibodies specifically stained G12D mutant K-Ras containing pancreatic cancers.

### Example 16: Validation of Antibody Drug Conjugates (ADC) for In Vivo Selective Killing of Cancer Cell Lines Through the Use of Mutation-Specific Antibodies

To study the ability of anti-K-Ras antibodies to provide control of K-Ras mutant tumors in vivo, we conjugated anti-K-Ras antibody 1, as well as an isotype control antibody to deruxtecan (DXd). DXd is a protease cleavable linker attached to a derivative of exatecan that acts as topoisomerase I inhibitor. DXd targeted to cancer cells by antibodies as an ADC has been used by FDA-approved ENHERTU@(fam-trastuzumab-deruxtecan-nxki), which is a conjugation of anti-Her-2 antibody and deruxtecan.

DXd was conjugated via stochastic maleimide conjugation to interchain cysteines of the antibodies. The antibodies were first reduced using 10× molar equivalents of 10 mM TCEP at 37° C. for 90 minutes with shaking. The reduced antibodies were then conjugated with 10× molar equivalents of 10 mM DXd at room temperature for 2 hours. Conjugated antibodies were evaluated for the drug to antibody ratio (DAR) by reducing 10 μg of the crude conjugate with DTT at 37° C. for 30 minutes. The final DAR for the conjugated antibodies was ˜8. The multimeric state of the conjugated antibodies was evaluated by size exclusion chromatography and confirmed to be monomeric. Conjugated antibodies were purified from unconjugated DXd by dialysis against PBS, pH 5.6 with a 10K molecular-weight cut-off (MWCO) membrane for 48 hours.

Next, we utilized a cell line-derived xenograft model of pancreatic cancer, where the human tumor cell line PANC-1 was implanted into Balb/c nude mice. The nude mouse is a naturally mutated mouse that lacks a thymus and hair (nude). The lack of a thymus in the nude mouse results in T-cell deficiency, thereby making the nude mouse immunodeficient and able to accept foreign tissue such as human tumor cells. Mice were injected subcutaneously with 5×10⁶ Panc-1 cells. The randomization started when the mean tumor size reached approximately 200 mm³. 60 mice were enrolled in the study. All animals were randomly allocated to 6 study groups, 10 mice in each group. Randomization was performed based on “Matched distribution” method. The experiment was performed as outlined in Table 5.

TABLE 5
In Vivo ADC Experimental Design

| | No. | | Dose | Dosing | Dosing | | Dosing |
|---|---|---|---|---|---|---|---|
| | of | | Level | Solution | Volume | | Frequency & |
| Group | mice | Treatment | (mg/kg) | (mg/ml) | (μL/g) | ROA* | Duration |
| 1 | 10 | Vehicle (PBS) | — | — | 10 | i.v. | QW × 2 |
| | | | | | | | weeks |
| 2 | 10 | Anti-K-Ras | 10 | 1 | 10 | i.v. | QW × 2 |
| | | antibody 1- | | | | | weeks |
| | | ADC | | | | | |
| 3 | 10 | Isotype | 10 | 1 | 10 | i.v. | QW × 2 |
| | | antibody-ADC | | | | | weeks |
| 4 | 10 | Anti-K-Ras | 10 | 1 | 10 | i.v. | QW × 2 |
| | | antibody 1 | | | | | weeks |
| 5 | 10 | Isotype | 10 | 1 | 10 | i.v. | QW × 2 |
| | | antibody | | | | | weeks |
| 6 | 10 | Gemcitabine | 50 | 5 | 10 | i.p. | BIW × 2 |
| | | | | | | | weeks |

*ROA = Route of Administration, i.v.—intravenous, i.p. = intraperitoneal

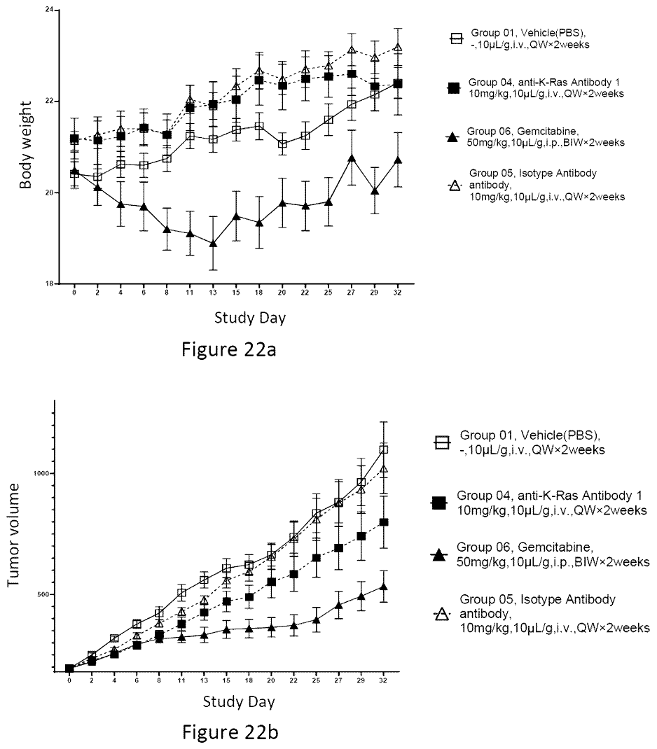

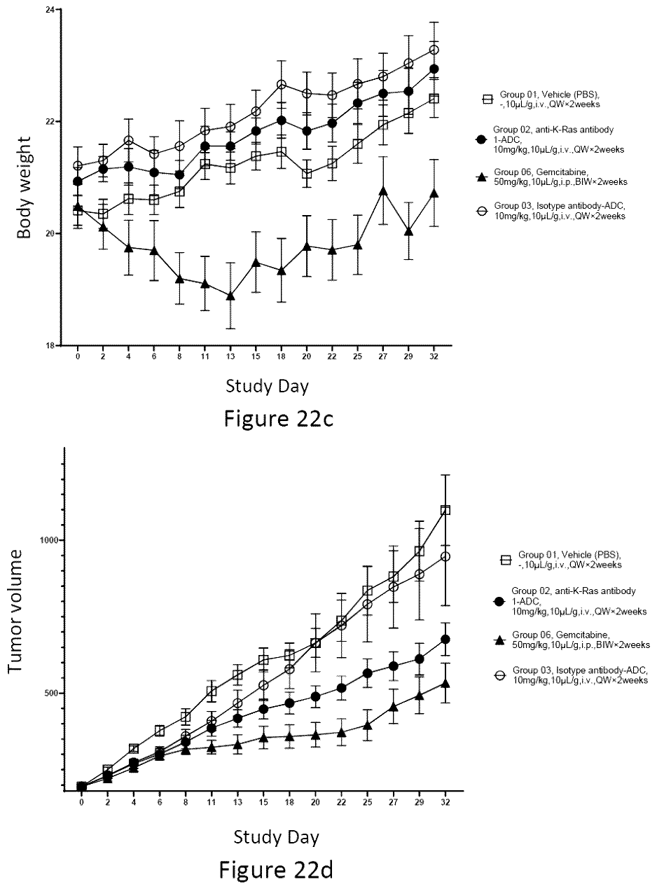

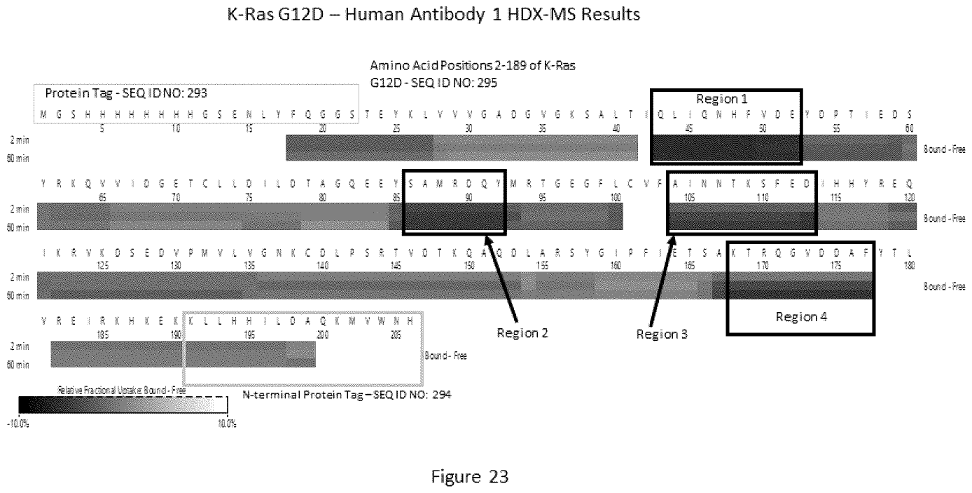

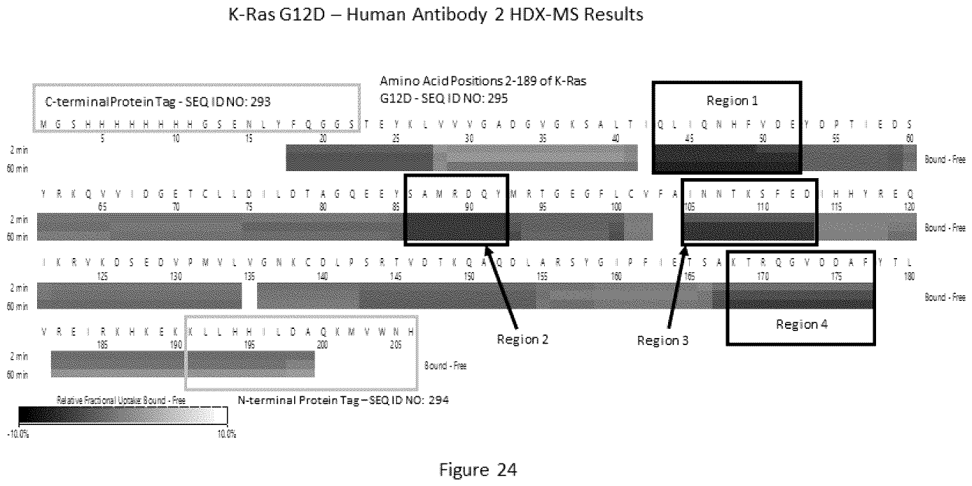

As can be seen in FIGS. 22a-22d, anti-Kras antibody 1 conjugated to DXd ADC slowed tumor growth over isotype-ADC and saline control treated mice (Group 1 vs 2 vs 3 vs 6)(FIGS. 22c-22d). Surprisingly, even mice treated with anti-K-Ras antibody 1 showed decreased tumor growth over isotype treated mice (FIGS. 22a-22b). While gemcitabine treatment slowed tumor growth over either ADC or antibody treatment mice treated with gemcitabine demonstrated distress based on weight loss while ADC or antibody treated mice gained weight at a rate similar to saline treated controls (FIGS. 22a-22d).

### Example 17. Using HDX-MS to Determine Antibody Sites Interacting with K-Ras

To determine the region(s) of K-Ras that interact with human anti-K-Ras Antibody 1 and human anti-K-Ras Antibody 2, and therefore are accessible on the cell surface, we utilized hydrogen-deuterium exchange mass spectrometry (HDX-MS). The experiments were carried out using the Trajan leap robotic platform, and Waters Cyclic IMS MS. Experiments were split into two phases: peptide mapping and HDX. Peptide mapping used only the free state of protein and identified peptides for HDX analysis, whereas HDX required both free and mAb-bound protein and compared the differences in deuterium uptake at each amino acid residue.

For the peptide mapping, the K-Ras G12D sample was diluted to 6 uM in 20 mM phosphate buffer, 150 mM NaCl, pH 7.4, (“H2O-based buffer”) and 6.5 uL of the protein solution was mixed with 43.5 uL of H₂O-based buffer. At the end of the reaction, 45 ul of the sample was mixed with the 45 μL of pre-dispensed 100 mM phosphate buffer, pH 2.4. 80 μL of the quenched sample was injected into the sample loop, and the quenched protein was digested using a Pepsin column (Affipro) for 210s at a flow rate of 0.200 mL/min before trapping and desalting, followed by separation through an analytical C18 column at 0.035 mL/min. The K-Ras G12D sample included isolated polypeptide. The polypeptide had an N-terminal protein tag (MGSHHHHHHHHGSENLYFQGGS—SEQ ID NO: 293) and a C-terminal protein tag (KLLHHILDAQKMVWNH—SEQ ID NO: 294) coupled to amino acid residues 2-189 of K-Ras G12D having SEQ ID NO: 295.

For HDX experiments, 6 uM of K-Ras G12D was analyzed in the free and monoclonal antibody (mAb)-bound states. In bound state measurements, 12 uM of intact each mAb was incubated with K-Ras G12D. The autosampler used the same volumes as in the peptide mapping experiments, but the H₂O buffer was swapped for the D20 buffer (20 mM phosphate buffer, 150 mM NaCl, pD 7.4). Three different labeling time points were used (2, 10, and 60 minutes) in both the free and mAb-bound states. All time points were collected in triplicate. The same flow rates were used for the liquid chromatography. PLGS was used to prepare a peptide library, and DynamX 3.0 was used for deuterium uptake analysis.

K-Ras G12D/antibody complexes were first prepared in 20 mM phosphate buffer, 150 nM NaCl, pH 7.4 for human anti-K-Ras antibody 1 and human anti-K-Ras antibody 2 before being exchanged into deuterium-containing buffer. K-Ras G12D/antibody complexes were exposed to deuterium for 2, 10, and 30 min before the deuterium exchange was quenched. Labeled K-Ras G12D/antibody complexes were then digested and analyzed by bottom-up mass spectrometry.

HDX-MS analysis of human anti-K-Ras antibody 1 and human anti-K-Ras antibody 2 showed notable decreases in deuterium incorporation in several portions of KRas G12D, shown in Table 6 for human anti-K-Ras antibody 1 and Table 7 for human anti-K-Ras antibody 2. Very similar regions were identified for potential interaction sites between either human anti-K-Ras antibody 1 and K-Ras or human anti-K-Ras antibody 2 and K-Ras. The decrease in deuterium uptake in these regions suggested that one or more of the regions was directly bound by the antibodies, and therefore are regions that are accessible when KRas is found at the cell surface.

TABLE 6
Regions of Deuterium Incorporation in K-Ras G12D in Complex with Human Anti-
K-Ras antibody 1.

| Region of | | Region of Deuterium | |
|---|---|---|---|
| Human | Relative Fractional | Incorporation of | |
| anti-K-Ras | Uptake: | KRAS G12D | |
| antibody 1 | Free-bound State | (SEQ ID NO: 295) | Notes |
| Region 1 | 6-7 | QLIQNHFVDE | Max difference |
| | | (SEQ ID NO: 296, residues | at 60 minutes |
| | | 22-31 of SEQ ID NO: 295) | |
| Region 2 | 5-6 | SAMRDQY | Max difference |
| | | (SEQ ID NO: 297, residues | at 60 minutes |
| | | 65-71 of SEQ ID NO: 295) | |
| Region 3 | 5-6 | AINNTKSFED | Max difference |
| | | (SEQ ID NO: 298, (residues | at 60 minutes |
| | | 83-92 of SEQ ID NO: 295) | |
| Region 4 | 5-6 | KTRQGVDDAF | Max difference |
| | | (SEQ ID NO: 299, residues | at 60 minutes |
| | | 157-166 of SEQ ID NO: 295) | |

TABLE 7
Regions of Deuterium Incorporation in K-Ras G12D in Complex with Human Anti-
K-Ras antibody 2.

| Region of | | | |
|---|---|---|---|
| Human | Relative Fractional | Region of Deuterium | |
| anti-K-Ras | Uptake: | Incorporation of | |
| antibody 2 | Free-bound State | KRAS G12D | Notes |
| Region 1 | 6-7 | QLIQNHFVDE | Max difference |
| | | (SEQ ID NO: 296, residues | at 60 minutes |
| | | 22-31 of SEQ ID NO: 295) | |
| Region 2 | 5-6 | SAMRDQY | Max difference |
| | | (SEQ ID NO: 297, residues | at 60 minutes |
| | | 65-71 of SEQ ID NO: 295) | |
| Region 3 | 5-6 | INNTKSFED | Max difference |
| | | (SEQ ID NO: 300, residues | at 60 minutes |
| | | 84-92 of SEQ ID NO: 295) | |
| Region 4 | 5-6 | KTRQGVDDAF | Max difference |
| | | SEQ ID NO: 299, (residues | at 60 minutes |
| | | 157-166 of SEQ ID NO: 295) | |

Regions identified by HDX-MS of K-Ras G12D interacting with either human anti-K-Ras antibody 1 or human anti-K-Ras antibody 2 include the sequences of QLIQNHFVDE, SAMRDQY, AlNNTKSFED, and KTRQGVDDAF.

To confirm the identity of regions of KRas G12D accessible on the cell surface, a similar experiment was completed using a K-Ras G12D/peptide R11.1.6 complex. As described in Example 1, R11.1.6 is a small cell-penetrating peptide that can bind to surface K-Ras. The crystal structure of KRas G12D (GppNHp) interacting with peptide R11.1.6 was downloaded from the PDB (5UFQ). The crystal structure was solved by Kauke et al. Sci Rep. 2017 Jul. 19; 7(1):5831. doi: 10.1038/s41598-017-05889-7 to identify high affinity, non-covalent inhibitors of K-Ras oncogenic mutants. Interacting residues between K-Ras G12D (chain A) and R11.1.6 (chain C) were determined using PyMol and the InterfaceResidues.py script (pymolwiki.org/index.php/InterfaceResidues). K-Ras G12D residues involved in interactions with R11.1.6 include: 3 GLU, 5 LYS, 6 LEU, 7 VAL, 36 ILE, 37 GLU, 38 ASP, 39 SER, 40 TYR, 41 ARG, 54 ASP, 56 LEU, 62 GLU, 63 GLU, 64 TYR, 66 ALA, 67 MET, 70 GLN, 71 TYR, 74 THR, and 75 GLY. This suggests that the regions of K-Ras G12D including residues 3-7, 36-41, 54-56, and 62-74 are accessible on the cell surface to interact with antibodies.

The foregoing description of specific embodiments of the present disclosure has been presented for purpose of illustration and description. The exemplary embodiments were chosen and described in order to best explain the principles of the invention and its practical application, to thereby enable others skilled in the art to best utilize the subject matter and various embodiments with modifications suited to the particular use contemplated. It should be understood that while some features may have only been described for a particular embodiment and not all other embodiments, that such features should not necessarily be limited to the particular embodiment in which they were described and may be applied to other embodiments as one of ordinary skill in the art may contemplate based on the present disclosure as a whole.

1.-47. (canceled)

48. A composition comprising:
a means for binding with specificity to a Surface K-Ras Antigen expressed on an external surface of a cancer cell; and
a therapeutic agent, wherein the means is linked to the therapeutic agent to form a therapeutic complex, wherein neither the composition nor the therapeutic complex comprises an intracellular delivery compound.

49. The composition of claim 48, wherein the Surface K-Ras Antigen comprises the amino acid sequence set forth in SEQ ID NOs: 1 or 2 with a mutation selected from the group consisting of G12A, G12D, G12C, G12V, G12R, G13D, Q61H, and Q61L.

50. The composition of claim 48, wherein the therapeutic agent is selected from the group consisting of a cytotoxic agent, a cytostatic agent, a toxin, or a radionuclide.

51. The composition of claim 48, wherein the means is linked to the therapeutic agent by a linker selected from the group consisting of a maleimidocaproyl linker, a peptide-based linker (including, but not limited to a valine-citrulline linker), a β-glucuronide linker, a SMCC linker, a disulfide linker, or an acid sensitive linker.

52. A method for treating, inhibiting or reducing proliferation of or killing a cancer cell in a subject comprising administering to the subject the composition of claim 48, wherein the subject has not been administered an intracellular delivery compound, or wherein the composition is not for administration or is not formulated for administration to the subject in conjunction with an intracellular delivery compound, and wherein the cancer cell expresses a Surface K-Ras Antigen on the external surface of the cancer cell.

53. The method of claim 52, wherein the cancer cells are selected from the group consisting of pancreatic cancer cells, lung cancer cells, cholangial cancer cells, ovarian cancer cells, endometrial cancer cells, or colorectal cancer cells.

54. A chimeric antigen receptor comprising an extracellular domain, a transmembrane domain, and an endodomain, wherein the extracellular domain comprises a means for binding with specificity to a Surface K-Ras Antigen expressed on an external surface of a cancer cell.

55. The chimeric antigen receptor of claim 54, wherein the Surface K-Ras Antigen comprises the amino acid sequence set forth in SEQ ID NOs: 1 or 2 with a mutation selected from the group consisting of G12A, G12D, G12C, G12V, G12R, G13D, Q61H, and Q61.

56. An immune cell expressing the chimeric antigen receptor of claim 54.

57. A method of treating a cancer in an individual comprising administering to the individual the immune cell of claim 56, wherein the cancer comprises cancer cells that express a Surface K-Ras Antigen on an external surface of the cancer cells.

58. A chimeric antigen receptor comprising an extracellular domain, a transmembrane domain, and an endodomain, wherein the extracellular domain comprises an antibody fragment, wherein the antibody fragment comprises a heavy chain variable domain comprising a heavy chain CDR set of the sequences of SEQ ID NO: 10, 11, and 12, and a light chain variable domain comprising a light chain CDR set of the sequences of SEQ ID NO: 14, 15, and 16.

59. An immune cell expressing the chimeric antigen receptor of claim 58.

60. A method of treating a cancer in an individual comprising administering to the individual the immune cell of claim 59, wherein the cancer comprises cancer cells that express a Surface K-Ras Antigen on an external surface of the cancer cells.

61. A bispecific binding complex comprising:
a first means for selectively binding to a Surface K-Ras Antigen expressed on an external surface of a cancer cell; and
a second means for selectively binding to an antigen expressed on a surface of an immune effector cell, wherein the first means is linked to the second means.

62. The bispecific binding complex of claim 61, wherein the Surface K-Ras Antigen comprises the amino acid sequence set forth in SEQ ID NOs: 1 or 2 with a mutation selected from the group consisting of G12A, G12D, G12C, G12V, G12R, G13D, Q61H, and Q61L.

63. The bispecific binding complex of claim 61, wherein the antigen expressed on a surface of an immune effector cell is CD3.

64. A method for treating, inhibiting or reducing proliferation of or killing a cancer cell in a subject comprising administering to the subject the bispecific antibody complex of claim 61, wherein the cancer cell expresses a Surface K-Ras Antigen on the external surface of the cancer cell.

65. The method of 64, wherein the cancer cells are selected from the group consisting of pancreatic cancer cells, lung cancer cells, cholangial cancer cells, ovarian cancer cells, endometrial cancer cells, or colorectal cancer cells.


SEQUENCE LISTING (300 sequences)

SEQ ID NO: 1 - protein, 188 aa, Homo sapiens [sequence omitted]

SEQ ID NO: 2 - protein, 189 aa, Homo sapiens [sequence omitted]

SEQ ID NO: 3 - protein, 9 aa, synthetic construct
RRRRRRRRR

SEQ ID NO: 4 - protein, 16 aa, synthetic construct
RQIKIWFQNRRMKWKK

SEQ ID NO: 5 - protein, 61 aa, synthetic construct
ATVKFTHQGEEKQVDISKIKWVIRWGQYIWFKYDEDGGAKGWGYVSEKDAPKELLQMLKKR

SEQ ID NO: 6 - protein, 61 aa, synthetic construct
ATVKFTHQGEEKQVDISKIKFVWRKGYVRIWGYDEDGGWGAGKYVSEKDAPKELLQMLKKR

SEQ ID NO: 7 - protein, 61 aa, synthetic construct
ATVKYTYRGEEKRVDISKIKWVNRWGQHLAFKYDKGGGAAGYGWVSEKDAPKELLQMLEKR

SEQ ID NO: 8 - protein, 61 aa, synthetic construct
ATVKFTYQGEEKQVDISKIMYVIRGGQRIAFGYDEGDGAWGDGIVSEKDAPKELLQMLEKQ

SEQ ID NO: 9 - protein, 446 aa, synthetic construct
EVQLVQSGGGVVQPGRSLRLSCAASGFTSRHPGMHWVRQAPGKGLEWVAVISHDGSKKYYADSVKGRFTISRDNSKNTLFVQLSSLRPEDTAVYYCATSLYSSMDLWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 10 - protein, 8 aa, synthetic construct
GFTSRHPG

SEQ ID NO: 11 - protein, 8 aa, synthetic construct
ISHDGSKK

SEQ ID NO: 12 - protein, 10 aa, synthetic construct
ATSLYSSMDL

SEQ ID NO: 13 - protein, 217 aa, synthetic construct
QSVVTQPPSVSAAPGQKVTISCSGSNSNIGKNYVSWFQQVPGTAPKLLIFEDNQRPSGIPDRFSASKSGTSASLAISGLQSEDEADYYCAAWDDKFGVHWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 14 - protein, 8 aa, synthetic construct
NSNIGKNY

SEQ ID NO: 15 - skipped

SEQ ID NO: 16 - protein, 12 aa, synthetic construct
AAWDDKFGVHWV

SEQ ID NO: 17 - protein, 455 aa, synthetic construct
EVQLLEPGGGVVQPGRSLRLSCTNSGFSFSGYAMHWVRQAPGKGLEWVAVISFDGSHKYYADSVKGRFTISRDNSKNTLYLHMNSLRAEDTAVYYCASGGNYYGSGTIVSHGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 18 - protein, 8 aa, synthetic construct
GFSFSGYA

SEQ ID NO: 19 - protein, 8 aa, synthetic construct
ISFDGSHK

SEQ ID NO: 20 - protein, 19 aa, synthetic construct
ASGGNYYGSGTIVSHGMDV

SEQ ID NO: 21 - protein, 217 aa, synthetic construct
QSVLTQPASVSGSPGQSITISCTGTSNDIGAYNYVSWYQQHPGKAPKLMIYDVNNRPSGVPDRFSGSKSGNMASLTISGLQAEDDADYYCSSYTSSSTLVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 22 - protein, 9 aa, synthetic construct
SNDIGAYNY

SEQ ID NO: 23 - skipped

SEQ ID NO: 24 - protein, 11 aa, synthetic construct
SSYTSSSTLVV

SEQ ID NO: 25 - protein, 18 aa, synthetic construct
GSTSGSGKPGSGEGSTKG

SEQ ID NO: 26 - protein, 446 aa, synthetic construct
EVQLVQSGGGVVQPGRSLRLSCAASGFTSRHPGMHWVRQAPGKGLEWVAVISHDGSKKYYADSVKGRFTISRDNSKNTLFVQLSSLRPEDTAVYYCATSLYSSMDLWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 27 - protein, 217 aa, synthetic construct; identical to SEQ ID NO: 13

SEQ ID NO: 28 - protein, 479 aa, synthetic construct
DIKLQQSGAELARPGASVKMSCKTSGYTFTRYTMIWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKGGGGSEPKSSDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 29 - protein, 449 aa, synthetic construct
QVTLRESGPALVKPTQTLTLTCTFSGFSLSTAGMSVGWIRQPPGKALEWLADIWWDDKKHYNPSLKDRLTISKDTSKNQVVLKVTNMDPADTATYYCARDMIFNFYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 30 - protein, 213 aa, synthetic construct
DIQMTQSPSTLSASVGDRVTITCSASSRVGYMHWYQQKPGKAPKLLIYDTSKLASGVPSRFSGSGSGTEFTLTISSLQPDDFATYYCFQGSGYPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - protein, 117 aa, synthetic construct
EVQLVQSGGGVVQPGRSLRLSCAASGFTSSNYGMHWVRQAPGKGLEWVAVISHDGSKKYYADSVKGRFTISRDNSKNTLFVQLSSLRPEDTAVYYCATSLYSSMDLWGQGTTVTVSS

SEQ ID NO: 32 - protein, 111 aa, synthetic construct
QSVVTQPPSVSAAPGQKVTISCSGSNSNIGKNYVSWFQQVPGTAPKLLIFEDNQRPSGIPDRFSASKSGTSASLAISGLQSEDEADYYCAAWDDSLNAHWVFGGGTKLTVL

SEQ ID NO: 33 - protein, 126 aa, synthetic construct
QVQLVESGGGVVQPGRSLRLSCTNSGFSFSGYAMHWVRQAPGKGLEWVAVISFDGSHKYYADSVKGRFTISRDNSKNTLYLHMNSLRAEDTAVYYCASGGNYYGSGTIVSHGLDFWGQGTTVTVSS

SEQ ID NO: 34 - protein, 107 aa, synthetic construct
DIVMTQSPSTLSASVGDRVTITCRASQSISSWLAWYQQKPGKAPKVLIYKASSLESGVPSRFSGSGSGTEFTLTISSLQPDDFAAYYCQHYNSYPYTFGQGTKLEIK

SEQ ID NO: 35 - protein, 126 aa, synthetic construct
QVQLVESGGGVVQPGRSLRLSCTNSGFSFSGYAMHWVRQAPGKGLEWVAVISFDGSHKYYADSVKGRFTISRDNSKNTLYLHMNSLRAEDTAVYYCASGGNYYGSGTIVSHGMDVWGQGTTVTVSS

SEQ ID NO: 36 - protein, 106 aa, synthetic construct
SSELTQDPAVSVALGQTVRITCQGDSLRSYYASWYQQKPGQAPVLVIYGKNNRPSGIPDRFSGSSSGNTASLTITGAQAEDEADYYCNSRDSSGNHRGGGTKLTVL

SEQ ID NO: 37 - protein, 122 aa, synthetic construct
QVQLVESGGGVVQPGRSLRLSCSNSGFSFSGYTMHWVRQAPGKGLEWVAVISFDGSHKYYADSVKGRFTISRDNSKNTLYLHMNSLRAEDTAVYYCAKKMHYGSGAYHFDLWGQGTLVTVSS

SEQ ID NO: 38 - protein, 107 aa, synthetic construct
DIVMTQSPSSLSASVGDRVTITCRASQSISNYLNWYQQKPGKAPKLLIYAASSLQSGVPSRFSGSGSGTDFTLTISSLQPEDFATYYCQQSYSSPFTFGPGTKVDIK

SEQ ID NO: 39 - protein, 126 aa, synthetic construct
EVQLLESGGGVVQPGRSLRLSCTNSGFSFSGYAMHWVRQAPGKGLEWVAVISFDGSHKYYADSVKGRFTISRDNSKNTLYLHMNSLTAEDTAVYYCASGGNYYGSGTIVSHGMDVWGQGTTVTVSS

SEQ ID NO: 40 - protein, 111 aa, synthetic construct
QSVLTQPASVSGSPGQSITISCTGTSSDVGGYNYVSWYQQHPGKAPKLMIYDVSNRPSGVSNRFSGSKSGNTASLTISGLQAEDEADYYCSSYTSSSTLVVFGGGTKLTVL

SEQ ID NO: 41 - protein, 126 aa, synthetic construct
EVQLLEPGGGVVQPGRSLRLSCTNSGFSFSGYAMHWVRQAPGKGLEWVAVISFDGSHKYYADSVKGRFTISRDNSKNTLYLHMNSLRAEDTAVYYCASGGNYYGSGTIVSHGMDVWGQGTTVTVSS

SEQ ID NO: 42 - protein, 110 aa, synthetic construct
QSALTQPRSVSGSPGQSVTISCTGTSSNVGGYNHVSWYQQHPGKAPKVIIYDVNKRPSGVSHRFSGSKSANTASLTISGLQAEDEADYYCSSYTTSSTYVFGTGTKLTVL

SEQ ID NO: 43 - protein, 126 aa, synthetic construct
QVQLVESAGGVVQPGRSLRLSCTNSGFSFSGYAMHWVRQAPGKGLEWVAVISFDGSHKYYADSVKGRFTISRDNSKNTLYLHMNSLTAEDTAVYYCASGGNYYGSGTIVSHGMDVWGQGTTVTVSS

SEQ ID NO: 44 - protein, 110 aa, synthetic construct
QSVLTQPASVSGSPGQSITISCTGTSSDVGGYKYVSWYQQYPGKAPKLMIYDVSKRPSGVSNRFSGSKSGNTASLTISGLQAEDEADYYCNSYTSSRIYVFGTGTKLTVL

SEQ ID NO: 45 - protein, 126 aa, synthetic construct; identical to SEQ ID NO: 41

SEQ ID NO: 46 - protein, 111 aa, synthetic construct
QSVLTQPASVSGSPGQSITISCTGTSNDIGAYNYVSWYQQHPGKAPKLMIYDVNNRPSGVPDRFSGSKSGNMASLTISGLQAEDDADYYCSSYTSSSTLVVFGGGTKLTVL

SEQ ID NO: 47 - protein, 126 aa, synthetic construct
QVQLVQSGGGVVQPGRSLRLSCTNSGFSFSGYAMHWVRQAPGKGLEWVAVISFDGSHKYYADSVKGRFTISRDNSKNTLYLHMNSVRAEDTAVYYCASGGNHYGSGTIVSHGMDVRGQGTTVTVSS

SEQ ID NO: 48 - protein, 110 aa, synthetic construct
QSVLTQPASVSGSPGQSITISCTGTSSDIGGSNWVSWYQQHPGKAPKLMIYDVSKRPSGVSNRFSGSKSGNTASLTISGLQAEDEADYYCSSYTSSSTYVFGTGTKLTVL

SEQ ID NO: 49 - protein, 119 aa, synthetic construct
QVQLVESGGGLVQPGGSLRLSCAASGFTFSSYWMSWVRQAPGKGLEWVGFIRSKAYGGTTEYAASVKGRFTISRDDSKSIAYLQMNSLKTEDTAVYYCTRDGGSYFDYWGQGTLVTVSS

SEQ ID NO: 50 - protein, 110 aa, synthetic construct
QAGLTQPASVSGSPGQSITISCTGTSSDVGGYNYVSWYQQHPGKAPKLMIYDVSKRPSGVSNRFSGSKSGNTASLTISGLQAEDEADYYCSSYTSSSTYVFGTGTKVTVL

SEQ ID NO: 51 - protein, 117 aa, synthetic construct
EVQLVQSGGGVVQPGRSLRLSCAASGFTSSLNGMHWVRQAPGKGLEWVAVISHDGSKKYYADSVKGRFTISRDNSKNTLFVQLSSLRPEDTAVYYCATSLYSSMDLWGQGTTVTVSS

SEQ ID NO: 52 - protein, 111 aa, synthetic construct
QSVVTQPPSVSAAPGQKVTISCSGSNSNIGKNYVSWFQQVPGTAPKLLIFEDNQRPSGIPDRFSASKSGTSASLAISGLQSEDEADYYCAAWDDYGSTHWVFGGGTKLTVL

SEQ ID NO: 53 - protein, 117 aa, synthetic construct
EVQLVQSGGGVVQPGRSLRLSCAASGFTSRQYGMHWVRQAPGKGLEWVAVISHDGSKKYYADSVKGRFTISRDNSKNTLFVQLSSLRPEDTAVYYCATSLYSSMDLWGQGTTVTVSS

SEQ ID NO: 54 - protein, 111 aa, synthetic construct
QSVVTQPPSVSAAPGQKVTISCSGSNSNIGKNYVSWFQQVPGTAPKLLIFEDNQRPSGIPDRFSASKSGTSASLAISGLQSEDEADYYCAAWDDGRKVHWVFGGGTKLTVL

SEQ ID NO: 55 - protein, 117 aa, synthetic construct
EVQLVQSGGGVVQPGRSLRLSCAASGFTSSTYGMHWVRQAPGKGLEWVAVISHDGSKKYYADSVKGRFTISRDNSKNTLFVQLSSLRPEDTAVYYCATSLYSSMDLWGQGTTVTVSS

SEQ ID NO: 56 - protein, 111 aa, synthetic construct
QSVVTQPPSVSAAPGQKVTISCSGSNSNIGKNYVSWFQQVPGTAPKLLIFEDNQRPSGIPDRFSASKSGTSASLAISGLQSEDEADYYCAAWDDSRTTHWVFGGGTKLTVL

SEQ ID NO: 57 - protein, 117 aa, synthetic construct
EVQLVQSGGGVVQPGRSLRLSCAASGFTSRPYGMHWVRQAPGKGLEWVAVISHDGSKKYYADSVKGRFTISRDNSKNTLFVQLSSLRPEDTAVYYCATSLYSSMDLWGQGTTVTVSS

SEQ ID NO: 58 - protein, 111 aa, synthetic construct
QSVVTQPPSVSAAPGQKVTISCSGSNSNIGKNYVSWFQQVPGTAPKLLIFEDNQRPSGIPDRFSASKSGTSASLAISGLQSEDEADYYCAAWDDFHSEHWVFGGGTKLTVL

SEQ ID NO: 59 - protein, 117 aa, synthetic construct
EVQLVQSGGGVVQPGRSLRLSCAASGFTSATYGMHWVRQAPGKGLEWVAVISHDGSKKYYADSVKGRFTISRDNSKNTLFVQLSSLRPEDTAVYYCATSLYSSMDLWGQGTTVTVSS

SEQ ID NO: 60 - protein, 111 aa, synthetic construct
QSVVTQPPSVSAAPGQKVTISCSGSNSNIGKNYVSWFQQVPGTAPKLLIFEDNQRPSGIPDRFSASKSGTSASLAISGLQSEDEADYYCAAWDDSSDTHWVFGGGTKLTVL

SEQ ID NO: 61 - protein, 117 aa, synthetic construct
EVQLVQSGGGVVQPGRSLRLSCAASGFTSSFHGMHWVRQAPGKGLEWVAVISHDGSKKYYADSVKGRFTISRDNSKNTLFVQLSSLRPEDTAVYYCATSLYSSMDLWGQGTTVTVSS

SEQ ID NO: 62 - protein, 111 aa, synthetic construct
QSVVTQPPSVSAAPGQKVTISCSGSNSNIGKNYVSWFQQVPGTAPKLLIFEDNQRPSGIPDRFSASKSGTSASLAISGLQSEDEADYYCAAWDDSGDVHWVFGGGTKLTVL

SEQ ID NO: 63 - protein, 117 aa, synthetic construct
EVQLVQSGGGVVQPGRSLRLSCAASGFTSRHPGMHWVRQAPGKGLEWVAVISHDGSKKYYADSVKGRFTISRDNSKNTLFVQLSSLRPEDTAVYYCATSLYSSMDLWGQGTTVTVSS

SEQ ID NO: 64 - protein, 111 aa, synthetic construct
QSVVTQPPSVSAAPGQKVTISCSGSNSNIGKNYVSWFQQVPGTAPKLLIFEDNQRPSGIPDRFSASKSGTSASLAISGLQSEDEADYYCAAWDDYRGPHWVFGGGTKLTVL

SEQ ID NO: 65 - protein, 117 aa, synthetic construct; identical to SEQ ID NO: 63

SEQ ID NO: 66 - protein, 111 aa, synthetic construct; identical to SEQ ID NO: 64

SEQ ID NO: 67 - protein, 117 aa, synthetic construct
EVQLVQSGGGVVQPGRSLRLSCAASGFTSRAPGMHWVRQAPGKGLEWVAVISHDGSKKYYADSVKGRFTISRDNSKNTLFVQLSSLRPEDTAVYYCATSLYSSMDLWGQGTTVTVSS

SEQ ID NO: 68 - protein, 111 aa, synthetic construct
QSVVTQPPSVSAAPGQKVTISCSGSNSNIGKNYVSWFQQVPGTAPKLLIFEDNQRPSGIPDRFSASKSGTSASLAISGLQSEDEADYYCAAWDDHNGEHWVFGGGTKLTVL

SEQ ID NO: 69 - protein, 117 aa, synthetic construct; identical to SEQ ID NO: 63

SEQ ID NO: 70 - protein, 111 aa, synthetic construct
QSVVTQPPSVSAAPGQKVTISCSGSNSNIGKNYVSWFQQVPGTAPKLLIFEDNQRPSGIPDRFSASKSGTSASLAISGLQSEDEADYYCAAWDDKFGVHWVFGGGTKLTVL

SEQ ID NO: 71 - protein, 117 aa, synthetic construct
EVQLVQSGGGVVQPGRSLRLSCAASGFTSRKYGMHWVRQAPGKGLEWVAVISHDGSKKYYADSVKGRFTISRDNSKNTLFVQLSSLRPEDTAVYYCATSLYSSMDLWGQGTTVTVSS

SEQ ID NO: 72 - protein, 111 aa, synthetic construct
QSVVTQPPSVSAAPGQKVTISCSGSNSNIGKNYVSWFQQVPGTAPKLLIFEDNQRPSGIPDRFSASKSGTSASLAISGLQSEDEADYYCAAWDDSPTIHWVFGGGTKLTVL

SEQ ID NO: 73 - protein, 117 aa, synthetic construct
EVQLVQSGGGVVQPGRSLRLSCAASGFTSHNKGMHWVRQAPGKGLEWVAVISHDGSKKYYADSVKGRFTISRDNSKNTLFVQLSSLRPEDTAVYYCATSLYSSMDLWGQGTTVTVSS

SEQ ID NO: 74 - protein, 111 aa, synthetic construct
QSVVTQPPSVSAAPGQKVTISCSGSNFNIGKNYVSWFQQVPGTAPKLLIFEDNQRPSGIPDRFSASKSGTSASLAISGLQSEDEADYYCAAWDDFAGVHWVFGGGTKLTVL

SEQ ID NO: 75 - protein, 117 aa, synthetic construct
EVQLVQSGGGVVQPGRSLRLSCAASGFTSYKYGMHWVRQAPGKGLEWVAVISHDGSKKYYADSVKGRFTISRDNSKNTLFVQLSSLRPEDTAVYYCASSLYSSMDLWGQGTTVTVSS

SEQ ID NO: 76 - protein, 111 aa, synthetic construct
QSVVTQPPSVSAAPGQKVTISCSGSNSNIGKNYVSWFQQVPGTAPKLLIFEDNQRPSGIPDRFSASKSGTSASLAISGLQSEDEADYYCAAWDDSFSEHWVFGGGTKLTVL

SEQ ID NO: 77 - protein, 117 aa, synthetic construct; identical to SEQ ID NO: 67

SEQ ID NO: 78 - protein, 111 aa, synthetic construct
QSVVTQPPSVSAAPGQKVTISCSGSNSNIGKNYVSWFQQVPGTAPKLLIFEDNQRPSGIPDRFSASKSGTSASLAISGLQSEDEADYYCAAWDDQNGVHWVFGGGTKLTVL

SEQ ID NO: 79 - protein, 117 aa, synthetic construct
EVQLVQSGGGVVQPGRSLRLSCAASGFTSRSPGMHWVRQAPGKGLEWVAVISHDGSKKYYADSVKGRFTISRDNSKNTLFVQLSSLRPEDTAVYYCATSLYSSMDLWGQGTTVTVSS

SEQ ID NO: 80 - protein, 111 aa, synthetic construct
QSVVTQPPSVSAAPGQKVTISCSGSNSNIGKNYVSWFQQVPGTAPKLLIFEDNQRPSGIPDRFSASKSGTSASLAISGLQSEDEADYYCAAWDDLRGVHWVFGGGTKLTVL

SEQ ID NO: 81 - protein, 117 aa, synthetic construct
EVQLVQSGGGVVQPGRSLRLSCAASGFTSRHHGMHWVRQAPGKGLEWVAVISHDGSKKYYADSVKGRFTISRDNSKNTLFVQLSSLRPEDTAVYYCATSLYSSMDLWGQGTTVTVSS

SEQ ID NO: 82 - protein, 111 aa, synthetic construct
QSVVTQPPSVSAAPGQKVTISCSGSNSNIGKNYVSWFQQVPGTAPKLLIFEDNQRPSGIPDRFSASKSGTSASLAISGLQSEDEADYYCAAWDDRHGTHWVFGGGTKLTVL

SEQ ID NO: 83 - protein, 117 aa, synthetic construct
EVQLVQSGGGVVQPGRSLRLSCAASGFTSRNPGMHWVRQAPGKGLEWVAVISHDGSKKYYADSVKGRFTISRDNSKNTLFVQLSSLRPEDTAVYYCATSLYSSMDLWGQGTTVTVSS

SEQ ID NO: 84 - protein, 111 aa, synthetic construct
QSVVTQPPSVSAAPGQKVTISCSGSNSNIGKNYVSWFQQVPGTAPKLLIFEDNQRPSGIPDRFSASKSGTSASLAISGLQSEDEADYYCAAWDDSTGVHWVFGGGTKLTVL

SEQ ID NO: 85 - protein, 117 aa, synthetic construct
EVQLVQSGGGVVQPGRSLRLSCAASGFTSRTYGMHWVRQAPGKGLEWVAVISHDGSKKYYADSVKGRFTISRDNSKNTLFVQLSSLRPEDTAVYYCATSLYSSMDLWGQGTTVTVSS

SEQ ID NO: 86 - protein, 111 aa, synthetic construct
QSVVTQPPSVSAAPGQKVTISCSGSNSNIGKNYVSWFQQVPGTAPKLLIFEDNQRPSGIPDRFSASKSGTSASLAISGLQSEDEADYYCAAWDDLPTDHWVFGGGTKLTVL

SEQ ID NO: 87 - protein, 117 aa, synthetic construct; identical to SEQ ID NO: 53

SEQ ID NO: 88 - protein, 111 aa, synthetic construct
QSVVTQPPSVSAAPGQKVTISCSGSNSNIGKNYVSWFQQVPGTAPKLLIFEDNQRPSGIPDRFSASKSGTSASLAISGLQSEDEADYYCAAWDDYSGVHWVFGGGTKLTVL

SEQ ID NO: 89 - protein, 117 aa, synthetic construct
EVQLVQSGGGVVQPGRSLRLSCAASGFTSSPYGMHWVRQAPGKGLEWVAVISHDGSKKYYADSVKGRFTISRDNSKNTLFVQLSSLRPEDTAVYYCATSLYSSMDLWGQGTTVTVSS

SEQ ID NO: 90 - protein, 111 aa, synthetic construct
QSVVTQPPSVSAAPGQKVTISCSGSNSNIGKNYVSWFQQVPGTAPKLLIFEDNQRPSGIPDRFSASKSGTSASLAISGLQSEDEADYYCAAWDDNPRDHWVFGGGTKLTVL

SEQ ID NO: 91 - protein, 117 aa, synthetic construct
EVQLVQSGGGVVQPGRSLRLSCAASGFTSSKPGMHWVRQAPGKGLEWVAVISHDGSKKYYADSVKGRFTISRDNSKNTLFVQLSSLRPEDTAVYYCATSLYSSMDLWGQGTTVTVSS

SEQ ID NO: 92 - protein, 111 aa, synthetic construct; identical to SEQ ID NO: 80

SEQ ID NO: 93 - protein, 117 aa, synthetic construct
EVQLVQSGGGVVQPGRSLRLSCAASGFTSKKYGMHWVRQAPGKGLEWVAVISHDGSKKYYADSVKGRFTISRDNSKNTLFVQLSSLRPEDTAVYYCATSLYSSMDLWGQGTTVTVSS

SEQ ID NO: 94 - protein, 111 aa, synthetic construct
QSVVTQPPSVSAAPGQKVTISCSGSNSNIGKNYVSWFQQVPGTAPKLLIFEDNQRPSGIPDRFSASKSGTSASLAISGLQSEDEADYYCAAWDDLGGDHWVFGGGTKLTVL

SEQ ID NO: 95 - protein, 117 aa, synthetic construct
EVQLVQSGGGVVQPGRSLRLSCAASGFTSTLPGMHWVRQAPGKGLEWVAVISHDGSKKYYADSVKGRFTISRDNSKNTLFVQLSSLRPEDTAVYYCATSLYSSMDLWGQGTTVTVSS

SEQ ID NO: 96 - protein, 111 aa, synthetic construct
QSVVTQPPSVSAAPGQKVTISCSGSNSNIGKNYVSWFQQVPGTAPKLLIFEDNQRPSGIPDRFSASKSGTSASLAISGLQSEDEADYYCAAWDDSAGVHWVFGGGTKLTVL

SEQ ID NO: 97 - protein, 117 aa, synthetic construct
EVQLVQSGGGVVQPGRSLRLSCAASGFTSKMPGMHWVRQAPGKGLEWVAVISHDGSKKYYADSVKGRFTISRDNSKNTLFVQLSSLRPEDTAVYYCATSLYSSMDLWGQGTTVTVSS

SEQ ID NO: 98 - protein, 111 aa, synthetic construct
QSVVTQPPSVSAAPGQKVTISCSGSNSNIGKNYVSWFQQVPGTAPKLLIFEDNQRPSGIPDRFSASKSGTSASLAISGLQSEDEADYYCAAWDDEHGVHWVFGGGTKLTVL

SEQ ID NO: 99 - protein, 117 aa, synthetic construct
EVQLVQSGGGVVQPGRSLRLSCAASGFTSSNYGMHWVRQAPGKGLEWVAVISHDGSKKYYADSVKGRFTISRDNSKNTLFVQLSSLRPEDTAVYYCAASLYSSMDLWGQGTTVTVSS

SEQ ID NO: 100 - protein, 111 aa, synthetic construct
QSVVTQPPSVSAAPGQKVTISCSGSNSNIGKNYVSWFQQVPGTAPKLLIFEDNQRPSGIPDRFSASKSGTSASLAISGLQSEDEADYYCAAWDDSLSAHWVFGGGTKLTVL

SEQ ID NO: 101 - protein, 117 aa, synthetic construct
EVQLVQSGGGVVQPGRSLRLSCAASGFTSSNYGMHWVRQAPGKGLEWVAVISHDGSKKYYADSVKGRFTISRDNSKNTLFVQLSSLRPEDTAVYYCATSMVSSMDLWGQGTTVTVSS

SEQ ID NO: 102 - protein, 111 aa, synthetic construct
QSVVTQPPSVSAAPGQKVTISCSGSNSNIGKNYVSWFQQVPGTAPKLLIFEDNQRPSGIPDRFSASKSGTSASLAISGLQSEDEADYYCAAWDDSLSTHWVFGGGTKLTVL

SEQ ID NO: 103 - protein, 117 aa, synthetic construct
EVQLVQSGGGVVQPGRSLRLSCAASGFTSSNYGMHWVRQAPGKGLEWVAVISHDGSKKYYADSVKGRFTISRDNSKNTLFVQLSSLRPEDTAVYYCATSMRSSMDLWGQGTTVTVSS

SEQ ID NO: 104 - protein, 111 aa, synthetic construct; identical to SEQ ID NO: 100

SEQ ID NO: 105 - protein, 117 aa, synthetic construct; identical to SEQ ID NO: 31

SEQ ID NO: 106 - protein, 111 aa, synthetic construct; identical to SEQ ID NO: 100

SEQ ID NO: 107 - protein, 119 aa, synthetic construct
EVQLVESGGGLVQPGGSLRLSCAASGFTFSSYSMNWVRQAPGKGLEWVSYISSSSSTIYYADSVKGRFTISRDNAKNSLYLQMNSLRAEDTAVYYCARGFYVRNWFDPWGQGTLVTVSS

SEQ ID NO: 108 - protein, 107 aa, synthetic construct
AIQMTQSPSSLSASVGDRVTITCRASQGIRNDLGWYQQKPGKAPKLLIYAASSLQSGVPSRFSGSGSGTDFTLTISSLQPEDFATYYCLQDHDYPLTFGQGTKVEIK

SEQ ID NO: 109 - protein, 124 aa, synthetic construct
EVQLVESGGGLVQPGGSLRLSCAASGFTFSSYAMSWVRQAPGKGLEWVSAISSSGSSTYYADSVKGRFTISRDNSKNTLYLQMNSLRAEDTAVYYCARDQGGYGYPGESWFDYWGQGTLVTVSS

SEQ ID NO: 110 - protein, 107 aa, synthetic construct
DIQMTQSPSSLSASVGDRVTITCRASQGISSYLAWYQQKPGKAPKLLIYAASSLQSGVPSRFSGSGSGTDFTLTISSLQPEDFATYYCQQYYSYPFTFGQGTKVEIK

SEQ ID NO: 111 - protein, 117 aa, synthetic construct
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARAFYSYMDVWGQGTLVTVSS

SEQ ID NO: 112 - protein, 107 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSPPWTFGQGTKVEIK

SEQ ID NO: 113 - protein, 122 aa, synthetic construct
EVQLQESGPGLVKPPGTLSLTCAVSGGSISSSNWWSWVRQPPGKGLEWIGEIYHSGSTNYNPSLKSRVTISVDKSKNQFSLKLSSVTAADTAVYYCARERTILTGYYGFDYWGQGTLVTVSS

SEQ ID NO: 114 - protein, 112 aa, synthetic construct
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPLTFGQGTKVEIK

SEQ ID NO: 115 - protein, 124 aa, synthetic construct
EVQLVQSGAEVKKPGSSVKVSCKASGGTFSSYAISWVRQAPGQGLEWMGGIIPIFGTANYAQKFQGRVTITADESTSTAYMELSSLRSEDTAVYYCARYYDFWSGYPGGLFDVWGQGTLVTVSS

SEQ ID NO: 116 - protein, 109 aa, synthetic construct
SVLTQPPSVSAAPGQKVTISCSGSSSNIGNNYVSWYQQLPGTAPKLLIYDNNKRPSGIPDRFSGSKSGTSATLGITGLQTGDEADYYCGTWDSSLTGYVFGGGTKLTVL

SEQ ID NO: 117 - protein, 124 aa, synthetic construct; identical to SEQ ID NO: 115

SEQ ID NO: 118 - protein, 109 aa, synthetic construct
SVLTQPPSVSAAPGQKVTISCSGSSSNIGNNYVSWYQQLPGTAPKLLIYDNNKRPSGIPDRFSGSKSGTSATLGITGLQTGDEADYYCGTWDSSLTGWVFGGGTKLTVL

SEQ ID NO: 119 - protein, 120 aa, synthetic construct
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARTNNYGYRYFDYWGQGTLVTVSS

SEQ ID NO: 120 - protein, 105 aa, synthetic construct
LTQDPAVSVALGQTVRITCQGDSLRSYYASWYQQKPGQAPVLVIYGKNNRPSGIPDRFSGSSSGNTASLTITGAQAEDEADYYCNSRDSSGNHWVFGGGTKLTVL

SEQ ID NO: 121 - protein, 120 aa, synthetic construct
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARATSSGYYYFDYWGQGTLVTVSS

SEQ ID NO: 122 - protein, 107 aa, synthetic construct
ELTQDPAVSVALGQTVRITCQGDSLRSYYASWYQQKPGQAPVLVIYGKNNRPSGIPDRFSGSSSGNTASLTITGAQAEDEADYYCNSRDSTDNHLWVFGGGTKLTVL

SEQ ID NO: 123 - protein, 122 aa, synthetic construct
EVQLQESGPGLVKPPGTLSLTCAVSGGSISSSNWWSWVRQPPGKGLEWIGEIYHSGSTNYNPSLKSRVTISVDKSKNQFSLKLSSVTAADTAVYYCARGSSSWYDLGPFDYWGQGTLVTVSS

SEQ ID NO: 124 - protein, 109 aa, synthetic construct
SVLTQPPSASGTPGQRVTISCSGSSSNIGSNYVYWYQQLPGTAPKLLIYRNNQRPSGVPDRFSGSKSGTSASLAISGLRSEDEADYYCAAWDERLSGWVFGGGTKLTVL

SEQ ID NO: 125 - protein, 123 aa, synthetic construct
EVQLVESGGGLVQPGGSLRLSCAASGFTFSSYSMNWVRQAPGKGLEWVSYISSSSSTIYYADSVKGRFTISRDNAKNSLYLQMNSLRAEDTAVYYCARGKGIVGWGFFGMDVWGQGTLVTVSS

SEQ ID NO: 126 - protein, 109 aa, synthetic construct
SVLTQPPSVSAAPGQKVTISCSGSSSNIGNNYVSWYQQLPGTAPKLLIYDNNKRPSGIPDRFSGSKSGTSATLGITGLQTGDEADYYCGTWDNSLSVWVFGGGTKLTVL

SEQ ID NO: 127 - protein, 118 aa, synthetic construct
EVQLVESGGGLVQPGGSLRLSCAASGFTFSSYSMNWVRQAPGKGLEWVSYISSSSSTIYYADSVKGRFTISRDNAKNSLYLQMNSLRAEDTAVYYCARSFGPYAFDVWGQGTLVTVSS

SEQ ID NO: 128 - protein, 108 aa, synthetic construct
VLTQPPSASGTPGQRVTISCSGSSSNIGSNYVYWYQQLPGTAPKLLIYRNNQRPSGVPDRFSGSKSGTSASLAISGLRSEDEADYYCAAWDDSLSGWVFGGGTKLTVL

SEQ ID NO: 129 - protein, 122 aa, synthetic construct
EVQLQESGPGLVKPPGTLSLTCAVSGSSIWSSNWWSWVRQPPGKGLEWIGEIYHSGSTNYNPSLKSRVTISVDKSKNQFSLKLSSVTAADTAVYYCARGSSSWYDLGPFDYWGQGTLVTVSS

SEQ ID NO: 130 - protein, 109 aa, synthetic construct; identical to SEQ ID NO: 124

SEQ ID NO: 131 - protein, 122 aa, synthetic construct
EVQLQESGPGLVKPPGTLSLTCAVSGSNISSSNWWSWVRQPPGKGLEWIGEIYHSGSTNYNPSLKSRVTISVDKSKNQFSLKLSSVTAADTAVYYCARGSSSWYDLGPFDYWGQGTLVTVSS

SEQ ID NO: 132 - protein, 109 aa, synthetic construct; identical to SEQ ID NO: 124

SEQ ID NO: 133 - protein, 122 aa, synthetic construct
EVQLQESGPGLVKPPGTLSLTCAVSGSSIFSSNWWSWVRQPPGKGLEWIGEIYHSGSTNYNPSLKSRVTISVDKSKNQFSLKLSSVTAADTAVYYCARGSSSWYDLGPFDYWGQGTLVTVSS

SEQ ID NO: 134 - protein, 109 aa, synthetic construct; identical to SEQ ID NO: 124

SEQ ID NO: 135 - protein, 122 aa, synthetic construct
EVQLQESGPGLVKPPGTLSLTCAVSGSSIMSSNWWSWVRQPPGKGLEWIGEIYHSGSTNYNPSLKSRVTISVDKSKNQFSLKLSSVTAADTAVYYCARGSSSWYDLGPFDYWGQGTLVTVSS

SEQ ID NO: 136 - protein, 109 aa, synthetic construct; identical to SEQ ID NO: 124

SEQ ID NO: 137 - protein, 122 aa, synthetic construct
EVQLQESGPGLVKPPGTLSLTCAVSGSSIYSSNWWSWVRQPPGKGLEWIGEIYHSGSTNYNPSLKSRVTISVDKSKNQFSLKLSSVTAADTAVYYCARGSSSWYDLGPFDYWGQGTLVTVSS

SEQ ID NO: 138 - protein, 109 aa, synthetic construct; identical to SEQ ID NO: 124

SEQ ID NO: 139 - protein, 122 aa, synthetic construct
EVQLQESGPGLVKPPGTLSLTCAVSGGNIWSSNWWSWVRQPPGKGLEWIGEIYHSGSTNYNPSLKSRVTISVDKSKNQFSLKLSSVTAADTAVYYCARGSSSWYDLGPFDYWGQGTLVTVSS

SEQ ID NO: 140 - protein, 109 aa, synthetic construct; identical to SEQ ID NO: 124

SEQ ID NO: 141 - protein, 122 aa, synthetic construct
EVQLQESGPGLVKPPGTLSLTCAVSKGSIWASHWWSWVRQPPGKGLEWIGEIYHSGSTNYNPSLKSRVTISVDKSKNQFSLKLSSVTAADTAVYYCARGSSSWYDLGPFDYWGQGTLVTVSS

SEQ ID NO: 142 - protein, 109 aa, synthetic construct; identical to SEQ ID NO: 124

SEQ ID NO: 143 - protein, 122 aa, synthetic construct
EVQLQESGPGLVKPPGTLSLTCAVSKGSIWSSNWWSWVRQPPGKGLEWIGEIYHSGSTNYNPSLKSRVTISVDKSKNQFSLKLSSVTAADTAVYYCARGSSSWYDLGPFDYWGQGTLVTVSS

SEQ ID NO: 144 - protein, 109 aa, synthetic construct; identical to SEQ ID NO: 124

SEQ ID NO: 145 - protein, 115 aa, synthetic construct
EVQLLESGGGLVQPGGSLRLSCAASGFTFSTFSMNWVRQAPGKGLEWVSYISRTSKTIYYADSVKGRFTISRDNSKNTLYLQMNSLRAEDTAVYYCARGRFFDYWGQGTLVTVSS

SEQ ID NO: 146 - protein, 104 aa, synthetic construct [sequence omitted]

SEQ ID NO: 147 - protein, 118 aa, synthetic construct
QIQLQESGPGLVKPSQSLSLTCTVTGYSITSDYAWNWIRQFPGNKLEWMGYINYSGSTSYNPSLKSRISITRDTSKNQFFLQLISVTSEDTATYYCAGSGYVRGFAYWGQGTLVTVSA

SEQ ID NO: 148 - protein, 107 aa, synthetic construct
DICKTQSHKFMSTSVGDRVSITCKASQDVGTAVAWYQQKPGQSPKLLIYWASTRHTGVPDRFTGSESGTDFTLTISNVQSEDLADYFCQQYSSYPYTFGGGTKLEIK

SEQ ID NO: 149 - protein, 117 aa, synthetic construct
EVQLQQSGPELVKPGASVKMSCKTSGYTFTDYTMHWVKQSHGKSLEWIGYINPNNGGTYYNQRFKGKATLTVSRSSSTAYIELRSLTSDDSAVYYCARGDYYGGGYWGQGTTLRVSS

SEQ ID NO: 150 - protein, 106 aa, synthetic construct
ENVLTQSPAIMSASPGEKVTMTCSAGSSVSYMHWYQQKSSTSPKLWIYDTSKLASGVPGRFSGSGSGNSYSLTISSMEAEDVATYYCFQGSGYPLTFGDGTKLELK

SEQ ID NO: 151 - protein, 117 aa, synthetic construct
EVQLQQSGPERVKPGASVKMSCKASGYTFTDYSMHWVKQSHGKSLEWIGYINPNNGGASYNQKFKGKATLTVNKSSSTAYMELRSLTSEDSAVYYCARGDYYGGGYWGQGTTLTVSS

SEQ ID NO: 152 - protein, 106 aa, synthetic construct
ENVLTQSPAIMSASPGEKVTMTCSASSSVSYMHWYQQKSSTSPKLWIYDTSKVASGVPGRFSGSGSGNSYSLTINSMEAEDVATYYCFQGSGYPLTFGAGTKLELK

SEQ ID NO: 153 - protein, 114 aa, synthetic construct
QVQLQQPGAEFVKPGASVKLSCKASGYTFTRYWMHWVKQRPGRGLEWIGRIDPNSGGTTFNEKFKSKATLTVDKPSSTAYMQLSSLTSEDSAVYYCAREVGGYWGQGTTLTVSS

SEQ ID NO: 154 - protein, 106 aa, synthetic construct
ENVLTQSPAIMSASPGEKVTMTCSAGSSVDYMHWYQQKSSTSPKLWIYDTSKLASGVPGRFSGSGSGNSYSLTISSMEAEDVATYYCFQGSGYPLTFGAGTKLELR

SEQ ID NO: 155 - protein, 119 aa, synthetic construct
QVTLKESGPGILQPSQTLSLTCSFSGFSLSTFGMGVGWIRQPSGKGLEWLAHIWWDDDKNYNPALKSRLTISKDTSKNQVFFKIANVDTADTATYYCVQLSTMGDGGYWGQGTLVTVSA

SEQ ID NO: 156 - protein, 107 aa, synthetic construct
NIVMTQSPKSMSMSVGERVTLSCKASENVGTYVSWYQQKPGQSPKLLIYGASNRYTGVPDRFTGSGSGTDFILTISSVQAEDLADYHCGQSYNYPFTFGSGTKLEIK

SEQ ID NO: 157 - protein, 122 aa, synthetic construct
QVTLKESGPGILQPSQTLSLTCSFSGFSLSTFGVGVAWIRQPSGKGLEWLAHIWWDDDKNYDPALKSRLTISKDTSKNQVFLKIANVDTTDSATYYCARIGSTLITPGDASWGQGTLVTVSA

SEQ ID NO: 158 - protein, 107 aa, synthetic construct
NIVMTQSPKSMSMSVGERVTLSCKASENVGTFVSWYQQKPDQSPKLLIYGASNRYTGVPDRFTGSGSATDFTLTISSVQAEDLADYHCGQSYSYPYTFGGGTKLEIK

SEQ ID NO: 159 - protein, 122 aa, synthetic construct; identical to SEQ ID NO: 157

SEQ ID NO: 160 - protein, 109 aa, synthetic construct
DIVLTQSPATLSVTPGDRVSLSCRASQSISDYLHWYQQKSHESPRLLIKFASQSISGIPSRFSGSGSGTDFTLSINSVETEDFGMYFCQQSNNWPHMYTFGGGTKLEIK

SEQ ID NO: 161 - protein, 113 aa, synthetic construct
DIQLQESGPGLVKPSQSLSLTCSVTGYSITSDYYWNWIRQFPGNKLEWMGYIGYDGTNNYNPSLKNRISITRDTSKNQFFLKLNSVTAEDTATYYCARLWDYWGQGTTLTVSS

SEQ ID NO: 162 - protein, 112 aa, synthetic construct
DVLMTQTPLSLPVSLGDQASISCRSSQTIVHGNGNTYLEWYLQKPGQSPKLLIYTVSNRFSGVPDRFSGSGSGTDFTLKISRVEAEDLGVYYCFQGSHAPYTFGGGTKLEIK

SEQ ID NO: 163 - protein, 114 aa, synthetic construct
QVQLQQSGAELVKPGASVKLSCKASGYTFTSYYMYWVKQRPGQGLEWIGEINPSNGGTNFNEKFKSKATLTVDKSSGTAYMQLSSLTSEDSAIYYCTRGGYGYWGQGTTLTVSS

SEQ ID NO: 164 - protein, 112 aa, synthetic construct
DIVITQDELSNPVISGESVSISCRSSKSLLYKDGKTYLNWFLQRPGQSPQLLIYLMSTRASGVSDRFSGSGSGTDFTLEISRVKAEDVGVYYCQQVVEYPRTFGSGTKLEIK

SEQ ID NO: 165 - protein, 120 aa, synthetic construct
QVQLQESGPGLVKPSETLSLTCAVSGYSISSGYYWGWIRQPPGKGLEWIGSIYHSGSTYYNPSLKSRVTISVDTSKNQFSLKLSSVTAADTAVYYCARQRYWSKSYFRPWGQGTLVTVSS

SEQ ID NO: 166 - protein, 107 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSDSYPLTFGGGTKVEIK

SEQ ID NO: 167 - protein, 125 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSS

SEQ ID NO: 168 - protein, 109 aa, synthetic construct
QTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 169 - protein, 125 aa, synthetic construct
EVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSS

SEQ ID NO: 170 - protein, 109 aa, synthetic construct
QAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVL

SEQ ID NO: 171 - protein, 118 aa, synthetic construct
DIKLQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSS

SEQ ID NO: 172 - protein, 106 aa, synthetic construct
DIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 173 - protein, 119 aa, synthetic construct
EVQLVQSGAEVKKPGASVKVSCKASGFTFTSYYIHWVRQAPGQGLEWIGWIYPENDNTKYNEKFKDRVTITADTSTSTAYLELSSLRSEDTAVYYCARDGYSRYYFDYWGQGTLVTVSS

SEQ ID NO: 174 - protein, 112 aa, synthetic construct
DIVMTQSPDSLAVSLGERATINCKSSQSLLNSRTRKNYLAWYQQKPGQSPKLLIYWTSTRKSGVPDRFSGSGSGTDFTLTISSLQAEDVAVYYCKQSFILRTFGQGTKVEIK

SEQ ID NO: 175 - protein, 121 aa, synthetic construct
QVQLVQSGAEVKKPGASVKVSCKASGYTFTEFGMNWVRQAPGQGLEWMGWINTKTGEATYVEEFKGRVTFTTDTSTSTAYMELRSLRSDDTAVYYCARWDFAYYVEAMDYWGQGTTVTVSS

SEQ ID NO: 176 - protein, 109 aa, synthetic construct; identical to SEQ ID NO: 170

SEQ ID NO: 177 - protein, 120 aa, synthetic construct
QVQLQQSGAELARPGASVKMSCKASGYTFTSYTMHWVKQRPGQGLEWIGYINPSSGYTNYIQRFKDKATLTADKSSSTAYMQVSSLTSEDSAVYYCARGSRYDYYGMDYWGQGTSVTVSS

SEQ ID NO: 178 - protein, 106 aa, synthetic construct
QIVLTQSPAIMSASPGEKVTMTCSASSSVSYMHWYQQKSGTSPKRWIYDTSKLASGVPARFSGSGSGTSYSLTISSMEAEDAATYYCQQWSSNPLTFGAGTKLELK

SEQ ID NO: 179 - protein, 125 aa, synthetic construct
EVQLVESGGGLVQPGGSLRLSCAASGFTFSTYAMNWVRQAPGKGLEWVGRIRSKYNNYATYYADSVKGRFTISRDDSKNSLYLQMNSLKTEDTAVYYCVRHGNFGNSYVSWFAYWGQGTLVTVSS

SEQ ID NO: 180 - protein, 109 aa, synthetic construct
QAVVTQEPSLTVSPGGTVTLTCRSSTGAVTTSNYANWVQQKPGQAPRGLIGGTNKRAPWTPARFSGSLLGGKAALTITGAQAEDEADYYCALWYSNLWVFGGGTKLTVL

SEQ ID NO: 181 - protein, 121 aa, synthetic construct
EVQLVESGGGLVQPGGSLRLSCAASGFTFSDYYMTWVRQAPGKGLEWVAFIRNRARGYTSDHNPSVKGRFTISRDNAKNSLYLQMNSLRAEDTAVYYCARDRPSYYVLDYWGQGTTVTVSS

SEQ ID NO: 182 - protein, 112 aa, synthetic construct
DIVMTQSPDSLAVSLGERATINCKSSQSLFNVRSRKNYLAWYQQKPGQPPKLLISWASTRESGVPDRFSGSGSGTDFTLTISSLQAEDVAVYYCKQSYDLFTFGSGTKLEIK

SEQ ID NO: 183 - protein, 125 aa, synthetic construct; identical to SEQ ID NO: 167

SEQ ID NO: 184 - protein, 109 aa, synthetic construct; identical to SEQ ID NO: 168

SEQ ID NO: 185 - protein, 125 aa, synthetic construct; identical to SEQ ID NO: 167

SEQ ID NO: 186 - protein, 109 aa, synthetic construct; identical to SEQ ID NO: 168

SEQ ID NO: 187 - protein, 120 aa, synthetic construct
EVQLVESGGGLVQPGGSLRLSCAASGFTISTNAMSWVRQAPGKGLEWIGVITGRDITYYASWAKGRFTISRDNSKNTLYLQMNSLRAEDTAVYYCARDGGSSAITSNNIWGQGTLVTVSS

SEQ ID NO: 188 - protein, 112 aa, synthetic construct
DVVMTQSPSTLSASVGDRVTINCQASESISSWLAWYQQKPGKAPKLLIYEASKLASGVPSRFSGSGSGTEFTLTISSLQPDDFATYYCQGYFYFISRTYVNSFGGGTKVEIK

SEQ ID NO: 189 - protein, 125 aa, synthetic construct; identical to SEQ ID NO: 167

SEQ ID NO: 190 - protein, 109 aa, synthetic construct; identical to SEQ ID NO: 168

SEQ ID NO: 191 - protein, 125 aa, synthetic construct
EVKLVESGGGLVQPGGSLRLSCAASGFTFNTYAMNWVRQAPGKGLEWVARIRSKYNNYATYYADSVKDRFTISRDDSKSSLYLQMNNLKTEDTAMYYCVRHGNFGNSYVSWFAYWGQGTLVTVSS

SEQ ID NO: 192 - protein, 110 aa, synthetic construct
QAVVTQEPSFSVSPGGTVTLTCRSSTGAVTTSNYANWVQQTPGQAFRGLIGGTNKRAPGVPARFSGSLIGDKAALTITGAGQADDESIYFCALWYSNLWVFGGGTKLTVL

SEQ ID NO: 193 - protein, 125 aa, synthetic construct; identical to SEQ ID NO: 167

SEQ ID NO: 194 - protein, 109 aa, synthetic construct; identical to SEQ ID NO: 168

SEQ ID NO: 195 - protein, 125 aa, synthetic construct
EVQLVESGGGLVQPGGSLRLSCAASGFTFSTYAMNWVRQAPGKGLEWVGRIRSKYNNYATYYADSVKDRFTISRDDSKNSLYLQMNSLKTEDTAVYYCVRHGNFGNSYVSWFAYWGQGTLVTVSS

SEQ ID NO: 196 - protein, 109 aa, synthetic construct; identical to SEQ ID NO: 180

SEQ ID NO: 197 - protein, 118 aa, synthetic construct [sequence omitted]

SEQ ID NO: 198 - protein, 108 aa, synthetic construct [sequence omitted]

SEQ ID NO: 199 - protein, 125 aa, synthetic construct; identical to SEQ ID NO: 169

SEQ ID NO: 200 - protein, 111 aa, synthetic construct
QAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVLSS

SEQ ID NO: 201 - protein, 125 aa, synthetic construct; identical to SEQ ID NO: 167

SEQ ID NO: 202 - protein, 109 aa, synthetic construct; identical to SEQ ID NO: 168

SEQ ID NO: 203 - protein, 120 aa, synthetic construct [sequence omitted]

SEQ ID NO: 204 - protein, 106 aa, synthetic construct [sequence omitted]

SEQ ID NO: 205 - protein, 124 aa, synthetic construct
EVQLVESGGGLVQPGRSLRLSCAASGFTFDDYSMHWVRQAPGKGLEWVSGISWNSGSKGYADSVKGRFTISRDNAKNSLYLQMNSLRAEDTALYYCAKYGSGYGKFYHYGLDVWGQGTTVTVSS

SEQ ID NO: 206 - protein, 108 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIK

SEQ ID NO: 207 - protein, 119 aa, synthetic construct
EVQLVQSGAEVKKPGASVKVSCKASGYTFTNYYIHWVRQAPGQGLEWIGWIYPGDGNTKYNEKFKGRATLTADTSTSTAYLELSSLRSEDTAVYYCARDSYSNYYFDYWGQGTLVTVSS

SEQ ID NO: 208 - protein, 112 aa, synthetic construct
DIVMTQSPDSLAVSLGERATINCKSSQSLLNSRTRKNYLAWYQQKPGQPPKLLIYWASTRESGVPDRFSGSGSGTDFTLTISSLQAEDVAVYYCTQSFILRTFGQGTKVEIK

SEQ ID NO: 209 - protein, 119 aa, synthetic construct [sequence omitted]

SEQ ID NO: 210 - protein, 106 aa, synthetic construct [sequence omitted]

SEQ ID NO: 211 - protein, 119 aa, synthetic construct
QVQLVQSGGGVVQPGRSLRLSCKASGYTFTRYTMHWVRQAPGKCLEWIGYINPSRGYTNYNQKFKDRFTISRDNSKNTAFLQMDSLRPEDTGVYFCARYYDDHYSLDYWGQGTPVTVSS

SEQ ID NO: 212 - protein, 106 aa, synthetic construct
DIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGCGTKLQIT

SEQ ID NO: 213 - protein, 125 aa, synthetic construct; identical to SEQ ID NO: 195

SEQ ID NO: 214 - protein, 109 aa, synthetic construct; identical to SEQ ID NO: 180

SEQ ID NO: 215 - protein, 123 aa, synthetic construct
EVQLVESGGGLVQPGRSLRLSCAASGFTFDDYTMHWVRQAPGKGLEWVSGISWNSGSIGYADSVKGRFTISRDNAKKSLYLQMNSLRAEDTALYYCAKDNSGYGHYYYGMDVWGQGTTVTVAS

SEQ ID NO: 216 - protein, 107 aa, synthetic construct
EIVMTQSPATLSVSPGERATLSCRASQSVSSNLAWYQQKPGQAPRLLIYGASTRATGIPARFSGSGSGTEFTLTISSLQSEDFAVYYCQHYINWPLTFGGGTKVEIK

SEQ ID NO: 217 - protein, 119 aa, synthetic construct [sequence omitted]

SEQ ID NO: 218 - protein, 110 aa, synthetic construct [sequence omitted]

SEQ ID NO: 219 - protein, 125 aa, synthetic construct; identical to SEQ ID NO: 167

SEQ ID NO: 220 - protein, 109 aa, synthetic construct; identical to SEQ ID NO: 168

SEQ ID NO: 221 - protein, 125 aa, synthetic construct; identical to SEQ ID NO: 167

SEQ ID NO: 222 - protein, 109 aa, synthetic construct; identical to SEQ ID NO: 168

SEQ ID NO: 223 - protein, 125 aa, synthetic construct; identical to SEQ ID NO: 167

SEQ ID NO: 224 - protein, 109 aa, synthetic construct; identical to SEQ ID NO: 168

SEQ ID NO: 225 - protein, 125 aa, synthetic construct
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSS

SEQ ID NO: 226 - protein, 112 aa, synthetic construct
QAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLEPK

SEQ ID NO: 227 - protein, 119 aa, synthetic construct; identical to SEQ ID NO: 207

SEQ ID NO: 228 - protein, 107 aa, synthetic construct [sequence omitted]

SEQ ID NO: 229 - protein, 119 aa, synthetic construct
DVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDHYCLDYWGQGTTVTVSS

SEQ ID NO: 230 - protein, 106 aa, synthetic construct
DIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIK

SEQ ID NO: 231 - protein, 125 aa, synthetic construct
EVQLVESGGGLVQPGGSLRLSCAASGFTFNTYAMNWVRQAPGKGLEWVARIRSKYNNYATYYAASVKGRFTISRDDSKNSLYLQMNSLKTEDTAVYYCARHGNFGNSYVSWFAYWGQGTLVTVSS

SEQ ID NO: 232 - protein, 109 aa, synthetic construct
QTVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVL

SEQ ID NO: 233 - protein, 125 aa, synthetic construct; identical to SEQ ID NO: 167

SEQ ID NO: 234 - protein, 109 aa, synthetic construct; identical to SEQ ID NO: 168

SEQ ID NO: 235 - protein, 122 aa, synthetic construct [sequence omitted]

SEQ ID NO: 236 - protein, 107 aa, synthetic construct
AIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIK

SEQ ID NO: 237 - protein, 125 aa, synthetic construct; identical to SEQ ID NO: 231

SEQ ID NO: 238 - protein, 109 aa, synthetic construct; identical to SEQ ID NO: 232

SEQ ID NO: 239 - protein, 119 aa, synthetic construct [sequence omitted]

SEQ ID NO: 240 - protein, 106 aa, synthetic construct [sequence omitted]

SEQ ID NO: 241 - protein, 118 aa, synthetic construct
QVQLVQSGGGVVQPGRSLRLSCVASGFTFSSYGMHWVRQAPGKGLEWVAAIWYNARKQDYADSVKGRFTISRDNSKNTLYLQMNSLRAEDTAVYYCTRGTGYNWFDPWGQGTLVTVSS

SEQ ID NO: 242 - protein, 107 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIK

SEQ ID NO: 243 - protein, 125 aa, synthetic construct; identical to SEQ ID NO: 225

SEQ ID NO: 244 - protein, 109 aa, synthetic construct
QAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVL

SEQ ID NO: 245 - protein, 124 aa, synthetic construct; identical to SEQ ID NO: 205

SEQ ID NO: 246 - protein, 108 aa, synthetic construct
DIQMTQSPSSLSASVGDRVTITCRASQSISTYLNWYQQKPGKAPKLLIYTASSLQSGVPSRFSGSGSGTDFTLTISSLQPEDFATYYCQQSYSTPPITFGQGTRLEIK

SEQ ID NO: 247 - protein, 125 aa, synthetic construct; identical to SEQ ID NO: 167

SEQ ID NO: 248 - protein, 109 aa, synthetic construct; identical to SEQ ID NO: 168

SEQ ID NO: 249 - protein, 125 aa, synthetic construct; identical to SEQ ID NO: 225

SEQ ID NO: 250 - protein, 112 aa, synthetic construct; identical to SEQ ID NO: 226

SEQ ID NO: 251 - protein, 120 aa, synthetic construct [sequence omitted]

SEQ ID NO: 252 - protein, 106 aa, synthetic construct [sequence omitted]

SEQ ID NO: 253 - protein, 122 aa, synthetic construct
QVQLVQSGAEVKKPGASVKVSCKASGYTFTSYDINWVRQAPGQGLEWMGWMNPNSGNTGFAQKFQGRVTMTRDTSTSTVYMELSSLRSEDTAVYYCARDRANTDYSLGMDVWGQGTLVTVSS

SEQ ID NO: 254 - protein, 110 aa, synthetic construct
DIQMTQSPSSLSASVGDRVTITCRSSTGAVTTSNYANWVQQKPGKAPKALIGGTNKRAPGVPSRFSGSLIGDKATLTISSLQPEDFATYYCALWYSNLWVFGQGTKVEIK

SEQ ID NO: 255 - protein, 125 aa, synthetic construct; identical to SEQ ID NO: 231

SEQ ID NO: 256 - protein, 109 aa, synthetic construct; identical to SEQ ID NO: 232

SEQ ID NO: 257 - protein, 122 aa, synthetic construct [sequence omitted]

SEQ ID NO: 258 - protein, 107 aa, synthetic construct; identical to SEQ ID NO: 236

SEQ ID NO: 259 - protein, 122 aa, synthetic construct [sequence omitted]

SEQ ID NO: 260 - protein, 107 aa, synthetic construct [sequence omitted]

SEQ ID NO: 261 - protein, 120 aa, synthetic construct
QVQLQQSGPRLVRPSQTLSLTCAISGDSVFNNNAAWSWIRQSPSRGLEWLGRTYYRSKWLYDYAVSVKSRITVNPDTSRNQFTLQLNSVTPEDTALYYCARGYSSSFDYWGQGTLVTVSS

SEQ ID NO: 262 - protein, 110 aa, synthetic construct
QSALTQPASVSGSPGQSITISCTGTSSNIGTYKFVSWYQQHPDKAPKVLLYEVSKRPSGVSSRFSGSKSGNTASLTISGLQAEDQADYHCVSYAGSGTLLFGGGTKLTVL

SEQ ID NO: 263 - protein, 120 aa, synthetic construct; identical to SEQ ID NO: 187

SEQ ID NO: 264 - protein, 112 aa, synthetic construct; identical to SEQ ID NO: 188

SEQ ID NO: 265 - protein, 123 aa, synthetic construct
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKDSRGYGDYRLGGAYWGQGTLVTVSS

SEQ ID NO: 266 - protein, 107 aa, synthetic construct
EIVMTQSPATLSVSPGERATLSCRASQSVSSNLAWYQQKPGQAPRLLIYGASTRATGIPARFSGSGSGTEFTLTISSLQSEDFAVYYCQQYNNWPWTFGQGTKVEIK

SEQ ID NO: 267 - protein, 125 aa, synthetic construct
EVQLLESGGGLVQPGGSLRLSCAASGFQFSSYAMNWVRQAPGKGLEWVSRIRSKYNNYATYYADSVKGRFTISRDDSKNTLYLQMNSLRAEDTAVYYCVRHTTFPSSYVSYYGYWGQGTLVTVSS

SEQ ID NO: 268 - protein, 109 aa, synthetic construct; identical to SEQ ID NO: 170

SEQ ID NO: 269 - protein, 125 aa, synthetic construct
EVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSS

SEQ ID NO: 270 - protein, 109 aa, synthetic construct
QTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVL

SEQ ID NO: 271 - protein, 119 aa, synthetic construct
EVQLVQSGAEVKKPGASVKVSCKASGFTFTSYYIHWVRKAPGQGLEWIGWIYPENDNTKYNEKFKDRVTITADTSTSTAYLELSSLRSEDTAVYYCARDGYSRYYFDYWGQGTLVTVSS

SEQ ID NO: 272 - protein, 112 aa, synthetic construct
DIVMTQSPDSLAVSLGERATINCKSSQSLLNSRTRKNYLAWYQEKPGQPPKLLIYWASTRESGVPDRFSGSGSGTDFTLTISSLQAEDVAVYYCTQSFILRTFGQGTKVEIK

SEQ ID NO: 273 - protein, 121 aa, synthetic construct
QVQLVQSGPEVKKPGSSVKVSCKASGYTFSRSTMHWVRQAPGQGLEWIGYINPSSAYTNYNQKFKDRVTITADKSTSTAYMELSSLRSEDTAVYYCARPQVHYDYNGFPYWGQGTLVTVSS

SEQ ID NO: 274 - protein, 106 aa, synthetic construct
DIQMTQSPSTLSASVGDRVTMTCSASSSVSYMNWYQQKPGKAPKRWIYDSSKLASGVPSRFSGSGSGTDYTLTISSLQPDDFATYYCQQWSRNPPTFGGGTKVEIK

SEQ ID NO: 275 - protein, 120 aa, synthetic construct; identical to SEQ ID NO: 187

SEQ ID NO: 276 - protein, 112 aa, synthetic construct; identical to SEQ ID NO: 188

SEQ ID NO: 277 - protein, 120 aa, synthetic construct; identical to SEQ ID NO: 261

SEQ ID NO: 278 - protein, 110 aa, synthetic construct; identical to SEQ ID NO: 262

SEQ ID NO: 279 - protein, 125 aa, synthetic construct
EVQLVESGGGLVQPGGSLKLSCAASGFTFNTYAMNWVRQAPGKGLEWVARIRSKYNNYATYYADSVKDRFTISRDDSKNTAYLQMNNLKTEDTAVYYCVRHGNFGNSYVSWFAYWGQGTLVTVSA

SEQ ID NO: 280 - protein, 109 aa, synthetic construct
EAVVTQEPSLTVSPGGTVTLTCRSSTGAVTTSNYANWVQEKPGQLPRGLIGGTNKRAPWVPARFSGSLLGGKAALTLSGAQPEDEAEYFCALWYSNLWVFGGGTKLTVL

SEQ ID NO: 281 - protein, 125 aa, synthetic construct; identical to SEQ ID NO: 269

SEQ ID NO: 282 - protein, 109 aa, synthetic construct; identical to SEQ ID NO: 270

SEQ ID NO: 283 - protein, 123 aa, synthetic construct
EVQLVESGGGLVQPGRSLRLSCAASGFTFHNYAMHWVRQAPGKGLEWVSGISWNSGSIGYADSVKGRFTISRDNAKNSLYLQMNSLRAEDTALYYCAKDSRGYGDYSLGGAYWGQGTLVTVSS

SEQ ID NO: 284 - protein, 107 aa, synthetic construct; identical to SEQ ID NO: 266

SEQ ID NO: 285 - protein, 116 aa, synthetic construct
DVQLQESGPSLVKPSQSLSLTCTVTGYSITSDFAWNWIRQFPGNKLEWMGYISYSGNTRYNPSLKSRISITRDTSKNQFFLQLNSVTIEDTATYYCVTAGRGFPYWGQGTLVTVSA

SEQ ID NO: 286 - protein, 107 aa, synthetic construct
DILMTQSPSSMSVSLGDTVSITCHSSQDINSNIGWLQQRPGKSFKGLIYHGTNLDDEVPSRFSGSGSGADYSLTISSLESEDFADYYCVQYAQFPWTFGGGTKLEIK

SEQ ID NO: 287 - protein, 124 aa, synthetic construct
EVQLVESGGGLVQPGRSLRLSCAASGFTFDDYSMHWVRQAPGKGLEWVSGISWNSGSIGYADSVKGRFTISRDNAKNSLYLQMNSLRAEDTALYYCAKYGSGYGKFYYYGMDVWGQGTTVTVSS

SEQ ID NO: 288 - protein, 108 aa, synthetic construct; identical to SEQ ID NO: 206

SEQ ID NO: 289 - protein, 125 aa, synthetic construct; identical to SEQ ID NO: 225

SEQ ID NO: 290 - protein, 109 aa, synthetic construct; identical to SEQ ID NO: 244

SEQ ID NO: 291 - protein, 125 aa, synthetic construct
EVKLVESGGGLVQPGGSLRLSCAASGFTFNTYAMNWVRQAPGKGLEWVARIRSKYNNYATYYADSVKDRFTISRDDSKSSLYLQMNNLKTEDTAMYYCVRGGNFGNSYVSWFAYWGQGTLVTVSS

SEQ ID NO: 292 - protein, 109 aa, synthetic construct
QAVVTQEPSFSVSPGGTVTLTCRSSTGAVTTSNYANWVQQTPGQAFRGLIGGTNKRAPGVPARFSGSLIGDKAALTITGAQADDESIYFCALWYSNLWVFGGGTKLTVL

SEQ ID NO: 293 - protein, 22 aa, synthetic construct
MGSHHHHHHHHGSENLYFQGGS

SEQ ID NO: 294 - protein, 16 aa, synthetic construct
KLLHHILDAQKMVWNH

SEQ ID NO: 295 - protein, 189 aa, Homo sapiens
MTEYKLVVVGADGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQRVEDAFYTLVREIRQYRLKKISKEEKTPGCVKIKKCIIM

SEQ ID NO: 296 - protein, 10 aa, synthetic construct
QLIQNHFVDE

SEQ ID NO: 297 - protein, 7 aa, synthetic construct
SAMRDQY

SEQ ID NO: 298 - protein, 10 aa, synthetic construct
AINNTKSFED

SEQ ID NO: 299 - protein, 10 aa, synthetic construct
KTRQGVDDAF

SEQ ID NO: 300 - protein, 9 aa, synthetic construct
INNTKSFED